(19) United States
(12) Patent Application Publication
(10) Pub. No.: US 2026/0069584 A1
(43) Pub. Date: Mar. 12, 2026

(54) METHODS OF TREATING CHRONIC MYELOID LEUKEMIA USING THE TYROSINE KINASE INHIBITOR VODOBATINIB

(71) Applicant: Sun Pharma Advanced Research Company Ltd., Mumbai (IN)

(72) Inventors: Geetanjali Chandrasekhar CHIMOTE, Thane, Maharashtra (IN); Ravi Sankar Prasad TALLURI, Navi Mumbai, Maharashtra (IN); Vikram RAMANATHAN, Vadodara, Gujarat (IN)

(21) Appl. No.: 19/106,200

(22) PCT Filed: Aug. 24, 2023
(86) PCT No.: PCT/IB2023/058409

§ 371 Date: Feb. 24, 2025

(30) Foreign Application Priority Data
Aug. 25, 2022 (IN) 202221048373
Aug. 25, 2022 (IN) 202221048415

(51) Int. Cl. A61K 31/47; A61P 35/02

(52) U.S. Cl. CPC A61K 31/47; A61P 35/02

(57) ABSTRACT

The present invention relates to methods of treating leukemia using Tyrosine Kinase inhibitors. The invention particularly relates to methods of treating CML and ALL using a compound of Formula I or a pharmaceutically acceptable salt thereof. The compound of Formula 1 has been shown to be efficacious safe and tolerable at a dose from 10 mg to 210 mg.

The present application claims the benefit of Indian Provisional Application No. 202221048373 and Indian Provisional Application No. 202221048415, filed on 25 Aug. 2022, the entire contents of which are hereby incorporated by reference.

### FIELD OF INVENTION

The present invention relates to methods of treating leukemia. In one aspect, the present invention relates to methods of treating chronic myeloid leukemia (CML) using a compound of Formula I or a pharmaceutically acceptable salt thereof, as shown below.

### BACKGROUND OF THE INVENTION

Chronic myeloid leukemia (CML) is a clonal myeloproliferative disorder representing ˜15-20% of adult leukemias [Apperley 2015; Deininger et al. 2003]. The underlying cause of CML is the breakpoint cluster region-Abelson leukemia (BCR-ABL1) fusion oncoprotein, which results from a reciprocal t (9;22) chromosomal translocation in hematopoietic stem cells. This translocation leads to the fusion of the breakpoint cluster region (BCR) coding sequence with the tyrosine kinase coding region of Abelson leukemia (ABL1) resulting in constitutive activation of ABL1 kinase activity. This translocation thus results in the activation of multiple downstream pathways that contribute to the growth and survival of leukemic cells [Hazlehurst et al. 2009]. CML is characterized and classified by phases: chronic phase (CP), accelerated phase (AP), or blast phase (BP). Patients in CP typically have less than 10% blasts in their blood or bone marrow samples. These patients usually have fairly mild symptoms. Most of the CML patients are diagnosed in the chronic phase. In AP, the patient's blood samples have 15% or more, but less than 30% blasts. In this phase, 20% of the blood cells consist of basophil, the platelet counts are low (100×1,000/mm³ or less) that are not caused by the treatment and there are chromosome changes in the leukemia cells with the Philadelphia chromosome. In the blast phase, bone marrow and/or blood samples have 20% or more blasts. Large clusters of blasts are seen in the bone marrow. The blast cells spread to tissues and organs beyond the bone marrow.

Philadelphia chromosome-positive acute lymphoblastic leukemia (Ph+ ALL) is a rare and aggressive form of ALL that is characterized by the presence of the BCR-ABL1 fusion. It accounts for 25% of the total patients with ALL (Nicholas J. Short, 2020).

Dasatinib is approved for the treatment of adults with Ph+ CML in chronic phase, and adults with Ph+ acute lymphocytic leukemia (ALL) with resistance or intolerance to prior therapy. Nilotinib is approved for the treatment of Ph+ CML in the chronic phase in adult and pediatric patients at least 1 year of age. Bosutinib is approved for the treatment of chronic phase Ph+ CML. Second-generation tyrosine kinase inhibitors (TKIs) such as dasatinib, nilotinib, and bosutinib generally offer improved patient tolerance over the first-generation TKI, imatinib (Ferdinand, 2012). However, about 10% of patients do not tolerate their initial treatments with TKIs and many subjects develop long-term treatment-emergent adverse events (TEAEs) such as cardiovascular, pulmonary, gastrointestinal, and endocrine toxicities, and secondary malignancies (Caldemeyer, 2016). Furthermore, point mutations arising in the BCR-ABL1 kinase domain impair TKI binding and lead to the development of TKI resistance (Deininger, 2015).

There is a need for improved TKIs, especially for patients who failed treatment with prior TKIs.

### DOCUMENTS

Siegel, R. L., Miller, K. D. and Jemal, A. (2019), Cancer statistics, 2019. CA A Cancer J Clin, 69:7-34. https://doi.org/10.3322/caac.21551
Hazlehurst L, Bewry N, Nair R, Pinilla-Ibarz J. Signaling networks associated with BCR-ABL1-dependent transformation. Cancer control 2009; 16 (2): 100-107.
Ferdinand R, Mitchell S A, Batson S, Tumur I. Treatments for chronic myeloid leukemia: a qualitative systematic review. J Blood Med. 2012; 3:51-76.
Caldemeyer L, Dugan M, Edwards J, Akard L. Long-Term Side Effects of Tyrosine Kinase Inhibitors in Chronic Myeloid Leukemia. Curr Hematol Malig Rep. 2016 April; 11 (2): 71-9.
Deininger M W. Diagnosing and Managing Advanced Chronic Myeloid Leukemia. American Society of Clinical Oncology Educational Book 2015:35, e381-e388.
Buoen C, Bjerrum O J, Thomsen M S. How First-time-in-human Studies are Being Performed: A survey of Phase I dose-escalation trials in healthy volunteers published between 1995 and 2004. J Clin Pharmacol. 2005 October; 45 (10): 1123-1136.
US Food and Drug Administration (FDA) Guidance for Industry (2005), Estimating the Maximum Safe Starting Dose in Initial Clinical Trials for Therapeutics in Adult Healthy Subjects.
Di Gion, P., Kanefendt, F., Lindauer, A. et al. Clinical Pharmacokinetics of Tyrosine Kinase Inhibitors. Clin Pharmacokinet. 2011; 50:551-603.
Nicholas J. Short, Poor Ph+ ALL outcomes require a re-examination of treatment standard, Hematology/Oncology News Aug. 17, 2020.

### BRIEF SUMMARY OF THE INVENTION

The use of a tyrosine kinase inhibitor (TKI) that targets BCR-ABL1 is a well-established and highly effective strategy for sustained disease control in CML, Ph+ CML and Ph+ ALL. A compound of Formula I is a novel BCR-ABL1 TKI under clinical development for the treatment of both refractory/intolerant chronic myeloid leukemia and newly diagnosed CML patients.

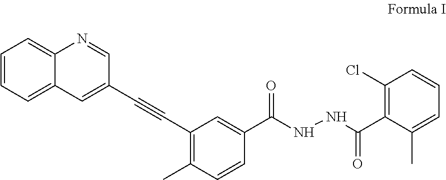 [SMILES: Cc1ccc(C(=O)NNC(=O)c2c(C)cccc2Cl)cc1C#Cc1cnc2ccccc2c1]

The compound of Formula I has been studied in in-vitro and in-vivo studies and has specific and highly potent activity on wild-type BCR-ABL1 and several BCR-ABL1 mutations. The present disclosure relates to a method for the treatment of adult subjects with chronic phase (CP), accelerated phase (AP), or blast phase (BP) CML or Ph+ CML or Ph+ ALL.

Accordingly, disclosed herein is a method for the treatment of a patient (such as an adult patient) with chronic myeloid leukemia (CML), comprising orally administering a therapeutically effective amount of a compound of Formula I or a pharmaceutically acceptable salt thereof

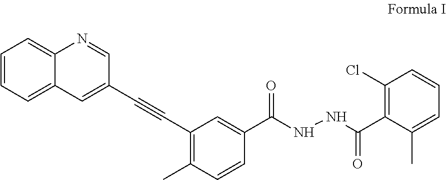 [SMILES: Cc1ccc(C(=O)NNC(=O)c2c(C)cccc2Cl)cc1C#Cc1cnc2ccccc2c1]

at an initial daily dose, and escalating or de-escalating the dose to achieve or maintain a complete hematologic response with no severe adverse reaction.

Accordingly, disclosed herein is a method for the treatment of an adult patient with CML, comprising orally administering a therapeutically effective amount of a compound of Formula I or a pharmaceutically acceptable salt thereof at an initial daily dose, and escalating or de-escalating the dose to achieve or maintain complete hematologic response with no severe adverse reaction, and wherein the daily dose of the compound of Formula I or a pharmaceutically acceptable salt thereof in a patient results in mean AUC₀₋₂₄ ranging from 6226±5827 ng*h/mL to 60373±55659 ng*h/mL.

Accordingly, disclosed herein is a method for the treatment of an adult patient with CML, comprising orally administering a therapeutically effective amount of a compound of Formula I or a pharmaceutically acceptable salt thereof at an initial daily dose, and escalating or de-escalating the dose to achieve or maintain complete hematologic response with no severe adverse reaction, and wherein the daily dose of the compound of Formula I or a pharmaceutically acceptable salt thereof results in a mean Cₘₐₓ ranging from 664±450 ng/ml to 5054±3051 ng/mL.

Accordingly, disclosed herein is a method for the treatment of an adult patient with CML, comprising orally administering a therapeutically effective amount of a compound of Formula I or a pharmaceutically acceptable salt thereof at an initial daily dose, and escalating or de-escalating the dose to achieve or maintain disease response, with no severe adverse reaction.

Accordingly, disclosed herein is a method for the treatment of an adult patient with CML, comprising orally administering a therapeutically effective amount of a compound of Formula I or a pharmaceutically acceptable salt thereof at an initial daily dose, and escalating or de-escalating the dose to achieve or maintain disease response, with no severe adverse reaction, and wherein the daily dose of the compound of Formula I or a pharmaceutically acceptable salt thereof in the patient results in mean AUC₀₋₂₄ ranging from 6226±5827 ng*h/mL to 60373±55659 ng*h/mL.

Accordingly, disclosed herein is a method for the treatment of an adult patient with CML, comprising orally administering a therapeutically effective amount of a compound of Formula I or a pharmaceutically acceptable salt thereof at an initial daily dose, and escalating or de-escalating the dose to achieve or maintain disease response, with no severe adverse reaction, and wherein the daily dose of the compound of Formula I or a pharmaceutically acceptable salt thereof results in a mean Cₘₐₓ ranging from 664±450 ng/ml to 5054±3051 ng/ml.

Accordingly, disclosed herein is a method for the treatment of an adult patient with CML, comprising orally administering a therapeutically effective amount of a compound of Formula I or a pharmaceutically acceptable salt thereof at an initial daily dose of between 10 mg and 200 mg and escalating the dose between 20 mg and 210 mg to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

Accordingly, disclosed herein is a method for the treatment of an adult patient with CML, comprising orally administering a therapeutically effective amount of a compound of Formula I or a pharmaceutically acceptable salt thereof at an initial daily dose of between 10 mg and 200 mg and escalating the dose between 20 mg and 210 mg to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof in the patient results in mean AUC₀₋₂₄ ranging from 6226±5827 ng*h/mL to 60373±55659 ng*h/mL.

Accordingly, disclosed herein is a method for the treatment of an adult patient with CML, comprising orally administering a therapeutically effective amount of a compound of Formula I or a pharmaceutically acceptable salt thereof at an initial daily dose of between 10 mg and 200 mg and escalating the dose between 20 mg and 210 mg to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof results in a mean Cₘₐₓ ranging from 664±450 ng/ml to 5054±3051 ng/mL.

Accordingly, disclosed herein is a method for the treatment of an adult patient with CML, comprising orally administering a therapeutically effective amount of a compound of Formula I or a pharmaceutically acceptable salt thereof at an initial daily dose of between 20 mg and 210 mg and de-escalating to the dose between 10 mg and 200 mg on the occurrence of severe adverse reaction, wherein the dose is de-escalated such that with the de-escalated dose patient achieves or maintains at least one of (a) complete hematological response, (b) complete hematological response and partial cytogenetic response, (c) complete hematological response and complete cytogenetic response, or (d) complete hematological response, complete cytogenetic response, and major molecular response.

Accordingly, disclosed herein is a method for the treatment of an adult patient with CML, comprising orally administering a therapeutically effective amount of a compound of Formula I or a pharmaceutically acceptable salt thereof at an initial daily dose of between 20 mg and 210 mg and de-escalating to the dose between 10 mg and 200 mg on the occurrence of severe adverse reaction, wherein the dose is de-escalated such that with the de-escalated dose patient achieves or maintains at least one of (a) complete hematological response, (b) complete hematological response and partial cytogenetic response, (c) complete hematological response and complete cytogenetic response, or (d) complete hematological response, complete cytogenetic response, and major molecular response, and wherein the daily dose of the compound of Formula I or a pharmaceutically acceptable salt thereof in the patient results in mean AUC₀₋₂₄ ranging from 6226±5827 ng*h/mL to 60373±55659 ng*h/mL.

Accordingly, disclosed herein is a method for the treatment of an adult patient with CML, comprising orally administering a therapeutically effective amount of a compound of Formula I or a pharmaceutically acceptable salt thereof at an initial daily dose of between 20 mg and 210 mg and de-escalating to the dose between 10 mg and 200 mg on the occurrence of severe adverse reaction, wherein the dose is de-escalated such that with the de-escalated dose patient achieves or maintains at least one of (a) complete hematological response, (b) complete hematological response and partial cytogenetic response, (c) complete hematological response and complete cytogenetic response, or (d) complete hematological response, complete cytogenetic response, and major molecular response, and wherein the daily dose of the compound of Formula I or a pharmaceutically acceptable salt thereof results in a mean Cₘₐₓ ranging from 664±450 ng/ml to 5054±3051 ng/ml.

Accordingly, disclosed herein is a method for the treatment of an adult patient with refractory CML comprising, administering a therapeutically effective amount of compound of Formula I or a pharmaceutically acceptable salt thereof at an initial daily dose of 174 mg, wherein the initial dose is escalated to 204 mg to achieve or maintain complete hematologic response (CHR) with no severe adverse reaction.

Accordingly, disclosed herein is a method for the treatment of an adult patient with refractory CML comprising, administering a therapeutically effective amount of compound of Formula I or a pharmaceutically acceptable salt thereof at an initial daily dose of 174 mg, wherein the initial dose is escalated to 204 mg to achieve or maintain complete hematologic response (CHR) with no severe adverse reaction, and wherein the daily dose of the compound of Formula I or a pharmaceutically acceptable salt thereof in the patient results in mean AUC₀₋₂₄ ranging from 6226±5827 ng*h/mL to 60373±55659 ng*h/mL.

Accordingly, disclosed herein is a method for the treatment of an adult patient with refractory CML comprising, administering a therapeutically effective amount of compound of Formula I or a pharmaceutically acceptable salt thereof at an initial daily dose of 174 mg, wherein the initial dose is escalated to 204 mg to achieve or maintain complete hematologic response (CHR) with no severe adverse reaction, and wherein the daily dose of the compound of Formula I or a pharmaceutically acceptable salt thereof results in a mean Cₘₐₓ ranging from 664±450 ng/ml to 5054±3051 ng/mL.

Accordingly, disclosed herein is a method for the treatment of an adult patient with refractory CML, comprising orally administering a therapeutically effective amount of a compound of Formula I or a pharmaceutically acceptable salt thereof at an initial daily dose of 174 mg, wherein the initial dose is escalated to 204 mg to achieve or maintain at least one of (a) complete hematological response, (b) complete hematological response and partial cytogenetic response, (c) complete hematological response and complete cytogenetic response, or (d) complete hematological response, complete cytogenetic response, and major molecular response; with no severe adverse reaction.

Accordingly, disclosed herein is a method for the treatment of an adult patient with refractory CML, comprising orally administering a therapeutically effective amount of a compound of Formula I or a pharmaceutically acceptable salt thereof at an initial daily dose of 174 mg, wherein the initial dose is escalated to 204 mg to achieve or maintain at least one of (a) complete hematological response, (b) complete hematological response and partial cytogenetic response, (c) complete hematological response and complete cytogenetic response, or (d) complete hematological response, complete cytogenetic response, and major molecular response; with no severe adverse reaction, and wherein the daily dose of the compound of Formula I or a pharmaceutically acceptable salt thereof in the patient results in mean AUC₀₋₂₄ ranging from 6226±5827 ng*h/mL to 60373±55659 ng*h/mL.

Accordingly, disclosed herein is a method for the treatment of an adult patient with refractory CML, comprising orally administering a therapeutically effective amount of a compound of Formula I or a pharmaceutically acceptable salt thereof at an initial daily dose of 174 mg, wherein the initial dose is escalated to 204 mg to achieve or maintain at least one of (a) complete hematological response, (b) complete hematological response and partial cytogenetic response, (c) complete hematological response and complete cytogenetic response, or (d) complete hematological response, complete cytogenetic response, and major molecular response; with no severe adverse reaction, and wherein the daily dose of the compound of Formula I or a pharmaceutically acceptable salt thereof results in a mean Cₘₐₓ ranging from 664±450 ng/ml to 5054±3051 ng/ml.

Accordingly, disclosed herein is a method for the treatment of an adult patient with refractory CML, wherein the method comprises administering to the patient a therapeutically effective amount of an initial daily dose of 174 mg of a compound of Formula I or a pharmaceutically acceptable salt thereof such that when the patient develops non-hematological toxicity:
a) an initial daily dose is withheld for at least 7 days and resumed after recovery of patient from said toxicity at 174 mg; or
b) an initial daily dose is withheld for at least 7 days and resumed at a reduced daily dose of 126 mg on first instance and to 90 mg, 66 mg and 48 mg on subsequent instances of recurrences of said toxicity.

Accordingly, disclosed herein is a method for the treatment of an adult patient with refractory CML, wherein the method comprises administering to the patient a therapeutically effective amount of an initial daily dose of 174 mg of a compound of Formula I or a pharmaceutically acceptable salt thereof such that when the patient develops non-hematological toxicity:
a) an initial daily dose is withheld for at least 7 days and resumed after recovery of patient from said toxicity at 174 mg; or
b) an initial daily dose is withheld for at least 7 days and resumed at a reduced daily dose of 126 mg on first instance and to 90 mg, 66 mg and 48 mg on subsequent instances of recurrences of said toxicity;
and wherein the daily dose of the compound of Formula I or a pharmaceutically acceptable salt thereof in the patient results in mean AUC₀₋₂₄ ranging from 6226±5827 ng*h/mL to 60373±55659 ng*h/mL.

Accordingly, disclosed herein is a method for the treatment of an adult patient with refractory CML, wherein the method comprises administering to the patient a therapeutically effective amount of an initial daily dose of 174 mg of a compound of Formula I or a pharmaceutically acceptable salt thereof such that when the patient develops non-hematological toxicity:
a) an initial daily dose is withheld for at least 7 days and resumed after recovery of patient from said toxicity at 174 mg; or
b) an initial daily dose is withheld for at least 7 days and resumed at a reduced daily dose of 126 mg on first instance and to 90 mg, 66 mg and 48 mg on subsequent instances of recurrences of said toxicity;
and wherein the daily dose of the compound of Formula I or a pharmaceutically acceptable salt thereof results in a mean Cₘₐₓ ranging from 664±450 ng/ml to 5054±3051 ng/mL.

Accordingly, disclosed herein is a method for the treatment of an adult patient with refractory CML, wherein the method comprises administering to said patient a therapeutically effective amount of an initial daily dose of 174 mg of a compound of Formula I or a pharmaceutically acceptable salt thereof such that when the patient develops non-hematologic toxicity:
a) the initial daily dose is withheld for at least 7 days and resumed after recovery of patient from said toxicity; or
b) the initial daily dose is withheld for at least 7 days and resumed at a reduced daily dose of 126 mg on first instance and to 90 mg, 66 mg and 48 mg on subsequent instances of recurrences of said toxicity;
and wherein, the reduced dose is optionally re-escalated to any one of the dose selected from 66 mg, 90 mg, 126 mg and 174 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

Accordingly, disclosed herein is a method for the treatment of an adult patient with refractory CML, wherein the method comprises administering to said patient a therapeutically effective amount of an initial daily dose of 174 mg of a compound of Formula I or a pharmaceutically acceptable salt thereof such that when the patient develops non-hematologic toxicity:
a) the initial daily dose is withheld for at least 7 days and resumed after recovery of patient from said toxicity; or
b) the initial daily dose is withheld for at least 7 days and resumed at a reduced daily dose of 126 mg on first instance and to 90 mg, 66 mg and 48 mg on subsequent instances of recurrences of said toxicity;
and wherein, the reduced dose is optionally re-escalated to any one of the dose selected from 66 mg, 90 mg, 126 mg and 174 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof in the patient results in mean AUC₀₋₂₄ ranging from 6226±5827 ng*h/mL to 60373±55659 ng*h/mL.

Accordingly, disclosed herein is a method for the treatment of an adult patient with refractory CML, wherein the method comprises administering to said patient a therapeutically effective amount of an initial daily dose of 174 mg of a compound of Formula I or a pharmaceutically acceptable salt thereof such that when the patient develops non-hematologic toxicity:
a) the initial daily dose is withheld for at least 7 days and resumed after recovery of patient from said toxicity; or
b) the initial daily dose is withheld for at least 7 days and resumed at a reduced daily dose of 126 mg on first instance and to 90 mg, 66 mg and 48 mg on subsequent instances of recurrences of said toxicity;
and wherein, the reduced dose is optionally re-escalated to any one of the dose selected from 66 mg, 90 mg, 126 mg and 174 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof results in a mean Cₘₐₓ ranging from 664±450 ng/ml to 5054±3051 ng/mL.

Accordingly, disclosed herein is a method for the treatment of an adult patient with refractory CML, wherein the method comprises administering to the patient a therapeutically effective amount of an initial daily dose of 174 mg of a compound of Formula I or a pharmaceutically acceptable salt thereof such that when the patient develops hematologic toxicity:
a) the initial daily dose is withheld for at least 14 days and resumed after recovery of patient from said toxicity; or
b) the initial daily dose is withheld for at least 14 days and resumed at a reduced daily dose of 126 mg on first instance and to 90 mg, 66 mg and 48 mg on subsequent instances of recurrence of said toxicity.

Accordingly, disclosed herein is a method for the treatment of an adult patient with refractory CML, wherein the method comprises administering to the patient a therapeutically effective amount of an initial daily dose of 174 mg of a compound of Formula I or a pharmaceutically acceptable salt thereof such that when the patient develops hematologic toxicity:
a) the initial daily dose is withheld for at least 14 days and resumed after recovery of patient from said toxicity; or
b) the initial daily dose is withheld for at least 14 days and resumed at a reduced daily dose of 126 mg on first instance and to 90 mg, 66 mg and 48 mg on subsequent instances of recurrence of said toxicity;
and wherein the daily dose of the compound of Formula I or a pharmaceutically acceptable salt thereof in the patient results in mean AUC₀₋₂₄ ranging from 6226±5827 ng*h/mL to 60373±55659 ng*h/mL.

Accordingly, disclosed herein is a method for the treatment of an adult patient with refractory CML, wherein the method comprises administering to the patient a therapeutically effective amount of an initial daily dose of 174 mg of a compound of Formula I or a pharmaceutically acceptable salt thereof such that when the patient develops hematologic toxicity:
a) the initial daily dose is withheld for at least 14 days and resumed after recovery of patient from said toxicity; or
b) the initial daily dose is withheld for at least 14 days and resumed at a reduced daily dose of 126 mg on first instance and to 90 mg, 66 mg and 48 mg on subsequent instances of recurrence of said toxicity;
and wherein the daily dose of the compound of Formula I or a pharmaceutically acceptable salt thereof results in a mean Cₘₐₓ ranging from 664±450 ng/ml to 5054±3051 ng/mL.

Accordingly, disclosed herein is a method for the treatment of an adult patient with refractory CML, wherein the method comprises administering to the patient a therapeutically effective amount of an initial daily dose of 174 mg of a compound of Formula I or a pharmaceutically acceptable salt thereof such that when the patient develops hematologic toxicity:
a) the initial daily dose is withheld for at least 14 days and resumed after recovery of patient from said toxicity; or
b) the initial daily dose is withheld for at least 14 days and resumed at a reduced daily dose of 126 mg on first instance and to 90 mg, 66 mg and 48 mg on subsequent instances of recurrence of said toxicity;
and wherein, the reduced dose is optionally re-escalated to any one of the doses selected from 66 mg, 90 mg, 126 mg and 174 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

Accordingly, disclosed herein is a method for the treatment of an adult patient with refractory CML, wherein the method comprises administering to the patient a therapeutically effective amount of an initial daily dose of 174 mg of a compound of Formula I or a pharmaceutically acceptable salt thereof such that when the patient develops
a) the initial daily dose is withheld for at least 14 days and resumed after recovery of patient from said toxicity; or
b) the initial daily dose is withheld for at least 14 days and resumed at a reduced daily dose of 126 mg on first instance and to 90 mg, 66 mg and 48 mg on subsequent instances of recurrence of said toxicity;
and wherein, the reduced dose is optionally re-escalated to any one of the dose selected from 66 mg, 90 mg, 126 mg and 174 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof in the patient results in mean AUC₀₋₂₄ ranging from 6226±5827 ng*h/mL to 60373±55659 ng*h/mL.

Accordingly, disclosed herein is a method for the treatment of an adult patient with refractory CML, wherein the method comprises administering to the patient a therapeutically effective amount of an initial daily dose of 174 mg of a compound of Formula I or a pharmaceutically acceptable salt thereof such that when the patient develops hematologic toxicity:
a) the initial daily dose is withheld for at least 14 days and resumed after recovery of patient from said toxicity; or
b) the initial daily dose is withheld for at least 14 days and resumed at a reduced daily dose of 126 mg on first instance and to 90 mg, 66 mg and 48 mg on subsequent instances of recurrence of said toxicity;
and wherein, the reduced dose is optionally re-escalated to any one of the dose selected from 66 mg, 90 mg, 126 mg and 174 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof results in a mean Cₘₐₓ ranging from 664±450 ng/ml to 5054±3051 ng/mL.

Accordingly, disclosed herein is a method for the treatment of an adult patient with refractory CML, wherein the method comprises administering to the patient a therapeutically effective amount of an initial daily dose of 174 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced dose of 126 mg on first instance and at 90 mg, 66 mg and 48 mg on subsequent instances of recurrence of said toxicity.

Accordingly, disclosed herein is a method for the treatment of an adult patient with refractory CML, wherein the method comprises administering to the patient a therapeutically effective amount of an initial daily dose of 174 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced dose of 126 mg on first instance and at 90 mg, 66 mg and 48 mg on subsequent instances of recurrence of said toxicity; and wherein the daily dose of the compound of Formula I or a pharmaceutically acceptable salt thereof in the patient results in mean AUC₀₋₂₄ ranging from 6226±5827 ng*h/mL to 60373±55659 ng*h/mL.

Accordingly, disclosed herein is a method for the treatment of an adult patient with refractory CML, wherein the method comprises administering to the patient a therapeutically effective amount of an initial daily dose of 174 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced dose of 126 mg on first instance and at 90 mg, 66 mg and 48 mg on subsequent instances of recurrence of said toxicity; and wherein the daily dose of the compound of Formula I or a pharmaceutically acceptable salt thereof results in a mean Cₘₐₓ ranging from 664±450 ng/mL to 5054±3051 ng/mL.

Also disclosed herein is a method for the treatment of an adult patient with refractory CML, wherein the method comprises administering to the patient a therapeutically effective amount of an initial daily dose of 174 mg of a compound of Formula I or a pharmaceutically acceptable salt thereof such that when the patient develops hematologic toxicity that is attributable to the compound of Formula I or a pharmaceutically acceptable salt thereof, the initial daily dose is withheld for up to four weeks to allow the recovery of patient from said toxicity and the treatment is continued at a reduced dose of 126 mg on first instance and at 90 mg, 66 mg and 48 mg on subsequent instances of recurrence of said toxicity, and wherein the reduced dose is optionally re-escalated to any one of the dose selected from 66 mg, 90 mg, 126 mg and 174 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

Also disclosed herein is a method for the treatment of an adult patient with refractory CML, wherein the method comprises administering to the patient a therapeutically effective amount of an initial daily dose of 174 mg of a compound of Formula I or a pharmaceutically acceptable salt thereof such that when the patient develops hematologic toxicity that is attributable to the compound of Formula I or a pharmaceutically acceptable salt thereof, the initial daily dose is withheld for up to four weeks to allow the recovery of patient from said toxicity and the treatment is continued at a reduced dose of 126 mg on first instance and at 90 mg, 66 mg and 48 mg on subsequent instances of recurrence of said toxicity, and wherein the reduced dose is optionally re-escalated to any one of the dose selected from 66 mg, 90 mg, 126 mg and 174 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof in the patient results in mean AUC₀₋₂₄ ranging from 6226±5827 ng*h/mL to 60373±55659 ng*h/mL.

Also disclosed herein is a method for the treatment of an adult patient with refractory CML, wherein the method comprises administering to the patient a therapeutically effective amount of an initial daily dose of 174 mg of a compound of Formula I or a pharmaceutically acceptable salt thereof such that when the patient develops hematologic toxicity that is attributable to the compound of Formula I or a pharmaceutically acceptable salt thereof, the initial daily dose is withheld for up to four weeks to allow the recovery of patient from said toxicity and the treatment is continued at a reduced dose of 126 mg on first instance and at 90 mg, 66 mg and 48 mg on subsequent instances of recurrence of said toxicity, and wherein the reduced dose is optionally re-escalated to any one of the dose selected from 66 mg, 90 mg, 126 mg and 174 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction,
and wherein the daily dose of the compound of Formula I or a pharmaceutically acceptable salt thereof results in a mean Cₘₐₓ ranging from 664±450 ng/ml to 5054±3051 ng/ml.

According to another embodiment, disclosed herein is a method for the treatment of newly diagnosed adult CML patient comprising, administering a therapeutically effective amount of compound of Formula I or a pharmaceutically acceptable salt thereof at an initial daily dose of 174 mg, wherein the initial dose is escalated to 204 mg to achieve or maintain complete hematologic response (CHR) with no severe adverse reaction.

According to one embodiment, disclosed herein is a method for the treatment of newly diagnosed adult CML patient comprising, administering a therapeutically effective amount of compound of Formula I or a pharmaceutically acceptable salt thereof at an initial daily dose of 174 mg, wherein the initial dose is escalated to 204 mg to achieve or maintain complete hematologic response (CHR) with no severe adverse reaction, and wherein the daily dose of the compound of Formula I or a pharmaceutically acceptable salt thereof in the patient results in mean AUC₀₋₂₄ ranging from 6226±5827 ng*h/mL to 60373±55659 ng*h/mL.

According to another embodiment, disclosed herein is a method for the treatment of newly diagnosed adult CML patient comprising, administering a therapeutically effective amount of compound of Formula I or a pharmaceutically acceptable salt thereof at an initial daily dose of 174 mg, wherein the initial dose is escalated to 204 mg to achieve or maintain complete hematologic response (CHR) with no severe adverse reaction, and wherein the daily dose of the compound of Formula I or a pharmaceutically acceptable salt thereof results in a mean Cₘₐₓ ranging from 664±450 ng/ml to 5054±3051 ng/ml.

Accordingly, disclosed herein is a method for the treatment of newly diagnosed adult CML patient comprising, orally administering a therapeutically effective amount of a compound of Formula I or a pharmaceutically acceptable salt thereof at an initial daily dose of 174 mg, wherein the initial dose is escalated to 204 mg to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

Accordingly, disclosed herein is a method for the treatment of a newly diagnosed adult CML patient, wherein the method comprises administering to the patient a therapeutically effective amount of an initial daily dose of 174 mg of a compound of Formula I or a pharmaceutically acceptable salt thereof such that when the patient develops non-hematological toxicity:
a) an initial daily dose is withheld for at least 7 days and resumed after recovery of patient from said toxicity 174 mg; or
b) an initial daily dose is withheld for at least 7 days and resumed at a reduced daily dose of 126 mg on first instance and to 90 mg, 66 mg, 48 mg, 24 mg and 12 mg on subsequent instances of recurrences of said toxicity.

Accordingly, disclosed herein is a method for the treatment of a newly diagnosed adult CML patient, wherein the method comprises administering to the patient a therapeutically effective amount of an initial daily dose of 174 mg of a compound of Formula I or a pharmaceutically acceptable salt thereof such that when the patient develops non-hematologic toxicity:
a) the initial daily dose is withheld for at least 7 days and resumed after recovery of patient from said toxicity; or
b) the initial daily dose is withheld for at least 7 days and resumed at a reduced daily dose of 126 mg on first instance and to 90 mg, 66 mg 48 mg, 24 mg and 12 mg on subsequent instances of recurrences of said toxicity;
and wherein, the reduced dose is optionally re-escalated to any one of the dose selected from 24 mg, 48 mg, 66 mg, 90 mg, 126 mg and 174 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

Accordingly, disclosed herein is a method for the treatment of a newly diagnosed adult CML patient, wherein the method comprises administering to the patient a therapeutically effective amount of an initial daily dose of 174 mg of a compound of Formula I or a pharmaceutically acceptable salt thereof such that when the patient develops hematologic toxicity:
a) the initial daily dose is withheld for at least 14 days and resumed after recovery of patient from said toxicity; or
b) the initial daily dose is withheld for at least 14 days and resumed at a reduced daily dose of 126 mg on first instance and to 90 mg, 66 mg, 48 mg, 24 mg and 12 mg on subsequent instances of recurrence of said toxicity.

Accordingly, disclosed herein is a method for the treatment of a newly diagnosed adult CML patient, wherein the method comprises administering to the patient a therapeutically effective amount of an initial daily dose of 174 mg of a compound of Formula I or a pharmaceutically acceptable salt thereof such that when the patient develops hematologic toxicity:
a) the initial daily dose is withheld for at least 14 days and resumed after recovery of patient from said toxicity; or
b) the initial daily dose is withheld for at least 14 days and resumed at a reduced daily dose of 126 mg on first instance and to 90 mg, 66 mg, 48 mg, 24 mg and 12 mg on subsequent instances of recurrence of said toxicity;
and wherein, the reduced dose is optionally re-escalated to any one of the dose selected from 24 mg, 48 mg, 66 mg, 90 mg, 126 mg and 174 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

Accordingly, disclosed herein is a method for the treatment of a newly diagnosed adult CML patient, wherein the method comprises administering to the patient a therapeutically effective amount of an initial daily dose of 174 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced dose of 126 mg on first instance and at 90 mg, 66 mg, 48 mg, 24 mg and 12 mg on subsequent instances of recurrence of said toxicity.

Also disclosed herein is a method for the treatment of a newly diagnosed adult CML patient, wherein the method comprises administering to the patient a therapeutically effective amount of an initial daily dose of 174 mg of a compound of Formula I or a pharmaceutically acceptable salt thereof such that when the patient develops hematologic toxicity that is attributable to the compound of Formula I or a pharmaceutically acceptable salt thereof,
the initial daily dose is withheld for up to four weeks to allow recovery of patient from said toxicity and the treatment is continued at a reduced dose of 126 mg on first instance and at 90 mg, 66 mg, 48 mg, 24 mg and 12 mg on subsequent instances of recurrence of the toxicity, and wherein
the reduced dose is optionally re-escalated to any one of the dose selected from 24 mg, 48 mg, 66 mg, 90 mg, 126 mg and 174 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no hematologic toxicity.

According to an embodiment, disclosed herein is a method for the treatment of an adult patient with chronic myeloid leukemia (CML), comprising orally administering a pharmaceutical composition comprising a therapeutic effective amount of a compound of Formula I or a pharmaceutically acceptable salt thereof, at an initial daily dose, and escalating or de-escalating the dose to achieve or maintain complete hematologic response, with no severe adverse reaction.

According to another embodiment, disclosed herein is a method for the treatment of an adult patient with chronic myeloid leukemia (CML), comprising orally administering a pharmaceutical composition comprising a therapeutic effective amount of a compound of Formula I or a pharmaceutically acceptable salt thereof, at an initial daily dose, and escalating or de-escalating the dose to achieve or maintain complete hematologic response, with no severe adverse reaction, wherein the composition is administered under fasting condition and wherein the composition results in a mean AUC₀₋₂₄ ranging from 6226±5827 ng*h/mL to 60373±55659 ng*h/mL.

According to another embodiment, disclosed herein is a method for the treatment of an adult patient with chronic myeloid leukemia (CML), comprising orally administering a pharmaceutical composition comprising a therapeutic effective amount of a compound of Formula I or a pharmaceutically acceptable salt thereof, at an initial daily dose, and escalating or de-escalating the dose to achieve or maintain complete hematologic response, with no severe adverse reaction, wherein the composition is administered under fasting condition and wherein the composition results in a mean Cₘₐₓ ranging from 664±450 ng/ml to 5054±3051 ng/ml.

Accordingly, disclosed herein is a method for the treatment of an adult patient with CML, comprising orally administering a pharmaceutical composition comprising a therapeutic effective amount of a compound of Formula I or a pharmaceutically acceptable salt thereof at an initial daily dose of between 10 mg and 200 mg and escalating to the dose between 20 mg and 210 mg to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction due to the composition comprising a compound of Formula I or a pharmaceutically acceptable salt thereof.

Accordingly, disclosed herein is a method for the treatment of an adult patient with CML, comprising orally administering a pharmaceutical composition comprising a therapeutic effective amount of a compound of Formula I or a pharmaceutically acceptable salt thereof at an initial daily dose of between 10 mg and 200 mg and escalating to the dose between 20 mg and 210 mg to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction due to the composition comprising a compound of Formula I or a pharmaceutically acceptable salt thereof, wherein the composition when administered under fasting condition results in a mean AUC₀₋₂₄ ranging from 6226±5827 ng*h/mL to 60373±55659 ng*h/mL and mean Cₘₐₓ ranging from 664±450 ng/ml to 5054±3051 ng/mL.

According to another embodiment, disclosed herein is a method for the treatment of an adult patient with CML, comprising orally administering a therapeutically effective amount of a compound of Formula I or a pharmaceutically acceptable salt thereof to the patient at a daily dose wherein such daily dose does not result in patient's QT interval of more than about 500 ms.

According to another embodiment, disclosed herein is a method for the treatment of an adult patient with CML, comprising orally administering a therapeutically effective amount of a compound of Formula I or a pharmaceutically acceptable salt thereof to the patient at a daily dose wherein such daily dose results in a mean AUC₀₋₂₄ ranging from 6226±5827 ng*h/mL to 60373±55659 ng*h/mL and mean Cₘₐₓ ranging from 664±450 ng/mL to 5054±3051 ng/ml and wherein the daily dose does not result in patient's QT interval of more than about 500 ms.

In another embodiment, disclosed herein is a method for the prevention of recurrence of CML in a patient that has previously undergone treatment using a tyrosine kinase inhibitor, wherein the method comprises orally administering a therapeutically effective amount of a compound of Formula I or a pharmaceutically acceptable salt thereof at an initial daily dose of 174 mg.

In yet another embodiment, disclosed herein is a method for the prevention of recurrence of CML in a patient that has previously undergone treatment using a tyrosine kinase inhibitor, wherein the method comprises orally administering a therapeutically effective amount of a compound of Formula I or a pharmaceutically acceptable salt thereof at an initial daily dose of 174 mg, wherein the initial daily dose results in a mean AUC₀₋₂₄ ranging from 6226±5827 ng*h/mL to 60373±55659 ng*h/mL and mean Cₘₐₓ ranging from 664±450 ng/mL to 5054±3051 ng/mL.

According to an embodiment, there is provided a method of treating an adult patient with CML by orally administering a therapeutically effective amount of a compound of Formula I or a pharmaceutically acceptable salt thereof under fasting conditions such that, a mean AUC₀₋₂₄ achieved in patient by administering the compound of Formula I or a pharmaceutically acceptable salt thereof under fasting conditions is 15% higher as compared to the mean AUC₀₋₂₄ obtained by administering the compound of Formula I or a pharmaceutically acceptable salt thereof under fed conditions.

Accordingly to an embodiment, there is provided a method of treating an adult patient with CML by orally administering a therapeutically effective amount of a compound of Formula I or a pharmaceutically acceptable salt thereof at a daily dose ranging from 10 mg to 210 mg, wherein the daily dose of the compound of Formula I or a pharmaceutically acceptable salt thereof results in a mean AUC₀₋₂₄ ranging from 6226±5827 ng*h/mL to 60373±55659 ng*h/mL, when administered under fasting conditions.

According to another embodiment, there is provided a method of treating an adult patient with CML by orally administering a therapeutically effective amount of a compound of Formula I or a pharmaceutically acceptable salt thereof under fasting conditions such that, a mean Cₘₐₓ achieved in patient by administering the compound of Formula I or a pharmaceutically acceptable salt thereof under fasting conditions is 20% higher as compared to the mean Cₘₐₓ achieved by administering the compound of Formula I or a pharmaceutically acceptable salt thereof under fed conditions.

Accordingly, to an embodiment, there is provided a method of treating an adult patient with CML by orally administering a therapeutically effective amount of a compound of Formula I or a pharmaceutically acceptable salt thereof at a daily dose ranging from 10 mg to 210 mg, wherein the daily dose of the compound of Formula I or a pharmaceutically acceptable salt thereof results in a mean Cₘₐₓ ranging from 664±450 ng/ml to 5054±3051 ng/mL, when administered under fasting conditions.

According to another embodiment, there is provided a method for the treatment of an adult patient with CML, wherein the method comprises administering a therapeutically effective amount of a compound of Formula I or a pharmaceutically acceptable salt thereof to the patient at a daily dose such that the plasma concentration of the compound of Formula I or a pharmaceutically acceptable salt thereof is not less than 50 ng/mL.

According to yet another embodiment, there is provided a method for the treatment of an adult patient with CML, comprising orally administering a therapeutically effective amount of a compound of Formula I or a pharmaceutically acceptable salt thereof at a predetermined daily dose, wherein said daily dose, (a) results in not less than 50 ng/mL of the compound of Formula I or a pharmaceutically acceptable salt thereof in the plasma; (b) results in mean AUC₀₋₂₄ ranging from 6226±5827 ng*h/mL to 60373±55659 ng*h/mL; (c) achieves a mean Cₘₐₓ ranging from 664±450 ng/ml to 5054±3051 ng/ml; (d) achieves 15% higher AUC₀₋₂₄ and 20% higher Cₘₐₓ when administered under fasting conditions as compared to when administered under fed conditions; and/or (e) results in patient's QT interval of less than about 500 ms.

According to yet another embodiment, there is provided a method for the treatment of an adult patient with CML, comprising orally administering a therapeutically effective amount of a compound of Formula I or a pharmaceutically acceptable salt thereof at a predetermined daily dose, wherein said daily dose, (a) results in not less than 25 ng/ml of the compound of Formula I or a pharmaceutically acceptable salt thereof in the plasma; (b) results in mean AUC₀₋₂₄ ranging from 6226±5827 ng*h/mL to 60373±55659 ng*h/mL; (c) achieves a mean Cₘₐₓ ranging from 664±450 ng/ml to 5054±3051 ng/ml; (d) achieves 15% higher AUC₀₋₂₄ and 20% higher Cₘₐₓ when administered under fasting conditions as compared to when administered under fed conditions; and/or (e) results in patient's QT interval of less than about 500 ms.

According to an embodiment, there is provided a method for the treatment of an adult patient with CML, comprising orally administering a therapeutically effective amount of a compound of Formula I or a pharmaceutically acceptable salt thereof at a predetermined daily dose, wherein the daily dose is escalated to achieve or maintain complete hematologic response or the daily dose is reduced when the patient experiences severe adverse reaction, and wherein the predetermined, escalated or reduced daily dose; (a) results in not less than 50 ng/ml of the compound of Formula I or a pharmaceutically acceptable salt thereof in the plasma; (b) results in mean AUC₀₋₂₄ ranging from 6226±5827 ng*h/mL to 60373±55659 ng*h/mL; (c) achieves a mean Cₘₐₓ ranging from 664±450 ng/ml to 5054±3051 ng/ml; (d) achieves 15% higher AUC₀₋₂₄ and 20% higher Cₘₐₓ when administered under fasting conditions as compared to when administered under fed conditions; and/or (e) results in patient's QT interval of less than about 500 ms.

According to another embodiment, there is provided a method of treating a CML patient having a relapsed or refractory CML condition, wherein the relapsed or refractory condition arises due to mutations in the kinase domain of BCR-ABL1 fusion oncoprotein, the method comprising administering a therapeutically effective amount of a compound of Formula I or a pharmaceutically acceptable salt thereof to said patient at a daily dose of 48 to 204 mg.

According to another embodiment, there is provided a method of treating a CML patient having a relapsed or refractory CML condition, wherein the relapsed or refractory condition arises due to mutations in the kinase domain of BCR-ABL1 fusion oncoprotein, the method comprising administration of a pharmaceutical composition comprising a therapeutically effective amount of a compound of Formula I or a pharmaceutically acceptable salt thereof in an amorphous form and a pharmaceutically acceptable excipient.

According to another embodiment of the present invention, there is a method of increasing a survival likelihood of a CML patient, comprising administering a therapeutically effective amount of a compound of Formula I or a pharmaceutically acceptable salt thereof to said patient, wherein prior to said administering, said CML patient failed at least 1 TKIs.

According to an embodiment of the present invention, there is provided a method for the treatment of an adult patient who had previously undergone treatment for T315I-positive CML and has a relapsed or refractory CML condition, wherein the method comprises orally administering a therapeutically effective amount of a compound of Formula I or a pharmaceutically acceptable salt thereof at an initial daily dose, and escalating or de-escalating the dose to achieve or maintain disease response, with no severe adverse reaction.

### BRIEF DESCRIPTION OF THE DRAWINGS

FIG. 1: SAD: individual, Mean, Median AUC₍₀₋₂₄₎ versus administered dose;

FIG. 2: Schematic for an outline of the study and timing of clinic visits for assessment

### DEFINITIONS

The term “baseline” as used herein is the same as that understood by the person having knowledge about clinical trials and the processes involved therein. It refers to the condition of patient in need of the treatment prior to initiation of said treatment. The conditions include but not limited to physical conditions, disease conditions, vital signs, mental abilities and patient daily activity performance status. In the case of cancer patients, the performance status is graded according to the standards listed by Eastern Cooperative Oncology Group (ECOG). For the purpose of the present study, the patients were ECOG grade 0 or grade 1 patients who were either fully active and could carry out all the day-to-day activities without restrictions or were restricted in physically strenuous activity and could carry out work of a light or sedentary nature. ECOG grade 2 patients who are ambulatory and are capable of all self-care but unable to carry out any work activities can also be considered for the present invention. The baseline analysis is necessary to compare the effect of treatment on the patient.

The terms “adverse event (AE)”, “severe adverse reaction”, “severe adverse event”, “adverse reaction”, “adverse effects”, “toxicity”, “treatment-emergent adverse event (TEAE)”, “side-effects' are used interchangeably and mean an event that was not present at baseline prior to the treatment or was present at a lesser intensity and has occurred or worsened after the treatment was initiated in the patient. Such events are undesirable, unacceptable, unfavorable and unintended signs, symptoms or diseases that can be attributed to the use of drugs or treatment. Based on the intensity and effect of adverse events the skilled person may have to modify the treatment conditions. The daily dose may be administered continuously until the progression of the disease. Toxicity resolved can be when there is less than or equal grade 1 and/or 2 toxicity as per CTCAE.

For the purpose of the present invention, the adverse events were graded as per the National Cancer Institute's, Common Terminology Criteria for Adverse Events (NCI-CTCAE) Version 4.03 and Version 5.0 (https://evs.nci.nih.gov/ftp1/CTCAE/About.html). It is a descriptive terminology which can be utilized for Adverse Event (AE) reporting. The CTCAE displays Grades 1 through 5 with unique clinical descriptions of severity for each AE based on below general guidelines:
a) Grade 1 mild; asymptomatic or mild symptoms; clinical or diagnostic observations only; intervention not indicated;
b) Grade 2 moderate; minimal, local or noninvasive intervention indicated; limiting age-appropriate instrumental Activities of Daily Living (ADL) such as preparing meals, shopping for groceries or clothes, using the telephone, managing money, etc.;
c) Grade 3 severe or medically significant but not immediately life-threatening; hospitalization or prolongation of hospitalization indicated; disabling; limiting self-care ADL such as bathing, dressing and undressing, feeding self, using the toilet, taking medications, and not bedridden;
d) Grade 4 life-threatening consequences; urgent intervention indicated; and
e) Grade 5 death related to AE.

For the purpose of the present invention adverse events (AEs) were assessed throughout the study until 30 days after discontinuation of the study drug. AEs were graded as per NCI-CTCAE Version 5.0. In the present study, along with others most common AEs for which patients were monitored were thrombocytopenia, neutropenia and anemia, any AE reported by the patient after the completion of AE collection period and considered related to the study drug was reported. AEs were followed to a satisfactory resolution until it became stable, or until it could be explained by another known cause (i.e. concurrent condition or medication) and clinical judgment indicated that further evaluation is not warranted.

Dose Limiting Toxicity (DLT) is defined as the occurrence of any of the following unless clearly and incontrovertibly related to the underlying disease. The hematological DLTs were defined as per the Phase of CML:
a) Grade 3 or higher, or other non-hematological toxicity, including nausea, vomiting and diarrhea, and refractory to standard antiemetic therapy except alopecia and nail disorders;
b) Missed ≥25% of doses over 28 days due to toxicity in Cycle 1; and
c) Any grade of toxicity requiring dose reduction or discontinuation of IMP within the 28-days DLT assessment period (Cycle 1).

Hematological DLTs for subjects in CML-Chronic Phase are grade 3 neutropenia and/or thrombocytopenia for ≥28 days off the study drug treatment or grade 4 neutropenia (absolute neutrophil count (ANC)<0.5×10⁹/L in peripheral blood) ≥7 days off the study drug (i.e.: after treatment interruption).

Hematological DLTs for subjects in CML-Accelerated Phase are grade 3 neutropenia and/or thrombocytopenia for ≥28 days off the study drug treatment or grade 4 neutropenia (ANC <0.5×10⁹/L in peripheral blood)≥7 days off the treatment (i.e.: after treatment interruption) in the absence of features of an accelerated phase (except cytogenetic changes) such as a persistent increase in blasts or basophils.

Hematological DLTs for subjects in CML-Blast Phase are Grade 3 neutropenia and/or thrombocytopenia in the absence of persistent leukemia or ANC ≤500/mm³ or thrombocytopenia <50,000/mm³ for >6 weeks with a bone marrow cellularity showing <5% blasts.

It would be appreciated by a skilled person in the art that the hematological DLT can be confirmed only after distinguishing between toxicity due to the compound of Formula I or a pharmaceutically acceptable salt thereof and anti-leukemic effect. This can be done by the methods known to the skilled person. For example, if the subject has neutropenia with clearance of leukemic cells or blasts, a bone marrow examination can be performed to distinguish between toxicity due to Formula I and the anti-leukemic effect, if the subject shows a normocellular marrow or persistence of the disease, can be said to be an anti-leukemic effect and the treatment can be continued, however, if there is evidence of drug toxicity such as hypocellular marrow, then the treatment is halted for a predetermined period.

The term ‘disease response’ or simply ‘response’ due to the compound of Formula I or a pharmaceutically acceptable salt thereof is understood as an effect of the compound of Formula I or a pharmaceutically acceptable salt thereof on the progression of the disease in the patient. The disease response is determined by screening the patient before and after initiation of the treatment.

The disease response is determined by screening the patient before and after initiation of the treatment. The screening may include both clinical and laboratory investigations such as physical examination, blood analysis, vital signs analysis for example temperature, heart rate, respiratory rate, and diastolic and systolic blood pressure, ECG, bone marrow aspiration, BCR-ABL mutational analysis, and BCR-ABL transcript analysis. From the screening results if it is evident that the compound of Formula I or a pharmaceutically acceptable salt thereof reduces or ameliorates or alleviates the disease or its symptoms, the patient is said to have a disease response. Particularly, in the case of leukemia, the disease response is confirmed when the patient exhibits hematological response (CHR), cytogenetic response, and molecular response, sequentially in the given order. For example, a CP CML patient is expected to initially exhibit a major or complete hematologic response and then show signs of cytogenetic response followed by molecular response. The patient with AP CML and BP CML is expected to initially exhibit complete hematologic response and then optionally exhibit partial or complete cytogenetic response. It is beneficial if the AP CML and BP CML patients show molecular responses as well. For the purpose of the present invention, the hematological response assessment was performed on the 1^{st}, 2^{nd}, 3^{rd}, 8^{th} day and every 8^{th} day thereafter, wherein the 1^{st} day is the day when the subject had 1^{st} dose of the compound of Formula I or a pharmaceutically acceptable salt thereof.

Bone marrow aspirates were utilized to determine cytogenetic response assessments. Aspirates were obtained after the completion of every 3 cycles (Example: After Cycle 3, the next aspirate was scheduled on completion of Cycle 6, and the next aspirate was scheduled on completion of Cycle 9, wherein each cycle consisted of 28 days of treatment. For patients who demonstrated complete cytogenetic response (CCyR) on 2 repeated assessments: Bone marrow aspirate samples were collected at 6 monthly intervals thereafter until disease progression, subject withdrawal of consent or discontinuation of the subject from the study.

Molecular response assessments were performed at the end of 3^{rd} cycle and thereafter every 3 cycles (e.g., after Cycle 3, the next sample collection was scheduled on completion of Cycle 6), wherein each cycle consisted of 28 days of treatment. Samples were collected at the end of every 3 cycles until major molecular response (MMR) was achieved in two consequent assessments and repeated thereafter only in case there was a 10 times increase in the MMR until disease progression or subject withdrawal or consent or discontinuation of the subject from the study.

For subjects who had achieved CCyR and MMR in the study on 2 repeated assessments: Bone marrow aspirate needs to be performed only when a 10-fold increase in BCR-ABL levels was detected. Additional bone marrow aspirations may be performed at unscheduled visits at Investigator's discretion.

The skilled person in the art would appreciate that under circumstances, of bone marrow sample insufficiency, BCR-ABL FISH assay for identification of Ph+ should be performed in lieu of conventional bone marrow cytogenetics and the percentage of cells with Ph+ Chromosome positivity should be reported. BCR-ABL FISH assay may also be performed for subjects with minimum residual disease.

The term ‘hematologic response’ (HR) or ‘hematological response’ is a normalization of the blood counts, particularly WBC counts due to the treatment using a compound of Formula I or a pharmaceutically acceptable salt thereof. This is the first noticeable indicator that treatment is beginning to work, though not necessarily in the bone marrow. The response can be a partial hematological response (PHR) wherein there is a reduction in WBC, but not down to normal range or complete hematological response (CHR) with all the blood counts normalized. A CHR is usually anticipated within a month of treatment, however, some subjects may take even up to 2 months to achieve this level. It was surprisingly observed that the patients that had undergone several previous treatments using other known TKIs and had stopped responding to such prior treatments, could easily achieve CHR on or before the 2-month mark with treatment using a compound of Formula I or a pharmaceutically acceptable salt thereof. Major haematologic response (MaHR) is inclusive of complete hematological response (CHR) and/or no evidence of leukemia (NEL). For the purpose of the present invention, the criteria of hematologic response are given in Table 01.

TABLE 01
Hematological response criteria:
Hematologic response criteria for subjects with AP CML and BP CML and Ph+ ALL

| | |
|---|---|
| CHR | White blood cells (WBCs) ≤ Institutional upper limit of normal as |
| | understood by the person skilled in the art. |
| | ANC ≥ 1000/mm³ |
| | Platelets ≥ 100,000/m³ |
| | No blasts or promyelocytes in peripheral blood |
| | <5% basophils on differential |
| | <5% myelocytes and metamyelocytes on peripheral blood |
| | No extra-medullary involvement (no hepatomegaly and/or splenomegaly) |
| NEL | White blood cells (WBCs) ≤ Institutional upper limit of normal |
| | No blasts or promyelocytes in peripheral blood |
| | <5% basophils on differential |
| | <5% myelocytes and metamyelocytes on peripheral blood |
| | No extra-medullary involvement (no hepatomegaly and/or splenomegaly) |
| | At least one of the following |
| | 20,000/mm³ ≤ Platelets ≤ 100,000/mm³ |
| | 500/mm³ ≤ ANC ≤ 1000/mm³ |

Complete hematological response (CHR) for subjects with CP CML

| | |
|---|---|
| CHR | Platelets < 450 × 10⁹/mm³ |
| | White blood cells (WBCs) ≤ Institutional upper limit of normal |
| | No blasts or promyelocytes in peripheral blood |
| | <5% basophils on differential |
| | <5% myelocytes and metamyelocytes on peripheral blood |
| | No extra-medullary involvement (no hepatomegaly and/or splenomegaly |

The term ‘cytogenetic response’ as used herein means a response to treatment with a compound of Formula I or a pharmaceutically acceptable salt thereof that occurs in the bone marrow, rather than just in the blood. It is a Philadelphia-positive (Ph+) chromosome reading obtained after administration of the compound of Formula I or a pharmaceutically acceptable salt thereof interpreted as described below:

There are 3 levels of cytogenetic response:
a) Partial Cytogenetic Response (PCyR): This indicates that only 1 to 35% of the sample contains Ph+ metaphases;
b) Complete Cytogenetic Response (CCyR): This indicates no Ph+ cells can be measured by either conventional or fluorescence in situ hybridization cytogenetic testing (though the PCR test may still be positive); and
c) Major Cytogenetic Response (MCyR) is cytogenetic response inclusive of PCyR and CCyR.

For the purpose of the present invention, the cytogenetic response was determined using bone marrow aspirate evaluation by Giemsa staining method (karyotyping) or Fluorescence in situ hybridization, FISH assay. As understood by the skilled person for confirmation of cytogenetic response at least 20 metaphases are evaluated. If fewer metaphases are reported, absolute percentage values are reported. Also, the peripheral blood cells are not considered for bone marrow aspirate evaluation. Under limited bone marrow, aspirate availability precluding cytogenetic evaluation by Giemsa staining, a FISH assay for evaluation of Ph+ Cells is performed. A complete cytogenetic response (CCyR) is reported for FISH when No Ph+ cells are observed or fewer than 1 out of 200 nuclei are BCR/ABL1-positive.

TABLE 02
Cytogenetic Response Criteria:

| | | |
|---|---|---|
| Major Cytogenetic | Complete Response (CCyR) | No Ph+ Cells |
| Response | Partial Response (PCyR) | 1-35% Ph+ Cells |

| | |
|---|---|
| Minor Response | 36-65% Ph+ Cells |
| Minimal Response | 66-95% Ph+ Cells |
| No Response | 96-100% Ph+ Cells |

Major Molecular Response (MMR): Molecular response is a response to the treatment that affects the number of BCR-ABL transcripts in blood cells of patients with CML or ALL. It is measured as the ratio of the reverse transcribed transcript of BCR-ABL to ABL. For major molecular response, the ratio is ≤0.1% on the international scale (IS) (equivalent to 3 log reduction in the transcript). It may be determined by polymerase chain reaction (PCR) or any other molecular test as known to the skilled person.

Major Molecular Response (MMR) is used to select and monitor patients who are eligible for treatment discontinuation of tyrosine kinase therapy. In another embodiment, the major molecular response rate is determined at 12 weeks of the treatment. In another embodiment, the major molecular response rate is determined at 24 weeks of the treatment. In another embodiment, the major molecular response rate is determined at 96 weeks of the treatment.

For the purpose of the present invention, the molecular response was determined using PCR. The patient is said to have major molecular response when the amount of BCR-ABL protein in the blood is very low that is if BCR-ABL transcripts are 0.1% by quantitative PCR (International scale (IS)) or more than or equal to 3-log reduction on BCR-ABL mRNA from the standardized baseline if quantitative PCR (IS) is not available. If no BCR-ABL mRNA is detectable by quantitative PCR (IS) using an assay with a sensitivity of at least 4.5 logs below the standardized baseline, it is said to be complete molecular response.

The phrase ‘Optimal management’ is used herein in relevance with adverse events or toxicities and means the steps taken for the reduction or elimination of adverse events. The management is based on the CTCAE grades. For example, grade 1 adverse events may be managed by holding the treatment for a few days, while CTCAE grade 2 AEs may require adjacent medications plus withholding of treatment. The skilled person is aware of the methods used for the treatment of adverse events including any medication used for such purpose.

As used herein, the term “subject” or “a human in need thereof” or “patient” means a human subject who is diagnosed with a disease such as leukemia and is in need of treatment. These terms can be interchangeable. Preferably the subject is diagnosed with acute lymphocytic leukemia (ALL), acute myeloid leukemia (AML), chronic lymphocytic leukemia (CLL), chronic myeloid leukemia (CML), Ph+ CML or other leukemia such as hairy cell leukemia, myelodysplastic syndromes or myeloproliferative disorders or combination thereof. More preferably the subject is diagnosed with ALL or CML and is in need of such treatment that reduces or ameliorates or alleviates the disease or its symptoms or cures and frees the subject from the disease or its symptoms. The subject may have newly diagnosed, refractory or relapsed leukemia, wherein newly diagnosed and relapsed has the meaning known to the skilled person. The subject or patient is said to have refractory leukemia when said subject has undergone treatment using known therapies preferably using tyrosine kinase inhibitors (TKIs) and is resistant or intolerant to such therapies.

For the purpose of the present invention, the refractory leukemia patient is the one who has undergone prior treatment using at least 1 TKI or at least 2 TKIs or at least 3 TKIs. In a particular embodiment, when the patient has undergone prior treatment using at least 3 TKIs, one of the TKIs is ponatinib. In a particular embodiment, when the patient has undergone prior treatment using at least 3 TKIs, one of the TKIs is bosutinib. In yet another embodiment, when the patient has undergone prior treatment using at least 3 TKIs, one of the TKIs is ponatinib or asciminib. In yet another embodiment, when the patient has undergone prior treatment using at least 3 TKIs, including treatment using ponatinib and asciminib.

The subject is said to be ‘resistant’ to the prior treatments using known TKIs if there is no response and improvement in the disease in the subject. For the purpose of the present invention, the subject is said to be resistant to the prior treatments or therapies in case of any of the following incidences as given in Table 03:

TABLE 03

| | CP CML | AP CML | BP CML |
|---|---|---|---|
| | (Subject must meet at | (Subject must meet at | (Subject must meet at |
| | least 1 of the following | least 1 of the following | least 1 of the following |
| | criteria) | criteria) | criteria) |
| Hematological | Failure to achieve CHR 3 | Failure to achieve MaHR | Failure to achieve |
| | months after initiation of | 3 months after initiation | MaHR 1 month after |
| | therapy | of therapy | initiation of therapy |
| | Or | Or | Or |
| | Loss of previously | Loss of previously | Loss of previously |
| | attained MaHR any time | attained MaHR at any | attained MaHR at any |
| | after initiation of TKI | time after the initiation of | time after the initiation |
| | therapy (confirmed in at | TKI therapy (confirmed | of TKI therapy |
| | least 2 consecutive | in at least 2 consecutive | (confirmed in at least 2 |
| | analyses at least 4 weeks | analyses at least 4 weeks | consecutive analyses at |
| | apart from initiation) | apart from initiation) | least 1 week apart from |
| | | | initiation) |
| Cytogenetic | No cytogenetic response | Not Applicable | Not Applicable |
| | (>95% Ph+ cells) - after | | |
| | 3 months | | |
| | Or | | |
| | Less than a minor | | |
| | cytogenetic response | | |
| | (>65% Ph+) - after 6 | | |
| | months | | |
| | Or | | |
| | Less than a PCyR (>35% | | |
| | Ph+) - after 12 months | | |
| | Or | | |
| | Loss of cytogenetic | | |
| | response at any time after | | |
| | initiation of treatment | | |
| | (including shift from | | |
| | complete (0%) to partial | | |
| | (1% to 35%) to minor | | |
| | (36% to 65%) to minimal | | |
| | (66% to 95%) | | |
| Molecular | BCR-ABL1(IS) >10% - | Not applicable | Not applicable |
| | after 3 months | | |
| | Or | | |
| | BCR-ABL1(IS) >1% - | | |
| | after 12 months | | |
| | Or | | |
| | Loss of Major Molecular | | |
| | response at any time | | |
| | during therapy | | |
| Other | At any time after | At any time after | At any time after |
| | initiation, development | initiation, development of | initiation, development |
| | of new BCR-ABL | new BCR-ABL mutations | of new BCR-ABL |
| | mutations with loss of | with loss of previously | mutations with loss of |
| | previously obtained | obtained response | previously obtained |
| | response | Or | response |
| | Or | At any time after | Or |
| | At any time after | initiation new clonal | At any time after |
| | initiation new clonal | chromosome | initiation new clonal |
| | chromosome | abnormalities in Ph+ cells | chromosome |
| | abnormalities in Ph+ | with loss of previously | abnormalities in Ph+ |
| | cells with loss of | obtained response on the | cells with loss of |
| | previously obtained | TKI | previously obtained |
| | response on the TKI | Or | response on the TKI |
| | Or | Any time after initiation | |
| | Any time after initiation | of therapy: Progression | |
| | of therapy: progression | from AP CML to BP | |
| | from CP CML to AP | CML | |
| | CML | | |

Further, the subject is said to be ‘intolerant to prior treatments’ when he/she develops toxicity or toxicities that are persistent and unresponsive to optimal management. The intolerance is categorized as hematologic or non-hematologic intolerance. The patient is said to have developed non-hematologic intolerance to the prior treatments using TKI(s) if the patient develops grade 3 or 4 toxicity while on therapy, or has persistent grade 2 toxicity that is unresponsive to optimal management, including dose adjustments to the lowest doses recommended by the manufacturer unless dose reduction is not considered in the best interest of the patient if the response in the absence of a CCyR for CP CML subjects or MaHR for AP and BP subjects.

The patient is said to have developed ‘hematologic intolerance’ to the prior treatments if the patient develops grade 3 or 4 toxicity while on therapy that is recurrent after dose reduction to the lowest doses recommended by the manufacturer unless dose reduction is not considered in the best interest of the patient in the absence of a CCyR for CP CML subjects or MaHR for AP CML and BP CML subjects.

As used herein, the treatment failure can be defined based on the following criteria as given in table 04:

TABLE 04

| | |
|---|---|
| Time Duration | Treatment failure |

| | | |
|---|---|---|
| 3 | months | No CHR |
| | | BCR-ABL1 ratio >10% |
| | | Ph+ >95% |
| 6 | months | Less than CHR |
| | | BCR-ABL1 ratio >10% |
| | | Ph+ >65% |
| 12 | months | BCR-ABL1 ratio >1% |
| | | Ph+ >35% |

| | |
|---|---|
| Any Time | loss of CHR¹ |
| | loss of CyCR |
| | Any new mutation* |

In patients with treatment failure patient compliance, drug interaction and mutational analysis is evaluated. Bone marrow cytogenetic analysis is to be considered to assess CCyR at 15 months if BCR-ABL1 transcript >1%-10%.

Disease Progression: The date of disease progression is defined as the date any of the criteria for disease progression aa given below in table 05:

TABLE 05
Loss of CHR:
Loss of CHR is defined by meeting any of the following:

| a) | WBC count >20 × 10⁹/L on two occasions at least one month apart in a patient who | |
|---|---|---|
| | never achieved a confirmed complete hematologic response despite having maximally | |
| | tolerated doses of therapy; | |
| b) | Platelet count ≥600 × 10⁹/L on two occasions at least 2 weeks apart; | |
| c) | Appearance of blasts or promyelocytes in peripheral blood; | |
| d) | Appearance of myelocytes + metamyelocytes ≥5% in peripheral blood; and | |
| e) | Progressive splenomegaly refractory to therapy (i.e. ≥5 cm below left intercostal | |
| | margin) | |
| | Disease transformation to Accelerated phase or Blast Phase at any time: | |
| I. | Accelerated Phase | |
| | a) | ≥15% blasts in the peripheral blood or bone marrow aspirate, but <30% blasts |
| | | in both the peripheral blood and bone marrow aspirate; |
| | b) | ≥30% blasts plus promyelocytes in peripheral blood or bone marrow aspirate, |
| | | but <30% blasts in both the peripheral blood and bone marrow aspirate; |
| | c) | ≥20% basophils in the peripheral blood; and |
| | d) | Thrombocytopenia (<100 × 109/L) that is unrelated to therapy* |

OR

| II. | Blast Phase | |
|---|---|---|
| | a) | ≥30% Blast in blood, marrow, or both; and |
| | b) | Extramedullary leukemic cells. |

OR
Death

Treatment Switch: The treatment switch refers to switching the patient from prior tyrosine kinase inhibitor(s) to compound of Formula I or a pharmaceutically acceptable salt thereof. Following are the criteria to switch a patient from prior tyrosine kinase inhibitor(s) to compound of Formula I or a pharmaceutically acceptable salt thereof:
a) failure to achieve CHR after 3 months of treatment initiation;
b) no cytogenetic response (>95% Ph+ cells) in three months after the initiation of therapy;
c) less than a minor cytogenetic response (>65% Ph+) in six months after the initiation of therapy;
d) less than a PCyR (>35% Ph+) in twelve months after the initiation of therapy;
e) loss of cytogenetic response at any time after initiation of therapy (i.e.: shift of the cytogenetic response to at least 1 grade worse from the patient's most recently performed bone marrow cytogenetics);
f) BCR-ABL1 ratio >10% in six months after the initiation of therapy;
g) BCR-ABL1 ratio >1% in twelve months after the initiation of therapy;
h) confirmed loss of MMR in 2 consecutive tests at any time during therapy;
i) loss of CHR, CCyR or PCyR at any time after the initiation of therapy; and
j) development of new clonal chromosome abnormalities in Ph+ cells or development of new BCR-ABL1 mutations with potential to cause resistance to study treatment or with loss of previously obtained response on the TKI (i.e.: Either hematological, cytogenetic or molecular response) at any time after the initiation of therapy.

As used herein, the term “about” when appearing before a range should be understood as referring to both endpoints of the range. In such instances the range should also be understood as including the range defined by the specific endpoints listed, and also including sub-ranges within the listed endpoints. In the instances where “about’ is appearing before a number, it should be understood as the number includes the range of +5%.

As used herein the term “between” when appearing before a range should be understood as referring to both endpoints of the range. In such instances the range should also be understood as including the range defined by the specific endpoints listed, and also including sub-ranges within the listed endpoints.

As used herein, the term “therapeutically effective amount” means an amount of a drug, for example, a compound of Formula I or a pharmaceutically acceptable salt thereof in case of the present invention, which has a desired effect of reducing, curing or alleviating the disease or its symptoms when administered to the subject in need thereof. For the purpose of the present invention, the therapeutically effective amount is the effective amount of compound of Formula I or a pharmaceutically acceptable salt thereof that is used in the treatment of CML or ALL. Such therapeutically effective amount of compound of Formula I or a pharmaceutically acceptable salt thereof can be administered to the patient in need thereof as such or in the form of pharmaceutical formulation. In particular, the compound of Formula I or a pharmaceutically acceptable salt thereof is administered as a solid oral dosage form. The terms “escalation” or re-escalation” can be interchangeable.

Pharmacokinetics (PK) evaluation is one of the endpoints of clinical trials. As known to the skilled person PK evaluation includes, for example, determination of Cₘᵢₙ, Cₘₐₓ, Tₘₐₓ, half-life, C_{avg}, C_{trough}, Terminal rate constant (Kel), AUC₀₋₁₂, AUC₀₋₂₄, Area under the concentration-time curve from time 0 to the last quantifiable time point (AUC₀₋ₜₐᵤ), oral clearance (CL/F), Apparent volume of distribution (V/F), and dose-normalized [AUC₍₀₋ₜₐᵤ)/dose or (Cₘₐₓ/dose)].

As used herein AUC₀₋₂₄ refers to the steady-state area under the plasma concentration versus the time curve from time zero to twenty-four hours after administration of drug (the compound of Formula I or a pharmaceutically acceptable salt thereof in the present case).

The plasma concentrations referred to as Cₘᵢₙ and Cₘₐₓ is the minimum and maximum steady-state effective concentration of the drug in plasma during a particular dosage interval. The time to reach the maximum plasma concentration after administration of the dose is referred to as Tₘₐₓ.

### DETAILED DESCRIPTION OF THE INVENTION

The present invention relates a method of treatment of leukemia. Particularly the present invention relates to the method of treatment of CML and ALL. In a particular embodiment, the method comprises treatment of CP CML, AP CML, BP CML, Ph+ CML and Ph+ ALL. The present invention also relates to method of treatment of Ph+ CML in chronic phase (CP), previously treated with two or more tyrosine kinase inhibitors, T315I-positive CML (chronic phase, accelerated phase, or blast phase) or T315I-positive Ph+ ALL, Newly-diagnosed chronic phase (CP) Ph+ CML, Chronic phase, accelerated phase (AP), or blast phase (BP) Ph+ CML with resistance or intolerance to prior therapy.

In an embodiment, the present invention relates to a method of treatment of an adult patient with refractory CML.

In another embodiment, the present invention relates to a method of treatment of an adult patient with newly diagnosed CML.

According to an embodiment of the present invention, the method comprises administration of a compound of Formula I or a pharmaceutically acceptable salt thereof.

The compound of Formula I, has the below chemical name and formula:
Chemical name: N′-(2-chloro-6-methylbenzoyl)-4-methyl-3-[2-(3-quinolyl) ethynyl]-benzohydrazide

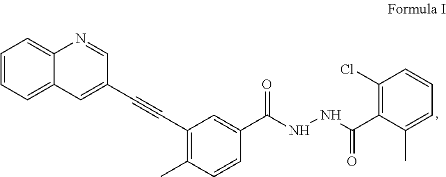 [SMILES: Cc1ccc(C(=O)NNC(=O)c2c(C)cccc2Cl)cc1C#Cc1cnc2ccccc2c1]

International Publication Nos. WO2012098416A1 which is hereby incorporated by reference, discloses the compound of Formula I and its process for preparation.

The compound of Formula I or a pharmaceutically acceptable salt thereof may be administered as an oral dosage form to a leukemic patient. In another embodiment, the compound of Formula I or a pharmaceutically acceptable salt thereof may be administered to a leukemic patient as an oral dosage form comprising the compound of Formula I or a pharmaceutically acceptable salt thereof in an amorphous form and a pharmaceutically acceptable excipient. According to another embodiment, the method comprises oral administration of the compound of Formula I or a pharmaceutically acceptable salt thereof at an initial daily dose to the subject in need thereof and escalating or de-escalating the dose to achieve or maintain a disease response.

The oral dosage form may include a compound of Formula I or a pharmaceutically acceptable salt thereof in its amorphous form, and may further include one or more pharmaceutically acceptable excipients. In some embodiments, the oral dosage form may be a hard gelatin capsule.

Pharmaceutically acceptable excipients that may be included in the oral dosage form of the present invention include, for example, one or more of polyvinyl caprolactam, polyvinyl acetate, polyethylene glycol graft co-polymer, silicon dioxide, sodium lauryl sulphate, silicified microcrystalline cellulose, crospovidone, and/or gelatin.

The desired dosage form of the compound of Formula I or a pharmaceutically acceptable salt thereof is an oral dosage form containing from 10 mg to 300 mg of the compound of Formula I or a pharmaceutically acceptable salt thereof, as measured according to a daily dose. The daily dose may be administered over the course of one to four daily administrations. In certain embodiments, the amount of the compound of Formula I or a pharmaceutically acceptable salt thereof in the dosage form is between 10 mg to 210 mg, as measured by the amount of the compound of Formula I or a pharmaceutically acceptable salt thereof administered daily. The dose may be administered as a single daily dose. The dose may be administered as a multiple daily dose.

The total daily dose of compound of Formula I or a pharmaceutically acceptable salt thereof administered to a subject may be between 10 mg to 210 mg. In some embodiments, the total daily dose of the compound of Formula I or a pharmaceutically acceptable salt thereof is 10 mg. In some embodiments, the total daily dose of the compound of Formula I or a pharmaceutically acceptable salt thereof is 12 mg. In some embodiments, the total daily dose of the compound of Formula I or a pharmaceutically acceptable salt thereof is 24 mg. In some embodiments, the total daily dose of the compound of Formula I or a pharmaceutically acceptable salt thereof is 48 mg. In other embodiments, the total daily dose of the compound of Formula I or a pharmaceutically acceptable salt thereof is 66 mg. In other embodiments, the total daily dose of the compound of Formula I or a pharmaceutically acceptable salt thereof is 90 mg. In other embodiments, the total daily dose of the compound of Formula I or a pharmaceutically acceptable salt thereof is 126 mg. In other embodiments, the total daily dose of the compound of Formula I or a pharmaceutically acceptable salt thereof is 174 mg. In other embodiments, the total daily dose of the compound of Formula I or a pharmaceutically acceptable salt thereof is 204 mg. In some embodiments, the total daily dose of the compound of Formula I or a pharmaceutically acceptable salt thereof is 210 mg. The present inventors found that according to their studies, the highest efficacious and tolerable daily dose of the compound of Formula I or a pharmaceutically acceptable salt thereof is 204 mg in patients with prior TKI exposure, mutations and ACAs, and dose-limiting toxicities were present at daily doses of 240 mg.

In an embodiment, for newly diagnosed CML patients, an effective dose of the compound of Formula I or a pharmaceutically acceptable salt thereof may be from 10 mg to 240 mg. More particularly the effective dose is from 10 mg to 210 mg.

In an embodiment, the daily dose of between 10 mg and 210 mg of compound of Formula I or a pharmaceutically acceptable salt thereof when administered to a subject, may result in a mean AUC₀₋₂₄ ranging from 1000 ng*h/mL to 120,000 ng*h/mL.

In an embodiment, the daily dose of between 10 mg and 210 mg of compound of Formula I or a pharmaceutically acceptable salt thereof when administered to a subject, may result in a mean Cₘₐₓ ranging from 100 ng/ml to 9000 ng/ml.

According to an embodiment, the method of present invention comprises oral administration of a compound of Formula I or a pharmaceutically acceptable salt thereof at an initial daily dose and escalating or de-escalating the dose to achieve or maintain a disease response, with no severe adverse reaction.

In an embodiment, the initial daily dose is selected from the dose between 10 mg to 210 mg. In another embodiment, the initial daily dose is selected from 12 mg to 210 mg. In a particular embodiment, the initial daily dose is selected from 12 mg, 24 mg, 48 mg, 66 mg, 90 mg, 126 mg, 174 mg and 204 mg. In a more particular embodiment, the initial daily dose is 174 mg.

In an aspect of the present invention, the initial daily dose is escalated to achieve or maintain a disease response.

According to an embodiment, the initial daily dose is escalated to subsequent higher daily dose. In some embodiments, the initial daily dose of 12 mg is escalated to a daily dose selected from 24 mg, 48 mg, 66 mg, 90 mg, 126 mg, 174 mg, and 204 mg. In some embodiments, the initial daily dose of 24 mg is escalated to a daily dose selected from 48 mg, 66 mg, 90 mg, 126 mg, 174 mg, and 204 mg. In some embodiments, the initial daily dose of 48 mg is escalated to a daily dose selected from 66 mg, 90 mg, 126 mg, 174 mg, and 204 mg. In some embodiments, the initial daily dose of 66 mg is escalated to a daily dose selected from 90 mg, 126 mg 174 mg, and 204 mg. In some embodiments, the initial daily dose of 90 mg is escalated to a daily dose selected from 126 mg 174 mg, and 204 mg. In some embodiments, the initial daily dose of 126 mg is escalated to a daily dose selected from 174 mg and 204 mg. In some embodiments, the initial daily dose of 174 mg is escalated to a daily dose of 204 mg.

According to an embodiment, an initial dose of a compound of Formula I or a pharmaceutically acceptable salt thereof was escalated to maintain or achieve a disease response, wherein the disease response includes hematologic response, cytogenetic response and molecular response. It would be appreciated by the person skilled in the art that the disease response is stepwise, wherein the first response in a subject is hematologic response which may be partial or complete hematologic response. The subject with complete hematologic response is then tested for cytogenetic response which may be again a partial or complete and the patient showing cytogenetic response is expected to have molecular response, specifically major molecular response.

In an embodiment, the refractory subject may maintain the same disease response that was achieved during previous treatments. For example, if the subject had achieved a complete hematologic response in the previous treatment, but had to discontinue the treatment due to an adverse event, the patient may end up achieving the same response using the compound of Formula I or a pharmaceutically acceptable salt thereof. In an embodiment the refractory subject may achieve complete hematologic response, complete cytogenetic response and major molecular response even if it was not achieved by any of the previous treatments. Using the methods of the present invention the newly diagnosed subjects are expected to achieve complete hematologic response, complete cytogenetic response and major molecular response, sequentially.

It was found that the subjects that were non-responsive to prior TKIs, when treated with the compound of Formula I or a pharmaceutically acceptable salt thereof, surprisingly showed good disease responses. For example, in refractory patients who had undergone prior treatments with 2 or more tyrosine kinase inhibitors (TKIs), complete hematologic response was seen in 29 (70.7%) out of 41 enrolled subjects and complete cytogenetic response was seen in 23 (56.1%) out of 41 enrolled subjects, while 18 (43.9%) of the 41 enrolled subjects reported major molecular response, open-label, dose-ranging, single-agent, multi-center, multi-dose, dose-escalation study using the compound of Formula I or a pharmaceutically acceptable salt thereof.

In an embodiment, the subject may achieve disease response at a particular daily dose in 7 days of treatment. In an embodiment, the subject may achieve disease response at a particular daily dose in 14 days of treatment. In an embodiment, the subject may achieve disease response at a particular daily dose in 21 days of treatment. In an embodiment, the subject may achieve disease response at a particular daily dose in 28 days of treatment. In an embodiment, the subject may achieve disease response at a particular dose in 3 months of treatment. In an embodiment, the subject may achieve disease response at a particular dose in 6 months of treatment.

In a particular embodiment, the subject may achieve complete hematologic response at a particular daily dose in 21 days of treatment. In a particular embodiment, the subject may achieve complete hematologic and partial cytogenetic response at a particular daily dose in 28 days of treatment. In a particular embodiment, the subject may achieve complete hematologic and complete cytogenetic response at a particular daily dose in 28 days of treatment. In a particular embodiment, the subject may achieve complete hematologic, complete cytogenetic and major molecular response at a particular daily dose in 3 months of treatment.

It will be understood by the skilled person that the disease response may vary in different patients based on for example patient's age, disease history, prior treatment, physical and vital conditions and dose of the compound of Formula I or a pharmaceutically acceptable salt thereof. Although the majority of the patients studied in the present invention developed at least a hematologic response (partial or complete) within the 1^{st} cycle (28 days) of the treatment, it can be expected that some of the patients may develop a disease response after 2 or 3 months of treatment. In an embodiment, the treatment at a particular daily dose using a compound of Formula I or a pharmaceutically acceptable salt thereof may be continued for at least 28 days even in the absence of disease response, before escalating the daily dose to the subsequent dose. In an embodiment, the treatment at a particular daily dose using the compound of Formula I or a pharmaceutically acceptable salt thereof may be continued for at least 3 months even in the absence of disease response, before escalating the daily dose to the subsequent dose. In an embodiment, the treatment at a particular daily dose using the compound of Formula I or a pharmaceutically acceptable salt thereof may be continued for at least 6 months even in the absence of disease response, before escalating the daily dose to the subsequent dose.

According to an embodiment, the initial daily dose is escalated to subsequent higher daily dose, wherein the escalated dose results in a mean AUC₀₋₂₄ ranging from 1000 ng*h/mL to 120,000 ng*h/mL.

According to an embodiment, the initial daily dose is escalated to subsequent higher daily dose, wherein the escalated dose results in a mean Cₘₐₓ ranging from 100 ng/mL to 9000 ng/mL.

According to an embodiment, the initial daily dose is escalated to achieve or maintain a disease response, with no severe adverse reaction.

The adverse reactions or adverse events (AEs) are categorized based on NCI-CTCAE v 5.0 (https://evs.nci.nih.gov/ftp1/CTCAE/About.html). For those AEs without assigned CTCAE grades, the recommendation in the CTCAE criteria that converts mild, moderate and severe events into CTCAE grades may be considered. The AEs in a patient may be ‘unrelated’ as they are developed due to extraneous causes such as medical history, demography details, disease and environment or are ‘unlikely’, which does not follow a reasonable temporal sequence or could also be explained by patient's concurrent disease, environmental factors, medical history and other concomitant drugs or chemicals including food drug interactions. It will be appreciated by the skilled person that the dose modification, specifically dose de-escalation would be necessary only if the adverse event is due to the drug (compound of Formula I or a pharmaceutically acceptable salt thereof). The unrelated and unlikely adverse event may not lead to dose modifications.

The methods of determining whether the AEs are due to the compound of Formula I or a pharmaceutically acceptable salt thereof, due to underlying disease or due to extraneous causes are within the scope of the knowledge of a person skilled in the art. For example, in AP CML and BP CML, patients having grade 3 or grade 4 myelosuppression might be attributable to disease rather than to the compound of Formula I or a pharmaceutically acceptable salt thereof. In such a case, a bone marrow biopsy was performed to distinguish between toxicity and anti-leukemic effects. If the patient had a normocellular marrow or persistence of the disease, the treatment was continued with supportive medication. If there was evidence of toxicity due to the compound of Formula I or a pharmaceutically acceptable salt thereof such as hypocellular marrow, then the treatment was stopped for up to four weeks to allow the event to ameliorate to ≤Grade 1 or baseline and re-initiated at reduced dose. Although bone marrow biopsy is one of the methods to distinguish between the toxicity due to the drug or symptoms of underlying disease, the embodiments of this application include all the methods known to the skilled person for this purpose. The skilled person may implement other known methods to distinguish between the toxicity due to the compound of Formula I or a pharmaceutically acceptable salt thereof or the symptoms of underlying diseases.

In an embodiment of the present invention, the daily dose is withheld when the patient experiences adverse events due to the compound of Formula I or a pharmaceutically acceptable salt thereof. In an embodiment, the daily dose of compound of Formula I or a pharmaceutically acceptable salt thereof may be withheld until the patient recovers from the adverse events. In another embodiment, the initial daily dose may be withheld for a period of at least 7 days. In an embodiment, the initial daily dose is withheld for a period of at least 14 days. In an embodiment, the initial daily dose is withheld for a period of at least 28 days. In an embodiment, the initial daily dose is withheld for a period of at least 48 days. In an embodiment, the initial daily dose is withheld for a period of at least 56 days.

When the patient experiences an AE due to the compound of Formula I or a pharmaceutically acceptable salt thereof at a given daily dose, said dose is withheld for a particular period and then the daily dose is resumed at the given dose or at de-escalated daily dose. In particular, the daily dose is resumed after the patient recovers from said AE. The subject was said to be recovered from the adverse event when the toxicity levels of that AE is reduced to grade 1 as per NCI-CTCAE version 5.0 or were completely resolved or were resolved to baseline. It will be understood by the skilled person that the patient is said to be recovered from the AE based on the comparison of patient's assessment reports before and after initiation of treatment using the compound of Formula I or a pharmaceutically acceptable salt thereof. In some embodiments, the patient may undergo treatment for the AE. In some embodiments, the patient may undergo optimal management of the AE. In some embodiments, the AE may be resolved unaided. The treatment for the AEs and the optimal management of the same is within the scope of the skilled person.

According to another embodiment, the initial daily dose is de-escalated to a subsequent dose when the patient develops severe adverse reaction.

According to an embodiment, an initial daily dose is de-escalated to a subsequent lower daily dose. In some embodiments, an initial daily dose of 24 mg is de-escalated to a daily dose of 12 mg. In some embodiments, an initial daily dose of 48 mg is de-escalated to a daily dose selected from 12 mg and 24 mg. In some embodiments, an initial daily dose of 66 mg is de-escalated to a daily dose selected from 12 mg, 24 mg and 48 mg. In some embodiments, an initial daily dose of 90 mg is de-escalated to a daily dose selected from 12 mg, 24 mg, 48 and 66 mg. In some embodiments, an initial daily dose of 126 mg is de-escalated to a daily dose selected from 12 mg, 24 mg, 48 mg, 66 mg and 90 mg. In some embodiments, an initial daily dose of 174 mg is de-escalated to a daily dose of 12 mg, 24 mg, 48 mg, 66 mg, 90 mg or 126 mg. In some embodiments, an initial daily dose of 204 mg is de-escalated to a daily dose of 12 mg, 24 mg, 48 mg, 66 mg, 90 mg, 126 mg or 174 mg. In some embodiments, an initial daily dose of 180 mg is de-escalated to a daily dose of 45 mg, 90 mg or 135 mg. In some embodiments, an initial daily dose of 174 mg is de-escalated to a daily dose of 43.5 mg, 87 mg or 130.5 mg. In some embodiments, an initial daily dose of 135 mg is de-escalated to a daily dose of 45 mg, and 90 mg. In some embodiments, an initial daily dose of 130.5 mg is de-escalated to a daily dose of 43.5 mg, and 87 mg.

According to an embodiment, the initial daily dose is de-escalated to a lower daily dose, wherein the de-escalated dose results in a mean AUC₀₋₂₄ ranging from 1000 ng*h/mL to 120,000 ng*h/mL.

According to an embodiment, the initial daily dose is de-escalated to the lower daily dose, wherein the de-escalated dose results in a mean Cₘₐₓ ranging from 100 ng/ml to 9000 ng/mL.

According to an embodiment, dose delays (dose withholding) or dose reductions or both were implemented for patients who experienced adverse drug reactions due to the administration of the compound of Formula I or a pharmaceutically acceptable salt thereof.

In some embodiments, patients that experience grade 1 or 2 hematological or non-hematological toxicity due to the compound of Formula I or a pharmaceutically acceptable salt thereof may continue the treatment without any dose delays or de-escalation. In some embodiments, patients that experience grade 1 or 2 hematological or non-hematological toxicity due to the compound of Formula I or a pharmaceutically acceptable salt thereof may continue the treatment without any dose delays or de-escalation, while undergoing supportive care and management of AEs.

In some embodiments, patients that experience grade 1 or 2 non-hematological toxicity due to the compound of Formula I or a pharmaceutically acceptable salt thereof may undergo dose delay for a period of at least 7 days. In some embodiments, patients that experience grade 1 or 2 non-hematological toxicity due to compound of Formula I or a pharmaceutically acceptable salt thereof may undergo dose delay for a period of at least 14 days. In some embodiments, patients that experience grade 1 or 2 non-hematological toxicity due to the compound of Formula I or a pharmaceutically acceptable salt thereof may undergo dose delay for a period of at least 28 days. In some embodiments, patients that experience grade 1 or 2 non-hematological toxicity due to the compound of Formula I or a pharmaceutically acceptable salt thereof may undergo dose delay for a period of not more than 28 days. A patient may have to delay the dose (or withhold) when grade 1 or 2 non-hematological toxicities are intolerable either due to clinical symptoms or due to interference with daily activities and if such toxicities are not controlled by optimal supportive care or optimal management.

In some embodiment, for a patient experiencing grade 3 hematological or non-hematological toxicity or grade 4 asymptomatic hematological toxicities, treatment may be withheld for at least 14 days. In some embodiment, for a patient experiencing grade 3 hematological or non-hematological toxicity or grade 4 asymptomatic hematological toxicities, treatment may be withheld for at least 28 days. In some embodiment, for a patient experiencing grade 3 hematological or non-hematological toxicity or grade 4 asymptomatic hematological toxicities, treatment may be withheld for at least 56 days. In some embodiment, for a patient experiencing grade 3 hematological or non-hematological toxicity or grade 4 asymptomatic hematological toxicities, treatment may be withheld for not more than 28 days. In some embodiment, for a patient experiencing grade 3 hematological or non-hematological toxicity or grade 4 asymptomatic hematological toxicities, treatment may be withheld for not more than 56 days.

In some embodiments, the daily dose may be resumed at the same daily dose. In some embodiments, the daily dose may be resumed at a de-escalated dose. In a particular embodiment, a patient experiencing a recurrence of AEs may undergo treatment using a de-escalated daily dose of the compound of Formula I or a pharmaceutically acceptable salt thereof.

According to an embodiment of the present invention, the de-escalated daily dose may be optionally re-escalated to a dose selected from 24 mg to 210 mg, provided the patient does not develop an AE. The escalation of the dose may be carried out as discussed above.

The method of treatment according to the present invention may be discontinued in case the subject does not have any disease response after treatment using a maximum tolerable dose for 3 to 6 months. The treatment may also be discontinued in a subject who is intolerant to the minimum amount of dose. A subject having un-manageable adverse events due to a daily dose of 12 mg in newly diagnosed patients or 48 mg in refractory patients may be discontinued from the treatment. The discontinuation may also occur when the subject voluntarily does so or in the event of death.

In a particular embodiment, the present invention relates to a method for the treatment of an adult patient with CML comprising, administering a therapeutically effective amount of a compound of Formula I or a pharmaceutically acceptable salt thereof at an initial daily dose of 174 mg, wherein the initial dose is escalated to 204 mg to achieve or maintain complete hematologic response (CHR), with no severe adverse reaction.

In another particular embodiment, the present invention relates to a method for the treatment of an adult patient with CML, comprising orally administering a therapeutically effective amount of a compound of Formula I or a pharmaceutically acceptable salt thereof at an initial daily dose of 174 mg, wherein the initial dose is escalated to 204 mg to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In a particular embodiment, the present invention relates to a method for the treatment of an adult patient with refractory CML, wherein the method comprises administering to the patient a therapeutically effective amount of an initial daily dose of 174 mg of the compound of Formula I or a pharmaceutically acceptable salt thereof such that when the patient develops non-hematological toxicity:
a) the initial daily dose is withheld for at least 7 days and resumed after recovery of patient from said toxicity; or
b) the initial daily dose is withheld for at least 7 days and resumed at a reduced daily dose of 126 mg on first instance and then to 90 mg, 66 mg and 48 mg on subsequent instances of recurrences of said toxicity.

In a particular embodiment, the present invention relates to a method for the treatment of an adult patient with refractory CML, wherein the method comprises administering to said patient a therapeutically effective amount of an initial daily dose of 174 mg of a compound of Formula I or a pharmaceutically acceptable salt thereof such that when the patient develops non-hematologic toxicity:
a) the initial daily dose is withheld for at least 7 days and resumed at 174 mg after recovery of patient from said toxicity; or
b) the initial daily dose is withheld for at least 7 days and resumed at a reduced daily dose of 130.5 mg on first instance and to 87 mg, and 43.5 mg on subsequent instances of recurrences of said toxicity.

In a particular embodiment, the present invention relates to a method for the treatment of an adult patient with newly diagnosed CML, wherein the method comprises administering to the patient a therapeutically effective amount of an initial daily dose of 174 mg of the compound of Formula I or a pharmaceutically acceptable salt thereof such that when the patient develops non-hematological toxicity:
a) the initial daily dose is withheld for at least 7 days and resumed after recovery of patient from said toxicity; or
b) the initial daily dose is withheld for at least 7 days and then resumed at a reduced daily dose of 126 mg on first instance and to 90 mg, 66 mg, 48 mg, 24 mg and 12 mg on subsequent instances of recurrences of said toxicity.

In a particular embodiment, the present invention relates to a method for the treatment of an adult patient with newly diagnosed CML, wherein the method comprises administering to said patient a therapeutically effective amount of an initial daily dose of 174 mg of a compound of Formula I or a pharmaceutically acceptable salt thereof such that when the patient develops non-hematologic toxicity:
a) the initial daily dose is withheld for at least 7 days and resumed at 174 mg after recovery of patient from said toxicity; or
b) the initial daily dose is withheld for less than 7 days and if toxicity is resolved then resumed at 174 mg; or
c) the initial daily dose is withheld for at least 7 days and resumed at a reduced daily dose of 130.5 mg on first instance and to 87 mg, and 43.5 mg on subsequent instances of recurrences of said toxicity.

In another particular embodiment, the present invention relates to a method for the treatment of an adult patient with newly diagnosed CML, wherein the method comprises administering to said patient a therapeutically effective amount of an initial daily dose of 174 mg of a compound of Formula I or a pharmaceutically acceptable salt thereof such that when the patient develops non-hematologic toxicity:
a) the initial daily dose is withheld for at least 7 days and resumed at 174 mg after recovery of patient from said toxicity; or
b) the initial daily dose is withheld for less than 7 days and if toxicity is resolved then resumed at 174 mg; or
c) the initial daily dose is withheld for at least 7 days and resumed at a reduced daily dose of 130.5 mg on first instance and to 87 mg, and 43.5 mg on subsequent instances of recurrences of said toxicity;
and wherein the daily dose of the compound of Formula I or a pharmaceutically acceptable salt thereof in the patient results in mean AUC₀₋₂₄ ranging from 28598 ng*h/mL to 42898±33827 ng*h/mL.

In one particular embodiment, the present invention provides a method for the treatment of a human patient with chronic myeloid leukemia (CML) (such as a newly diagnosed chronic myeloid leukemia (CML) or a refractory chronic myeloid leukemia (CML)), comprising administering to the patient a therapeutically effective amount of compound of Formula I:

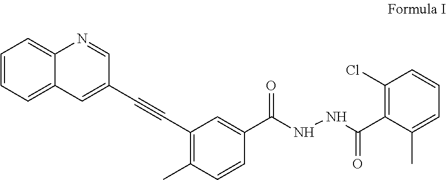 [SMILES: Cc1ccc(C(=O)NNC(=O)c2c(C)cccc2Cl)cc1C#Cc1cnc2ccccc2c1]

or a pharmaceutically acceptable salt thereof,
wherein the chronic myeloid leukemia (CML) is chronic, accelerated, or blast phase Philadelphia Chromosome Positive Chronic Myeloid Leukemia (Ph+ CML).

According to any one of the embodiments described herein, the therapeutically effective amount of compound of Formula I or its pharmaceutically salt is sufficient to achieve a mean AUC₀₋₂₄ ranging from 6226±5827 ng*h/mL to 60373±55659 ng*h/mL or a mean Cₘₐₓ ranging from 664±450 ng/mL to 5054±3051 ng/mL.

According to any one of the embodiments described herein, the compound of Formula I or its pharmaceutically salt is administered at an initial daily dose of 10 mg to 204 mg.

According to any one of the embodiments described herein, the compound of Formula I or its pharmaceutically salt is administered at an initial daily dose selected from 66 mg, 87 mg, 90 mg, 126 mg, 130.5 mg, 135 mg, 174 mg and 180 mg.

According to any one of the embodiments described herein, the initial daily dose is escalated or de-escalated to achieve or maintain at least one of (a) a complete hematological response, (b) a complete hematological response and partial cytogenetic response, (c) a complete hematological response and complete cytogenetic response, or (d) a complete hematological response, complete cytogenetic response, and major molecular response.

According to any one of the embodiments described herein, the initial daily dose is escalated or de-escalated with no severe adverse reaction.

According to any one of the embodiments described herein, the patient is resistant or intolerant to at least one tyrosine kinase inhibitor.

According to any one of the embodiments described herein, the at least one tyrosine kinase inhibitor is a second-generation tyrosine kinase inhibitor.

According to any one of the embodiments described herein, the at least one tyrosine kinase inhibitor is selected from dasatinib, nilotinib, radotinib and bosutinib.

According to any one of the embodiments described herein, the at least one tyrosine kinase inhibitor is a third-generation tyrosine kinase inhibitor.

According to any one of the embodiments described herein, the at least one tyrosine kinase inhibitor is selected from ponatinib and asciminib.

According to any one of the embodiments described herein, the patient has one or more of the following mutations in the BCR-ABL1 kinase domain: M244V, L248V, E499E, Q252H, P223S, A337T, F359C, E255V, Y253F, F317L, Y253H, V299L, E255V, F317L, E255V, F359V, G250E, E255K, F359V, M351T, G250E, H369R, H396R, and V359I.

According to any one of the embodiments described herein, the patient has one or more of the following characteristics:
a) 15% blasts in peripheral blood and bone marrow;
b) <30% blasts plus promyelocytes in peripheral blood and bone marrow;
c) <20% basophils in the peripheral blood;
d) ≥50×10⁹/L (≥50,000/mm³) platelets;
e) Transient prior therapy related thrombocytopenia (<50,000/mm³ for ≤30 days prior to screening);
f) No evidence of extramedullary leukemic involvement, with the exception of hepatosplenomegaly;
g) ≥15 to <30% blasts in peripheral blood or bone marrow;
h) ≥20% basophils in peripheral blood or bone marrow;
i) ≥30% (blasts+promyelocytes) in peripheral blood or bone marrow (but <30% blasts);
j) ≤100×109 platelets/L in peripheral blood unrelated to therapy;
k) Additional clonal cytogenetic abnormalities in Ph+ cells;
l) ≥30% blasts in peripheral blood, bone marrow or both; and
m) extra-medullary disease.

According to any one of the embodiments described herein, the patient does not have T315I-positive CML.

According to any one of the embodiments described herein, the treatment is withheld for a period of at least 7 days, when the patient exhibits a grade 1 or grade 2 non-hematological adverse event, and then reinitiated.

According to any one of the embodiments described herein, the treatment is withheld for a period of up to 56 days (e.g., 14 days to 56 days, or 28 days to 56 days), when the patient exhibits a grade 3 hematological or non-hematological adverse event or grade 4 asymptomatic hematological adverse event, and then reinitiated.

According to any one of the embodiments described herein, the treatment is discontinued when the patient does not recover to a grade 1 adverse event or less after a withholding period of 56 days.

In one particular embodiment, the present invention provides a method of treating a treatment-resistant human patient having chronic myeloid leukemia (CML) comprising administering a therapeutically effective amount of a compound of Formula I:

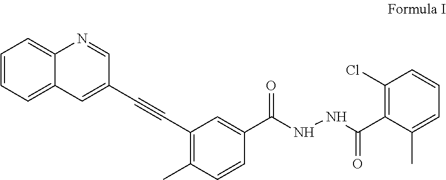 [SMILES: Cc1ccc(C(=O)NNC(=O)c2c(C)cccc2Cl)cc1C#Cc1cnc2ccccc2c1]

or a pharmaceutically acceptable salt thereof to the patient, wherein
   i. the method includes orally administering 174 to 200 mg of the compound of Formula I daily, and
   ii. the patient is resistant or intolerant to at least one tyrosine kinase inhibitor prior to the administration of the compound of Formula I.

According to any one of the embodiments described herein, the patient has Ph+ CML.

According to any one of the embodiments described herein, the patient is in the chronic phase of CML.

According to any one of the embodiments described herein, the at least one tyrosine kinase inhibitor is a second-generation tyrosine kinase inhibitor.

According to any one of the embodiments described herein, the second-generation tyrosine kinase inhibitor is selected from dasatinib, nilotinib, radotinib and bosutinib.

According to any one of the embodiments described herein, the at least one tyrosine kinase inhibitor is a third-generation tyrosine kinase inhibitor.

According to any one of the embodiments described herein, the third-generation tyrosine kinase inhibitor is selected from ponatinib and asciminib.

According to any one of the embodiments described herein, the patient has one or more of the following mutations in the BCR-ABL1 kinase domain: M244V, L248V, E499E, Q252H, P223S, A337T, F359C, E255V, Y253F, F317L, Y253H, V299L, E255V, F317L, E255V, F359V, G250E, E255K, F359V, M351T, G250E, H369R, H396R, and V359I.

According to any one of the embodiments described herein, the patient does not have T315I-positive CML.

According to any one of the embodiments described herein, the compound of Formula I is orally administered in the form of oral capsules.

According to any one of the embodiments described herein, the capsules each contain 43.5 mg, 45 mg, 48 mg or 50 mg of the compound of Formula I.

According to any one of the embodiments described herein, the compound of Formula I is administered each morning and optionally with two hours of fasting before and after administration of the compound of Formula I.

According to any one of the embodiments described herein, upon the patient exhibiting a grade 1 or grade 2 non-hematological adverse event which was intolerable due to clinical symptoms or interference with daily activities, the treatment is withheld for a period of time and then reinitiated.

According to any one of the embodiments described herein, the period of time during which the treatment is withheld is 7 days.

According to any one of the embodiments described herein, the period of time during which the treatment is withheld is 14 days.

According to any one of the embodiments described herein, upon the patient exhibiting a grade 3 hematological or non-hematological adverse event or grade 4 asymptomatic hematological adverse event, the treatment is withheld for a period of time and then reinitiated.

According to any one of the embodiments described herein, the treatment is withheld for up to 56 days (e.g., 14 days to 56 days, or 28 days to 56 days) for recovery to a grade 1 adverse event or less; and where the patient does not recover to a grade 1 adverse event or less, treatment with the compound of Formula I is discontinued.

According to any one of the embodiments described herein, the method includes orally administering one or more oral capsules daily, where (i) each capsule contains the same amount of the compound of Formula I, (ii) the amount is selected from 43.5 mg, 45 mg, 48 mg and 50 mg of the compound of Formula I, and (iii) upon the occurrence of certain adverse events not requiring discontinuation or temporary withholding treatment, the daily dosage is reduced by administering a fewer number of the same oral capsules daily.

According to any one of the embodiments described herein, prior to a dose reduction, 4 oral capsules are administered daily.

In one particular embodiment, the present invention provides a method for the treatment of a human patient with chronic myeloid leukemia (CML) (such as a newly diagnosed chronic myeloid leukemia (CML) or a refractory chronic myeloid leukemia (CML)) comprising administering to the patient a therapeutically effective amount of compound of Formula I:

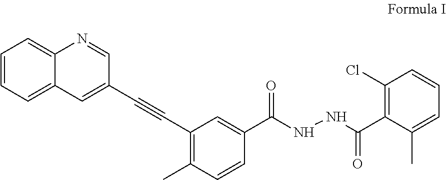 [SMILES: Cc1ccc(C(=O)NNC(=O)c2c(C)cccc2Cl)cc1C#Cc1cnc2ccccc2c1]

or a pharmaceutically acceptable salt thereof at an initial daily dose of 50 mg to 200 mg (e.g. any one of the dose selected from 50 mg, 66 mg, 87 mg, 90 mg, 96 mg, 100 mg, 126 mg, 130.5 mg, 135 mg, 150 mg, 174 mg, 180 mg, 192 mg and 200 mg) such that when the patient develops hematologic and/or non-hematologic toxicity:
   a) the initial daily dose is withheld for at least 7 days and resumed at the initial daily dose of 50 mg to 200 mg (e.g. any one of the dose selected from 50 mg, 66 mg, 87 mg, 90 mg, 96 mg, 100 mg, 126 mg, 130.5 mg, 135 mg, 150 mg, 174 mg, 180 mg, 192 mg and 200 mg); or
   b) the initial daily dose is withheld for less than 7 days and if the toxicity is resolved, then the treatment is resumed at the initial daily dose of 50 mg to 200 mg (e.g. any one of the dose selected from 50 mg, 66 mg, 87 mg, 90 mg, 96 mg, 100 mg, 126 mg, 130.5 mg, 135 mg, 150 mg, 174 mg, 180 mg, 192 mg and 200 mg); or
   c) the initial daily dose is withheld for at least 7 days and resumed at a reduced daily dose of 43.5 mg to 192 mg (e.g. any one of the selected from 43.5 mg, 45 mg, 48 mg, 50 mg, 66 mg, 87 mg, 90 mg, 126 mg, 130.5 mg, 135 mg, 174 mg, 180 mg and 192 mg);
      and wherein, the reduced daily dose is optionally re-escalated to a re-escalated daily dose of 50 mg to 200 mg (e.g. any one of the dose selected from 50 mg, 66 mg, 87 mg, 90 mg, 96 mg, 100 mg, 126 mg, 130.5 mg, 135 mg, 150 mg, 174 mg, 180 mg, 192 mg, and 200 mg); or
   d) the initial daily dose is withheld for at least 7 days and resumed at a reduced daily dose of 43.5 mg to 192 mg (e.g. any one of the dose selected from 43.5 mg, 45 mg, 48 mg, 50 mg, 66 mg, 87 mg, 90 mg, 126 mg, 130.5 mg, 135 mg, 174 mg, 180 mg and 192 mg);
      and wherein, the reduced daily dose is further reduced to a subsequent reduced daily dose of 43.5 mg to 180 mg (e.g. any one of the dose selected from 43.5 mg, 45 mg, 48 mg, 50 mg, 66 mg, 87 mg, 90 mg, 126 mg, 130.5 mg, 135 mg, 174 mg and 180 mg) on subsequent instances of recurrences of the toxicity;
      and wherein, the reduced daily dose or the subsequent reduced daily dose is optionally re-escalated to a re-escalated daily dose of 50 mg to 200 mg (e.g. any one of the dose selected from 50 mg, 66 mg, 87 mg, 90 mg, 126 mg, 130.5 mg, 135 mg, 174 mg, 180 mg, 192 mg, and 200 mg).

In another particular embodiment, the present invention provides a method for the treatment of a human patient with chronic myeloid leukemia (CML) (such as a newly diagnosed chronic myeloid leukemia (CML) or a refractory chronic myeloid leukemia (CML)) comprising administering to the patient a therapeutically effective amount of compound of Formula I:

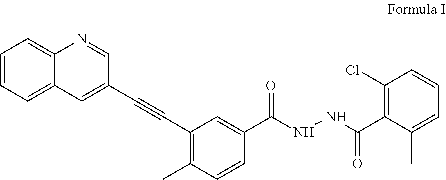 [SMILES: Cc1ccc(C(=O)NNC(=O)c2c(C)cccc2Cl)cc1C#Cc1cnc2ccccc2c1]

or a pharmaceutically acceptable salt thereof at an initial daily dose of 174 mg such that when the patient develops hematologic and/or non-hematologic toxicity:
   a) the initial daily dose is withheld for at least 7 days and resumed at the initial daily dose of 174 mg; or
   b) the initial daily dose is withheld for less than 7 days and if the toxicity is resolved, then the treatment is resumed at the initial daily dose of 174 mg; or c) the initial daily dose is withheld for at least 7 days and resumed at a reduced daily dose of 43.5 mg to 135 mg (e.g. any one of the dose selected from 43.5 mg, 45 mg, 48 mg, 50 mg, 66 mg, 87 mg, 90 mg, 126 mg, 130.5 mg, and 135 mg), preferably the reduced daily dose is 87 mg;
      and wherein, the reduced daily dose is optionally re-escalated to a re-escalated daily dose up to 200 mg (e.g. any one of the dose selected from 50 mg, 66 mg, 87 mg, 90 mg, 96 mg, 100 mg, 126 mg, 130.5 mg, 135 mg, 150 mg, 174 mg, 180 mg, 192 mg, and 200 mg), preferably the re-escalated daily dose is 200 mg; or
   d) the initial daily dose is withheld for at least 7 days and resumed at a reduced daily dose of 43.5 mg to 135 mg (e.g. any one of the dose selected from 43.5 mg, 45 mg, 48 mg, 50 mg, 66 mg, 87 mg, 90 mg, 126 mg, 130.5 mg, and 135 mg), preferably the reduced daily dose is 130.5 mg; or
      and wherein, the reduced daily dose is optionally further reduced to a subsequent reduced daily dose of 43.5 mg to 130.5 mg (e.g. any one of the dose selected from 43.5 mg, 45 mg, 48 mg, 50 mg, 66 mg, 87 mg, 90 mg, 126 mg, and 130.5 mg) on subsequent instances of recurrences of the toxicity, preferably the subsequent reduced daily dose is 87 mg;
      and wherein, the reduced daily dose or the subsequent reduced daily dose is optionally re-escalated to a re-escalated daily dose up to 200 mg (e.g. any one of the dose selected from 50 mg, 66 mg, 87 mg, 90 mg, 126 mg, 130.5 mg, 135 mg, 174 mg, 180 mg, 192 mg, and 200 mg), preferably the re-escalated daily dose is 200 mg.

In another particular embodiment, the present invention provides, a method for the treatment of a human patient with chronic myeloid leukemia (CML) (such as a newly diagnosed chronic myeloid leukemia (CML) or a refractory chronic myeloid leukemia (CML)) comprising administering to the patient a therapeutically effective amount of compound of Formula I:

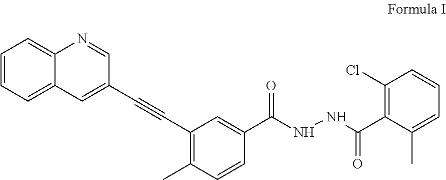 [SMILES: Cc1ccc(C(=O)NNC(=O)c2c(C)cccc2Cl)cc1C#Cc1cnc2ccccc2c1]

or a pharmaceutically acceptable salt thereof at an initial daily dose of 87 mg such that when the patient develops hematologic and/or non-hematologic toxicity:
   a) the initial daily dose is withheld for at least 7 days and resumed at the initial daily dose of 87 mg; or
   b) the initial daily dose is withheld for less than 7 days and if the toxicity is resolved, then the treatment is resumed at the initial daily dose of 87 mg; or
   c) the initial daily dose is withheld for at least 7 days and resumed at a reduced daily dose of 43.5 mg or 50 mg, preferably the reduced daily dose is 43.5 mg;
      and wherein, the reduced daily dose is optionally re-escalated to a re-escalated daily dose up to 200 mg (e.g. any one of the dose selected from 50 mg, 66 mg, 87 mg, 90 mg, 96 mg, 100 mg, 126 mg, 130.5 mg, 135 mg, 150 mg, 174 mg, 180 mg, 192 mg, and 200 mg), preferably the re-escalated daily dose is 174 mg.

In one aspect, the present invention relates to a method for the treatment of a human patient with chronic myeloid leukemia (CML) (such as a newly diagnosed chronic myeloid leukemia (CML) or a refractory chronic myeloid leukemia (CML)) comprising administering to the patient a therapeutically effective amount of compound of Formula I:

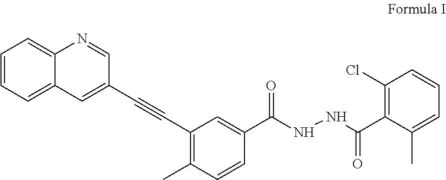 [SMILES: Cc1ccc(C(=O)NNC(=O)c2c(C)cccc2Cl)cc1C#Cc1cnc2ccccc2c1]

or a pharmaceutically acceptable salt thereof at an initial daily dose of 50 mg to 200 mg (e.g. any one of the dose selected from 50 mg, 66 mg, 87 mg, 90 mg, 96 mg, 100 mg, 126 mg, 130.5 mg, 135 mg, 150 mg, 174 mg, 180 mg, 192 mg and 200 mg).

In an embodiment, the initial daily is any one of the dose selected from 87 mg, 130.5 mg and 174 mg. In a preferred embodiment, the initial daily dose is 130.5 mg. In a more preferred embodiment, the initial daily dose is 87 mg.

In an embodiment, the patient is monitored for any hematologic and/or non-hematologic toxicities. The hematologic and/or non-hematologic toxicities are commonly known to a person skilled in the art e.g. as discussed as per National Cancer Institute's, Common Terminology Criteria for Adverse Events (NCI-CTCAE) Version 4.03 and Version 5.0 (https://evs.nci.nih.gov/ftp1/CTCAE/About.html).

In an embodiment, when a patient develops hematologic and/or non-hematologic toxicity with the initial daily dose, the initial daily dose is withheld for at least 7 days and resumed at the initial daily dose of 50 mg to 200 mg (e.g. any one of the dose selected from 50 mg, 66 mg, 87 mg, 90 mg, 96 mg, 100 mg, 126 mg, 130.5 mg, 135 mg, 150 mg, 174 mg, 180 mg, 192 mg and 200 mg).

In an embodiment, when a patient is at an initial daily dose of 174 mg and develops any hematologic and/or non-hematologic toxicity, the treatment is withheld for at least 7 days (such as 14 day, 21 days, 28 days etc.) and then the treatment is resumed again at the initial dose of 174 mg. Similarly, when a patient is treated at an initial daily dose of 130.5 mg and develops any hematologic and/or non-hematologic toxicity, the treatment is withheld for at least 7 days (such as 14 day, 21 days, 28 days etc.) and then the treatment is resumed at the initial dose of 130.5 mg.

In another embodiment, when a patient is at an initial daily dose of 87 mg and develops any hematologic and/or non-hematologic toxicity, the treatment is withheld for at least 7 days (such as 14 day, 21 days, 28 days etc.) and then the treatment is resumed again at the initial dose of 87 mg.

In one aspect, when the patient develops hematologic and/or non-hematologic toxicity with the initial daily dose, the initial daily dose is withheld for less than 7 days (such as for 4 days) and if the toxicity is resolved, then the treatment is resumed at the initial daily dose of 50 mg to 200 mg (e.g. selected from 50 mg, 66 mg, 87 mg, 90 mg, 96 mg, 100 mg, 126 mg, 130.5 mg, 135 mg, 150 mg, 174 mg, 180 mg, 192 mg and 200 mg).

In an embodiment, when a patient is treated at an initial daily dose of 174 mg and develops any hematologic and/or non-hematologic toxicity, the treatment is withheld for less than 7 days (such as for 4 days) and then the treatment is resumed again at the initial dose of 174 mg. Similarly, when a patient is treated at an initial daily dose of 130.5 mg and develops any hematologic and/or non-hematologic toxicity, the treatment is withheld for less than 7 days (such as 4 days) and then the treatment is resumed again at the initial dose of 130.5 mg.

In a preferred embodiment, when a patient is treated at an initial daily dose of 87 mg and develops any hematologic and/or non-hematologic toxicity, the treatment is withheld for less than 7 days (such as for 4 days) and then the treatment is resumed again at the initial dose of 87 mg.

In one aspect, when a patient develops hematologic and/or non-hematologic toxicity with the initial daily dose, the initial daily dose is withheld for at least 7 days and resumed at a reduced daily dose of 43.5 mg to 192 mg (e.g. selected from 43.5 mg, 45 mg, 48 mg, 50 mg, 66 mg, 87 mg, 90 mg, 126 mg, 130.5 mg, 135 mg, 174 mg, 180 mg and 192 mg); and wherein, the reduced daily dose is optionally re-escalated to a daily dose of 50 mg to 200 mg (e.g. selected from 50 mg, 66 mg, 87 mg, 90 mg, 96 mg, 100 mg, 126 mg, 130.5 mg, 135 mg, 150 mg, 174 mg, 180 mg, 192 mg, and 200 mg).

In an embodiment, when a patient develops hematologic and/or non-hematologic toxicity with the initial daily dose of 174 mg, the initial daily dose is withheld for at least 7 days and resumed at a reduced daily dose of 43.5 mg to 130.5 mg (e.g. selected from 43.5 mg, 45 mg, 48 mg, 50 mg, 66 mg, 87 mg, 90 mg, 126 mg, 130.5 mg, 135 mg, 174 mg, 180 mg and 192 mg); preferably the reduced daily dose is 130.5 mg. Further, the reduced daily dose can be optionally escalated or re-escalated to a daily dose of 50 mg to 200 mg (e.g. selected from 50 mg, 66 mg, 87 mg, 90 mg, 96 mg, 100 mg, 126 mg, 130.5 mg, 135 mg, 150 mg, 174 mg, 180 mg, 192 mg, and 200 mg), preferably the escalated or re-escalated daily dose is 200 mg.

In another embodiment, when a patient develops hematologic and/or non-hematologic toxicity with the initial daily dose of 130.5 mg, the initial daily dose is withheld for at least 7 days and resumed at a reduced daily dose of 43.5 mg to 130.5 mg (e.g. selected from 43.5 mg, 45 mg, 48 mg, 50 mg, 66 mg, 87 mg, 90 mg, 126 mg, and 130.5 mg); preferably the reduced daily dose is 87 mg. Further, the reduced daily dose can be optionally escalated or re-escalated to a daily dose of 50 mg to 130.5 mg (e.g. selected from 50 mg, 66 mg, 87 mg, 90 mg, 96 mg, 100 mg, 126 mg, and 130.5 mg), preferably the escalated or re-escalated daily dose is 174 mg or 200 mg, more preferably the escalated or re-escalated daily dose is 174 mg.

In another embodiment, when a patient develops hematologic and/or non-hematologic toxicity with the initial daily dose of 87 mg, the initial daily dose is withheld for at least 7 days and resumed at a reduced daily dose of 43.5 mg to 126 mg (e.g. selected from 43.5 mg, 45 mg, 48 mg, 50 mg, 66 mg, 87 mg, 90 mg, and 126); preferably the reduced daily dose is 43.5 mg. Further, the reduced daily dose can be optionally escalated or re-escalated to a daily dose of 50 mg to 200 mg (e.g. selected from 50 mg, 66 mg, 87 mg, 90 mg, 96 mg, 100 mg, 126 mg, 130.5 mg, 135 mg, 174 mg, 180 mg and 200 mg), preferably the escalated or re-escalated daily dose is any one of the dose selected from 130.5 mg, 174 mg and 200 mg, more preferably the escalated or re-escalated daily dose is 174 mg.

In one aspect, when a patient develops hematologic and/or non-hematologic toxicity with the initial daily dose, the initial daily dose is withheld for at least 7 days and resumed at a reduced daily dose of 43.5 mg to 192 mg (e.g. selected from 43.5 mg, 45 mg, 48 mg, 50 mg, 66 mg, 87 mg, 90 mg, 126 mg, 130.5 mg, 135 mg, 174 mg, 180 mg and 192 mg). The reduced daily dose can be further optionally be reduced to a subsequent reduced daily dose of 43.5 mg to 180 mg (e.g. selected from 43.5 mg, 45 mg, 48 mg, 50 mg, 66 mg, 87 mg, 90 mg, 126 mg, 130.5 mg, 135 mg, 174 mg and 180 mg) on subsequent instances of recurrences of the toxicity. The reduced daily dose or the subsequent reduced daily dose can be optionally escalated or re-escalated to a dose of 50 mg to 200 mg (e.g. any one of the dose selected from 50 mg, 66 mg, 87 mg, 90 mg, 126 mg, 130.5 mg, 135 mg, 174 mg, 180 mg, 192 mg, and 200 mg).

In one embodiment, when a patient develops hematologic and/or non-hematologic toxicity with the initial daily dose of 174 mg, the initial daily dose is withheld for at least 7 days and resumed at a reduced daily dose of 43.5 mg to 135 mg (e.g. selected from 43.5 mg, 45 mg, 48 mg, 50 mg, 66 mg, 87 mg, 90 mg, 126 mg, 130.5 mg, and 135 mg), preferably the reduced daily dose is 130.5 mg. The reduced daily dose can be further optionally be reduced to a subsequent reduced daily dose of 43.5 mg to 126 mg (e.g. selected from 43.5 mg, 45 mg, 48 mg, 50 mg, 66 mg, 87 mg, 90 mg, and 126 mg) on subsequent instances of recurrences of the toxicity, preferably the subsequent reduced dose is 87 mg. The reduced daily dose or the subsequent reduced daily dose can be optionally escalated or re-escalated to a dose up to 200 mg (e.g. any one of the dose selected from 50 mg, 66 mg, 87 mg, 90 mg, 126 mg, 130.5 mg, 135 mg, 174 mg, 180 mg, 192 mg, and 200 mg).

In one embodiment, when a patient develops hematologic and/or non-hematologic toxicity with the initial daily dose of 130.5 mg, the initial daily dose is withheld for at least 7 days and resumed at a reduced daily dose of 43.5 mg to 126 mg (e.g. selected from 43.5 mg, 45 mg, 48 mg, 50 mg, 66 mg, 87 mg, 90 mg, and 126 mg), preferably the reduced daily dose is 87 mg. The reduced daily dose can be further optionally be reduced to a subsequent reduced daily dose of 43.5 mg to 66 mg (e.g. selected from 43.5 mg, 45 mg, 48 mg, 50 mg, and 66 mg) on subsequent instances of recurrences of the toxicity, preferably the subsequent reduced dose is 43.5 mg. The reduced daily dose or the subsequent reduced daily dose can be optionally escalated or re-escalated to a dose up to 200 mg (e.g. any one of the dose selected from 50 mg, 66 mg, 87 mg, 90 mg, 126 mg, 130.5 mg, 135 mg, 174 mg, 180 mg, 192 mg, and 200 mg), preferably the escalated or re-escalated daily dose is 174 mg.

In one embodiment, when a patient develops hematologic and/or non-hematologic toxicity with the initial daily dose of 87 mg, the initial daily dose is withheld for at least 7 days and resumed at a reduced daily dose of 43.5 mg. The reduced daily dose or the subsequent reduced daily dose can be optionally escalated or re-escalated to a dose up to 200 mg (e.g. any one of the dose selected from 50 mg, 66 mg, 87 mg, 90 mg, 126 mg, 130.5 mg, 135 mg, 174 mg, 180 mg, 192 mg, and 200 mg), preferably the escalated or re-escalated dose is 130.5 mg. More preferably, the escalated or re-escalated daily dose is 174 mg.

According to any one of the embodiments described herein, wherein the initial daily dose is escalated or de-escalated with no severe adverse reaction.

According to any one of the embodiments described herein, wherein the patient is resistant or intolerant to at least one tyrosine kinase inhibitor.

According to any one of the embodiments described herein, wherein the at least one tyrosine kinase inhibitor is a second-generation tyrosine kinase inhibitor.

According to any one of the embodiments described herein, wherein the at least one tyrosine kinase inhibitor is selected from rodatinib, dasatinib, nilotinib, and bosutinib.

According to any one of the embodiments described herein, wherein the at least one tyrosine kinase inhibitor is a third-generation tyrosine kinase inhibitor.

According to any one of the embodiments described herein, wherein the at least one tyrosine kinase inhibitor is selected from ponatinib and asciminib.

According to any one of the embodiments described herein, the patient has one or more of the following mutations in the BCR-ABL1 kinase domain: M244V, L248V, E499E, Q252H, P223S, A337T, F359C, E255V, Y253F, F317L, Y253H, V299L, E255V, F317L, E255V, F359V, G250E, E255K, F359V, M351T, G250E, H369R, H396R, and V359I.

According to any one of the embodiments described herein, wherein the chronic myeloid leukemia (CML) is chronic, accelerated, or blast phase Philadelphia Chromosome Positive Chronic Myeloid Leukemia (Ph+ CML).

According to any one of the embodiments described herein, the initial daily dose is escalated or de-escalated with no severe adverse reaction.

According to any one of the embodiments described herein, the patient is resistant or intolerant to at least one tyrosine kinase inhibitor.

According to any one of the embodiments described herein, the at least one tyrosine kinase inhibitor is a second-generation tyrosine kinase inhibitor.

According to any one of the embodiments described herein, the at least one tyrosine kinase inhibitor is selected from dasatinib, nilotinib, radotinib and bosutinib.

According to any one of the embodiments described herein, the at least one tyrosine kinase inhibitor is a third-generation tyrosine kinase inhibitor.

According to any one of the embodiments described herein, the at least one tyrosine kinase inhibitor is selected from ponatinib and asciminib.

According to any one of the embodiments described herein, the patient has one or more of the following mutations in the BCR-ABL1 kinase domain: M244V, L248V, E499E, Q252H, P223S, A337T, F359C, E255V, Y253F, F317L, Y253H, V299L, E255V, F317L, E255V, F359V, G250E, E255K, F359V, M351T, G250E, H369R, H396R, and V359I.

According to any one of the embodiments described herein, the chronic myeloid leukemia (CML) is chronic, accelerated, or blast phase Philadelphia Chromosome Positive Chronic Myeloid Leukemia (Ph+ CML).

In another particular embodiment, the present invention relates to a method for the treatment of an adult patient with newly diagnosed CML, wherein the method comprises administering to said patient a therapeutically effective amount of an initial daily dose of 174 mg of a compound of Formula I or a pharmaceutically acceptable salt thereof such that when the patient develops non-hematologic toxicity:
a) the initial daily dose is withheld for at least 7 days and resumed at 174 mg after recovery of patient from said toxicity; or
b) the initial daily dose is withheld for less than 7 days and if toxicity is resolved then resumed at 174 mg; or
c) the initial daily dose is withheld for at least 7 days and resumed at a reduced daily dose of 130.5 mg on first instance and to 87 mg, and 43.5 mg on subsequent instances of recurrences of said toxicity;
and wherein the daily dose of the compound of Formula I or a pharmaceutically acceptable salt thereof in the patient results in mean Cₘₐₓ ranging from 3370 ng/ml to 5054±3051 ng/mL.

In a particular embodiment, the present invention relates to a method for the treatment of an adult patient with refractory CML, wherein the method comprises administering to the patient a therapeutically effective amount of an initial daily dose of 174 mg of the compound of Formula I or a pharmaceutically acceptable salt thereof such that when the patient develops non-hematologic toxicity:
a) the initial daily dose is withheld for at least 7 days and resumed after recovery of the patient from said toxicity; or
b) the initial daily dose is withheld for less than 7 days and if toxicity is resolved then resumed at 174 mg; or
c) the initial daily dose is withheld for at least 7 days and resumed at reduced daily dose of 126 mg on first instance and to 90 mg, 66 mg and 48 mg on subsequent instances of recurrences of said toxicity;
and wherein, the reduced dose is optionally re-escalated to any one of the dose selected from 66 mg, 90 mg, 126 mg and 174 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response; with no severe adverse reaction.

In a particular embodiment, the present invention relates to a method for the treatment of an adult patient with refractory CML, wherein the method comprises administering to said patient a therapeutically effective amount of an initial daily dose of 174 mg of a compound of Formula I or a pharmaceutically acceptable salt thereof such that when the patient develops non-hematologic toxicity:
a) the initial daily dose is withheld for at least 7 days and resumed at 174 mg after recovery of patient from said toxicity; or
b) the initial daily dose is withheld for less than 7 days and if toxicity is resolved then resumed at 174 mg; or
c) the initial daily dose is withheld for at least 7 days and resumed at a reduced daily dose of 130.5 mg on first instance and to 87 mg, and 43.5 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In a particular embodiment, the present invention relates to a method for the treatment of an adult patient with newly diagnosed CML, wherein the method comprises administering to the patient a therapeutically effective amount of an initial daily dose of 174 mg of the compound of Formula I or a pharmaceutically acceptable salt thereof such that when the patient develops non-hematologic toxicity:
a) the initial daily dose is withheld for at least 7 days and resumed after recovery of the patient from said toxicity; or
b) the initial daily dose is withheld for at least 7 days and resumed at reduced daily dose of 126 mg on first instance and to 90 mg, 66 mg 48 mg, 24 mg and 12 mg on subsequent instances of recurrences of the toxicity;
and wherein, the reduced dose is optionally re-escalated to any one of the dose selected from 24 mg, 48 mg, 66 mg, 90 mg, 126 mg and 174 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another embodiment, the present invention relates to a method for the treatment of an adult patient with newly diagnosed CML, wherein the method comprises administering to said patient a therapeutically effective amount of an initial daily dose of 174 mg of a compound of Formula I or a pharmaceutically acceptable salt thereof such that when the patient develops non-hematologic toxicity:
a) the initial daily dose is withheld for at least 7 days and resumed at 174 mg after recovery of patient from said toxicity; or
b) the initial daily dose is withheld for less than 7 days and if toxicity is resolved then resumed at 174 mg; or
c) the initial daily dose is withheld for at least 7 days and resumed at a reduced daily dose of 130.5 mg on first instance and to 87 mg, and 43.5 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another particular embodiment, the present invention relates to a method for the treatment of an adult patient with newly diagnosed CML, wherein the method comprises administering to said patient a therapeutically effective amount of an initial daily dose of 174 mg of a compound of Formula I or a pharmaceutically acceptable salt thereof such that when the patient develops non-hematologic toxicity:
a) the initial daily dose is withheld for at least 7 days and resumed at 174 mg after recovery of patient from said toxicity; or
b) the initial daily dose is withheld for less than 7 days and if toxicity is resolved then resumed at 174 mg; or
c) the initial daily dose is withheld for at least 7 days and resumed at a reduced daily dose of 130.5 mg on first instance and to 87 mg, and 43.5 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction,
and wherein the daily dose of the compound of Formula I or a pharmaceutically acceptable salt thereof in the patient results in mean AUC₀₋₂₄ ranging from 28598 ng*h/mL to 42898±33827 ng*h/mL.

In another particular embodiment, the present invention relates to a method for the treatment of an adult patient with newly diagnosed CML, wherein the method comprises administering to said patient a therapeutically effective amount of an initial daily dose of 174 mg of a compound of Formula I or a pharmaceutically acceptable salt thereof such that when the patient develops non-hematologic toxicity:
a) the initial daily dose is withheld for at least 7 days and resumed at 174 mg after
b) the initial daily dose is withheld for less than 7 days and if toxicity is resolved then resumed at 174 mg; or
c) the initial daily dose is withheld for at least 7 days and resumed at a reduced daily dose of 130.5 mg on first instance and to 87 mg, and 43.5 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction,
and wherein the daily dose of the compound of Formula I or a pharmaceutically acceptable salt thereof in the patient results in mean AUC₀₋₂₄ ranging from 28598 ng*h/mL to 42898±33827 ng*h/mL.

In another particular embodiment, the present invention relates to a method for the treatment of an adult patient with newly diagnosed CML, wherein the method comprises administering to said patient a therapeutically effective amount of an initial daily dose of 174 mg of a compound of Formula I or a pharmaceutically acceptable salt thereof such that when the patient develops non-hematologic toxicity:
a) the initial daily dose is withheld for at least 7 days and resumed at 174 mg after recovery of patient from said toxicity; or
b) the initial daily dose is withheld for less than 7 days and if toxicity is resolved then resumed at 174 mg; or
c) the initial daily dose is withheld for at least 7 days and resumed at a reduced daily dose of 130.5 mg on first instance and to 87 mg, and 43.5 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction,
and wherein the daily dose of the compound of Formula I or a pharmaceutically acceptable salt thereof in the patient results in mean Cₘₐₓ ranging from 3370 ng/ml to 5054±3051 ng/mL.

In a particular embodiment, the present invention relates to a method for the treatment of an adult patient with refractory CML, wherein the method comprises administering to the patient a therapeutically effective amount of an initial daily dose of 174 mg of the compound of Formula I or a pharmaceutically acceptable salt thereof such that when the patient develops hematologic toxicity:
a) the initial daily dose is withheld for at least 14 days and resumed after recovery of the patient from said toxicity; or
b) the initial daily dose is withheld for at least 14 days and resumed at a reduced daily dose of 126 mg on first instance and to 90 mg, 66 mg and 48 mg on subsequent instances of recurrence of the toxicity.

In one embodiment, the present invention relates to a method for the treatment of an adult patient with refractory CML, wherein the method comprises administering to said patient a therapeutically effective amount of an initial daily dose of 174 mg of a compound of Formula I or a pharmaceutically acceptable salt thereof such that when the patient develops non-hematologic toxicity:
a) the initial daily dose is withheld for at least 14 days and resumed at 174 mg after recovery of patient from said toxicity; or
b) the initial daily dose is withheld for less than 14 days and if toxicity is resolved then resumed at 174 mg; or
c) the initial daily dose is withheld for at least 14 days and resumed at a reduced daily dose of 130.5 mg on first instance and to 87 mg, and 43.5 mg on subsequent instances of recurrences of said toxicity.

In a particular embodiment, the present invention relates to a method for the treatment of an adult patient with newly diagnosed CML, wherein the method comprises administering to the patient a therapeutically effective amount of an initial daily dose of 174 mg of the compound of Formula I or a pharmaceutically acceptable salt thereof such that when the patient develops hematologic toxicity:
a) the initial daily dose is withheld for at least 14 days and resumed after recovery of the patient from said toxicity; or
b) the initial daily dose is withheld for at least 14 days and resumed at a reduced daily dose of 126 mg on first instance and to 90 mg, 66 mg, 48 mg, 24 mg and 12 mg on subsequent instances of recurrence of the toxicity.

In a particular embodiment, the present invention relates to a method for the treatment of an adult patient with newly diagnosed CML, wherein the method comprises administering to said patient a therapeutically effective amount of an initial daily dose of 174 mg of a compound of Formula I or a pharmaceutically acceptable salt thereof such that when the patient develops non-hematologic toxicity:
a) the initial daily dose is withheld for at least 14 days and resumed at 174 mg after recovery of patient from said toxicity; or
b) the initial daily dose is withheld for less than 14 days and if toxicity is resolved then resumed at 174 mg; or
c) the initial daily dose is withheld for at least 14 days and resumed at a reduced daily dose of 130.5 mg on first instance and to 87 mg, and 43.5 mg on subsequent instances of recurrences of said toxicity.

In another particular embodiment, the present invention relates to a method for the treatment of an adult patient with newly diagnosed CML, wherein the method comprises administering to said patient a therapeutically effective amount of an initial daily dose of 174 mg of a compound of Formula I or a pharmaceutically acceptable salt thereof such that when the patient develops non-hematologic toxicity:
a) the initial daily dose is withheld for at least 14 days and resumed at 174 mg after recovery of patient from said toxicity; or
b) the initial daily dose is withheld for less than 14 days and if toxicity is resolved then resumed at 174 mg; or
c) the initial daily dose is withheld for at least 14 days and resumed at a reduced daily dose of 130.5 mg on first instance and to 87 mg, and 43.5 mg on subsequent instances of recurrences of said toxicity;
and wherein the daily dose of the compound of Formula I or a pharmaceutically acceptable salt thereof in the patient results in mean AUC₀₋₂₄ ranging from 28598 ng*h/mL to 42898±33827 ng*h/mL.

In another particular embodiment, the present invention relates to a method for the treatment of an adult patient with newly diagnosed CML, wherein the method comprises administering to said patient a therapeutically effective amount of an initial daily dose of 174 mg of a compound of Formula I or a pharmaceutically acceptable salt thereof such that when the patient develops non-hematologic toxicity:
a) the initial daily dose is withheld for at least 14 days and resumed at 174 mg after recovery of patient from said toxicity; or
b) the initial daily dose is withheld for less than 14 days and if toxicity is resolved then resumed at 174 mg; or
c) the initial daily dose is withheld for at least 14 days and resumed at a reduced daily dose of 130.5 mg on first instance and to 87 mg, and 43.5 mg on subsequent instances of recurrences of said toxicity;
and wherein the daily dose of the compound of Formula I or a pharmaceutically acceptable salt thereof in the patient results in mean Cₘₐₓ ranging from 3370 ng/ml to 5054±3051 ng/mL.

In another particular embodiment, the present invention relates to a method for the treatment of an adult patient with newly diagnosed CML, wherein the method comprises administering to said patient a therapeutically effective amount of an initial daily dose of 174 mg of a compound of Formula I or a pharmaceutically acceptable salt thereof such that when the patient develops non-hematologic toxicity:
a) the initial daily dose is withheld for at least 14 days and resumed at 174 mg after recovery of patient from said toxicity; or
b) the initial daily dose is withheld for less than 14 days and if toxicity is resolved then resumed at 174 mg; or
c) the initial daily dose is withheld for at least 14 days and resumed at a reduced daily dose of 130.5 mg on first instance and to 87 mg, and 43.5 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction,
and wherein the daily dose of the compound of Formula I or a pharmaceutically acceptable salt thereof in the patient results in mean AUC₀₋₂₄ ranging from 28598 ng*h/mL to 42898±33827 ng*h/mL.

In another particular embodiment, the present invention relates to a method for the treatment of an adult patient with newly diagnosed CML, wherein the method comprises administering to said patient a therapeutically effective amount of an initial daily dose of 174 mg of a compound of Formula I or a pharmaceutically acceptable salt thereof such that when the patient develops non-hematologic toxicity:
a) the initial daily dose is withheld for at least 14 days and resumed at 174 mg after recovery of patient from said toxicity; or
b) the initial daily dose is withheld for less than 14 days and if toxicity is resolved then resumed at 174 mg; or
c) the initial daily dose is withheld for at least 14 days and resumed at a reduced daily dose of 130.5 mg on first instance and to 87 mg, and 43.5 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction,
and wherein the daily dose of the compound of Formula I or a pharmaceutically acceptable salt thereof in the patient results in mean Cₘₐₓ ranging from 3370 ng/ml to 5054±3051 ng/mL.

In a particular embodiment, the present invention relates to a method for the treatment of an adult patient with refractory CML, wherein the method comprises administering to the patient a therapeutically effective amount of an initial daily dose of 174 mg of the compound of Formula I or a pharmaceutically acceptable salt thereof such that when the patient develops hematologic toxicity:
a) the initial daily dose is withheld for at least 14 days and resumed after recovery of the patient from said toxicity; or
b) the initial daily dose is withheld for at least 14 days and resumed at reduced daily dose of 126 mg on first instance and to 90 mg, 66 mg and 48 mg on subsequent instances of recurrence of the toxicity;
and wherein, the reduced dose is optionally re-escalated to any one of the doses selected from 66 mg, 90 mg, 126 mg and 174 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response; with no severe adverse reaction.

In a particular embodiment, the present invention relates to a method for the treatment of an adult patient with refractory CML, wherein the method comprises administering to said patient a therapeutically effective amount of an initial daily dosage of 174 mg of a compound of Formula I or a pharmaceutically acceptable salt thereof such that when the patient develops non-hematologic toxicity:
a) the initial daily dose is withheld for at least 14 days and resumed at 174 mg after recovery of patient from said toxicity; or
b) the initial daily dose is withheld for less than 14 days and if toxicity is resolved then resumed at 174 mg; or
c) the initial daily dose is withheld for at least 14 days and resumed at a reduced daily dose of 130.5 mg on first instance and to 87 mg, and 43.5 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In a particular embodiment, the present invention relates to a method for the treatment of an adult patient with newly diagnosed CML, wherein the method comprises administering to the patient a therapeutically effective amount of an initial daily dose of 174 mg of the compound of Formula I or a pharmaceutically acceptable salt thereof such that when the patient develops hematologic toxicity:
a) the initial daily dose is withheld for at least 14 days and resumed after recovery of the patient from said toxicity; or
b) the initial daily dose is withheld for at least 14 days and resumed at reduced daily dose of 126 mg on first instance and to 90 mg, 66 mg, 48 mg, 24 mg and 12 mg on subsequent instances of recurrence of the toxicity.
and wherein, the reduced dose is optionally re-escalated to any one of the dose selected from 24 mg, 48 mg, 66 mg, 90 mg, 126 mg and 174 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response; with no severe adverse reaction.

In a particular embodiment, the present invention relates to a method for the treatment of an adult patient with newly diagnosed CML, wherein the method comprises administering to said patient a therapeutically effective amount of an initial daily dose of 174 mg of a compound of Formula I or a pharmaceutically acceptable salt thereof such that when the patient develops non-hematologic toxicity:
a) the initial daily dose is withheld for at least 14 days and resumed at 174 mg after recovery of patient from said toxicity; or
b) the initial daily dose is withheld for less than 14 days and if toxicity is resolved then resumed at 174 mg; or
c) the initial daily dose is withheld for at least 14 days and resumed at a reduced daily dose of 130.5 mg on first instance and to 87 mg, and 43.5 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In a particular embodiment, the present invention relates to a method for the treatment of an adult patient with refractory CML, wherein the method comprises administering to the patient a therapeutically effective amount of an initial daily dose of 174 mg of the compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of the patient from said toxicity and the treatment is continued at a reduced dose of 126 mg on first instance and at 90 mg, 66 mg and 48 mg on subsequent instances of recurrence of the toxicity.

In a particular embodiment, the present invention relates to a method for the treatment of an adult patient with refractory CML, wherein the method comprises administering to the patient a therapeutically effective amount of an initial daily dose of 174 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose of 130.5 mg on first instance and to 87 mg, and 43.5 mg on subsequent instances of recurrences of said toxicity.

In a particular embodiment, the present invention relates to a method for the treatment of an adult patient with newly diagnosed CML, wherein the method comprises administering to the patient a therapeutically effective amount of an initial daily dose of 174 mg of the compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of the patient from said toxicity and the treatment is continued at a reduced dose of 126 mg on first instance and at 90 mg, 66 mg, 48 mg, 24 mg and 12 mg on subsequent instances of recurrence of the toxicity.

In one embodiment, the present invention relates to a method for the treatment of an adult patient with newly diagnosed CML, wherein the method comprises administering to the patient a therapeutically effective amount of an initial daily dose of 174 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose of 130.5 mg on first instance and to 87 mg, and 43.5 mg on subsequent instances of recurrences of said toxicity.

In a particular embodiment, the present invention relates to a method for the treatment of an adult patient with refractory CML, wherein the method comprises administering to the patient a therapeutically effective amount of an initial daily dose of 174 mg of the compound of Formula I or a pharmaceutically acceptable salt thereof such that when the patient develops hematologic toxicity that is attributable to the compound of Formula I or a pharmaceutically acceptable salt thereof,
the initial daily dose is withheld for up to four weeks to allow the recovery of the patient from said toxicity and the treatment is continued at a reduced dose of 126 mg on first instance and at 90 mg, 66 mg and 48 mg on subsequent instances of recurrence of said toxicity, and wherein
the reduced dose is optionally re-escalated to any one of the dose selected from 66 mg, 90 mg, 126 mg and 174 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In a particular embodiment, the present invention relates to a method for the treatment of an adult patient with refractory CML, wherein the method comprises administering to the patient a therapeutically effective amount of an initial daily dose of 174 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose of 130.5 mg on first instance and to 87 mg, and 43.5 mg on subsequent instances of recurrences of said toxicity and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In a particular embodiment, the present invention relates to a method for the treatment of an adult patient with newly diagnosed CML, wherein the method comprises administering to the patient a therapeutically effective amount of an initial daily dose of 174 mg of the compound of Formula I or a pharmaceutically acceptable salt thereof such that when the patient develops hematologic toxicity that is attributable to the compound of Formula I or a pharmaceutically acceptable salt thereof,
the initial daily dose is withheld for up to four weeks to allow the recovery of the patient from said toxicity and the treatment is continued at a reduced dose of 126 mg on first instance and at 90 mg, 66 mg, 48 mg, 24 mg and 12 on subsequent instances of recurrence of said toxicity, and wherein
the reduced dose is optionally re-escalated to any one of the dose selected from 24 mg, 48 mg, 66 mg, 90 mg, 126 mg and 174 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no hematologic toxicity.

In a particular embodiment, the present invention relates to a method for the treatment of an adult patient with newly diagnosed CML, wherein the method comprises administering to the patient a therapeutically effective amount of an initial daily dose of 174 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose of 130.5 mg on first instance and to 87 mg, and 43.5 mg on subsequent instances of recurrences of said toxicity and wherein,
the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another particular embodiment, the present invention relates to a method for the treatment of an adult patient with newly diagnosed CML, wherein the method comprises administering to the patient a therapeutically effective amount of an initial daily dose of 174 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose of 130.5 mg on first instance and to 87 mg, and 43.5 mg on subsequent instances of recurrences of said toxicity, and wherein the daily dose of the compound of Formula I or a pharmaceutically acceptable salt thereof in the patient results in mean AUC₀₋₂₄ ranging from 28598 ng*h/mL to 42898±33827 ng*h/mL.

In another particular embodiment, the present invention relates to a method for the treatment of an adult patient with newly diagnosed chronic myeloid leukemia (CML), wherein the method comprises administering to the patient a therapeutically effective amount of an initial daily dose of 174 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose of 130.5 mg on first instance and to 87 mg, and 43.5 mg on subsequent instances of recurrences of said toxicity, and wherein the daily dose of the compound of Formula I or a pharmaceutically acceptable salt thereof in the patient results in mean Cₘₐₓ ranging from 3370 ng/ml to 5054±3051 ng/ml.

In another particular embodiment, the present invention relates to a method for the treatment of an adult patient with newly diagnosed CML, wherein the method comprises administering to the patient a therapeutically effective amount of an initial daily dose of 174 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose of 130.5 mg on first instance and to 87 mg, and 43.5 mg on subsequent instances of recurrences of said toxicity and wherein,
the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof in the patient results in mean AUC₀₋₂₄ ranging from 28598 ng*h/mL to 42898±33827 ng*h/mL.

In another particular embodiment, the present invention relates to a method for the treatment of an adult patient with newly diagnosed CML, wherein the method comprises administering to the patient a therapeutically effective amount of an initial daily dose of 174 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose of 130.5 mg on first instance and to 87 mg, and 43.5 mg on subsequent instances of recurrences of said toxicity and wherein,
the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction,
and wherein the daily dose of the compound of Formula I or a pharmaceutically acceptable salt thereof in the patient results in mean Cₘₐₓ ranging from 3370 ng/ml to 5054±3051 ng/mL.

In a particular embodiment, the present invention relates to a method for the treatment of an adult patient with CML, comprising orally administering a therapeutically effective amount of a compound of Formula I or a pharmaceutically acceptable salt thereof at an initial daily dose, and escalating or de-escalating the dose to achieve or maintain complete hematologic response with no severe adverse reaction, and wherein the daily dose of the compound of Formula I or a pharmaceutically acceptable salt thereof in a patient results in mean AUC₀₋₂₄ ranging from 6226±5827 ng*h/mL to 60373±55659 ng*h/mL.

In another particular embodiment, the present invention relates to a method for the treatment of an adult patient with CML, comprising orally administering a therapeutically effective amount of a compound of Formula I or a pharmaceutically acceptable salt thereof at an initial daily dose, and escalating or de-escalating the dose to achieve or maintain complete hematologic response with no severe adverse reaction, and wherein the daily dose of the compound of Formula I or a pharmaceutically acceptable salt thereof results in a mean Cₘₐₓ ranging from 664±450 ng/ml to 5054±3051 ng/mL.

In yet another particular embodiment, the present invention relates to a method for the treatment of an adult patient with CML, comprising orally administering a therapeutically effective amount of a compound of Formula I or a pharmaceutically acceptable salt thereof at an initial daily dose, and escalating or de-escalating the dose to achieve or maintain disease response, with no severe adverse reaction, and wherein the daily dose of the compound of Formula I or a pharmaceutically acceptable salt thereof in the patient results in mean AUC₀₋₂₄ ranging from 6226±5827 ng*h/mL to 60373±55659 ng*h/mL.

In another particular embodiment, the present invention provides a method for the treatment of an adult patient with CML, comprising orally administering a therapeutically effective amount of a compound of Formula I or a pharmaceutically acceptable salt thereof at an initial daily dose, and escalating or de-escalating the dose to achieve or maintain disease response, with no severe adverse reaction, and wherein the daily dose of the compound of Formula I or a pharmaceutically acceptable salt thereof results in a mean Cₘₐₓ ranging from 664±450 ng/mL to 5054±3051 ng/mL.

In a particular embodiment, the present invention provides a method for the treatment of an adult patient with CML, comprising orally administering a therapeutically effective amount of a compound of Formula I or a pharmaceutically acceptable salt thereof at an initial daily dose of between 10 mg and 200 mg and escalating to the dose between 20 mg and 210 mg to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof in the patient results in mean AUC₀₋₂₄ ranging from 6226±5827 ng*h/mL to 60373±55659 ng*h/mL.

In another particular embodiment, the present invention relates to a method for the treatment of an adult patient with CML, comprising orally administering a therapeutically effective amount of a compound of Formula I or a pharmaceutically acceptable salt thereof at an initial daily dose of between 10 mg and 200 mg and escalating to the dose between 20 mg and 210 mg to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof results in a mean Cₘₐₓ ranging from 664±450 ng/ml to 5054±3051 ng/mL.

In yet another particular embodiment, the present invention relates to a method for the treatment of an adult patient with CML, comprising orally administering a therapeutically effective amount of a compound of Formula I or a pharmaceutically acceptable salt thereof at an initial daily dose of between 20 mg and 210 mg and de-escalating to the dose between 10 mg and 200 mg on occurrence of severe adverse reaction, wherein the dose is de-escalated such that with the de-escalated dose the patient achieves or maintains at least one of (a) complete hematological response, (b) complete hematological response and partial cytogenetic response, (c) complete hematological response and complete cytogenetic response, or (d) complete hematological response, complete cytogenetic response, and major molecular response, and wherein the daily dose of the compound of Formula I or a pharmaceutically acceptable salt thereof in the patient results in mean AUC₀₋₂₄ ranging from 6226±5827 ng*h/mL to 60373±55659 ng*h/mL.

In yet another particular embodiment, the present invention relates to a method for the treatment of an adult patient with CML, comprising orally administering a therapeutically effective amount of a compound of Formula I or a pharmaceutically acceptable salt thereof at an initial daily dose of between 20 mg and 210 mg and de-escalating to the dose between 10 mg and 200 mg on occurrence of severe adverse reaction, wherein the dose is de-escalated such that with the de-escalated dose the patient achieves or maintains at least one of (a) complete hematological response, (b) complete hematological response and partial cytogenetic response, (c) complete hematological response and complete cytogenetic response, or (d) complete hematological response, complete cytogenetic response, and major molecular response, and wherein the daily dose of the compound of Formula I or a pharmaceutically acceptable salt thereof results in a mean Cₘₐₓ ranging from 664±450 ng/mL to 5054±3051 ng/ml.

In yet another particular embodiment, the present invention relates to a method for the treatment of an adult patient with refractory CML comprising, administering a therapeutically effective amount of compound of Formula I or a pharmaceutically acceptable salt thereof at an initial daily dose of 174 mg, wherein the initial dose is escalated to 204 mg to achieve or maintain complete hematologic response (CHR) with no severe adverse reaction, and wherein the daily dose of the compound of Formula I or a pharmaceutically acceptable salt thereof in the patient results in mean AUC₀₋₂₄ ranging from 6226±5827 ng*h/mL to 60373±55659 ng*h/mL.

In a particular embodiment, the present invention relates to a method for the treatment of an adult patient with refractory CML comprising, administering a therapeutically effective amount of compound of Formula I or a pharmaceutically acceptable salt thereof at an initial daily dose of 174 mg, wherein the initial dose is escalated to 204 mg to achieve or maintain complete hematologic response (CHR) with no severe adverse reaction, and wherein the daily dose of the compound of Formula I or a pharmaceutically acceptable salt thereof results in a mean Cₘₐₓ ranging from 664±450 ng/mL to 5054±3051 ng/mL.

In yet another particular embodiment, the present invention relates to a method for the treatment of an adult patient with refractory CML, comprising orally administering a therapeutically effective amount of a compound of Formula I or a pharmaceutically acceptable salt thereof at an initial daily dose of 174 mg, wherein the initial dose is escalated to 204 mg to achieve or maintain at least one of (a) complete hematological response, (b) complete hematological response and partial cytogenetic response, (c) complete hematological response and complete cytogenetic response, or (d) complete hematological response, complete cytogenetic response, and major molecular response; with no severe adverse reaction, and wherein the daily dose of the compound of Formula I or a pharmaceutically acceptable salt thereof in the patient results in mean AUC₀₋₂₄ ranging from 6226±5827 ng*h/mL to 60373±55659 ng*h/mL.

In yet another particular embodiment, the present invention relates to a method for the treatment of an adult patient with refractory CML, comprising orally administering a therapeutically effective amount of a compound of Formula I or a pharmaceutically acceptable salt thereof at an initial daily dose of 174 mg, wherein the initial dose is escalated to 204 mg to achieve or maintain at least one of (a) complete hematological response, (b) complete hematological response and partial cytogenetic response, (c) complete hematological response and complete cytogenetic response, or (d) complete hematological response, complete cytogenetic response, and major molecular response; with no severe adverse reaction, and wherein the daily dose of the compound of Formula I or a pharmaceutically acceptable salt thereof results in a mean Cₘₐₓ ranging from 664±450 ng/ml to 5054±3051 ng/ml.

In another particular embodiment, the present invention relates to a method for the treatment of an adult patient with refractory CML, wherein the method comprises administering to the patient an initial daily dose of 174 mg of a compound of Formula I or a pharmaceutically acceptable salt thereof such that when the patient develops non-hematological toxicity:
a) an initial daily dose is withheld for at least 7 days and resumed after recovery of patient from said toxicity at 174 mg; or
b) an initial daily dose is withheld for at least 7 days and resumed at a reduced daily dose of 126 mg on first instance and to 90 mg, 66 mg and 48 mg on subsequent instances of recurrences of said toxicity;
and wherein the daily dose of the compound of Formula I or a pharmaceutically acceptable salt thereof in the patient results in mean AUC₀₋₂₄ ranging from 6226±5827 ng*h/mL to 60373±55659 ng*h/mL.

In a particular embodiment, the present invention relates to a method for the treatment of an adult patient with refractory CML, wherein the method comprises administering to the patient an initial daily dose of 174 mg of a compound of Formula I or a pharmaceutically acceptable salt thereof such that when the patient develops non-hematological toxicity:
a) the initial daily dose is withheld for at least 7 days and resumed at 174 mg after recovery of patient from said toxicity; or
b) the initial daily dose is withheld for less than 7 days and if toxicity is resolved then resumed at 174 mg; or
c) the initial daily dose is withheld for at least 7 days and resumed at a reduced daily dose of 130.5 mg on first instance and to 87 mg, and 43.5 mg on subsequent instances of recurrences of said toxicity;
and wherein the daily dose of the compound of Formula I or a pharmaceutically acceptable salt thereof in the patient results in mean AUC₀₋₂₄ ranging from 28598 ng*h/mL to 42898±33827 ng*h/mL.

According to a particular embodiment, the present invention relates to a method for the treatment of an adult patient with refractory CML, wherein the method comprises administering to the patient an initial daily dose of 174 mg of a compound of Formula I or a pharmaceutically acceptable salt thereof such that when the patient develops non-hematological toxicity:
a) an initial daily dose is withheld for at least 7 days and resumed after recovery of patient from said toxicity at 174 mg; or
b) an initial daily dose is withheld for at least 7 days and resumed at a reduced daily dose of 126 mg on first instance and to 90 mg, 66 mg and 48 mg on subsequent instances of recurrences of said toxicity;
and wherein the daily dose of the compound of Formula I or a pharmaceutically acceptable salt thereof results in a mean Cₘₐₓ ranging from 664±450 ng/ml to 5054±3051 ng/ml.

In a particular embodiment, the present invention relates to a method for the treatment of an adult patient with refractory CML, wherein the method comprises administering to the patient an initial daily dose of 174 mg of a compound of Formula I or a pharmaceutically acceptable salt thereof such that when the patient develops non-hematological toxicity:
a) the initial daily dose is withheld for at least 7 days and resumed at 174 mg after recovery of patient from said toxicity; or
b) the initial daily dose is withheld for less than 7 days and if toxicity is resolved then resumed at 174 mg; or
c) the initial daily dose is withheld for at least 7 days and resumed at a reduced daily dose of 130.5 mg on first instance and to 87 mg, and 43.5 mg on subsequent instances of recurrences of said toxicity;
and wherein the daily dose of the compound of Formula I or a pharmaceutically acceptable salt thereof in the patient results in mean Cₘₐₓ ranging from 3370 ng/ml to 5054±3051 ng/mL.

According to a particular embodiment, the present invention provides a method for the treatment of an adult patient with refractory CML, wherein the method comprises administering to said patient an initial daily dose of 174 mg of a compound of Formula I or a pharmaceutically acceptable salt thereof such that when the patient develops non-hematologic toxicity:
a) the initial daily dose is withheld for at least 7 days and resumed after recovery of patient from said toxicity; or
b) the initial daily dose is withheld for at least 7 days and resumed at reduced daily dose of 126 mg on first instance and to 90 mg, 66 mg and 48 mg on subsequent instances of recurrences of said toxicity;
and wherein, the reduced dose is optionally re-escalated to any one of the dose selected from 66 mg, 90 mg, 126 mg and 174 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof in the patient results in mean AUC₀₋₂₄ ranging from 6226±5827 ng*h/mL to 60373±55659 ng*h/mL.

In a particular embodiment, the present invention provides a method for the treatment of an adult patient with refractory CML, wherein the method comprises administering to said patient an initial daily dose of 174 mg of a compound of Formula I or a pharmaceutically acceptable salt thereof such that when the patient develops non-hematologic toxicity:
a) the initial daily dose is withheld for at least 7 days and resumed at 174 mg after recovery of patient from said toxicity; or
b) the initial daily dose is withheld for less than 7 days and if toxicity is resolved then resumed at 174 mg; or
c) the initial daily dose is withheld for at least 7 days and resumed at a reduced daily dose of 130.5 mg on first instance and to 87 mg, and 43.5 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction,
and wherein the daily dose of the compound of Formula I or a pharmaceutically acceptable salt thereof in the patient results in mean AUC₀₋₂₄ ranging from 28598 ng*h/mL to 42898±33827 ng*h/mL.

According to another particular embodiment, the present invention provides a method for the treatment of an adult patient with refractory CML, wherein the method comprises administering to said patient an initial daily dose of 174 mg of a compound of Formula I or a pharmaceutically acceptable salt thereof such that when the patient develops non-hematologic toxicity:
a) the initial daily dose is withheld for at least 7 days and resumed after recovery of patient from said toxicity; or
b) the initial daily dose is withheld for at least 7 days and resumed at reduced daily dose of 126 mg on first instance and to 90 mg, 66 mg and 48 mg on subsequent instances of recurrences of said toxicity;
and wherein, the reduced dose is optionally re-escalated to any one of the dose selected from 66 mg, 90 mg, 126 mg and 174 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof results in a mean Cₘₐₓ ranging from 664±450 ng/ml to 5054±3051 ng/mL.

In a particular embodiment, the present invention provides a method for the treatment of an adult patient with refractory CML, wherein the method comprises administering to said patient an initial daily dose of 174 mg of a compound of Formula I or a pharmaceutically acceptable salt thereof such that when the patient develops non-hematologic toxicity:
a) the initial daily dose is withheld for at least 7 days and resumed at 174 mg after recovery of patient from said toxicity; or
b) the initial daily dose is withheld for less than 7 days and if toxicity is resolved then resumed at 174 mg; or
c) the initial daily dose is withheld for at least 7 days and resumed at a reduced daily dose of 130.5 mg on first instance and to 87 mg, and 43.5 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction,
and wherein the daily dose of the compound of Formula I or a pharmaceutically acceptable salt thereof in the patient results in mean Cₘₐₓ ranging from 3370 ng/ml to 5054±3051 ng/mL.

According to another particular embodiment, the present invention provides a method for the treatment of an adult patient with refractory CML, wherein the method comprises administering to the patient an initial daily dose of 174 mg of a compound of Formula I or a pharmaceutically acceptable salt thereof such that when the patient develops hematologic toxicity:
a) the initial daily dose is withheld for at least 14 days and resumed after recovery of patient from said toxicity; or
b) the initial daily dose is withheld for at least 14 days and resumed at reduced daily dose of 126 mg on first instance and to 90 mg, 66 mg and 48 mg on subsequent instances of recurrence of said toxicity;
and wherein the daily dose of the compound of Formula I or a pharmaceutically acceptable salt thereof in the patient results in mean AUC₀₋₂₄ ranging from 6226±5827 ng*h/mL to 60373±55659 ng*h/mL.

In a particular embodiment, the present invention relates to a method for the treatment of an adult patient with refractory CML, wherein the method comprises administering to said patient an initial daily dose of 174 mg of a compound of Formula I or a pharmaceutically acceptable salt thereof such that when the patient develops non-hematologic toxicity:
a) the initial daily dose is withheld for at least 14 days and resumed at 174 mg after recovery of patient from said toxicity; or
b) the initial daily dose is withheld for less than 14 days and if toxicity is resolved then resumed at 174 mg; or
c) the initial daily dose is withheld for at least 14 days and resumed at a reduced daily dose of 130.5 mg on first instance and to 87 mg, and 43.5 mg on subsequent instances of recurrences of said toxicity;
and wherein the daily dose of the compound of Formula I or a pharmaceutically acceptable salt thereof in the patient results in mean AUC₀₋₂₄ ranging from 28598 ng*h/mL to 42898±33827 ng*h/mL.

In another particular embodiment, the present invention provides a method for the treatment of an adult patient with refractory CML, wherein the method comprises administering to the patient an initial daily dose of 174 mg of a compound of Formula I or a pharmaceutically acceptable salt thereof such that when the patient develops hematologic toxicity:
a) the initial daily dose is withheld for at least 14 days and resumed after recovery of patient from said toxicity; or
b) the initial daily dose is withheld for at least 14 days and resumed at reduced daily dose of 126 mg on first instance and to 90 mg, 66 mg and 48 mg on subsequent instances of recurrence of said toxicity;
and wherein the daily dose of the compound of Formula I or a pharmaceutically acceptable salt thereof results in a mean Cₘₐₓ ranging from 664±450 ng/ml to 5054±3051 ng/mL.

In another particular embodiment, the present invention relates to a method for the treatment of an adult patient with refractory CML, wherein the method comprises administering to said patient an initial daily dose of 174 mg of a compound of Formula I or a pharmaceutically acceptable salt thereof such that when the patient develops non-hematologic toxicity:
a) the initial daily dose is withheld for at least 14 days and resumed at 174 mg after recovery of patient from said toxicity; or
b) the initial daily dose is withheld for less than 14 days and if toxicity is resolved then resumed at 174 mg; or
c) the initial daily dose is withheld for at least 14 days and resumed at a reduced daily dose of 130.5 mg on first instance and to 87 mg, and 43.5 mg on subsequent instances of recurrences of said toxicity;
and wherein the daily dose of the compound of Formula I or a pharmaceutically acceptable salt thereof in the patient results in mean Cₘₐₓ ranging from 3370 ng/ml to 5054±3051 ng/ml.

In another particular embodiment, the present invention relates to a method for the treatment of an adult patient with refractory CML, wherein the method comprises administering to the patient an initial daily dose of 174 mg of a compound of Formula I or a pharmaceutically acceptable salt thereof such that when the patient develops hematologic toxicity:
a) the initial daily dose is withheld for at least 14 days and resumed after recovery of patient from said toxicity; or
b) the initial daily dose is withheld for at least 14 days and resumed at reduced daily dose of 126 mg on first instance and to 90 mg, 66 mg and 48 mg on subsequent instances of recurrence of said toxicity;
and wherein, the reduced dose is optionally re-escalated to any one of the dose selected from 66 mg, 90 mg, 126 mg and 174 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction,
and wherein the daily dose of the compound of Formula I or a pharmaceutically acceptable salt thereof in the patient results in mean AUC₀₋₂₄ ranging from 6226±5827 ng*h/mL to 60373±55659 ng*h/mL.

In a particular embodiment, the present invention relates to a method for the treatment of an adult patient with refractory CML, wherein the method comprises administering to said patient an initial daily dose of 174 mg of a compound of Formula I or a pharmaceutically acceptable salt thereof such that when the patient develops non-hematologic toxicity:
a) the initial daily dose is withheld for at least 14 days and resumed at 174 mg after recovery of patient from said toxicity; or
b) the initial daily dose is withheld for less than 14 days and if toxicity is resolved then resumed at 174 mg; or
c) the initial daily dose is withheld for at least 14 days and resumed at a reduced daily dose of 130.5 mg on first instance and to 87 mg, and 43.5 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction,
and wherein the daily dose of the compound of Formula I or a pharmaceutically acceptable salt thereof in the patient results in mean AUC₀₋₂₄ ranging from 28598 ng*h/mL to 42898±33827 ng*h/mL.

In a particular embodiment, the present invention relates to a method for the treatment of an adult patient with refractory CML, wherein the method comprises administering to the patient an initial daily dose of 174 mg of a compound of Formula I or a pharmaceutically acceptable salt thereof such that when the patient develops hematologic toxicity:
a) the initial daily dose is withheld for at least 14 days and resumed after recovery of patient from said toxicity; or
b) the initial daily dose is withheld for at least 14 days and resumed at reduced daily dose of 126 mg on first instance and to 90 mg, 66 mg and 48 mg on subsequent instances of recurrence of said toxicity;
and wherein, the reduced dose is optionally re-escalated to any one of the dose selected from 66 mg, 90 mg, 126 mg and 174 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction,
and wherein the daily dose of the compound of Formula I or a pharmaceutically acceptable salt thereof results in a mean Cₘₐₓ ranging from 664±450 ng/ml to 5054±3051 ng/mL.

In another particular embodiment, the present invention relates to a method for the treatment of an adult patient with refractory CML, wherein the method comprises administering to said patient an initial daily dose of 174 mg of a compound of Formula I or a pharmaceutically acceptable salt thereof such that when the patient develops non-hematologic toxicity:
a) the initial daily dose is withheld for at least 14 days and resumed at 174 mg after recovery of patient from said toxicity; or
b) the initial daily dose is withheld for less than 14 days and if toxicity is resolved then resumed at 174 mg; or
c) the initial daily dose is withheld for at least 14 days and resumed at a reduced daily dose of 130.5 mg on first instance and to 87 mg, and 43.5 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction,
and wherein the daily dose of the compound of Formula I or a pharmaceutically acceptable salt thereof results in a mean Cₘₐₓ ranging from 3370 ng/ml to 5054±3051 ng/ml.

In a particular embodiment, the present invention relates to a method for the treatment of an adult patient with refractory CML, wherein the method comprises administering to the patient an initial daily dose of 174 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced dose of 126 mg on first instance and at 90 mg, 66 mg and 48 mg on subsequent instances of recurrence of said toxicity;
and wherein the daily dose of the compound of Formula I or a pharmaceutically acceptable salt thereof in the patient results in mean AUC₀₋₂₄ ranging from 6226±5827 ng*h/mL to 60373±55659 ng*h/mL.

In another particular embodiment, the present invention relates to a method for the treatment of an adult patient with refractory CML, wherein the method comprises administering to the patient an initial daily dose of 174 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose of 130.5 mg on first instance and to 87 mg, and 43.5 mg on subsequent instances of recurrences of said toxicity, and wherein the daily dose of the compound of Formula I or a pharmaceutically acceptable salt thereof in the patient results in mean AUC0-24 ranging from 28598 ng*h/mL to 42898±33827 ng*h/mL.

In another particular embodiment, the present invention relates to a method for the treatment of an adult patient with refractory CML, wherein the method comprises administering to the patient an initial daily dose of 174 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced dose of 126 mg on first instance and at 90 mg, 66 mg and 48 mg on subsequent instances of recurrence of said toxicity;
and wherein the daily dose of the compound of Formula I or a pharmaceutically acceptable salt thereof results in a mean Cₘₐₓ ranging from 664±450 ng/mL to 5054±3051 ng/ml.

In another particular embodiment, the present invention relates to a method for the treatment of an adult patient with refractory CML, wherein the method comprises administering to the patient an initial daily dose of 174 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose of 130.5 mg on first instance and to 87 mg, and 43.5 mg on subsequent instances of recurrences of said toxicity,
and wherein the daily dose of the compound of Formula I or a pharmaceutically acceptable salt thereof in the patient results in mean Cₘₐₓ ranging from 3370 ng/ml to 5054±3051 ng/mL.

In another particular embodiment, the present invention relates to a method for the treatment of an adult patient with refractory CML, wherein the method comprises administering to the patient an initial daily dose of 174 mg of a compound of Formula I or a pharmaceutically acceptable salt thereof such that when the patient develops hematologic toxicity that is attributable to the compound of Formula I or a pharmaceutically acceptable salt thereof,
the initial daily dose is withheld for up to four weeks to allow the recovery of patient from said toxicity and the treatment is continued at a reduced dose of 126 mg on first instance and at 90 mg, 66 mg and 48 mg on subsequent instances of recurrence of said toxicity, and wherein
the reduced dose is optionally re-escalated to any one of the dose selected from 66 mg, 90 mg, 126 mg and 174 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction,
and wherein the daily dose of the compound of Formula I or a pharmaceutically acceptable salt thereof in the patient results in mean AUC₀₋₂₄ ranging from 6226±5827 ng*h/mL to 60373±55659 ng*h/mL.

In a particular embodiment, the present invention relates to a method for the treatment of an adult patient with refractory CML, wherein the method comprises administering to the patient an initial daily dose of 174 mg of a compound of Formula I or a pharmaceutically acceptable salt thereof such that when the patient develops hematologic toxicity that is attributable to the compound of Formula I or a pharmaceutically acceptable salt thereof,
the initial daily dose is withheld for up to four weeks to allow the recovery of patient from said toxicity and the treatment is continued at a reduced dose of 126 mg on first instance and at 90 mg, 66 mg and 48 mg on subsequent instances of recurrence of said toxicity, and wherein
the reduced dose is optionally re-escalated to any one of the dose selected from 66 mg, 90 mg, 126 mg and 174 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction,
and wherein the daily dose of the compound of Formula I or a pharmaceutically acceptable salt thereof results in a mean Cₘₐₓ ranging from 664±450 ng/ml to 5054±3051 ng/ml.

In another particular embodiment, the present invention relates to a method for the treatment of an adult patient with refractory CML, wherein the method comprises administering to the patient an initial daily dose of 174 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose of 130.5 mg on first instance and to 87 mg, and 43.5 mg on subsequent instances of recurrences of said toxicity and wherein,
the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another particular embodiment, the present invention relates to a method for the treatment of an adult patient with refractory CML, wherein the method comprises administering to the patient an initial daily dose of 174 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose of 130.5 mg on first instance and to 87 mg, and 43.5 mg on subsequent instances of recurrences of said toxicity and wherein,
the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof in the patient results in mean AUC₀₋₂₄ ranging from 28598 ng*h/mL to 42898±33827 ng*h/mL.

In another particular embodiment, the present invention relates to a method for the treatment of an adult patient with refractory CML, wherein the method comprises administering to the patient an initial daily dose of 174 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose of 130.5 mg on first instance and to 87 mg, and 43.5 mg on subsequent instances of recurrences of said toxicity and wherein,
the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof results in a mean Cₘₐₓ ranging from 3370 ng/mL to 5054±3051 ng/ml.

In a particular embodiment, the present invention relates to a method for the treatment of newly diagnosed adult CML patient comprising, administering compound of Formula I or a pharmaceutically acceptable salt thereof at an initial daily dose of 174 mg, wherein the initial dose is escalated to 204 mg to achieve or maintain complete hematologic response (CHR) with no severe adverse reaction, and wherein the daily dose of the compound of Formula I or a pharmaceutically acceptable salt thereof in the patient results in mean AUC₀₋₂₄ ranging from 6226±5827 ng*h/mL to 60373±55659 ng*h/mL.

In another particular embodiment, the present invention relates to a method for the treatment of newly diagnosed adult CML patient comprising, administering compound of Formula I or a pharmaceutically acceptable salt thereof at an initial daily dose of 174 mg, wherein the initial dose is escalated to 204 mg to achieve or maintain complete hematologic response (CHR) with no severe adverse reaction, and wherein the daily dose of the compound of Formula I or a pharmaceutically acceptable salt thereof results in a mean Cₘₐₓ ranging from 664±450 ng/mL to 5054±3051 ng/mL.

In another particular embodiment, the present invention relates to a method for the treatment of an adult patient with chronic myeloid leukemia (CML), comprising orally administering a therapeutic effective amount of a pharmaceutical composition comprising a compound of Formula I or a pharmaceutically acceptable salt thereof, at an initial daily dose, and escalating or de-escalating the dose to achieve or maintain complete hematologic response, with no severe adverse reaction, wherein the composition is administered under fasting condition and wherein the composition results in a mean AUC₀₋₂₄ ranging from 6226±5827 ng*h/mL to 60373±55659 ng*h/mL.

In another particular embodiment, the present invention relates to a method for the treatment of an adult patient with chronic myeloid leukemia (CML), comprising orally administering a pharmaceutical composition comprising a therapeutic effective amount of a compound of Formula I or a pharmaceutically acceptable salt thereof, at an initial daily dose, and escalating or de-escalating the dose to achieve or maintain complete hematologic response, with no severe adverse reaction, wherein the composition is administered under fasting condition and wherein the composition results in a mean Cₘₐₓ ranging from 664±450 ng/ml to 5054±3051 ng/mL.

In a particular embodiment, the present invention relates to a method for the treatment of an adult patient with CML, comprising orally administering a pharmaceutical composition comprising a therapeutic effective amount of a compound of Formula I or a pharmaceutically acceptable salt thereof at an initial daily dose of between 10 mg and 200 mg and escalating to the dose between 20 mg and 210 mg to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction due to the composition comprising compound of Formula I or a pharmaceutically acceptable salt thereof, wherein the composition when administered under fasting condition results in a mean AUC₀₋₂₄ ranging from 6226±5827 ng*h/mL to 60373±55659 ng*h/mL and mean Cₘₐₓ ranging from 664±450 ng/mL to 5054±3051 ng/mL.

In yet another particular embodiment, the present invention relates to a method for the treatment of an adult patient with CML, comprising orally administering a therapeutically effective amount of a compound of Formula I or a pharmaceutically acceptable salt thereof to the patient at a daily dose wherein such daily dose results in a mean AUC₀₋₂₄ ranging from 6226±5827 ng*h/mL to 60373±55659 ng*h/mL and mean Cₘₐₓ ranging from 664±450 ng/ml to 5054±3051 ng/ml and wherein the daily dose does not result in patient's QT interval of more than about 500 ms.

In a particular embodiment, the present invention relates to a method for the prevention of the recurrence of CML in a patient that has previously undergone treatment using a tyrosine kinase inhibitor, wherein the method comprises orally administering a compound of Formula I or a pharmaceutically acceptable salt thereof at an initial daily dose of 174 mg, wherein the initial daily dose results in a mean AUC₀₋₂₄ ranging from 6226±5827 ng*h/mL to 60373±55659 ng*h/mL and mean Cₘₐₓ ranging from 664±450 ng/ml to 5054±3051 ng/ml.

In one particular embodiment, the present invention relates to a method for the treatment of an adult patient with newly diagnosed CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 7 days and resumed at 90 mg after recovery of patient from said toxicity; or
b) the initial daily dose is withheld for less than 7 days and if toxicity is resolved then resumed at 90 mg; or
c) the initial daily dose is withheld for at least 7 days and resumed at a reduced daily dose selected from 45 mg, 48 mg and 66 mg on subsequent instances of recurrences of said toxicity.

In another particular embodiment, the present invention relates to a method for the treatment of an adult patient with newly diagnosed CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 7 days and resumed at 90 mg after recovery of patient from said toxicity; or
b) the initial daily dose is withheld for less than 7 days and if toxicity is resolved then resumed at 90 mg; or
c) the initial daily dose is withheld for at least 7 days and resumed at a reduced daily dose selected from 45 mg, 48 mg and 66 mg on subsequent instances of recurrences of said toxicity
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another particular embodiment, the present invention relates to a method for the treatment of an adult patient with newly diagnosed CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 7 days and resumed at 90 mg after recovery of patient from said toxicity; or
b) the initial daily dose is withheld for less than 7 days and if toxicity is resolved then resumed at 90 mg; or
c) the initial daily dose is withheld for at least 7 days and resumed at a reduced daily dose selected from 45 mg, 48 mg and 66 mg on subsequent instances of recurrences of said toxicity;
and wherein the daily dose of the compound of Formula I or a pharmaceutically acceptable salt thereof in the patient results in mean AUC₀₋₂₄ ranging from 18237 ng*h/mL to 27355±2782 ng*h/mL.

In another particular embodiment, the present invention relates to a method for the treatment of an adult patient with newly diagnosed CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 7 days and resumed at 90 mg after recovery of patient from said toxicity; or
b) the initial daily dose is withheld for less than 7 days and if toxicity is resolved then resumed at 90 mg; or
c) the initial daily dose is withheld for at least 7 days and resumed at a reduced daily dose selected from 45 mg, 48 mg and 66 mg on subsequent instances of recurrences of said toxicity;
and wherein the daily dose of the compound of Formula I or a pharmaceutically acceptable salt thereof in the patient results in mean Cₘₐₓ ranging from 2277 ng/ml to 3415±1566 ng/mL.

In another embodiment, the present invention relates to a method for the treatment of an adult patient with newly diagnosed CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 7 days and resumed at 90 mg after recovery of patient from said toxicity; or
b) the initial daily dose is withheld for less than 7 days and if toxicity is resolved then resumed at 90 mg; or
c) the initial daily dose is withheld for at least 7 days and resumed at a reduced daily dose selected from 45 mg, 48 mg and 66 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction,
and wherein the daily dose of the compound of Formula I or a pharmaceutically acceptable salt thereof in the patient results in mean AUC₀₋₂₄ ranging from 18237 ng*h/mL to 27355±2782 ng*h/mL.

In another particular embodiment, the present invention relates to a method for the treatment of an adult patient with newly diagnosed CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 7 days and resumed at 90 mg after recovery of patient from said toxicity; or
b) the initial daily dose is withheld for less than 7 days and if toxicity is resolved then resumed at 90 mg; or
c) the initial daily dose is withheld for at least 7 days and resumed at a reduced daily dose selected from 45 mg, 48 mg and 66 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction,
and wherein the daily dose of the compound of Formula I or a pharmaceutically acceptable salt thereof in the patient results in mean Cₘₐₓ ranging from 2277 ng/ml to 3415±1566 ng/mL.

In one particular embodiment, the present invention relates to a method for the treatment of an adult patient with newly diagnosed CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 14 days and resumed at 90 mg after recovery of patient from said toxicity; or
b) the initial daily dose is withheld for less than 14 days and if toxicity is resolved then resumed at 90 mg; or
c) the initial daily dose is withheld for at least 14 days and resumed at a reduced daily dose selected from 45 mg, 48 mg and 66 mg on subsequent instances of recurrences of said toxicity.

In another particular embodiment, the present invention relates to a method for the treatment of an adult patient with newly diagnosed CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 14 days and resumed at 90 mg after recovery of patient from said toxicity; or
b) the initial daily dose is withheld for less than 14 days and if toxicity is resolved then resumed at 90 mg; or
c) the initial daily dose is withheld for at least 14 days and resumed at a reduced daily dose selected from 45 mg, 48 mg and 66 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another particular embodiment, the present invention relates to a method for the treatment of an adult patient with newly diagnosed CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 14 days and resumed at 90 mg after recovery of patient from said toxicity; or
b) the initial daily dose is withheld for less than 14 days and if toxicity is resolved then resumed at 90 mg; or
c) the initial daily dose is withheld for at least 14 days and resumed at a reduced daily dose selected from 45 mg, 48 mg and 66 mg on subsequent instances of recurrences of said toxicity;
and wherein the daily dose of the compound of Formula I or a pharmaceutically acceptable salt thereof in the patient results in mean AUC₀₋₂₄ ranging from 18237 ng*h/mL to 27355±2782 ng*h/mL.

In another particular embodiment, the present invention relates to a method for the treatment of an adult patient with newly diagnosed CML, wherein the method comprises administering to said patient an initial daily dose of 90 mg of a compound of Formula I or a pharmaceutically acceptable salt thereof such that when the patient develops non-hematological toxicity:
a) the initial daily dose is withheld for at least 14 days and resumed at 90 mg after recovery of patient from said toxicity; or
b) the initial daily dose is withheld for less than 14 days and if toxicity is resolved then resumed at 90 mg; or
c) the initial daily dose is withheld for at least 14 days and resumed at a reduced daily dose selected from 45 mg, 48 mg and 66 mg on subsequent instances of recurrences of said toxicity;
and wherein the daily dose of the compound of Formula I or a pharmaceutically acceptable salt thereof in the patient results in mean Cₘₐₓ ranging from 2277 ng/ml to 3415±1566 ng/mL.

In another particular embodiment, the present invention relates to a method for the treatment of an adult patient with newly diagnosed CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 14 days and resumed at 90 mg after recovery of patient from said toxicity; or
b) the initial daily dose is withheld for less than 14 days and if toxicity is resolved then resumed at 90 mg; or
c) the initial daily dose is withheld for at least 7 days and resumed at a reduced daily dose selected from 45 mg, 48 mg and 66 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction,
and wherein the daily dose of the compound of Formula I or a pharmaceutically acceptable salt thereof in the patient results in mean AUC₀₋₂₄ ranging from 18237 ng*h/mL to 27355±2782 ng*h/mL.

In another particular embodiment, the present invention relates to a method for the treatment of an adult patient with newly diagnosed CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 14 days and resumed at 90 mg after recovery of patient from said toxicity; or
b) the initial daily dose is withheld for less than 14 days and if toxicity is resolved then resumed at 90 mg; or
c) the initial daily dose is withheld for at least 7 days and resumed at a reduced daily dose selected from 45 mg, 48 mg and 66 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction,
and wherein the daily dose of the compound of Formula I or a pharmaceutically acceptable salt thereof in the patient results in mean Cₘₐₓ ranging from 2277 ng/ml to 3415±1566 ng/mL.

In one particular embodiment, the present invention relates to a method for the treatment of an adult patient with newly diagnosed CML, wherein the method comprises administering to the patient an initial daily dose of 90 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose selected from 45 mg, 48 mg and 66 mg on subsequent instances of recurrences of said toxicity.

In another particular embodiment, the present invention relates to a method for the treatment of an adult patient with newly diagnosed chronic myeloid leukemia (CML), wherein the method comprises administering to the patient an initial daily dose of 90 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose selected from 45 mg, 48 mg and 66 mg on subsequent instances of recurrences of said toxicity and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another embodiment, the present invention relates to a method for the treatment of an adult patient with newly diagnosed CML, wherein the method comprises administering to the patient an initial daily dose of 90 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose selected from 45 mg, 48 mg and 66 mg on subsequent instances of recurrences of said toxicity, and wherein the daily dose of the compound of Formula I or a pharmaceutically acceptable salt thereof in the patient results in mean AUC₀₋₂₄ ranging from 18237 ng*h/mL to 27355±2782 ng*h/mL.

In another embodiment, the present invention relates to a method for the treatment of an adult patient with newly diagnosed chronic myeloid leukemia (CML), wherein the method comprises administering to the patient an initial daily dose of 90 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose selected from 45 mg, 48 mg and 66 mg on subsequent instances of recurrences of said toxicity, and wherein the daily dose of the compound of Formula I or a pharmaceutically acceptable salt thereof in the patient results in mean Cₘₐₓ ranging from 2277 ng/mL to 3415±1566 ng/mL.

In another particular embodiment, the present invention relates to a method for the treatment of an adult patient with newly diagnosed CML, wherein the method comprises administering to the patient an initial daily dose of 90 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose selected from 45 mg, 48 mg and 66 mg on subsequent instances of recurrences of said toxicity and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof in the patient results in mean AUC₀₋₂₄ ranging from 18237 ng*h/mL to 27355±2782 ng*h/mL.

In another particular embodiment, the present invention relates to a method for the treatment of an adult patient with newly diagnosed CML, wherein the method comprises administering to the patient an initial daily dose of 90 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose selected from 45 mg, 48 mg and 66 mg on subsequent instances of recurrences of said toxicity and wherein,
the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof in the patient results in mean Cₘₐₓ ranging from 2277 ng/ml to 3415±1566 ng/ml.

In one embodiment, the present invention relates to a method for the treatment of an adult patient with refractory CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 7 days and resumed at 90 mg after recovery of patient from said toxicity; or
b) the initial daily dose is withheld for less than 7 days and if toxicity is resolved then resumed at 90 mg; or
c) the initial daily dose is withheld for at least 7 days and resumed at a reduced daily dose selected from 45 mg, 48 mg and 66 mg on subsequent instances of recurrences of said toxicity.

In another particular embodiment, the present invention relates to a method for the treatment of an adult patient with refractory CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 7 days and resumed at 90 mg after recovery of patient from said toxicity; or
b) the initial daily dose is withheld for less than 7 days and if toxicity is resolved then resumed at 90 mg; or
c) the initial daily dose is withheld for at least 7 days and resumed at a reduced daily dose selected from 45 mg, 48 mg and 66 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another particular embodiment, the present invention relates to a method for the treatment of an adult patient with refractory CML, wherein the method comprises administering to the patient an initial daily dose of 90 mg of a compound of Formula I or a pharmaceutically acceptable salt thereof such that when the patient develops non-hematological toxicity:
a) the initial daily dose is withheld for at least 7 days and resumed at 90 mg after recovery of patient from said toxicity; or
b) the initial daily dose is withheld for less than 7 days and if toxicity is resolved then resumed at 90 mg; or
c) the initial daily dose is withheld for at least 7 days and resumed at a reduced daily dose selected from 45 mg, 48 mg and 66 mg on subsequent instances of recurrences of said toxicity;
and wherein the daily dose of the compound of Formula I or a pharmaceutically acceptable salt thereof in the patient results in mean AUC₀₋₂₄ ranging from 18237 ng*h/mL to 27355±2782 ng*h/mL.

In another particular embodiment, the present invention relates to a method for the treatment of an adult patient with refractory chronic myeloid leukemia (CML), wherein the method comprises administering to the patient an initial daily dose of 90 mg of a compound of Formula I or a pharmaceutically acceptable salt thereof such that when the patient develops non-hematological toxicity:
a) the initial daily dose is withheld for at least 7 days and resumed at 90 mg after recovery of patient from said toxicity; or
b) the initial daily dose is withheld for less than 7 days and if toxicity is resolved then resumed at 90 mg; or
c) the initial daily dose is withheld for at least 7 days and resumed at a reduced daily dose selected from 45 mg, 48 mg and 66 mg on subsequent instances of recurrences of said toxicity;
and wherein the daily dose of the compound of Formula I or a pharmaceutically acceptable salt thereof in the patient results in mean Cₘₐₓ ranging from 2277 ng/ml to 3415±1566 ng/mL.

In another particular embodiment, the present invention provides a method for the treatment of an adult patient with refractory CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 7 days and resumed at 90 mg after recovery of patient from said toxicity; or
b) the initial daily dose is withheld for less than 7 days and if toxicity is resolved then resumed at 90 mg; or
c) the initial daily dose is withheld for at least 7 days and resumed at a reduced daily dose selected from 45 mg, 48 mg and 66 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction,
and wherein the daily dose of the compound of Formula I or a pharmaceutically acceptable salt thereof in the patient results in mean AUC₀₋₂₄ ranging from 18237 ng*h/mL to 27355±2782 ng*h/mL.

In another particular embodiment, the present invention provides a method for the treatment of an adult patient with refractory CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 7 days and resumed at 90 mg after recovery of patient from said toxicity; or
b) the initial daily dose is withheld for less than 7 days and if toxicity is resolved then resumed at 90 mg; or
c) the initial daily dose is withheld for at least 7 days and resumed at a reduced daily dose selected from 45 mg, 48 mg and 66 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction,
and wherein the daily dose of the compound of Formula I or a pharmaceutically acceptable salt thereof in the patient results in mean Cₘₐₓ ranging from 2277 ng/ml to 3415±1566 ng/mL.

In one particular embodiment, the present invention relates to a method for the treatment of an adult patient with refractory CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 14 days and resumed at 90 mg after recovery of patient from said toxicity; or
b) the initial daily dose is withheld for less than 14 days and if toxicity is resolved then resumed at 90 mg; or
c) the initial daily dose is withheld for at least 14 days and resumed at a reduced daily dose selected from 45 mg, 48 mg and 66 mg on subsequent instances of recurrences of said toxicity.

In another particular embodiment, the present invention relates to a method for the treatment of an adult patient with refractory CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 14 days and resumed at 90 mg after recovery of patient from said toxicity; or
b) the initial daily dose is withheld for less than 7 days and if toxicity is resolved then resumed at 90 mg; or
c) the initial daily dose is withheld for at least 7 days and resumed at a reduced daily dose selected from 45 mg, 48 mg and 66 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another particular embodiment, the present invention relates to a method for the treatment of an adult patient with refractory CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 14 days and resumed at 90 mg after recovery of patient from said toxicity; or
b) the initial daily dose is withheld for less than 14 days and if toxicity is resolved then resumed at 90 mg; or
c) the initial daily dose is withheld for at least 14 days and resumed at a reduced daily dose selected from 45 mg, 48 mg and 66 mg on subsequent instances of recurrences of said toxicity;
and wherein the daily dose of the compound of Formula I or a pharmaceutically acceptable salt thereof in the patient results in mean AUC₀₋₂₄ ranging from 18237 ng*h/mL to 27355±2782 ng*h/mL.

In another particular embodiment, the present invention relates to a method for the treatment of an adult patient with refractory CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 14 days and resumed at 90 mg after recovery of patient from said toxicity; or
b) the initial daily dose is withheld for less than 14 days and if toxicity is resolved then resumed at 90 mg; or
c) the initial daily dose is withheld for at least 14 days and resumed at a reduced daily dose selected from 45 mg, 48 mg and 66 mg on subsequent instances of recurrences of said toxicity;
and wherein the daily dose of the compound of Formula I or a pharmaceutically acceptable salt thereof in the patient results in mean Cₘₐₓ ranging from 2277 ng/ml to 3415±1566 ng/ml.

In another particular embodiment, the present invention relates to a method for the treatment of an adult patient with refractory CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 14 days and resumed at 90 mg after recovery of patient from said toxicity; or
b) the initial daily dose is withheld for less than 14 days and if toxicity is resolved then resumed at 90 mg; or
c) the initial daily dose is withheld for at least 14 days and resumed at a reduced daily dose selected from 45 mg, 48 mg and 66 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction,
and wherein the daily dose of the compound of Formula I or a pharmaceutically acceptable salt thereof in the patient results in mean AUC₀₋₂₄ ranging from 18237 ng*h/mL to 27355±2782 ng*h/mL.

In another particular embodiment, the present invention relates to a method for the treatment of an adult patient with refractory CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 14 days and resumed at 90 mg after recovery of patient from said toxicity; or
b) the initial daily dose is withheld for less than 14 days and if toxicity is resolved then resumed at 90 mg; or
c) the initial daily dose is withheld for at least 14 days and resumed at a reduced daily dose selected from 45 mg, 48 mg and 66 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction,
and wherein the daily dose of the compound of Formula I or a pharmaceutically acceptable salt thereof results in a mean Cₘₐₓ ranging from 2277 ng/mL to 3415±1566 ng/ml.

In another particular embodiment, the present invention relates to a method for the treatment of an adult patient with refractory CML, wherein the method comprises administering to the patient an initial daily dose of 90 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose selected from 45 mg, 48 mg and 66 mg on subsequent instances of recurrences of said toxicity.

In another particular embodiment, the present invention relates to a method for the treatment of an adult patient with refractory CML, wherein the method comprises administering to the patient an initial daily dose of 90 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose selected from 45 mg, 48 mg and 66 mg on subsequent instances of recurrences of said toxicity and wherein,
the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another particular embodiment, the present invention relates to a method for the treatment of an adult patient with refractory CML, wherein the method comprises administering to the patient an initial daily dose of 90 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose selected from 45 mg, 48 mg and 66 mg on subsequent instances of recurrences of said toxicity, and wherein the daily dose of the compound of Formula I or a pharmaceutically acceptable salt thereof in the patient results in mean AUC₀₋₂₄ ranging from 18237 ng*h/mL to 27355±2782 ng*h/mL.

In another particular embodiment, the present invention relates to a method for the treatment of an adult patient with refractory CML, wherein the method comprises administering to the patient an initial daily dose of 90 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose selected from 45 mg, 48 mg and 66 mg on subsequent instances of recurrences of said toxicity, and wherein the daily dose of the compound of Formula I or a pharmaceutically acceptable salt thereof in the patient results in mean Cₘₐₓ ranging from 2277 ng/ml to 3415±1566 ng/ml.

In another particular embodiment, the present invention relates to a method for the treatment of an adult patient with refractory CML, wherein the method comprises administering to the patient an initial daily dose of 90 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose selected from 45 mg, 48 mg and 66 mg on subsequent instances of recurrences of said toxicity and wherein,
the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another particular embodiment, the present invention relates to a method for the treatment of an adult patient with refractory CML, wherein the method comprises administering to the patient an initial daily dose of 90 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose selected from 45 mg, 48 mg and 66 mg on subsequent instances of recurrences of said toxicity and wherein,
the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof in the patient results in mean AUC₀₋₂₄ ranging from 18237 ng*h/mL to 27355±2782 ng*h/mL.

In another particular embodiment, the present invention relates to a method for the treatment of an adult patient with refractory CML, wherein the method comprises administering to the patient an initial daily dose of 90 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose selected from 45 mg, 48 mg and 66 mg on subsequent instances of recurrences of said toxicity and wherein,
the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof results in a mean Cₘₐₓ ranging from 2277 ng/ml to 3415±1566 ng/ml.

In one particular embodiment, the present invention relates to a method for the treatment of an adult patient with newly diagnosed CML, wherein the method comprises administering to said patient an initial daily dose of 87 mg of a compound of Formula I or a pharmaceutically acceptable salt thereof such that when the patient develops non-hematologic toxicity:
a) the initial daily dose is withheld for at least 7 days and resumed at 87 mg after recovery of patient from said toxicity; or
b) the initial daily dose is withheld for less than 7 days and if toxicity is resolved then resumed at 87 mg; or
c) the initial daily dose is withheld for at least 7 days and resumed at a reduced daily dose of 43.5 mg for said toxicity.

In another embodiment, the present invention relates to a method for the treatment of an adult patient with newly diagnosed CML, wherein the method comprises administering to said patient an initial daily dose of 87 mg of a compound of Formula I or a pharmaceutically acceptable salt thereof such that when the patient develops non-hematologic toxicity:
a) the initial daily dose is withheld for at least 7 days and resumed at 87 mg after recovery of patient from said toxicity; or
b) the initial daily dose is withheld for less than 7 days and if toxicity is resolved then resumed at 87 mg; or
c) the initial daily dose is withheld for at least 7 days and resumed at a reduced daily dose of 43.5 mg for said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In one particular embodiment, the present invention relates to a method for the treatment of an adult patient with newly diagnosed CML, wherein the method comprises administering to said patient an initial daily dose of 87 mg of a compound of Formula I or a pharmaceutically acceptable salt thereof such that when the patient develops non-hematologic toxicity:
a) the initial daily dose is withheld for at least 14 days and resumed at 87 mg after recovery of patient from said toxicity; or
b) the initial daily dose is withheld for less than 14 days and if toxicity is resolved then resumed at 87 mg; or
c) the initial daily dose is withheld for at least 14 days and resumed at a reduced daily dose of 43.5 mg for said toxicity.

In another particular embodiment, the present invention relates to a method for the treatment of an adult patient with newly diagnosed CML, wherein the method comprises administering to said patient an initial daily dose of 87 mg of a compound of Formula I or a pharmaceutically acceptable salt thereof such that when the patient develops non-hematological toxicity:
a) the initial daily dose is withheld for at least 14 days and resumed at 87 mg after recovery of patient from said toxicity; or
b) the initial daily dose is withheld for less than 14 days and if toxicity is resolved then resumed at 87 mg; or
c) the initial daily dose is withheld for at least 14 days and resumed at a reduced daily dose of 43.5 mg for said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In one particular embodiment, the present invention relates to a method for the treatment of an adult patient with newly diagnosed CML, wherein the method comprises administering to the patient an initial daily dose of 87 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose of 43.5 mg on subsequent instances of recurrences of said toxicity.

In another particular embodiment, the present invention relates to a method for the treatment of an adult patient with newly diagnosed CML, wherein the method comprises administering to the patient an initial daily dose of 87 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose of 43.5 mg on subsequent instances of recurrences of said toxicity and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In one particular embodiment, the present invention relates to a method for the treatment of an adult patient with refractory CML, wherein the method comprises administering to said patient an initial daily dose of 87 mg of a compound of Formula I or a pharmaceutically acceptable salt thereof such that when the patient develops non-hematologic toxicity:
a) the initial daily dose is withheld for at least 7 days and resumed at 87 mg after recovery of patient from said toxicity; or
b) the initial daily dose is withheld for less than 7 days and if toxicity is resolved then resumed at 87 mg; or
c) the initial daily dose is withheld for at least 7 days and resumed at a reduced daily dose of 43.5 mg for said toxicity.

In another particular embodiment, the present invention relates to a method for the treatment of an adult patient with refractory CML, wherein the method comprises administering to said patient an initial daily dose of 87 mg of a compound of Formula I or a pharmaceutically acceptable salt thereof such that when the patient develops non-hematologic toxicity:
a) the initial daily dose is withheld for at least 7 days and resumed at 87 mg after recovery of patient from said toxicity; or
b) the initial daily dose is withheld for less than 7 days and if toxicity is resolved then resumed at 87 mg; or
c) the initial daily dose is withheld for at least 7 days and resumed at a reduced daily dose of 43.5 mg for said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In one particular embodiment, the present invention relates to a method for the treatment of an adult patient with refractory CML, wherein the method comprises administering to said patient an initial daily dose of 87 mg of a compound of Formula I or a pharmaceutically acceptable salt thereof such that when the patient develops non-hematologic toxicity:
a) the initial daily dose is withheld for at least 14 days and resumed at 87 mg after recovery of patient from said toxicity; or
b) the initial daily dose is withheld for less than 14 days and if toxicity is resolved then resumed at 87 mg; or
c) the initial daily dose is withheld for at least 14 days and resumed at a reduced daily dose of 43.5 mg for said toxicity.

In another particular embodiment, the present invention relates to a method for the treatment of an adult patient with refractory CML, wherein the method comprises administering to said patient an initial daily dose of 87 mg of a compound of Formula I or a pharmaceutically acceptable salt thereof such that when the patient develops non-hematologic toxicity:
a) the initial daily dose is withheld for at least 14 days and resumed at 87 mg after recovery of patient from said toxicity; or
b) the initial daily dose is withheld for less than 14 days and if toxicity is resolved then resumed at 87 mg; or
c) the initial daily dose is withheld for at least 14 days and resumed at a reduced daily dose of 43.5 mg for said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In one particular embodiment, the present invention relates to a method for the treatment of an adult patient with refractory CML, wherein the method comprises administering to the patient an initial daily dose of 87 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose of 43.5 mg on subsequent instances of recurrences of said toxicity.

In another particular embodiment, the present invention relates to a method for the treatment of an adult patient with refractory CML, wherein the method comprises administering to the patient an initial daily dose of 87 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose of 43.5 mg on subsequent instances of recurrences of said toxicity and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another particular embodiment, the present invention relates to a method for the treatment of an adult patient with refractory CML, wherein the method comprises administering to the patient an initial daily dose of 87 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose of 43.5 mg on subsequent instances of recurrences of said toxicity and wherein,
the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In one embodiment, the present invention relates to a method for the treatment of an adult patient with newly diagnosed CML, wherein the method comprises administering to said patient an initial daily dose of 130.5 mg of a compound of Formula I or a pharmaceutically acceptable salt thereof such that when the patient develops non-hematologic toxicity:
a) the initial daily dose is withheld for at least 7 days and resumed at 130.5 mg after recovery of patient from said toxicity; or
b) the initial daily dose is withheld for less than 7 days and if toxicity is resolved then resumed at 130.5 mg; or
c) the initial daily dose is withheld for at least 7 days and resumed at a reduced daily dose of 87 mg on first instance and to 43.5 mg, on subsequent instances of recurrences of said toxicity.

In another particular embodiment, the present invention relates to a method for the treatment of an adult patient with newly diagnosed CML, wherein the method comprises administering to said patient an initial daily dose of 130.5 mg of a compound of Formula I or a pharmaceutically acceptable salt thereof such that when the patient develops non-hematologic toxicity:
a) the initial daily dose is withheld for at least 7 days and resumed at 130.5 mg after recovery of patient from said toxicity; or
b) the initial daily dose is withheld for less than 7 days and if toxicity is resolved then resumed at 130.5 mg; or
c) the initial daily dose is withheld for at least 7 days and resumed at a reduced daily dose of 87 mg on first instance and to 43.5 mg, on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In one embodiment, the present invention relates to a method for the treatment of an adult patient with newly diagnosed CML, wherein the method comprises administering to said patient an initial daily dose of 130.5 mg of a compound of Formula I or a pharmaceutically acceptable salt thereof such that when the patient develops non-hematologic toxicity:
a) the initial daily dose is withheld for at least 14 days and resumed at 130.5 mg after recovery of patient from said toxicity; or
b) the initial daily dose is withheld for less than 14 days and if toxicity is resolved then resumed at 130.5 mg; or
c) the initial daily dose is withheld for at least 14 days and resumed at a reduced daily dose of 87 mg on first instance and to 43.5 mg, on subsequent instances of recurrences of said toxicity.

In another particular embodiment, the present invention relates to a method for the treatment of an adult patient with newly diagnosed CML, wherein the method comprises administering to said patient an initial daily dose of 130.5 mg of a compound of Formula I or a pharmaceutically acceptable salt thereof such that when the patient develops non-hematologic toxicity:
a) the initial daily dose is withheld for at least 14 days and resumed at 130.5 mg after recovery of patient from said toxicity; or
b) the initial daily dose is withheld for less than 14 days and if toxicity is resolved then resumed at 130.5 mg; or
c) the initial daily dose is withheld for at least 14 days and resumed at a reduced daily dose of 87 mg on first instance and to 43.5 mg, on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In one embodiment, the present invention relates to a method for the treatment of an adult patient with newly diagnosed CML, wherein the method comprises administering to the patient an initial daily dose of 130.5 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose of 87 mg on first instance and to 43.5 mg on subsequent instances of recurrences of said toxicity.

In another particular embodiment, the present invention relates to a method for the treatment of an adult patient with newly diagnosed CML, wherein the method comprises administering to the patient an initial daily dose of 130.5 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose of 87 mg on first instance and to 43.5 mg on subsequent instances of recurrences of said toxicity and wherein,
the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In one embodiment, the present invention relates to a method for the treatment of an adult patient with refractory CML, wherein the method comprises administering to said patient an initial daily dose of 130.5 mg of a compound of Formula I or a pharmaceutically acceptable salt thereof such that when the patient develops non-hematologic toxicity:
a) the initial daily dose is withheld for at least 7 days and resumed at 130.5 mg after
b) the initial daily dose is withheld for less than 7 days and if toxicity is resolved then resumed at 130.5 mg; or
c) the initial daily dose is withheld for at least 7 days and resumed at a reduced daily dose of 87 mg on first instance and to 43.5 mg, on subsequent instances of recurrences of said toxicity.

In another particular embodiment, the present invention relates to a method for the treatment of an adult patient with refractory CML, wherein the method comprises administering to said patient an initial daily dose of 130.5 mg of a compound of Formula I or a pharmaceutically acceptable salt thereof such that when the patient develops non-hematologic toxicity:
a) the initial daily dose is withheld for at least 7 days and resumed at 130.5 mg after recovery of patient from said toxicity; or
b) the initial daily dose is withheld for less than 7 days and if toxicity is resolved then resumed at 130.5 mg; or
c) the initial daily dose is withheld for at least 7 days and resumed at a reduced daily dose of 87 mg on first instance and to 43.5 mg, on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In one particular embodiment, the present invention relates to a method for the treatment of an adult patient with refractory CML, wherein the method comprises administering to said patient an initial daily dose of 130.5 mg of a compound of Formula I or a pharmaceutically acceptable salt thereof such that when the patient develops non-hematologic toxicity:
a) the initial daily dose is withheld for at least 14 days and resumed at 130.5 mg after
b) the initial daily dose is withheld for less than 14 days and if toxicity is resolved then resumed at 130.5 mg; or
c) the initial daily dose is withheld for at least 14 days and resumed at a reduced daily dose of 87 mg on first instance and to 43.5 mg, on subsequent instances of recurrences of said toxicity.

In another particular embodiment, the present invention relates to a method for the treatment of an adult patient with refractory CML, wherein the method comprises administering to said patient an initial daily dose of 130.5 mg of a compound of Formula I or a pharmaceutically acceptable salt thereof such that when the patient develops non-hematologic toxicity;
a) the initial daily dose is withheld for at least 14 days and resumed at 130.5 mg after recovery of patient from said toxicity; or
b) the initial daily dose is withheld for less than 14 days and if toxicity is resolved then resumed at 130.5 mg; or
c) the initial daily dose is withheld for at least 14 days and resumed at a reduced daily dose of 87 mg on first instance and to 43.5 mg, on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In one embodiment, the present invention relates to a method for the treatment of an adult patient with refractory chronic myeloid leukemia (CML), wherein the method comprises administering to the patient an initial daily dose of 130.5 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose of 87 mg on first instance and to 43.5 mg on subsequent instances of recurrences of said toxicity.

In another particular embodiment, the present invention relates to a method for the treatment of an adult patient with refractory chronic myeloid leukemia (CML), wherein the method comprises administering to the patient an initial daily dose of 130.5 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose of 87 mg on first instance and to 43.5 mg on subsequent instances of recurrences of said toxicity and wherein,
the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another particular embodiment, the present invention relates to a method for the treatment of an adult patient with refractory CML, wherein the method comprises administering to the patient an initial daily dose of 130.5 mg of a compound of Formula such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose of 87 mg on first instance and to 43.5 mg on subsequent instances of recurrences of said toxicity and wherein,
the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In one particular embodiment, the present invention relates to a method for the treatment of an adult patient with newly diagnosed CML, wherein the method comprises administering to said patient an initial daily dose of 126 mg of a compound of Formula I or a pharmaceutically acceptable salt thereof such that when the patient develops non-hematologic toxicity:
a) the initial daily dose is withheld for at least 7 days and resumed at 126 mg after recovery of patient from said toxicity; or
b) the initial daily dose is withheld for less than 7 days and if toxicity is resolved then resumed at 126 mg; or
c) the initial daily dose is withheld for at least 7 days and resumed at a reduced daily dose of 90 mg on first instance and to 66 mg, 48 mg, on subsequent instances of recurrences of said toxicity.

In another particular embodiment, the present invention relates to a method for the treatment of an adult patient with newly diagnosed CML, wherein the method comprises administering to said patient an initial daily dose of 126 mg of a compound of Formula I or a pharmaceutically acceptable salt thereof such that when the patient develops non-hematologic toxicity:
a) the initial daily dose is withheld for at least 7 days and resumed at 126 mg after recovery of patient from said toxicity; or
b) the initial daily dose is withheld for less than 7 days and if toxicity is resolved then resumed at 126 mg; or
c) the initial daily dose is withheld for at least 7 days and resumed at a reduced daily dose of 90 mg on first instance and to 66 mg, 48 mg, on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another particular embodiment, the present invention relates to a method for the treatment of an adult patient with newly diagnosed CML, wherein the method comprises administering to said patient an initial daily dose of 126 mg of a compound of Formula I or a pharmaceutically acceptable salt thereof such that when the patient develops non-hematologic toxicity:
a) the initial daily dose is withheld for at least 7 days and resumed at 126 mg after recovery of patient from said toxicity; or
b) the initial daily dose is withheld for less than 7 days and if toxicity is resolved then resumed at 126 mg; or
c) the initial daily dose is withheld for at least 7 days and resumed at a reduced daily dose of 90 mg on first instance and to 66 mg, 48 mg, on subsequent instances of recurrences of said toxicity;
and wherein the daily dose of the compound of Formula I or a pharmaceutically acceptable salt thereof in the patient results in mean AUC₀₋₂₄ ranging from 40249 ng*h/mL to 60373±55659 ng*h/mL.

In another particular embodiment, the present invention relates to a method for the treatment of an adult patient with newly diagnosed CML, wherein the method comprises administering to said patient an initial daily dose of 126 mg of a compound of Formula I or a pharmaceutically acceptable salt thereof such that when the patient develops non-hematologic toxicity:
a) the initial daily dose is withheld for at least 7 days and resumed at 126 mg after recovery of patient from said toxicity; or
b) the initial daily dose is withheld for less than 7 days and if toxicity is resolved then resumed at 126 mg; or
c) the initial daily dose is withheld for at least 7 days and resumed at a reduced daily dose of 90 mg on first instance and to 66 mg, 48 mg, on subsequent instances of recurrences of said toxicity;
and wherein the daily dose of the compound of Formula I or a pharmaceutically acceptable salt thereof in the patient results in mean Cₘₐₓ ranging from 3282 ng/ml to 4924±3289 ng/mL.

In another particular embodiment, the present invention relates to a method for the treatment of an adult patient with newly diagnosed CML, wherein the method comprises administering to said patient an initial daily dose of 126 mg of a compound of Formula I or a pharmaceutically acceptable salt thereof such that when the patient develops non-hematologic toxicity:
a) the initial daily dose is withheld for at least 7 days and resumed at 126 mg after recovery of patient from said toxicity; or
b) the initial daily dose is withheld for less than 7 days and if toxicity is resolved then resumed at 126 mg; or
c) the initial daily dose is withheld for at least 7 days and resumed at a reduced daily dose of 90 mg on first instance and to 66 mg, 48 mg, on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof in the patient results in mean AUC₀₋₂₄ ranging from 40249 ng*h/mL to 60373±55659 ng*h/mL.

In another particular embodiment, the present invention relates to a method for the treatment of an adult patient with newly diagnosed CML, wherein the method comprises administering to said patient an initial daily dose of 126 mg of a compound of Formula I or a pharmaceutically acceptable salt thereof such that when the patient develops non-hematological toxicity:
a) the initial daily dose is withheld for at least 7 days and resumed at 126 mg after
b) the initial daily dose is withheld for at least 7 days and resumed at a reduced daily dose of 90 mg on first instance and to 66 mg, 48 mg, on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof in the patient results in mean Cₘₐₓ ranging from 3282 ng/ml to 4924±3289 ng/mL.

In another particular embodiment, the present invention relates to a method for the treatment of an adult patient with newly diagnosed CML, wherein the method comprises administering to said patient an initial daily dose of 126 mg of a compound of Formula I or a pharmaceutically acceptable salt thereof such that when the patient develops non-hematologic toxicity:
a) the initial daily dose is withheld for at least 14 days and resumed at 126 mg after recovery of patient from said toxicity; or
b) the initial daily dose is withheld for less than 14 days and if toxicity is resolved then resumed at 126 mg; or
c) the initial daily dose is withheld for at least 14 days and resumed at a reduced daily dose of 90 mg on first instance and to 66 mg, 48 mg, on subsequent instances of recurrences of said toxicity.

In another particular embodiment, the present invention relates to a method for the treatment of an adult patient with newly diagnosed CML, wherein the method comprises administering to said patient an initial daily dose of 126 mg of a compound of Formula I or a pharmaceutically acceptable salt thereof such that when the patient develops non-hematological toxicity:
a) the initial daily dose is withheld for at least 14 days and resumed at 126 mg after recovery of patient from said toxicity; or
b) the initial daily dose is withheld for less than 14 days and if toxicity is resolved then resumed at 126 mg; or
c) the initial daily dose is withheld for at least 14 days and resumed at a reduced daily dose of 90 mg on first instance and to 66 mg, 48 mg, on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another particular embodiment, the present invention relates to a method for the treatment of an adult patient with newly diagnosed CML, wherein the method comprises administering to said patient an initial daily dose of 126 mg of a compound of Formula I or a pharmaceutically acceptable salt thereof such that when the patient develops non-hematologic toxicity:
a) the initial daily dose is withheld for at least 14 days and resumed at 126 mg after recovery of patient from said toxicity; or
b) the initial daily dose is withheld for less than 14 days and if toxicity is resolved then resumed at 126 mg; or
c) the initial daily dose is withheld for at least 14 days and resumed at a reduced daily dose of 90 mg on first instance and to 66 mg, 48 mg, on subsequent instances of recurrences of said toxicity;
and wherein the daily dose of the compound of Formula I or a pharmaceutically acceptable salt thereof in the patient results in mean AUC₀₋₂₄ ranging from 40249 ng*h/mL to 60373±55659 ng*h/mL.

In another particular embodiment, the present invention relates to a method for the treatment of an adult patient with newly diagnosed CML, wherein the method comprises administering to said patient an initial daily dose of 126 mg of a compound of Formula I or a pharmaceutically acceptable salt thereof such that when the patient develops non-hematologic toxicity:
a) the initial daily dose is withheld for at least 14 days and resumed at 126 mg after recovery of patient from said toxicity; or
b) the initial daily dose is withheld for less than 14 days and if toxicity is resolved then resumed at 126 mg; or
c) the initial daily dose is withheld for at least 14 days and resumed at a reduced daily dose of 90 mg on first instance and to 66 mg, 48 mg, on subsequent instances of recurrences of said toxicity;
and wherein the daily dose of the compound of Formula I or a pharmaceutically acceptable salt thereof in the patient results in mean Cₘₐₓ ranging from 3282 ng/mL to 4924±3289 ng/mL.

In another particular embodiment, the present invention relates to a method for the treatment of an adult patient with newly diagnosed CML, wherein the method comprises administering to said patient an initial daily dose of 126 mg of a compound of Formula I or a pharmaceutically acceptable salt thereof such that when the patient develops non-hematologic toxicity:
a) the initial daily dose is withheld for at least 14 days and resumed at 126 mg after recovery of patient from said toxicity; or
b) the initial daily dose is withheld for less than 14 days and if toxicity is resolved then resumed at 126 mg; or
c) the initial daily dose is withheld for at least 14 days and resumed at a reduced daily dose of 90 mg on first instance and to 66 mg, 48 mg, on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof in the patient results in mean AUC₀₋₂₄ ranging from 40249 ng*h/mL to 60373±55659 ng*h/mL.

In another particular embodiment, the present invention relates to a method for the treatment of an adult patient with newly diagnosed CML, wherein the method comprises administering to said patient an initial daily dose of 126 mg of a compound of Formula I or a pharmaceutically acceptable salt thereof such that when the patient develops non-hematologic toxicity:
a) the initial daily dose is withheld for at least 14 days and resumed at 126 mg after recovery of patient from said toxicity; or
b) the initial daily dose is withheld for less than 14 days and if toxicity is resolved then resumed at 126 mg; or
c) the initial daily dose is withheld for at least 14 days and resumed at a reduced daily dose of 90 mg on first instance and to 66 mg, 48 mg, on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof in the patient results in mean Cₘₐₓ ranging from 3282 ng/mL to 4924±3289 ng/mL.

In one particular embodiment, the present invention relates to a method for the treatment of an adult patient with newly diagnosed CML, wherein the method comprises administering to the patient an initial daily dose of 126 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose of 90 mg on first instance and to 66 mg, 48 mg, on subsequent instances of recurrences of said toxicity.

In another particular embodiment, the present invention relates to a method for the treatment of an adult patient with newly diagnosed CML, wherein the method comprises administering to the patient an initial daily dose of 126 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose of 90 mg on first instance and to 66 mg, 48 mg, on subsequent instances of recurrences of said toxicity and wherein,
the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another particular embodiment, the present invention relates to a method for the treatment of an adult patient with newly diagnosed CML, wherein the method comprises administering to the patient an initial daily dose of 126 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose of 90 mg on first instance and to 66 mg, 48 mg, on subsequent instances of recurrences of said toxicity, and wherein the daily dose of the compound of Formula I or a pharmaceutically acceptable salt thereof in the patient results in mean AUC₀₋₂₄ ranging from 40249 ng*h/mL to 60373±55659 ng*h/mL.

In another particular embodiment, the present invention relates to a method for the treatment of an adult patient with newly diagnosed chronic myeloid leukemia (CML), wherein the method comprises administering to the patient an initial daily dose of 126 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose of 90 mg on first instance and to 66 mg, 48 mg, on subsequent instances of recurrences of said toxicity, and wherein the daily dose of the compound of Formula I or a pharmaceutically acceptable salt thereof in the patient results in mean Cₘₐₓ ranging from 3282 ng/mL to 4924±3289 ng/mL.

In another particular embodiment, the present invention relates to a method for the treatment of an adult patient with newly diagnosed CML, wherein the method comprises administering to the patient an initial daily dose of 126 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose of 90 mg on first instance and to 66 mg, 48 mg, on subsequent instances of recurrences of said toxicity and wherein,
the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof in the patient results in mean AUC₀₋₂₄ ranging from 40249 ng*h/mL to 60373±55659 ng*h/mL.

In another particular embodiment, the present invention relates to a method for the treatment of an adult patient with newly diagnosed CML, wherein the method comprises administering to the patient an initial daily dose of 126 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose of 90 mg on first instance and to 66 mg, 48 mg, on subsequent instances of recurrences of said toxicity and wherein,
the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof in the patient results in mean Cₘₐₓ ranging from 3282 ng/ml to 4924±3289 ng/ml.

In one particular embodiment, the present invention relates to a method for the treatment of an adult patient with refractory CML, wherein the method comprises administering to said patient an initial daily dose of 126 mg of a compound of Formula I or a pharmaceutically acceptable salt thereof such that when the patient develops non-hematologic toxicity:
a) the initial daily dose is withheld for at least 7 days and resumed at 126 mg after recovery of patient from said toxicity; or
b) the initial daily dose is withheld for less than 7 days and if toxicity is resolved then resumed at 126 mg; or
c) the initial daily dose is withheld for at least 7 days and resumed at a reduced daily dose of 90 mg on first instance and to 66 mg, 48 mg, on subsequent instances of recurrences of said toxicity.

In another particular embodiment, the present invention relates to a method for the treatment of an adult patient with refractory CML, wherein the method comprises administering to said patient an initial daily dose of 126 mg of a compound of Formula I or a pharmaceutically acceptable salt thereof such that when the patient develops non-hematologic toxicity:
a) the initial daily dose is withheld for at least 7 days and resumed at 126 mg after recovery of patient from said toxicity; or
b) the initial daily dose is withheld for less than 7 days and if toxicity is resolved then resumed at 126 mg; or
c) the initial daily dose is withheld for at least 7 days and resumed at a reduced daily dose of 90 mg on first instance and to 66 mg, 48 mg, on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another particular embodiment, the present invention relates to a method for the treatment of an adult patient with refractory CML, wherein the method comprises administering to the patient initial daily dose of 126 mg of a compound of Formula I or a pharmaceutically acceptable salt thereof such that when the patient develops non-hematological toxicity:
a) the initial daily dose is withheld for at least 7 days and resumed at 126 mg after recovery of patient from said toxicity; or
b) the initial daily dose is withheld for less than 7 days and if toxicity is resolved then resumed at 126 mg; or
c) the initial daily dose is withheld for at least 7 days and resumed at a reduced daily dose of 90 mg on first instance and to 66 mg, 48 mg, on subsequent instances of recurrences of said toxicity;
and wherein the daily dose of the compound of Formula I or a pharmaceutically acceptable salt thereof in the patient results in mean AUC₀₋₂₄ ranging from 40249 ng*h/mL to 60373±55659 ng*h/mL.

In another particular embodiment, the present invention relates to a method for the treatment of an adult patient with refractory CML, wherein the method comprises administering to the patient initial daily dose of 126 mg of a compound of Formula I or a pharmaceutically acceptable salt thereof such that when the patient develops non-hematological toxicity:
a) the initial daily dose is withheld for at least 7 days and resumed at 126 mg after recovery of patient from said toxicity; or
b) the initial daily dose is withheld for less than 7 days and if toxicity is resolved then resumed at 126 mg; or
c) the initial daily dose is withheld for at least 7 days and resumed at a reduced daily dose of 90 mg on first instance and to 66 mg, 48 mg, on subsequent instances of recurrences of said toxicity;
and wherein the daily dose of the compound of Formula I or a pharmaceutically acceptable salt thereof in the patient results in mean Cₘₐₓ ranging from 3282 ng/ml to 4924±3289 ng/mL.

In another particular embodiment, the present invention provides a method for the treatment of an adult patient with refractory CML, wherein the method comprises administering to said patient an initial daily dose of 126 mg of a compound of Formula I or a pharmaceutically acceptable salt thereof such that when the patient develops non-hematologic toxicity:
a) the initial daily dose is withheld for at least 7 days and resumed at 126 mg after recovery of patient from said toxicity; or
b) the initial daily dose is withheld for less than 7 days and if toxicity is resolved then resumed at 126 mg; or
c) the initial daily dose is withheld for at least 7 days and resumed at a reduced daily dose of 90 mg on first instance and to 66 mg, 48 mg, on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof in the patient results in mean AUC₀₋₂₄ ranging from 40249 ng*h/mL to 60373±55659 ng*h/mL.

In another particular embodiment, the present invention provides a method for the treatment of an adult patient with refractory CML, wherein the method comprises administering to said patient an initial daily dose of 126 mg of a compound of Formula I or a pharmaceutically acceptable salt thereof such that when the patient develops non-hematologic toxicity:
a) the initial daily dose is withheld for at least 7 days and resumed at 126 mg after recovery of patient from said toxicity; or
b) the initial daily dose is withheld for less than 7 days and if toxicity is resolved then resumed at 126 mg; or
c) the initial daily dose is withheld for at least 7 days and resumed at a reduced daily dose of 90 mg on first instance and to 66 mg, 48 mg, on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof in the patient results in mean Cₘₐₓ ranging from 3282 ng/mL to 4924±3289 ng/mL.

In one another particular embodiment, the present invention relates to a method for the treatment of an adult patient with refractory CML, wherein the method comprises administering to said patient an initial daily dose of 126 mg of a compound of Formula I or a pharmaceutically acceptable salt thereof such that when the patient develops non-hematologic toxicity:
a) the initial daily dose is withheld for at least 14 days and resumed at 126 mg after recovery of patient from said toxicity; or
b) the initial daily dose is withheld for less than 14 days and if toxicity is resolved then resumed at 126 mg; or
c) the initial daily dose is withheld for at least 14 days and resumed at a reduced daily dose of 90 mg on first instance and to 66 mg, 48 mg, on subsequent instances of recurrences of said toxicity.

In another particular embodiment, the present invention relates to a method for the treatment of an adult patient with refractory CML, wherein the method comprises administering to said patient an initial daily dose of 126 mg of a compound of Formula I or a pharmaceutically acceptable salt thereof such that when the patient develops non-hematologic toxicity:
a) the initial daily dose is withheld for at least 14 days and resumed at 126 mg after recovery of patient from said toxicity; or
b) the initial daily dose is withheld for less than 14 days and if toxicity is resolved then resumed at 126 mg; or
c) the initial daily dose is withheld for at least 14 days and resumed at a reduced daily dose of 90 mg on first instance and to 66 mg, 48 mg, on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another particular embodiment, the present invention relates to a method for the treatment of an adult patient with refractory CML, wherein the method comprises administering to said patient an initial daily dose of 126 mg of a compound of Formula I or a pharmaceutically acceptable salt thereof such that when the patient develops non-hematologic toxicity:
a) the initial daily dose is withheld for at least 14 days and resumed at 126 mg after recovery of patient from said toxicity; or
b) the initial daily dose is withheld for less than 14 days and if toxicity is resolved then resumed at 126 mg; or
c) the initial daily dose is withheld for at least 14 days and resumed at a reduced daily dose of 90 mg on first instance and to 66 mg, 48 mg, on subsequent instances of recurrences of said toxicity;
and wherein the daily dose of the compound of Formula I or a pharmaceutically acceptable salt thereof in the patient results in mean AUC₀₋₂₄ ranging from 40249 ng*h/mL to 60373±55659 ng*h/mL.

In another particular embodiment, the present invention relates to a method for the treatment of an adult patient with refractory CML, wherein the method comprises administering to said patient an initial daily dose of 126 mg of a compound of Formula I or a pharmaceutically acceptable salt thereof such that when the patient develops non-hematologic toxicity:
a) the initial daily dose is withheld for at least 14 days and resumed at 126 mg after recovery of patient from said toxicity; or
b) the initial daily dose is withheld for less than 14 days and if toxicity is resolved then resumed at 126 mg; or
c) the initial daily dose is withheld for at least 14 days and resumed at a reduced daily dose of 90 mg on first instance and to 66 mg, 48 mg, on subsequent instances of recurrences of said toxicity;
and wherein the daily dose of the compound of Formula I or a pharmaceutically acceptable salt thereof in the patient results in mean Cₘₐₓ ranging from 3282 ng/ml to 4924±3289 ng/mL.

In another particular embodiment, the present invention relates to a method for the treatment of an adult patient with refractory CML, wherein the method comprises administering to said patient an initial daily dose of 126 mg of a compound of Formula I or a pharmaceutically acceptable salt thereof such that when the patient develops non-hematologic toxicity:
a) the initial daily dose is withheld for at least 14 days and resumed at 126 mg after recovery of patient from said toxicity; or
b) the initial daily dose is withheld for less than 14 days and if toxicity is resolved then resumed at 126 mg; or
c) the initial daily dose is withheld for at least 14 days and resumed at a reduced daily dose of 90 mg on first instance and to 66 mg, 48 mg, on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof in the patient results in mean AUC₀₋₂₄ ranging from 40249 ng*h/mL to 60373±55659 ng*h/mL.

In another particular embodiment, the present invention relates to a method for the treatment of an adult patient with refractory CML, wherein the method comprises administering to said patient an initial daily dose of 126 mg of a compound of Formula I or a pharmaceutically acceptable salt thereof such that when the patient develops non-hematologic toxicity:
a) the initial daily dose is withheld for at least 14 days and resumed at 126 mg after recovery of patient from said toxicity; or
b) the initial daily dose is withheld for less than 14 days and if toxicity is resolved then resumed at 126 mg; or
c) the initial daily dose is withheld for at least 14 days and resumed at a reduced daily dose of 90 mg on first instance and to 66 mg, 48 mg, on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof results in a mean Cₘₐₓ ranging from 3282 ng/mL to 4924±3289 ng/mL.

In one particular embodiment, the present invention relates to a method for the treatment of an adult patient with refractory CML, wherein the method comprises administering to the patient an initial daily dosage of 126 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose of 90 mg on first instance and to 66 mg, 48 mg, on subsequent instances of recurrences of said toxicity.

In another particular embodiment, the present invention relates to a method for the treatment of an adult patient with refractory CML, wherein the method comprises administering to the patient an initial daily dose of 126 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose of the initial daily dose is withheld for at least 7 days and resumed at a reduced daily dose of 90 mg on first instance and to 66 mg, 48 mg, on subsequent instances of recurrences of said toxicity and wherein,
the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another particular embodiment, the present invention relates to a method for the treatment of an adult patient with refractory CML, wherein the method comprises administering to the patient an initial daily dose of 126 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose of 90 mg on first instance and to 66 mg, 48 mg, on subsequent instances of recurrences of said toxicity, and wherein the daily dose of the compound of Formula I or a pharmaceutically acceptable salt thereof in the patient results in mean AUC₀₋₂₄ ranging from 40249 ng*h/mL to 60373±55659 ng*h/mL.

In another particular embodiment, the present invention relates to a method for the treatment of an adult patient with refractory CML, wherein the method comprises administering to the patient an initial daily dose of 126 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose of 90 mg on first instance and to 66 mg, 48 mg, on subsequent instances of recurrences of said toxicity, and wherein the daily dose of the compound of Formula I or a pharmaceutically acceptable salt thereof in the patient results in mean Cₘₐₓ ranging from 3282 ng/ml to 4924±3289 ng/mL.

In another particular embodiment, the present invention relates to a method for the treatment of an adult patient with refractory CML, wherein the method comprises administering to the patient an initial daily dose of 126 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose of 90 mg on first instance and to 66 mg, 48 mg, on subsequent instances of recurrences of said toxicity and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another particular embodiment, the present invention relates to a method for the treatment of an adult patient with refractory CML, wherein the method comprises administering to the patient an initial daily dose of 126 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose of 90 mg on first instance and to 66 mg, 48 mg, on subsequent instances of recurrences of said toxicity and wherein,
the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof in the patient results in mean AUC₀₋₂₄ ranging from 40249 ng*h/mL to 60373±55659 ng*h/mL.

In another particular embodiment, the present invention relates to a method for the treatment of an adult patient with refractory CML, wherein the method comprises administering to the patient an initial daily dosage of 126 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose of 90 mg on first instance and to 66 mg, 48 mg, on subsequent instances of recurrences of said toxicity and wherein,
the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof results in a mean Cₘₐₓ ranging from 3282 ng/ml to 4924±3289 ng/mL.

In one particular embodiment, the present invention relates to a method for the treatment of an adult patient with newly diagnosed CML, wherein the method comprises administering to said patient an initial daily dose of 66 mg of a compound of Formula I or a pharmaceutically acceptable salt thereof such that when the patient develops non-hematologic toxicity:
a) the initial daily dose is withheld for at least 7 days and resumed at 66 mg after
b) the initial daily dose is withheld for less than 7 days and if toxicity is resolved then resumed at 66 mg; or
c) the initial daily dose is withheld for at least 7 days and resumed at a reduced daily dose of 48 mg for said toxicity.

In another particular embodiment, the present invention relates to a method for the treatment of an adult patient with newly diagnosed CML, wherein the method comprises administering to said patient an initial daily dose of 66 mg of a compound of Formula I such that when the patient develops non-hematologic toxicity:
a) the initial daily dose is withheld for at least 7 days and resumed at 66 mg after recovery of patient from said toxicity; or
b) the initial daily dose is withheld for less than 7 days and if toxicity is resolved then resumed at 66 mg; or
c) the initial daily dose is withheld for at least 7 days and resumed at a reduced daily dose of 48 mg for said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another particular embodiment, the present invention relates to a method for the treatment of an adult patient with newly diagnosed CML, wherein the method comprises administering to said patient an initial daily dose of 66 mg of a compound of Formula I or a pharmaceutically acceptable salt thereof such that when the patient develops non-hematologic toxicity:
a) the initial daily dose is withheld for at least 7 days and resumed at 66 mg after recovery of patient from said toxicity; or
b) the initial daily dose is withheld for less than 7 days and if toxicity is resolved then resumed at 66 mg; or
c) the initial daily dose is withheld for at least 7 days and resumed at a reduced daily dose of 48 mg for said toxicity;
and wherein the daily dose of the compound of Formula I or a pharmaceutically acceptable salt thereof in the patient results in mean AUC₀₋₂₄ ranging from 6226 ng*h/mL to 9338±5827 ng*h/mL.

In another particular embodiment, the present invention relates to a method for the treatment of an adult patient with newly diagnosed CML, wherein the method comprises administering to said patient an initial daily dose of 66 mg of a compound of Formula I or a pharmaceutically acceptable salt thereof: such that when the patient develops non-hematologic toxicity:
a) the initial daily dose is withheld for at least 7 days and resumed at 66 mg after recovery of patient from said toxicity; or
b) the initial daily dose is withheld for less than 7 days and if toxicity is resolved then resumed at 66 mg; or
c) the initial daily dose is withheld for at least 7 days and resumed at a reduced daily dose of 48 mg for said toxicity;
and wherein the daily dose of the compound of Formula I or a pharmaceutically acceptable salt thereof in the patient results in mean Cₘₐₓ ranging from 664 ng/ml to 996±450 ng/mL.

In another particular embodiment, the present invention relates to a method for the treatment of an adult patient with newly diagnosed CML, wherein the method comprises administering to said patient an initial daily dose of 66 mg of a compound of Formula I or a pharmaceutically acceptable salt thereof such that when the patient develops non-hematologic toxicity:
a) the initial daily dose is withheld for at least 7 days and resumed at 66 mg after recovery of patient from said toxicity; or
b) the initial daily dose is withheld for less than 7 days and if toxicity is resolved then resumed at 66 mg; or
c) the initial daily dose is withheld for at least 7 days and resumed at a reduced daily dose of 48 mg for said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof in the patient results in mean AUC₀₋₂₄ ranging from 6226 ng*h/mL to 9338±5827 ng*h/mL.

In another particular embodiment, the present invention relates to a method for the treatment of an adult patient with newly diagnosed CML, wherein the method comprises administering to said patient an initial daily dose of 66 mg of a compound of Formula I or a pharmaceutically acceptable salt thereof such that when the patient develops non-hematologic toxicity:
a) the initial daily dose is withheld for at least 7 days and resumed at 66 mg after recovery of patient from said toxicity; or
b) the initial daily dose is withheld for less than 7 days and if toxicity is resolved then resumed at 66 mg; or
c) the initial daily dose is withheld for at least 7 days and resumed at a reduced daily dose of 48 mg for said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof in the patient results in mean Cₘₐₓ ranging from 664 ng/ml to 996±450 ng/mL.

In one particular embodiment, the present invention relates to a method for the treatment of an adult patient with newly diagnosed CML, wherein the method comprises administering to said patient an initial daily dose of 66 mg of a compound of Formula I or a pharmaceutically acceptable salt thereof such that when the patient develops non-hematologic toxicity:
a) the initial daily dose is withheld for at least 14 days and resumed at 66 mg after recovery of patient from said toxicity; or
b) the initial daily dose is withheld for less than 14 days and if toxicity is resolved then resumed at 66 mg; or
c) the initial daily dose is withheld for at least 14 days and resumed at a reduced daily dose of 48 mg for said toxicity.

In another particular embodiment, the present invention relates to a method for the treatment of an adult patient with newly diagnosed CML, wherein the method comprises administering to said patient an initial daily dose of 66 mg of a compound of Formula I or a pharmaceutically acceptable salt thereof such that when the patient develops non-hematologic toxicity:
a) the initial daily dose is withheld for at least 14 days and resumed at 66 mg after recovery of patient from said toxicity; or
b) the initial daily dose is withheld for less than 14 days and if toxicity is resolved then resumed at 66 mg; or
c) the initial daily dose is withheld for at least 14 days and resumed at a reduced daily dose of 48 mg for said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another particular embodiment, the present invention relates to a method for the treatment of an adult patient with newly diagnosed CML, wherein the method comprises administering to said patient an initial daily dose of 66 mg of a compound of Formula I or a pharmaceutically acceptable salt thereof such that when the patient develops non-hematologic toxicity:
a) the initial daily dose is withheld for at least 14 days and resumed at 66 mg after recovery of patient from said toxicity; or
b) the initial daily dose is withheld for less than 14 days and if toxicity is resolved then resumed at 66 mg; or
c) the initial daily dose is withheld for at least 14 days and resumed at a reduced daily dose of 48 mg for said toxicity;
and wherein the daily dose of the compound of Formula I in the patient results in mean AUC₀₋₂₄ ranging from 6226 ng*h/mL to 9338±5827 ng*h/mL.

In another particular embodiment, the present invention relates to a method for the treatment of an adult patient with newly diagnosed CML, wherein the method comprises administering to said patient an initial daily dose of 66 mg of a compound of Formula I or a pharmaceutically acceptable salt thereof such that when the patient develops non-hematologic toxicity:
a) the initial daily dose is withheld for at least 14 days and resumed at 66 mg after recovery of patient from said toxicity; or
b) the initial daily dose is withheld for less than 14 days and if toxicity is resolved then resumed at 66 mg; or
c) the initial daily dose is withheld for at least 14 days and resumed at a reduced daily dose of 48 mg for said toxicity;
and wherein the daily dose of the compound of Formula I or a pharmaceutically acceptable salt thereof in the patient results in mean Cₘₐₓ ranging from 664 ng/mL to 996±450 ng/mL.

In another particular embodiment, the present invention relates to a method for the treatment of an adult patient with newly diagnosed CML, wherein the method comprises administering to said patient an initial daily dose of 66 mg of a compound of Formula I or a pharmaceutically acceptable salt thereof such that when the patient develops non-hematologic toxicity:
a) the initial daily dose is withheld for at least 14 days and resumed at 66 mg after recovery of patient from said toxicity; or
b) the initial daily dose is withheld for less than 14 days and if toxicity is resolved then resumed at 66 mg; or
c) the initial daily dose is withheld for at least 14 days and resumed at a reduced daily dose of 48 mg for said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof in the patient results in mean AUC₀₋₂₄ ranging from 6226 ng*h/mL to 9338±5827 ng*h/mL.

In another particular embodiment, the present invention relates to a method for the treatment of an adult patient with newly diagnosed CML, wherein the method comprises administering to said patient an initial daily dose of 66 mg of a compound of Formula I or a pharmaceutically acceptable salt thereof such that when the patient develops non-hematologic toxicity:
a) the initial daily dose is withheld for at least 14 days and resumed at 66 mg after recovery of patient from said toxicity; or
b) the initial daily dose is withheld for less than 14 days and if toxicity is resolved then resumed at 66 mg; or
c) the initial daily dose is withheld for at least 14 days and resumed at a reduced daily dose of 48 mg for said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof in the patient results in mean Cₘₐₓ ranging from 664 ng/ml to 996±450 ng/mL.

In one particular embodiment, the present invention relates to a method for the treatment of an adult patient with newly diagnosed CML, wherein the method comprises administering to the patient an initial daily dose of 66 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose of 48 mg for said toxicity.

In another particular embodiment, the present invention relates to a method for the treatment of an adult patient with newly diagnosed CML, wherein the method comprises administering to the patient an initial daily dose of 66 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose of 48 mg for said toxicity, and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another particular embodiment, the present invention relates to a method for the treatment of an adult patient with newly diagnosed CML, wherein the method comprises administering to the patient an initial daily dose of 66 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose of 48 mg for said toxicity, and wherein the daily dose of the compound of Formula I or a pharmaceutically acceptable salt thereof in the patient results in mean AUC₀₋₂₄ ranging from 6226 ng*h/mL to 9338±5827 ng*h/mL.

In another particular embodiment, the present invention relates to a method for the treatment of an adult patient with newly diagnosed CML, wherein the method comprises administering to the patient an initial daily dose of 66 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose of 48 mg for said toxicity, and wherein the daily dose of the compound of Formula I or a pharmaceutically acceptable salt thereof in the patient results in mean Cₘₐₓ ranging from 664 ng/mL to 996±450 ng/mL.

In another particular embodiment, the present invention relates to a method for the treatment of an adult patient with newly diagnosed CML, wherein the method comprises administering to the patient an initial daily dose of 66 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose of 48 mg for said toxicity and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof in the patient results in mean AUC₀₋₂₄ ranging from 6226 ng*h/mL to 9338±5827 ng*h/mL.

In another particular embodiment, the present invention relates to a method for the treatment of an adult patient with newly diagnosed CML, wherein the method comprises administering to the patient an initial daily dose of 66 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose of 48 mg for said toxicity and wherein,
the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof in the patient results in mean Cₘₐₓ ranging from 664 ng/ml to 996±450 ng/ml.

In one particular embodiment, the present invention relates to a method for the treatment of an adult patient with refractory CML, wherein the method comprises administering to said patient an initial daily dose of 66 mg of a compound of Formula I or a pharmaceutically acceptable salt thereof such that when the patient develops non-hematologic toxicity:
a) the initial daily dose is withheld for at least 7 days and resumed at 66 mg after recovery of patient from said toxicity; or
b) the initial daily dose is withheld for less than 7 days and if toxicity is resolved then resumed at 66 mg; or
c) the initial daily dose is withheld for at least 7 days and resumed at a reduced daily dose of 48 mg for said toxicity.

In another particular embodiment, the present invention relates to a method for the treatment of an adult patient with refractory CML, wherein the method comprises administering to said patient an initial daily dose of 66 mg of a compound of Formula I or a pharmaceutically acceptable salt thereof such that when the patient develops non-hematologic toxicity:
a) the initial daily dose is withheld for at least 7 days and resumed at 66 mg after recovery of patient from said toxicity; or
b) the initial daily dose is withheld for less than 7 days and if toxicity is resolved then resumed at 66 mg; or
c) the initial daily dose is withheld for at least 7 days and resumed at a reduced daily dose of 48 mg for said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another particular embodiment, the present invention relates to a method for the treatment of an adult patient with refractory CML, wherein the method comprises administering to the patient initial daily dose of 66 mg of a compound of Formula I or a pharmaceutically acceptable salt thereof such that when the patient develops non-hematological toxicity:
a) the initial daily dose is withheld for at least 7 days and resumed at 66 mg after recovery of patient from said toxicity; or
b) the initial daily dose is withheld for less than 7 days and if toxicity is resolved then resumed at 66 mg; or
c) the initial daily dose is withheld for at least 7 days and resumed at a reduced daily dose of 48 mg for said toxicity;
and wherein the daily dose of the compound of Formula I or a pharmaceutically acceptable salt thereof in the patient results in mean AUC₀₋₂₄ ranging from 6226 ng*h/mL to 9338±5827 ng*h/mL.

In another particular embodiment, the present invention relates to a method for the treatment of an adult patient with refractory CML, wherein the method comprises administering to the patient initial daily dose of 66 mg of a compound of Formula I or a pharmaceutically acceptable salt thereof such that when the patient develops non-hematological toxicity:
a) the initial daily dose is withheld for at least 7 days and resumed at 66 mg after recovery of patient from said toxicity; or
b) the initial daily dose is withheld for less than 7 days and if toxicity is resolved then resumed at 66 mg; or
c) the initial daily dose is withheld for at least 7 days and resumed at a reduced daily dose of 48 mg for said toxicity;
and wherein the daily dose of the compound of Formula I or a pharmaceutically acceptable salt thereof in the patient results in mean Cₘₐₓ ranging from 664 ng/mL to 996±450 ng/ml.

In another particular embodiment, the present invention provides a method for the treatment of an adult patient with refractory CML, wherein the method comprises administering to said patient an initial daily dose of 66 mg of a compound of Formula I or a pharmaceutically acceptable salt thereof such that when the patient develops non-hematologic toxicity:
a) the initial daily dose is withheld for at least 7 days and resumed at 66 mg after recovery of patient from said toxicity; or
b) the initial daily dose is withheld for less than 7 days and if toxicity is resolved then resumed at 66 mg; or
c) the initial daily dose is withheld for at least 7 days and resumed at a reduced daily dose of 48 mg for said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof in the patient results in mean AUC₀₋₂₄ ranging from 6226 ng*h/mL to 9338±5827 ng*h/mL.

In another particular embodiment, the present invention provides a method for the treatment of an adult patient with refractory CML, wherein the method comprises administering to said patient an initial daily dose of 66 mg of a compound of Formula I or a pharmaceutically acceptable salt thereof such that when the patient develops non-hematologic toxicity:
a) the initial daily dose is withheld for at least 7 days and resumed at 66 mg after recovery of patient from said toxicity; or
b) the initial daily dose is withheld for less than 7 days and if toxicity is resolved then resumed at 66 mg; or
c) the initial daily dose is withheld for at least 7 days and resumed at a reduced daily dose of 48 mg for said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof in the patient results in mean Cₘₐₓ ranging from 664 ng/mL to 996±450 ng/ml.

In one particular embodiment, the present invention relates to a method for the treatment of an adult patient with refractory CML, wherein the method comprises administering to said patient an initial daily dose of 66 mg of a compound of Formula I or a pharmaceutically acceptable salt thereof such that when the patient develops non-hematologic toxicity:
a) the initial daily dose is withheld for at least 14 days and resumed at 66 mg after recovery of patient from said toxicity; or
b) the initial daily dose is withheld for less than 14 days and if toxicity is resolved then resumed at 66 mg; or
c) the initial daily dose is withheld for at least 14 days and resumed at a reduced daily dose of 48 mg for said toxicity.

In another particular embodiment, the present invention relates to a method for the treatment of an adult patient with refractory CML, wherein the method comprises administering to said patient an initial daily dose of 66 mg of a compound of Formula I or a pharmaceutically acceptable salt thereof such that when the patient develops non-hematologic toxicity:
a) the initial daily dose is withheld for at least 14 days and resumed at 66 mg after recovery of patient from said toxicity; or
b) the initial daily dose is withheld for less than 14 days and if toxicity is resolved then resumed at 66 mg; or
c) the initial daily dose is withheld for at least 14 days and resumed at a reduced daily dose of 48 mg for said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another particular embodiment, the present invention relates to a method for the treatment of an adult patient with refractory CML, wherein the method comprises administering to said patient an initial daily dose of 66 mg of a compound of Formula I or a pharmaceutically acceptable salt thereof such that when the patient develops non-hematologic toxicity:
a) the initial daily dose is withheld for at least 14 days and resumed at 66 mg after recovery of patient from said toxicity; or
b) the initial daily dose is withheld for less than 14 days and if toxicity is resolved then resumed at 66 mg; or
c) the initial daily dose is withheld for at least 14 days and resumed at a reduced daily dose of 48 mg for said toxicity;
and wherein the daily dose of the compound of Formula I or a pharmaceutically acceptable salt thereof in the patient results in mean AUC₀₋₂₄ ranging from 6226 ng*h/mL to 9338±5827 ng*h/mL.

In another particular embodiment, the present invention relates to a method for the treatment of an adult patient with refractory CML, wherein the method comprises administering to said patient an initial daily dose of 66 mg of a compound of Formula I or a pharmaceutically acceptable salt thereof such that when the patient develops non-hematologic toxicity:
a) the initial daily dose is withheld for at least 14 days and resumed at 66 mg after recovery of patient from said toxicity; or
b) the initial daily dose is withheld for less than 14 days and if toxicity is resolved then resumed at 66 mg; or
c) the initial daily dose is withheld for at least 14 days and resumed at a reduced daily dose of 48 mg for said toxicity;
and wherein the daily dose of the compound of Formula I or a pharmaceutically acceptable salt thereof in the patient results in mean Cₘₐₓ ranging from 664 ng/mL to 996±450 ng/mL.

In another particular embodiment, the present invention relates to a method for the treatment of an adult patient with refractory CML, wherein the method comprises administering to said patient an initial daily dose of 66 mg of a compound of Formula I or a pharmaceutically acceptable salt thereof such that when the patient develops non-hematologic toxicity:
a) the initial daily dose is withheld for at least 14 days and resumed at 66 mg after recovery of patient from said toxicity; or
b) the initial daily dose is withheld for less than 14 days and if toxicity is resolved then resumed at 66 mg; or
c) the initial daily dose is withheld for at least 14 days and resumed at a reduced daily dose of 48 mg for said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse, and wherein the daily dose of the compound of Formula I or a pharmaceutically acceptable salt thereof in the patient results in mean AUC₀₋₂₄ ranging from 6226 ng*h/mL to 9338±5827 ng*h/mL.

In another particular embodiment, the present invention relates to a method for the treatment of an adult patient with refractory CML, wherein the method comprises administering to said patient an initial daily dose of 66 mg of a compound of Formula I or a pharmaceutically acceptable salt thereof such that when the patient develops non-hematologic toxicity:
a) the initial daily dose is withheld for at least 14 days and resumed at 66 mg after recovery of patient from said toxicity; or
b) the initial daily dose is withheld for less than 14 days and if toxicity is resolved then resumed at 66 mg; or
c) the initial daily dose is withheld for at least 14 days and resumed at a reduced daily dose of 48 mg for said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof results in a mean Cₘₐₓ ranging from 664 ng/ml to 996±450 ng/ml.

In one particular embodiment, the present invention relates to a method for the treatment of an adult patient with refractory CML, wherein the method comprises administering to the patient an initial daily dose of 66 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose of 48 mg for said toxicity.

In another particular embodiment, the present invention relates to a method for the treatment of an adult patient with refractory CML, wherein the method comprises administering to the patient an initial daily dose of 66 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose of 48 mg for said toxicity and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another particular embodiment, the present invention relates to a method for the treatment of an adult patient with refractory CML, wherein the method comprises administering to the patient an initial daily dose of 66 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose of 48 mg for said toxicity, and wherein the daily dose of the compound of Formula I or a pharmaceutically acceptable salt thereof in the patient results in mean AUC₀₋₂₄ ranging from 6226 ng*h/mL to 9338±5827 ng*h/mL.

In another particular embodiment, the present invention relates to a method for the treatment of an adult patient with refractory CML, wherein the method comprises administering to the patient an initial daily dose of 66 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose of 48 mg for said toxicity, and wherein the daily dose of the compound of Formula I or a pharmaceutically acceptable salt thereof in the patient results in mean Cₘₐₓ ranging from 664 ng/mL to 996±450 ng/ml.

In another particular embodiment, the present invention relates to a method for the treatment of an adult patient with refractory CML, wherein the method comprises administering to the patient an initial daily dose of 66 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose of 48 mg for said toxicity and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another particular embodiment, the present invention relates to a method for the treatment of an adult patient with refractory CML, wherein the method comprises administering to the patient an initial daily dose of 66 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose of 48 mg for said toxicity and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof in the patient results in mean AUC0-24 ranging from 6226 ng*h/mL to 9338±5827 ng*h/mL.

In another particular embodiment, the present invention relates to a method for the treatment of an adult patient with refractory CML, wherein the method comprises administering to the patient an initial daily dose of 66 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose of 48 mg for said toxicity and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof results in a mean Cₘₐₓ ranging from 664 ng/mL to 996±450 ng/ml.

In one particular embodiment, the present invention relates to a method for the treatment of an adult patient with newly diagnosed CML, wherein the method comprises administering to said patient an initial daily dose of 180 mg of a compound of Formula I or a pharmaceutically acceptable salt thereof such that when the patient develops non-hematologic toxicity:
a) the initial daily dose is withheld for at least 7 days and resumed at 180 mg after recovery of patient from said toxicity; or
b) the initial daily dose is withheld for less than 7 days and if toxicity is resolved then resumed at 180 mg; or
c) the initial daily dose is withheld for at least 7 days and resumed at a reduced daily dose of 135 mg on first instance and to 90 mg, 45 mg, on subsequent instances of recurrences of said toxicity.

In another particular embodiment, the present invention relates to a method for the treatment of an adult patient with newly diagnosed CML, wherein the method comprises administering to said patient an initial daily dose of 180 mg of a compound of Formula I or a pharmaceutically acceptable salt thereof such that when the patient develops non-hematologic toxicity:
a) the initial daily dose is withheld for at least 7 days and resumed at 180 mg after recovery of patient from said toxicity; or
b) the initial daily dose is withheld for less than 7 days and if toxicity is resolved then resumed at 180 mg; or
c) the initial daily dose is withheld for at least 7 days and resumed at a reduced daily dose of 135 mg on first instance and to 90 mg, 45 mg, on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another particular embodiment, the present invention relates to a method for the treatment of an adult patient with newly diagnosed CML, wherein the method comprises administering to said patient an initial daily dose of 180 mg of a compound of Formula I or a pharmaceutically acceptable salt thereof such that when the patient develops non-hematologic toxicity:
a) the initial daily dose is withheld for at least 7 days and resumed at 180 mg after recovery of patient from said toxicity; or
b) the initial daily dose is withheld for less than 7 days and if toxicity is resolved then resumed at 180 mg; or
c) the initial daily dose is withheld for at least 7 days and resumed at a reduced daily dose of 135 mg on first instance and to 90 mg, 45 mg, on subsequent instances of recurrences of said toxicity;
and wherein the daily dose of the compound of Formula I or a pharmaceutically acceptable salt thereof in the patient results in mean AUC₀₋₂₄ ranging from 28598 ng*h/mL to 42898±33827 ng*h/mL.

In another particular embodiment, the present invention relates to a method for the treatment of an adult patient with newly diagnosed CML, wherein the method comprises administering to said patient an initial daily dose of 180 mg of a compound of Formula I or a pharmaceutically acceptable salt thereof such that when the patient develops non-hematologic toxicity:
a) the initial daily dose is withheld for at least 7 days and resumed at 180 mg after recovery of patient from said toxicity; or
b) the initial daily dose is withheld for less than 7 days and if toxicity is resolved then resumed at 180 mg; or
c) the initial daily dose is withheld for at least 7 days and resumed at a reduced daily dose of 135 mg on first instance and to 90 mg, 45 mg, on subsequent instances of recurrences of said toxicity;
and wherein the daily dose of the compound of Formula I or a pharmaceutically acceptable salt thereof in the patient results in mean Cₘₐₓ ranging from 3370 ng/ml to 5054±3051 ng/mL.

In another particular embodiment, the present invention relates to a method for the treatment of an adult patient with newly diagnosed CML, wherein the method comprises administering to said patient an initial daily such that when the patient develops non-hematologic toxicity:
a) the initial daily dose is withheld for at least 7 days and resumed at 180 mg after
b) the initial daily dose is withheld for less than 7 days and if toxicity is resolved then resumed at 180 mg; or
c) the initial daily dose is withheld for at least 7 days and resumed at a reduced daily dose of 135 mg on first instance and to 90 mg, 45 mg, on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction and wherein the daily dose of the compound of Formula I or a pharmaceutically acceptable salt thereof in the patient results in mean AUC₀₋₂₄ ranging from 28598 ng*h/mL to 42898±33827 ng*h/mL.

In another particular embodiment, the present invention relates to a method for the treatment of an adult patient with newly diagnosed CML, wherein the method comprises administering to said patient an initial daily dose of 180 mg of a compound of Formula I or a pharmaceutically acceptable salt thereof such that when the patient develops non-hematologic toxicity:
a) the initial daily dose is withheld for at least 7 days and resumed at 180 mg after recovery of patient from said toxicity; or
b) the initial daily dose is withheld for less than 7 days and if toxicity is resolved then resumed at 180 mg; or
c) the initial daily dose is withheld for at least 7 days and resumed at a reduced daily dose of 135 mg on first instance and to 90 mg, 45 mg, on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction and wherein the daily dose of the compound of Formula I or a pharmaceutically acceptable salt thereof in the patient results in mean Cₘₐₓ ranging from 3370 ng/ml to 5054±3051 ng/ml.

In one particular embodiment, the present invention relates to a method for the treatment of an adult patient with newly diagnosed CML, wherein the method comprises administering to said patient an initial daily dose of 180 mg of a compound of Formula I or a pharmaceutically acceptable salt thereof such that when the patient develops non-hematologic toxicity:
a) the initial daily dose is withheld for at least 14 days and resumed at 180 mg after recovery of patient from said toxicity; or
b) the initial daily dose is withheld for less than 14 days and if toxicity is resolved then resumed at 180 mg; or
c) the initial daily dose is withheld for at least 14 days and resumed at a reduced daily dose of 135 mg on first instance and to 90 mg, 45 mg, on subsequent instances of recurrences of said toxicity.

In another particular embodiment, the present invention relates to a method for the treatment of an adult patient with newly diagnosed CML, wherein the method comprises administering to said patient an initial daily dose of 180 mg of a compound of Formula I or a pharmaceutically acceptable salt thereof such that when the patient develops non-hematologic toxicity:
a) the initial daily dose is withheld for at least 14 days and resumed at 180 mg after recovery of patient from said toxicity; or
b) the initial daily dose is withheld for less than 14 days and if toxicity is resolved then resumed at 180 mg; or
c) the initial daily dose is withheld for at least 14 days and resumed at a reduced daily dose of 135 mg on first instance and to 90 mg, 45 mg, on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another particular embodiment, the present invention relates to a method for the treatment of an adult patient with newly diagnosed CML, wherein the method comprises administering to said patient an initial daily dose of 180 mg of a compound of Formula I or a pharmaceutically acceptable salt thereof such that when the patient develops non-hematologic toxicity:
a) the initial daily dose is withheld for at least 14 days and resumed at 180 mg after recovery of patient from said toxicity; or
b) the initial daily dose is withheld for less than 14 days and if toxicity is resolved then resumed at 180 mg; or
c) the initial daily dose is withheld for at least 14 days and resumed at a reduced daily dose of 135 mg on first instance and to 90 mg, 45 mg, on subsequent instances of recurrences of said toxicity;
and wherein the daily dose of the compound of Formula I or a pharmaceutically acceptable salt thereof in the patient results in mean AUC₀₋₂₄ ranging from 28598 ng*h/mL to 42898±33827 ng*h/mL.

In another particular embodiment, the present invention relates to a method for the treatment of an adult patient with newly diagnosed CML, wherein the method comprises administering to said patient an initial daily dose of 180 mg of a compound of Formula I or a pharmaceutically acceptable salt thereof such that when the patient develops non-hematologic toxicity:
a) the initial daily dose is withheld for at least 14 days and resumed at 180 mg after recovery of patient from said toxicity; or
b) the initial daily dose is withheld for less than 14 days and if toxicity is resolved then resumed at 180 mg; or
c) the initial daily dose is withheld for at least 14 days and resumed at a reduced daily dose of 135 mg on first instance and to 90 mg, 45 mg, on subsequent instances of recurrences of said toxicity;
and wherein the daily dose of the compound of Formula I or a pharmaceutically acceptable salt thereof in the patient results in mean Cₘₐₓ ranging from 3370 ng/ml to 5054±3051 ng/mL.

In another particular embodiment, the present invention relates to a method for the treatment of an adult patient with newly diagnosed CML, wherein the method comprises administering to said patient an initial daily dose of 180 mg of a compound of Formula I or a pharmaceutically acceptable salt thereof such that when the patient develops non-hematologic toxicity:
a) the initial daily dose is withheld for at least 14 days and resumed at 180 mg after recovery of patient from said toxicity; or
b) the initial daily dose is withheld for less than 14 days and if toxicity is resolved then resumed at 180 mg; or
c) the initial daily dose is withheld for at least 14 days and resumed at a reduced daily dose of 135 mg on first instance and to 90 mg, 45 mg, on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction and wherein the daily dose of the compound of Formula I or a pharmaceutically acceptable salt thereof in the patient results in mean AUC₀₋₂₄ ranging from 28598 ng*h/mL to 42898±33827 ng*h/mL.

In another particular embodiment, the present invention relates to a method for the treatment of an adult patient with newly diagnosed CML, wherein the method comprises administering to said patient an initial daily dose of 180 mg of a compound of Formula I or a pharmaceutically acceptable salt thereof such that when the patient develops non-hematologic toxicity:
a) the initial daily dose is withheld for at least 14 days and resumed at 180 mg after
b) the initial daily dose is withheld for less than 14 days and if toxicity is resolved then resumed at 180 mg; or
c) the initial daily dose is withheld for at least 14 days and resumed at a reduced daily dose of 135 mg on first instance and to 90 mg, 45 mg, on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction and wherein the daily dose of the compound of Formula I or a pharmaceutically acceptable salt thereof in the patient results in mean Cₘₐₓ ranging from 3370 ng/mL to 5054±3051 ng/mL.

In one particular embodiment, the present invention relates to a method for the treatment of an adult patient with newly diagnosed CML, wherein the method comprises administering to the patient an initial daily dose of 180 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose of 135 mg on first instance and to 90 mg, 45 mg, on subsequent instances of recurrences of said toxicity.

In another embodiment, the present invention relates to a method for the treatment of an adult patient with newly diagnosed CML, wherein the method comprises administering to the patient an initial daily dose of 180 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose of 135 mg on first instance and to 90 mg, 45 mg, on subsequent instances of recurrences of said toxicity and wherein,
the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another particular embodiment, the present invention relates to a method for the treatment of an adult patient with newly diagnosed CML, wherein the method comprises administering to the patient an initial daily dose of 180 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose of 135 mg on first instance and to 90 mg, 45 mg, on subsequent instances of recurrences of said toxicity, and wherein the daily dose of the compound of Formula I or a pharmaceutically acceptable salt thereof in the patient results in mean AUC₀₋₂₄ ranging from 28598 ng*h/mL to 42898±33827 ng*h/mL.

In another embodiment, the present invention relates to a method for the treatment of an adult patient with newly diagnosed CML, wherein the method comprises administering to the patient an initial daily dose of 180 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose of 135 mg on first instance and to 90 mg, 45 mg, on subsequent instances of recurrences of said toxicity, and wherein the daily dose of the compound of Formula I or a pharmaceutically acceptable salt thereof in the patient results in mean Cₘₐₓ ranging from 3370 ng/ml to 5054±3051 ng/mL.

In another particular embodiment, the present invention relates to a method for the treatment of an adult patient with newly diagnosed CML, wherein the method comprises administering to the patient an initial daily dose of 180 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose of 135 mg on first instance and to 90 mg, 45 mg, on subsequent instances of recurrences of said toxicity and wherein,
the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction and wherein the daily dose of the compound of Formula I or a pharmaceutically acceptable salt thereof in the patient results in mean AUC₀₋₂₄ ranging from 28598 ng*h/mL to 42898±33827 5827 ng*h/mL.

In another particular embodiment, the present invention relates to a method for the treatment of an adult patient with newly diagnosed CML, wherein the method comprises administering to the patient an initial daily dose of 180 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose of 135 mg on first instance and to 90 mg, 45 mg, on subsequent instances of recurrences of said toxicity and wherein,
the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction and wherein the daily dose of the compound of Formula I or a pharmaceutically acceptable salt thereof in the patient results in mean Cₘₐₓ ranging from 3370 ng/ml to 5054±3051 ng/mL.

In one embodiment, the present invention relates to a method for the treatment of an adult patient with refractory CML, wherein the method comprises administering to said patient an initial daily dose of 180 mg of a compound of Formula I or a pharmaceutically acceptable salt thereof such that when the patient develops non-hematologic toxicity:
a) the initial daily dose is withheld for at least 7 days and resumed at 180 mg after recovery of patient from said toxicity; or
b) the initial daily dose is withheld for less than 7 days and if toxicity is resolved then resumed at 180 mg; or
c) the initial daily dose is withheld for at least 7 days and resumed at a reduced daily dose of 135 mg on first instance and to 90 mg, 45 mg, on subsequent instances of recurrences of said toxicity.

In another particular embodiment, the present invention relates to a method for the treatment of an adult patient with refractory CML, wherein the method comprises administering to said patient an initial daily dose of 180 mg of a compound of Formula I or a pharmaceutically acceptable salt thereof such that when the patient develops non-hematologic toxicity:
a) the initial daily dose is withheld for at least 7 days and resumed at 180 mg after recovery of patient from said toxicity; or
b) the initial daily dose is withheld for less than 7 days and if toxicity is resolved then resumed at 180 mg; or
c) the initial daily dose is withheld for at least 7 days and resumed at a reduced daily dose of 135 mg on first instance and to 90 mg, 45 mg, on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another particular embodiment, the present invention relates to a method for the treatment of an adult patient with refractory CML, wherein the method comprises administering to the patient an initial daily dose of 180 mg of a compound of Formula I or a pharmaceutically acceptable salt thereof such that when the patient develops non-hematological toxicity:
a) the initial daily dose is withheld for at least 7 days and resumed at 180 mg after recovery of patient from said toxicity; or
b) the initial daily dose is withheld for less than 7 days and if toxicity is resolved then resumed at 180 mg; or
c) the initial daily dose is withheld for at least 7 days and resumed at a reduced daily dose of 135 mg on first instance and to 90 mg, 45 mg, on subsequent instances of recurrences of said toxicity;
and wherein the daily dose of the compound of Formula I or a pharmaceutically acceptable salt thereof in the patient results in mean AUC₀₋₂₄ ranging from 28598 ng*h/mL to 42898±33827 5827 ng*h/mL.

In another particular embodiment, the present invention relates to a method for the treatment of an adult patient with refractory CML, wherein the method comprises administering to the patient an initial daily dose of 180 mg of a compound of Formula I or a pharmaceutically acceptable salt thereof such that when the patient develops non-hematological toxicity:
a) the initial daily dose is withheld for at least 7 days and resumed at 180 mg after recovery of patient from said toxicity; or
b) the initial daily dose is withheld for less than 7 days and if toxicity is resolved then resumed at 180 mg; or
c) the initial daily dose is withheld for at least 7 days and resumed at a reduced daily dose of 135 mg on first instance and to 90 mg, 45 mg, on subsequent instances of recurrences of said toxicity;
and wherein the daily dose of the compound of Formula I or a pharmaceutically acceptable salt thereof in the patient results in mean Cₘₐₓ ranging from 3370 ng/ml to 5054±3051 ng/mL.

In another particular embodiment, the present invention provides a method for the treatment of an adult patient with refractory CML, wherein the method comprises administering to said patient an initial daily dose of 180 mg of a compound of Formula I or a pharmaceutically acceptable salt thereof such that when the patient develops non-hematological toxicity:
a) the initial daily dose is withheld for at least 7 days and resumed at 180 mg after recovery of patient from said toxicity; or
b) the initial daily dose is withheld for less than 7 days and if toxicity is resolved then resumed at 180 mg; or
c) the initial daily dose is withheld for at least 7 days and resumed at a reduced daily dose of 135 mg on first instance and to 90 mg, 45 mg, on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction and wherein the daily dose of the compound of Formula I or a pharmaceutically acceptable salt thereof in the patient results in mean AUC₀₋₂₄ ranging from 28598 ng*h/mL to 42898±33827 5827 ng*h/mL.

In another particular embodiment, the present invention provides a method for the treatment of an adult patient with refractory CML, wherein the method comprises administering to said patient an initial daily dose of 180 mg of a compound of Formula I or a pharmaceutically acceptable salt thereof such that when the patient develops non-hematologic toxicity:
a) the initial daily dose is withheld for at least 7 days and resumed at 180 mg after recovery of patient from said toxicity; or
b) the initial daily dose is withheld for less than 7 days and if toxicity is resolved then resumed at 180 mg; or
c) the initial daily dose is withheld for at least 7 days and resumed at a reduced daily dose of 135 mg on first instance and to 90 mg, 45 mg, on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction and wherein the daily dose of the compound of Formula I or a pharmaceutically acceptable salt thereof in the patient results in mean Cₘₐₓ ranging from 3370 ng/ml to 5054±3051 ng/mL.

In one embodiment, the present invention relates to a method for the treatment of an adult patient with refractory CML, wherein the method comprises administering to said patient an initial daily dose of 180 mg of a compound of Formula I or a pharmaceutically acceptable salt thereof such that when the patient develops non-hematologic toxicity:
a) the initial daily dose is withheld for at least 14 days and resumed at 180 mg after recovery of patient from said toxicity; or
b) the initial daily dose is withheld for less than 14 days and if toxicity is resolved then resumed at 180 mg; or
c) the initial daily dose is withheld for at least 14 days and resumed at a reduced daily dose of 135 mg on first instance and to 90 mg, 45 mg, on subsequent instances of recurrences of said toxicity.

In another particular embodiment, the present invention relates to a method for the treatment of an adult patient with refractory CML, wherein the method comprises administering to said patient an initial daily dose of 180 mg of a compound of Formula I or a pharmaceutically acceptable salt thereof such that when the patient develops non-hematologic toxicity:
a) the initial daily dose is withheld for at least 14 days and resumed at 180 mg after recovery of patient from said toxicity; or
b) the initial daily dose is withheld for less than 14 days and if toxicity is resolved then resumed at 180 mg; or
c) the initial daily dose is withheld for at least 14 days and resumed at a reduced daily dose of 135 mg on first instance and to 90 mg, 45 mg, on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another particular embodiment, the present invention relates to a method for the treatment of an adult patient with refractory CML, wherein the method comprises administering to said patient an initial daily dose of 180 mg of a compound of Formula I or a pharmaceutically acceptable salt thereof such that when the patient develops non-hematologic toxicity:
a) the initial daily dose is withheld for at least 14 days and resumed at 180 mg after recovery of patient from said toxicity; or
b) the initial daily dose is withheld for less than 14 days and if toxicity is resolved then resumed at 180 mg; or
c) the initial daily dose is withheld for at least 14 days and resumed at a reduced daily dose of 135 mg on first instance and to 90 mg, 45 mg, on subsequent instances of recurrences of said toxicity;
and wherein the daily dose of the compound of Formula I or a pharmaceutically acceptable salt thereof in the patient results in mean AUC₀₋₂₄ ranging from 28598 ng*h/mL to 42898±33827 5827 ng*h/mL.

In another particular embodiment, the present invention relates to a method for the treatment of an adult patient with refractory CML, wherein the method comprises administering to said patient an initial daily dose of 180 mg of a compound of Formula I or a pharmaceutically acceptable salt thereof such that when the patient develops non-hematologic toxicity:
a) the initial daily dose is withheld for at least 14 days and resumed at 180 mg after recovery of patient from said toxicity; or
b) the initial daily dose is withheld for less than 14 days and if toxicity is resolved then resumed at 180 mg; or
c) the initial daily dose is withheld for at least 14 days and resumed at a reduced daily dose of 135 mg on first instance and to 90 mg, 45 mg, on subsequent instances of recurrences of said toxicity;
and wherein the daily dose of the compound of Formula I or a pharmaceutically acceptable salt thereof in the patient results in mean Cₘₐₓ ranging from 3370 ng/mL to 5054±3051 ng/mL.

In another particular embodiment, the present invention relates to a method for the treatment of an adult patient with refractory CML, wherein the method comprises administering to said patient an initial daily dose of 180 mg of a compound of Formula I or a pharmaceutically acceptable salt thereof such that when the patient develops non-hematologic toxicity:
a) the initial daily dose is withheld for at least 14 days and resumed at 180 mg after recovery of patient from said toxicity; or
b) the initial daily dose is withheld for less than 14 days and if toxicity is resolved then resumed at 180 mg; or
c) the initial daily dose is withheld for at least 14 days and resumed at a reduced daily dose of 135 mg on first instance and to 90 mg, 45 mg, on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction and wherein the daily dose of the compound of Formula I or a pharmaceutically acceptable salt thereof in the patient results in mean AUC₀₋₂₄ ranging from 28598 ng*h/mL to 42898±33827 5827 ng*h/mL.

In another particular embodiment, the present invention relates to a method for the treatment of an adult patient with refractory CML, wherein the method comprises administering to said patient an initial daily dose of 180 mg of a compound of Formula I or a pharmaceutically acceptable salt thereof such that when the patient develops non-hematological toxicity:
a) the initial daily dose is withheld for at least 14 days and resumed at 180 mg after recovery of patient from said toxicity; or
b) the initial daily dose is withheld for less than 14 days and if toxicity is resolved then resumed at 180 mg; or
c) the initial daily dose is withheld for at least 14 days and resumed at a reduced daily dose of 135 mg on first instance and to 90 mg, 45 mg, on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction and wherein the daily dose of the compound of Formula I or a pharmaceutically acceptable salt thereof results in a mean Cₘₐₓ ranging from 3370 ng/mL to 5054±3051 ng/mL.

In one embodiment, the present invention relates to a method for the treatment of an adult patient with refractory CML, wherein the method comprises administering to the patient an initial daily dose of 180 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose of 135 mg on first instance and to 90 mg, 45 mg, on subsequent instances of recurrences of said toxicity.

In another particular embodiment, the present invention relates to a method for the treatment of an adult patient with refractory CML, wherein the method comprises administering to the patient an initial daily dose of 180 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose of the initial daily dose is withheld for at least 7 days and resumed at a reduced daily dose of 135 mg on first instance and to 90 mg, 45 mg, on subsequent instances of recurrences of said toxicity and wherein,
the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another particular embodiment, the present invention relates to a method for the treatment of an adult patient with refractory CML, wherein the method comprises administering to the patient an initial daily dose of 180 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose of 135 mg on first instance and to 90 mg, 45 mg, on subsequent instances of recurrences of said toxicity, and wherein the daily dose of the compound of Formula I or a pharmaceutically acceptable salt thereof in the patient results in mean AUC₀₋₂₄ ranging from 28598 ng*h/mL to 42898±33827 5827 ng*h/mL.

In another particular embodiment, the present invention relates to a method for the treatment of an adult patient with refractory CML, wherein the method comprises administering to the patient an initial daily dose of 180 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose of 135 mg on first instance and to 90 mg, 45 mg, on subsequent instances of recurrences of said toxicity, and wherein
the daily dose of the compound of Formula I or a pharmaceutically acceptable salt thereof in the patient results in mean Cₘₐₓ ranging from 3370 ng/ml to 5054±3051 ng/ml.

In another particular embodiment, the present invention relates to a method for the treatment of an adult patient with refractory chronic myeloid leukemia (CML), wherein the method comprises administering to the patient an initial daily dose of 180 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose of 135 mg on first instance and to 90 mg, 45 mg, on subsequent instances of recurrences of said toxicity and wherein,
the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another particular embodiment, the present invention relates to a method for the treatment of an adult patient with refractory CML, wherein the method comprises administering to the patient an initial daily dose of 180 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose of 135 mg on first instance and to 90 mg, 45 mg, on subsequent instances of recurrences of said toxicity and wherein,
the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof in the patient results in mean AUC₀₋₂₄ ranging from 28598 ng*h/mL to 42898±33827 5827 ng*h/mL.

In another particular embodiment, the present invention relates to a method for the treatment of an adult patient with refractory CML, wherein the method comprises administering to the patient an initial daily dose of 180 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose of 135 mg on first instance and to 90 mg, 45 mg, on subsequent instances of recurrences of said toxicity and wherein,
the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction, and wherein the daily dose of the compound of Formula I or a pharmaceutically acceptable salt thereof results in a mean Cₘₐₓ ranging from 3370 ng/ml to 5054±3051 ng/mL.

In one embodiment, the present invention relates to a method for the treatment of an adult patient with newly diagnosed CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 7 days and resumed at 90 mg after recovery of patient from said toxicity; or
b) the initial daily dose is withheld for less than 7 days and if toxicity is resolved then resumed at 90 mg; or
c) the initial daily dose is withheld for at least 7 days and resumed at a reduced daily dose selected from 45 mg, 48 mg and 66 mg on subsequent instances of recurrences of said toxicity.

In another particular embodiment, the present invention relates to a method for the treatment of an adult patient with newly diagnosed CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 7 days and resumed at 90 mg after recovery of patient from said toxicity; or
b) the initial daily dose is withheld for less than 7 days and if toxicity is resolved then resumed at 90 mg; or
c) the initial daily dose is withheld for at least 7 days and resumed at a reduced daily dose selected from 45 mg, 48 mg and 66 mg on subsequent instances of recurrences of said toxicity;
and wherein the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another embodiment, the present invention relates to a method for the treatment of an adult patient with newly diagnosed CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 7 days and resumed at 90 mg after recovery of patient from said toxicity; or
b) the initial daily dose is withheld for less than 7 days and if toxicity is resolved then resumed at 90 mg; or
c) the initial daily dose is withheld for at least 7 days and resumed at a reduced daily dose selected from 45 mg, 48 mg and 66 mg on subsequent instances of recurrences of said toxicity;
and wherein the daily dose of the compound of Formula I or a pharmaceutically acceptable salt thereof in the patient results in mean AUC₀₋₂₄ ranging from 18237 ng*h/mL to 27355±2782 5827 ng*h/mL.

In another particular embodiment, the present invention relates to a method for the treatment of an adult patient with newly diagnosed CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 7 days and resumed at 90 mg after recovery of patient from said toxicity; or
b) the initial daily dose is withheld for less than 7 days and if toxicity is resolved then resumed at 90 mg; or
c) at a reduced daily dose, the initial daily dose is withheld for at least 7 days and resumed at a reduced daily dose selected from 45 mg, 48 mg and 66 mg on subsequent instances of recurrences of said toxicity;
and wherein the daily dose of the compound of Formula I or a pharmaceutically acceptable salt thereof in the patient results in mean Cₘₐₓ ranging from 2277 ng/ml to 3415±1566 ng/mL.

In another particular embodiment, the present invention relates to a method for the treatment of an adult patient with newly diagnosed CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 7 days and resumed at 90 mg after recovery of patient from said toxicity; or
b) the initial daily dose is withheld for less than 7 days and if toxicity is resolved then resumed at 90 mg; or
c) at a reduced daily dose, the initial daily dose is withheld for at least 7 days and resumed at a reduced daily dose selected from 45 mg, 48 mg and 66 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction and wherein the daily dose of the compound of Formula I or a pharmaceutically acceptable salt thereof in the patient results in mean AUC₀₋₂₄ ranging from 18237 ng*h/mL to 27355±2782 5827 ng*h/mL.

In another particular embodiment, the present invention relates to a method for the treatment of an adult patient with newly diagnosed CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 7 days and resumed at 90 mg after recovery of patient from said toxicity; or
b) the initial daily dose is withheld for less than 7 days and if toxicity is resolved then resumed at 90 mg; or
c) the initial daily dose is withheld for at least 7 days and resumed at a reduced daily dose selected from 45 mg, 48 mg and 66 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction and wherein the daily dose of the compound of Formula I or a pharmaceutically acceptable salt thereof in the patient results in mean Cₘₐₓ ranging from 2277 ng/mL to 3415±1566 ng/ml.

In one embodiment, the present invention relates to a method for the treatment of an adult patient with newly diagnosed CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 14 days and resumed at 90 mg after recovery of patient from said toxicity; or
b) the initial daily dose is withheld for less than 14 days and if toxicity is resolved then resumed at 90 mg; or
c) the initial daily dose is withheld for at least 14 days and resumed at a reduced daily dose selected from 45 mg, 48 mg and 66 mg on subsequent instances of recurrences of said toxicity.

In another particular embodiment, the present invention relates to a method for the treatment of an adult patient with newly diagnosed CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 14 days and resumed at 90 mg after recovery of patient from said toxicity; or
b) the initial daily dose is withheld for less than 14 days and if toxicity is resolved then resumed at 90 mg; or
c) the initial daily dose is withheld for at least 14 days and resumed at a reduced daily dose selected from 45 mg, 48 mg and 66 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another particular embodiment, the present invention relates to a method for the treatment of an adult patient with newly diagnosed CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 14 days and resumed at 90 mg after recovery of patient from said toxicity; or
b) the initial daily dose is withheld for less than 14 days and if toxicity is resolved then resumed at 90 mg; or
c) at a reduced daily dose, the initial daily dose is withheld for at least 14 days and resumed at a reduced daily dose selected from 45 mg, 48 mg and 66 mg on subsequent instances of recurrences of said toxicity;
and wherein the daily dose of the compound of Formula I or a pharmaceutically acceptable salt thereof in the patient results in mean AUC₀₋₂₄ ranging from 18237 ng*h/mL to 27355±2782 5827 ng*h/mL.

In another particular embodiment, the present invention relates to a method for the treatment of an adult patient with newly diagnosed CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 14 days and resumed at 90 mg after recovery of patient from said toxicity; or
b) the initial daily dose is withheld for less than 14 days and if toxicity is resolved then resumed at 90 mg; or
c) at a reduced daily dose, the initial daily dose is withheld for at least 14 days and resumed at a reduced daily dose selected from 45 mg, 48 mg and 66 mg on subsequent instances of recurrences of said toxicity;
and wherein the daily dose of the compound of Formula I or a pharmaceutically acceptable salt thereof in the patient results in mean Cₘₐₓ ranging from 2277 ng/mL to 3415±1566 ng/mL.

In another particular embodiment, the present invention relates to a method for the treatment of an adult patient with newly diagnosed CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 14 days and resumed at 90 mg after recovery of patient from said toxicity; or
b) the initial daily dose is withheld for less than 14 days and if toxicity is resolved then resumed at 90 mg; or
c) at a reduced daily dose, the initial daily dose is withheld for at least 14 days and resumed at a reduced daily dose selected from 45 mg, 48 mg and 66 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction and wherein the daily dose of the compound of Formula I or a pharmaceutically acceptable salt thereof in the patient results in mean AUC₀₋₂₄ ranging from 18237 ng*h/mL to 27355±2782 5827 ng*h/mL.

In another particular embodiment, the present invention relates to a method for the treatment of an adult patient with newly diagnosed CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 14 days and resumed at 90 mg after recovery of patient from said toxicity; or
b) the initial daily dose is withheld for less than 14 days and if toxicity is resolved then resumed at 90 mg; or
c) at a reduced daily dose, the initial daily dose is withheld for at least 7 days and resumed at a reduced daily dose selected from 45 mg, 48 mg and 66 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction and wherein the daily dose of the compound of Formula I or a pharmaceutically acceptable salt thereof in the patient results in mean Cₘₐₓ ranging from 2277 ng/ml to 3415±1566 ng/mL.

In one particular embodiment, the present invention relates to a method for the treatment of an adult patient with newly diagnosed CML, wherein the method comprises administering to the patient an initial daily dose of 90 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose selected from 45 mg, 48 mg and 66 mg on subsequent instances of recurrences of said toxicity.

In another particular embodiment, the present invention relates to a method for the treatment of an adult patient with newly diagnosed CML, wherein the method comprises administering to the patient an initial daily dose of 90 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose selected from 45 mg, 48 mg and 66 mg on subsequent instances of recurrences of said toxicity and wherein,
the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another particular embodiment, the present invention relates to a method for the treatment of an adult patient with newly diagnosed CML, wherein the method comprises administering to the patient an initial daily dose of 90 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose selected from 45 mg, 48 mg and 66 mg on subsequent instances of recurrences of said toxicity, and wherein the daily dose of the compound of Formula I or a pharmaceutically acceptable salt thereof in the patient results in mean AUC₀₋₂₄ ranging from 18237 ng*h/mL to 27355±2782 5827 ng*h/mL.

In another particular embodiment, the present invention relates to a method for the treatment of an adult patient with newly diagnosed CML, wherein the method comprises administering to the patient an initial daily dose of 90 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose selected from 45 mg, 48 mg and 66 mg on subsequent instances of recurrences of said toxicity, and wherein the daily dose of the compound of Formula I or a pharmaceutically acceptable salt thereof in the patient results in mean Cₘₐₓ ranging from 2277 ng/ml to 3415±1566 ng/ml.

In another particular embodiment, the present invention relates to a method for the treatment of an adult patient with newly diagnosed CML, wherein the method comprises administering to the patient an initial daily dose of 90 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose selected from 45 mg, 48 mg and 66 mg on subsequent instances of recurrences of said toxicity and wherein,
the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction and wherein the daily dose of the compound of Formula I or a pharmaceutically acceptable salt thereof in the patient results in mean AUC₀₋₂₄ ranging from 18237 ng*h/mL to 27355±2782 5827 ng*h/mL.

In another particular embodiment, the present invention relates to a method for the treatment of an adult patient with newly diagnosed CML, wherein the method comprises administering to the patient an initial daily dose of 90 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose selected from 45 mg, 48 mg and 66 mg on subsequent instances of recurrences of said toxicity and wherein,
the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction and wherein the daily dose of the compound of Formula I or a pharmaceutically acceptable salt thereof in the patient results in mean Cₘₐₓ ranging from 2277 ng/mL to 3415±1566 ng/ml.

In one particular embodiment, the present invention relates to a method for the treatment of an adult patient with refractory CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 7 days and resumed at 90 mg after recovery of patient from said toxicity; or
b) the initial daily dose is withheld for less than 7 days and if toxicity is resolved then resumed at 90 mg; or
c) the initial daily dose is withheld for at least 7 days and resumed at a reduced daily dose selected from 45 mg, 48 mg and 66 mg on subsequent instances of recurrences of said toxicity.

In another particular embodiment, the present invention relates to a method for the treatment of an adult patient with refractory CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 7 days and resumed at 90 mg after recovery of patient from said toxicity; or
b) the initial daily dose is withheld for less than 7 days and if toxicity is resolved then resumed at 90 mg; or
c) the initial daily dose is withheld for at least 7 days and resumed at a reduced daily dose selected from 45 mg, 48 mg and 66 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another particular embodiment, the present invention relates to a method for the treatment of an adult patient with refractory CML, wherein the method comprises administering to the patient an initial daily dose of 90 mg of a compound of Formula I such that when the patient develops non-hematological toxicity:
a) the initial daily dose is withheld for at least 7 days and resumed at 90 mg after recovery of patient from said toxicity; or
b) the initial daily dose is withheld for less than 7 days and if toxicity is resolved then resumed at 90 mg; or
c) the initial daily dose is withheld for at least 7 days and resumed at a reduced daily dose selected from 45 mg, 48 mg and 66 mg on subsequent instances of recurrences of said toxicity;
and wherein the daily dose of the compound of Formula I or a pharmaceutically acceptable salt thereof in the patient results in mean AUC₀₋₂₄ ranging from 18237 ng*h/mL to 27355±2782 5827 ng*h/mL.

In another particular embodiment, the present invention relates to a method for the treatment of an adult patient with refractory CML, wherein the method comprises administering to the patient an initial daily dose of 90 mg of a compound of Formula I or a pharmaceutically acceptable salt thereof such that when the patient develops non-hematological toxicity:
a) the initial daily dose is withheld for at least 7 days and resumed at 90 mg after recovery of patient from said toxicity; or
b) the initial daily dose is withheld for less than 7 days and if toxicity is resolved then resumed at 90 mg; or
c) the initial daily dose is withheld for at least 7 days and resumed at a reduced daily dose selected from 45 mg, 48 mg and 66 mg on subsequent instances of recurrences of said toxicity;
and wherein the daily dose of the compound of Formula I or a pharmaceutically acceptable salt thereof in the patient results in mean Cₘₐₓ ranging from 2277 ng/ml to 3415±1566 ng/mL.

In another particular embodiment, the present invention provides a method for the treatment of an adult patient with refractory CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 7 days and resumed at 90 mg after recovery of patient from said toxicity; or
b) the initial daily dose is withheld for less than 7 days and if toxicity is resolved then resumed at 90 mg; or
c) the initial daily dose is withheld for at least 7 days and resumed at a reduced daily dose selected from 45 mg, 48 mg and 66 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction and wherein the daily dose of the compound of Formula I or a pharmaceutically acceptable salt thereof in the patient results in mean AUC₀₋₂₄ ranging from 18237 ng*h/mL to 27355±2782 5827 ng*h/mL.

In another particular embodiment, the present invention provides a method for the treatment of an adult patient with refractory CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 7 days and resumed at 90 mg after recovery of patient from said toxicity; or
b) the initial daily dose is withheld for less than 7 days and if toxicity is resolved then resumed at 90 mg; or
c) the initial daily dose is withheld for at least 7 days and resumed at a reduced daily dose selected from 45 mg, 48 mg and 66 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction and wherein the daily dose of the compound of Formula I or a pharmaceutically acceptable salt thereof in the patient results in mean Cₘₐₓ ranging from 2277 ng/ml to 3415±1566 ng/mL.

In one particular embodiment, the present invention relates to a method for the treatment of an adult patient with refractory CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 14 days and resumed at 90 mg after recovery of patient from said toxicity; or
b) the initial daily dose is withheld for less than 14 days and if toxicity is resolved then resumed at 90 mg; or
c) the initial daily dose is withheld for at least 14 days and resumed at a reduced daily dose selected from 45 mg, 48 mg and 66 mg on subsequent instances of recurrences of said toxicity.

In another particular embodiment, the present invention relates to a method for the treatment of an adult patient with refractory CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 14 days and resumed at 90 mg after recovery of patient from said toxicity; or
b) the initial daily dose is withheld for less than 14 days and if toxicity is resolved then resumed at 90 mg; or
c) the initial daily dose is withheld for at least 14 days and resumed at a reduced daily dose selected from 45 mg, 48 mg and 66 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another particular embodiment, the present invention relates to a method for the treatment of an adult patient with refractory CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 14 days and resumed at 90 mg after recovery of patient from said toxicity; or
b) the initial daily dose is withheld for less than 14 days and if toxicity is resolved then resumed at 90 mg; or
c) the initial daily dose is withheld for at least 14 days and resumed at a reduced daily dose selected from 45 mg, 48 mg and 66 mg on subsequent instances of recurrences of said toxicity;
and wherein the daily dose of the compound of Formula I or a pharmaceutically acceptable salt thereof in the patient results in mean AUC₀₋₂₄ ranging from 18237 ng*h/mL to 27355±2782 5827 ng*h/mL.

In another particular embodiment, the present invention relates to a method for the treatment of an adult patient with refractory CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 14 days and resumed at 90 mg after recovery of patient from said toxicity; or
b) the initial daily dose is withheld for less than 14 days and if toxicity is resolved then resumed at 90 mg; or
c) the initial daily dose is withheld for at least 14 days and resumed at a reduced daily dose selected from 45 mg, 48 mg and 66 mg on subsequent instances of recurrences of said toxicity;
and wherein the daily dose of the compound of Formula I or a pharmaceutically acceptable salt thereof in the patient results in mean Cₘₐₓ ranging from 2277 ng/ml to 3415±1566 ng/mL.

In another particular embodiment, the present invention relates to a method for the treatment of an adult patient with refractory CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 14 days and resumed at 90 mg after recovery of patient from said toxicity; or
b) the initial daily dose is withheld for less than 14 days and if toxicity is resolved then resumed at 90 mg; or
c) the initial daily dose is withheld for at least 14 days and resumed at a reduced daily dose selected from 45 mg, 48 mg and 66 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction and wherein the daily dose of the compound of Formula I or a pharmaceutically acceptable salt thereof in the patient results in mean AUC₀₋₂₄ ranging from 18237 ng*h/mL to 27355±2782 5827 ng*h/mL.

In another particular embodiment, the present invention relates to a method for the treatment of an adult patient with refractory CML, wherein the method comprises administering to said patient an initial daily dose of 90 mg of a compound of Formula I or a pharmaceutically acceptable salt thereof such that when the patient develops non-hematologic toxicity:
a) the initial daily dose is withheld for at least 14 days and resumed at 90 mg after recovery of patient from said toxicity; or
b) the initial daily dose is withheld for less than 14 days and if toxicity is resolved then resumed at 90 mg; or
c) the initial daily dose is withheld for at least 14 days and resumed at a reduced daily dose selected from 45 mg, 48 mg and 66 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction and wherein the daily dose of the compound of Formula I or a pharmaceutically acceptable salt thereof results in a mean Cₘₐₓ ranging from 2277 ng/ml to 3415±1566 ng/mL.

In one particular embodiment, the present invention relates to a method for the treatment of an adult patient with refractory CML, wherein the method comprises administering to the patient an initial daily dose of 90 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose of selected from 45 mg, 48 mg and 66 mg on subsequent instances of recurrences of said toxicity.

In another particular embodiment, the present invention relates to a method for the treatment of an adult patient with refractory CML, wherein the method comprises administering to the patient an initial daily dose of 90 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose selected from 45 mg, 48 mg and 66 mg on subsequent instances of recurrences of said toxicity and wherein,
the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another particular embodiment, the present invention relates to a method for the treatment of an adult patient with refractory CML, wherein the method comprises administering to the patient an initial daily dose of 90 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose selected from 45 mg, 48 mg and 66 mg on subsequent instances of recurrences of said toxicity, and wherein the daily dose of the compound of Formula I or a pharmaceutically acceptable salt thereof in the patient results in mean AUC₀₋₂₄ ranging from 18237 ng*h/mL to 27355±2782 5827 ng*h/mL.

In another particular embodiment, the present invention relates to a method for the treatment of an adult patient with refractory CML, wherein the method comprises administering to the patient an initial daily dose of 90 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose selected from 45 mg, 48 mg and 66 mg on subsequent instances of recurrences of said toxicity, and wherein the daily dose of the compound of Formula I or a pharmaceutically acceptable salt thereof in the patient results in mean Cₘₐₓ ranging from 2277 ng/ml to 3415±1566 ng/mL.

In another particular embodiment, the present invention relates to a method for the treatment of an adult patient with refractory CML, wherein the method comprises administering to the patient an initial daily dose of 90 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose selected from 45 mg, 48 mg and 66 mg on subsequent instances of recurrences of said toxicity and wherein,
the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another particular embodiment, the present invention relates to a method for the treatment of an adult patient with refractory CML, wherein the method comprises administering to the patient an initial daily dose of 90 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose selected from 45 mg, 48 mg and 66 mg on subsequent instances of recurrences of said toxicity and wherein,
the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction and wherein the daily dose of the compound of Formula I or a pharmaceutically acceptable salt thereof in the patient results in mean AUC₀₋₂₄ ranging from 18237 ng*h/mL to 27355±2782 5827 ng*h/mL.

In another particular embodiment, the present invention relates to a method for the treatment of an adult patient with refractory CML, wherein the method comprises administering to the patient an initial daily dose of 90 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose selected from 45 mg, 48 mg and 66 mg on subsequent instances of recurrences of said toxicity and wherein,
the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction and wherein the daily dose of the compound of Formula I or a pharmaceutically acceptable salt thereof results in a mean Cₘₐₓ ranging from 2277 ng/mL to 3415±1566 ng/ml.

In a particular embodiment, the present invention relates to a method for the treatment of an adult patient with newly diagnosed CML, wherein the method comprises administering to said patient an initial daily dose of 135 mg of a compound of Formula I or a pharmaceutically acceptable salt thereof such that when the patient develops non-hematologic toxicity:
a) the initial daily dose is withheld for at least 7 days and resumed at 135 mg after recovery of patient from said toxicity; or
b) the initial daily dose is withheld for less than 7 days and if toxicity is resolved then resumed at 135 mg; or
c) the initial daily dose is withheld for at least 7 days and resumed at a reduced daily dose of 90 mg on first instance and to 45 mg on subsequent instances of recurrences of said toxicity.

In another particular embodiment, the present invention relates to a method for the treatment of an adult patient with newly diagnosed CML, wherein the method comprises administering to said patient an initial daily dose of 135 mg of a compound of Formula I or a pharmaceutically acceptable salt thereof such that when the patient develops non-hematologic toxicity:
a) the initial daily dose is withheld for at least 7 days and resumed at 135 mg after recovery of patient from said toxicity; or
b) the initial daily dose is withheld for less than 7 days and if toxicity is resolved then resumed at 135 mg; or
c) the initial daily dose is withheld for at least 7 days and resumed at a reduced daily dose of 90 mg on first instance and to 45 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In a particular embodiment, the present invention relates to a method for the treatment of an adult patient with newly diagnosed CML, wherein the method comprises administering to said patient an initial daily dose of 135 mg of a compound of Formula I or a pharmaceutically acceptable salt thereof such that when the patient develops non-hematologic toxicity:
a) the initial daily dose is withheld for at least 14 days and resumed at 135 mg after recovery of patient from said toxicity; or
b) the initial daily dose is withheld for less than 14 days and if toxicity is resolved then resumed at 135 mg; or
c) the initial daily dose is withheld for at least 14 days and resumed at a reduced daily dose of 90 mg on first instance and to 45 mg on subsequent instances of recurrences of said toxicity.

In another particular embodiment, the present invention relates to a method for the treatment of an adult patient with newly diagnosed CML, wherein the method comprises administering to said patient an initial daily dose of 135 mg of a compound of Formula I or a pharmaceutically acceptable salt thereof such that when the patient develops non-hematologic toxicity:
a) the initial daily dose is withheld for at least 14 days and resumed at 135 mg after recovery of patient from said toxicity; or
b) the initial daily dose is withheld for less than 14 days and if toxicity is resolved then resumed at 135 mg; or
c) the initial daily dose is withheld for at least 14 days and resumed at a reduced daily dose of 90 mg on first instance and to 45 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In one embodiment, the present invention relates to a method for the treatment of an adult patient with newly diagnosed CML, wherein the method comprises administering to the patient an initial daily dose of 135 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose of 90 mg on first instance and to 45 mg on subsequent instances of recurrences of said toxicity.

In another particular embodiment, the present invention relates to a method for the treatment of an adult patient with newly diagnosed CML, wherein the method comprises administering to the patient an initial daily dose of 135 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose of 90 mg on first instance and to 45 mg on subsequent instances of recurrences of said toxicity and wherein,
the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In a particular embodiment, the present invention relates to a method for the treatment of an adult patient with refractory CML, wherein the method comprises administering to said patient an initial daily dose of 135 mg of a compound of Formula I or a pharmaceutically acceptable salt thereof such that when the patient develops non-hematologic toxicity:
a) the initial daily dose is withheld for at least 7 days and resumed at 135 mg after recovery of patient from said toxicity; or
b) the initial daily dose is withheld for less than 7 days and if toxicity is resolved then resumed at 135 mg; or
c) the initial daily dose is withheld for at least 7 days and resumed at a reduced daily dose of 90 mg on first instance and to 45 mg on subsequent instances of recurrences of said toxicity.

In another particular embodiment, the present invention relates to a method for the treatment of an adult patient with refractory CML, wherein the method comprises administering to said patient an initial daily dose of 135 mg of a compound of Formula I or a pharmaceutically acceptable salt thereof such that when the patient develops non-hematologic toxicity:
a) the initial daily dose is withheld for at least 7 days and resumed at 135 mg after recovery of patient from said toxicity; or
b) the initial daily dose is withheld for less than 7 days and if toxicity is resolved then resumed at 135 mg; or
c) the initial daily dose is withheld for at least 7 days and resumed at a reduced daily dose of 90 mg on first instance and to 45 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In one particular embodiment, the present invention relates to a method for the treatment of an adult patient with refractory CML, wherein the method comprises administering to said patient an initial daily dose of 135 mg of a compound of Formula I or a pharmaceutically acceptable salt thereof such that when the patient develops non-hematologic toxicity:
a) the initial daily dose is withheld for at least 14 days and resumed at 135 mg after recovery of patient from said toxicity; or
b) the initial daily dose is withheld for less than 14 days and if toxicity is resolved then resumed at 135 mg; or
c) the initial daily dose is withheld for at least 14 days and resumed at a reduced daily dose selected from 90 mg and 45 mg on subsequent instances of recurrences of said toxicity.

In another particular embodiment, the present invention relates to a method for the treatment of an adult patient with refractory CML, wherein the method comprises administering to said patient an initial daily dose of 135 mg of a compound of Formula I or a pharmaceutically acceptable salt thereof such that when the patient develops non-hematologic toxicity:
a) the initial daily dose is withheld for at least 14 days and resumed at 135 mg after recovery of patient from said toxicity; or
b) the initial daily dose is withheld for less than 14 days and if toxicity is resolved then resumed at 135 mg; or
c) the initial daily dose is withheld for at least 14 days and resumed at a reduced daily dose of 90 mg on first instance and to 45 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In one particular embodiment, the present invention relates to a method for the treatment of an adult patient with refractory CML, wherein the method comprises administering to the patient an initial daily dose of 135 mg of a compound of Formula I or a pharmaceutically acceptable salt thereof such that when the patient develops hematologic toxicity attributable to the compound of Formula I or a pharmaceutically acceptable salt thereof, the initial daily dose is withheld for up to four weeks to allow recovery of patient from said toxicity and the treatment is continued at a reduced daily dose of 90 mg on first instance and to 45 mg on subsequent instances of recurrences of said toxicity.

In another particular embodiment, the present invention relates to a method for the treatment of an adult patient with refractory CML, wherein the method comprises administering to the patient an initial daily dose of 135 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose of 90 mg on first instance and to 45 mg on subsequent instances of recurrences of said toxicity and wherein,
the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In another particular embodiment, the present invention relates to a method for the treatment of an adult patient with refractory CML, wherein the method comprises administering to the patient an initial daily dose of 135 mg of a compound of Formula I or a pharmaceutically acceptable salt thereof such that when the patient develops hematologic toxicity attributable to the compound of Formula I or a pharmaceutically acceptable salt thereof,
the initial daily dose is withheld for up to four weeks to allow recovery of patient from said toxicity and the treatment is continued at a reduced daily dose of 90 mg on first instance and to 45 mg on subsequent instances of recurrences of said toxicity and wherein,
the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In yet another particular embodiment, the present invention provides a method of treating an adult patient with CML by orally administering a compound of Formula I or a pharmaceutically acceptable salt thereof under fasting conditions such that, a mean AUC₀₋₂₄ achieved in patient by administering the compound of Formula I or a pharmaceutically acceptable salt thereof under fasting conditions is 15% higher as compared to the mean AUC₀₋₂₄ obtained by administering the compound of Formula I or a pharmaceutically acceptable salt thereof under fed conditions.

According to an embodiment, the present invention provides a method for the treatment of an adult patient with CML, wherein the method comprises administering a compound of Formula I or a pharmaceutically acceptable salt thereof to the patient at a daily dose such that the plasma concentration of the compound of Formula I or a pharmaceutically acceptable salt thereof is not less than 50 ng/mL.

In an embodiment, the present invention provides a method for the treatment of an adult patient with CML, wherein the method comprises administering a compound of Formula I or a pharmaceutically acceptable salt thereof to the patient at a daily dose such that the plasma concentration of the compound of Formula I or a pharmaceutically acceptable salt thereof is not less than 70 ng/mL.

In another embodiment, the present invention provides a method for the treatment of an adult patient with CML, wherein the method comprises administering a compound of Formula I or a pharmaceutically acceptable salt thereof to the patient at a daily dosage such that the plasma concentration of the compound of Formula I or a pharmaceutically acceptable salt thereof is not less than 90 ng/ml.

In another particular embodiment, the present invention provides a method for the treatment of an adult patient with CML, wherein the method comprises administering a compound of Formula I or a pharmaceutically acceptable salt thereof to the patient at a daily dose such that the plasma concentration of the compound of Formula I or a pharmaceutically acceptable salt thereof is not less than 100 ng/mL.

In another particular embodiment, the present invention provides a method for the treatment of an adult patient with CML, comprising orally administering a compound of Formula I or a pharmaceutically acceptable salt thereof at a predetermined daily dose, wherein said daily dose, (a) results in not less than 50 ng/ml of the compound of Formula I or a pharmaceutically acceptable salt thereof in the plasma; (b) results in mean AUC₀₋₂₄ ranging from 6226±5827 ng*h/mL to 60373±55659 ng*h/mL; (c) achieves a mean Cₘₐₓ ranging from 664±450 ng/ml to 5054±3051 ng/ml; (d) achieves 15% higher AUC₀₋₂₄ and 20% higher Cₘₐₓ when administered under fasting conditions as compared to when administered under fed conditions; and/or (e) results in patient's QT interval of less than about 500 ms.

In another particular embodiment, the present invention relates to a method for the treatment of an adult patient with CML, comprising orally administering a compound of Formula I or a pharmaceutically acceptable salt thereof at a predetermined daily dose, wherein the daily dose is escalated to achieve or maintain complete hematologic response or the daily dose is reduced when the patient experiences severe adverse reaction and wherein the predetermined, escalated or reduced daily dose; (a) results in not less than 50 ng/ml of the compound of Formula I or a pharmaceutically acceptable salt thereof in the plasma; (b) results in mean AUC₀₋₂₄ ranging from 6226±5827 ng*h/mL to 60373±55659 ng*h/mL; (c) achieves a mean Cₘₐₓ ranging from 664±450 ng/ml to 5054±3051 ng/ml; (d) achieves 15% higher AUC₀₋₂₄ and 20% higher Cₘₐₓ when administered under fasting conditions as compared to when administered under fed conditions; and/or (e) results in patient's QT interval of less than about 500 ms.

According to an embodiment, the present invention relates to a method for the treatment of an adult patient who had previously undergone treatment for T315I-positive CML and has a relapsed or refractory CML condition, wherein the method comprises orally administering a compound of Formula I or a pharmaceutically acceptable salt thereof at an initial daily dose, and escalating or de-escalating the dose to achieve or maintain disease response, with no severe adverse reaction.

According to an embodiment, the patient having T315I-positive CML may have been previously treated using TKIs such as ponatinib or asciminib.

According to an embodiment, the patient having T315I-positive CML may have been previously treated using TKIs such as ponatinib and asciminib.

According to a particular embodiment, the present invention relates to a method for the treatment of an adult patient who had previously undergone treatment using ponatinib and/or asciminib having a relapsed or refractory CML condition, wherein the method comprises orally administering a compound of Formula I or a pharmaceutically acceptable salt thereof at an initial daily dose, and escalating or de-escalating the dose to achieve or maintain disease response, with no severe adverse reaction.

The compound of the present invention was surprisingly found to be highly efficacious, safe and tolerable at daily dose from 10 mg to 210 mg. The compound was efficacious, safe and tolerable at daily dose between 12 mg to 210 mg. It has surprisingly found that the dose of 174 mg was effective and tolerable.

Based on phase I, open-label, single-agent, multi-center, multi-dose, dose-escalation study in subjects with Philadelphia chromosome-positive Ph+ CML (CP CML, AP CML and BP CML) and Ph+ ALL and phase 2, open-label, multicenter, single-arm, a study in subjects with Ph+ CML (CP CML, AP CML and BP CML) using the compound of Formula I, it was found that major cytogenetic response (MCyR, combining both complete and partial cytogenetic responses) and major molecular response (MMR) were 61% and 33.9%, respectively across all subjects enrolled into the study. Additionally, 71.2% of all the subjects were refractory and/or intolerant to ≥3 TKIs. The MCyR and MMR in subjects with CP CML were 63% and 34.8%, respectively, and those with more aggressive disease viz. AP CML, BP CML and Ph+ ALL were 53.8% and 30.8%, respectively.

Of the 59 subjects enrolled in the study, 29 had prior treatment with ponatinib. Of these 29 patients, MCyR and MMR were observed in 51.7% and 37.9%, of subjects respectively. The compound of Formula I demonstrated comparable efficacy across subjects independent of prior ponatinib treatment.

In a subgroup of CP CML patients, efficacy study using the compound of Formula I with patients previously treated using more than 3 TKIs including ponatinib and with patients previously treated using more than 3 TKIs including ponatinib and asciminib, showed CCyR as best response in 46.7% and 33.3%, respectively. Major cytogenetic response as best response was reported in 66.7% and 33.3% of patients in the ponatinib and ponatinib plus asciminib group.

Particularly at a 174 mg initial dose, 20 out of 24 subjects (83.3%) subjects had completed hematological response. All subjects belonging to the CP CML cohort demonstrated both MCyR and MMR, which was their best response with previous treatments. Major molecular response was further deepened to complete molecular response in 2 (9.1%) subjects.

Temporary dose interruptions and dose modification occurred in 37.7% and 20.7% of patients, respectively. AEs leading to permanent discontinuation occurred in 6 pts (11.3%)-1 pt each due to ICH; gastritis and decreased appetite; recurrence of Ph+ ALL; disease progression; sudden death; and suspected COVID-19. There were 5 deaths during the study out of which only 1 was considered related to the compound of Formula I. Others included sudden death, disease progression, fungal pneumonia and suspected COVID-19 (1 each). Safety and tolerability were assessed through physical examination, analysis of frequency, and intensity of adverse events (AE), vital signs, physical examinations, electrocardiogram, and clinical laboratory tests. Adverse events were assessed based on the National Cancer Institute Common Terminology Criteria for Adverse Events and categorized as serious AEs or non-serious AEs. The relationship of AEs to the compound of Formula I was assessed by the Investigator per the protocol-defined criteria for attribution assessment.

According to an embodiment, in patients under treatment TEAEs were reported by 96.2% patients and grade 3 or 4 TEAEs were reported by 64.1% of patients. Out of 64.1% of patients with grade 3 or 4 AEs, 6 (33.4%) patients had previous 2 TKI treatments, 28 (80%) had 3 or more TKI treatments and 19 (86.3%) of those were treated with ponatinib. Most frequently reported TEAEs included thrombocytopenia which was seen in 41.5%, out of which 18.8% were grade 3 or 4, diarrhea which was reported in 22% out of which only 2% were grade 3 or 4, anemia was reported in 20.8% out of which only 13% were grade 3 or 4 and cough was reported in 20.8%.

In an embodiment, the compound of formula I at daily dose from 10 mg to 210 mg was surprisingly found to have long-term safety against any of the cardiovascular adverse events. Generally, cardiovascular toxicity is a main and serious adverse event caused by anti-cancer agents. Heart issues may arise due to the use of TKIs such as imatinib mesylate, dasatinib, nilotinib, sunitinib, sorafenib and lapatinib while using them for treating CML or ALL. The present inventors surprisingly found that the compound of Formula I when used even at the highest dose of 204 mg for even for as long as 24 months did not result in major CVAEs.

As can be seen in Tables 12 and 13, thirty patients had medical history of cardiovascular adverse events (CVAEs) of which 12 patients reported CVAEs on study. Out of the 12 patients, 10 CVAEs were unrelated and only 2 were related to the compound of Formula I. Overall, 14 patients (26.4%) had CVAEs out of which 5.6% had grade 3 or 4 AEs. Among the 14 patients 1 (5.5%) had undergone treatment using less than or equal to 2 TKIs, 13 (37.1%) had undergone treatment using more than or equal to 3 TKIs, and 10 (45.4%) patients had prior treatment using ponatinib. The two CVAEs considered related to the compound of Formula I was grade 2 hypertension and one fatal intracranial hemorrhage (ICH) with concomitant disease progression to BP CML. No subject reached the subject withdrawal criteria based on QTcF prolongation or safety laboratory tests.

In one particular embodiment, the present invention provides a method for the treatment of a patient with chronic myeloid leukemia (CML), comprising orally administering to said patient a therapeutically effective amount of compound of Formula I:

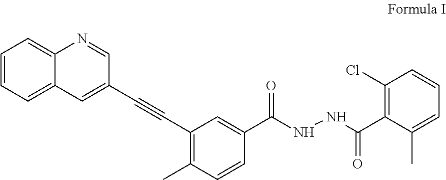 [SMILES: Cc1ccc(C(=O)NNC(=O)c2c(C)cccc2Cl)cc1C#Cc1cnc2ccccc2c1]

or a pharmaceutically acceptable salt thereof on a continuous initial daily dose until progression of chronic myeloid leukemia (CML) or unacceptable toxicity.

In one particular embodiment, the present invention provides a method for the treatment of a patient with chronic myeloid leukemia (CML), comprising orally administering to said patient a therapeutically effective amount of a compound of Formula I:

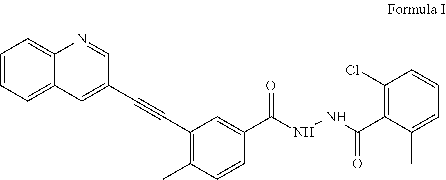 [SMILES: Cc1ccc(C(=O)NNC(=O)c2c(C)cccc2Cl)cc1C#Cc1cnc2ccccc2c1]

or a pharmaceutically acceptable salt thereof at an initial daily dose, and escalating or de-escalating the dose to achieve or maintain at least one of (a) complete hematological response, (b) complete hematological response and partial cytogenetic response, (c) complete hematological response and complete cytogenetic response, or (d) complete hematological response, complete cytogenetic response, and major molecular response.

In one particular embodiment, the present invention provides a method for the treatment of a patient with chronic myeloid leukemia (CML), comprising orally administering to said patient a therapeutically effective amount of compound of Formula I:

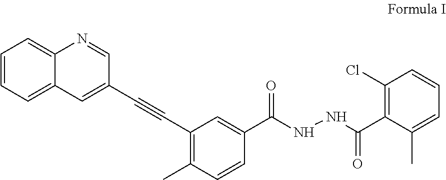 [SMILES: Cc1ccc(C(=O)NNC(=O)c2c(C)cccc2Cl)cc1C#Cc1cnc2ccccc2c1]

or a pharmaceutically acceptable salt thereof at an initial daily dose, and escalating or de-escalating the dose to achieve or maintain complete hematologic response with no severe adverse reaction.

In one particular embodiment, the present invention provides a method for the treatment of a patient with chronic myeloid leukemia (CML), comprising orally administering a therapeutically effective amount of a compound of Formula I:

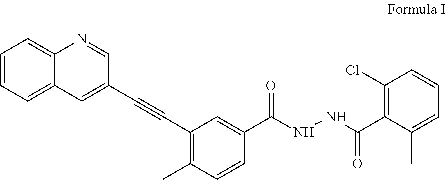 [SMILES: Cc1ccc(C(=O)NNC(=O)c2c(C)cccc2Cl)cc1C#Cc1cnc2ccccc2c1]

or a pharmaceutically acceptable salt thereof at a daily dose from 10 mg to 204 mg.

In one particular embodiment, the present invention provides a method for the treatment of a patient with chronic myeloid leukemia (CML), comprising orally administering a therapeutically effective amount of a compound of Formula I:

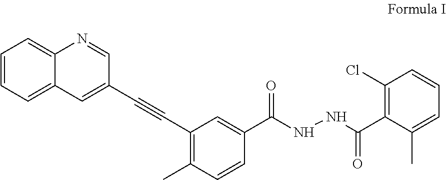 [SMILES: Cc1ccc(C(=O)NNC(=O)c2c(C)cccc2Cl)cc1C#Cc1cnc2ccccc2c1]

or a pharmaceutically acceptable salt thereof at an initial daily dose from 10 mg to 204 mg.

In one particular embodiment, the present invention provides a method for the treatment of a patient with chronic myeloid leukemia (CML), comprising orally administering a therapeutically effective amount of a compound of Formula I:

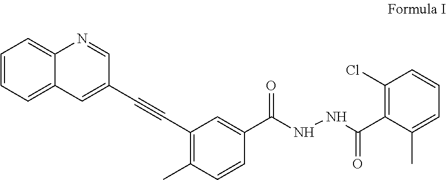 [SMILES: Cc1ccc(C(=O)NNC(=O)c2c(C)cccc2Cl)cc1C#Cc1cnc2ccccc2c1]

or a pharmaceutically acceptable salt thereof at an initial daily dose from 10 mg to 180 mg, wherein the initial dose is escalated up to 204 mg to achieve or maintain complete hematologic response.

In one particular embodiment, the present invention provides a method for the treatment of a patient with chronic myeloid leukemia (CML), comprising orally administering a therapeutically effective amount of a compound of Formula I:

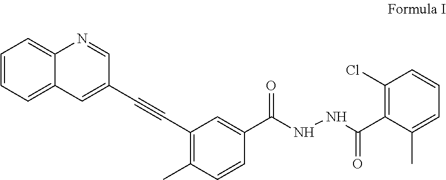 [SMILES: Cc1ccc(C(=O)NNC(=O)c2c(C)cccc2Cl)cc1C#Cc1cnc2ccccc2c1]

or a pharmaceutically acceptable salt thereof at an initial daily dose from 24 mg to 204 mg, wherein the initial dose is de-escalated between 12 mg to 174 mg to achieve or maintain complete hematologic response.

In one particular embodiment, the present invention provides a method for the treatment of a patient with chronic myeloid leukemia (CML), comprising orally administering a therapeutically effective amount of a compound of Formula I:

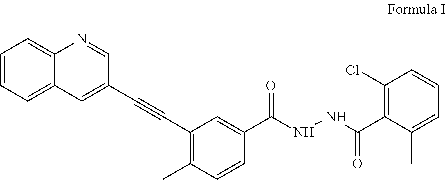 [SMILES: Cc1ccc(C(=O)NNC(=O)c2c(C)cccc2Cl)cc1C#Cc1cnc2ccccc2c1]

or a pharmaceutically acceptable salt thereof at an initial daily dose from 10 mg to 174 mg, wherein the initial dose is escalated up to 204 mg to achieve or maintain complete hematologic response with no severe adverse reaction.

In one particular embodiment, the present invention provides a method for the treatment of a patient with chronic myeloid leukemia (CML), wherein the method comprises administering to said patient an initial daily dose selected from 12 mg, 24 mg, 43.5 mg, 45 mg, 48 mg, 66 mg, 87 mg, 90 mg, 126 mg, 130.5 mg, 135 mg, 174 mg, 180 mg, 200 mg and 204 mg of a compound of Formula I:

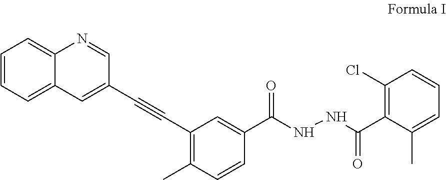 [SMILES: Cc1ccc(C(=O)NNC(=O)c2c(C)cccc2Cl)cc1C#Cc1cnc2ccccc2c1]

or a pharmaceutically acceptable salt thereof such that when the patient develops toxicity:
   a) the initial daily dose is withheld for at least 7 days and resumed at daily dose selected from 12 mg, 24 mg, 43.5 mg, 45 mg, 48 mg, 66 mg, 87 mg, 90 mg, 126 mg, 130.5 mg, 135 mg, 174 mg, 180 mg, 200 mg and 204 mg; or
   b) the initial daily dose is withheld for less than 7 days and if toxicity is resolved then resumed at daily dose selected from 12 mg, 24 mg, 43.5 mg, 45 mg, 48 mg, 66 mg, 87 mg, 90 mg, 126 mg, 130.5 mg, 135 mg, 174 mg, 180 mg, 200 mg and 204 mg; or
   c) an initial daily dose is withheld for at least 7 days and resumed at a reduced daily dose selected from 12 mg, 24 mg, 43.5 mg, 45 mg, 48 mg, 66 mg, 87 mg, 90 mg, 126 mg, 130.5 mg, 135 mg, 174 mg, and 180 mg.

In one particular embodiment, the present invention provides a method for the treatment of a patient with chronic myeloid leukemia (CML), wherein the method comprises administering to said patient an initial daily dose selected from 12 mg, 24 mg, 43.5 mg, 45 mg, 48 mg, 66 mg, 87 mg, 90 mg, 126 mg, 130.5 mg, 135 mg, 174 mg, 180 mg, 200 mg and 204 mg of a compound of Formula I:

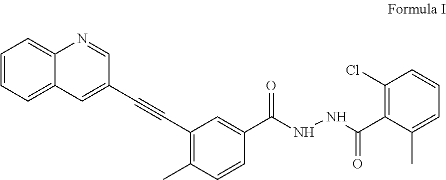 [SMILES: Cc1ccc(C(=O)NNC(=O)c2c(C)cccc2Cl)cc1C#Cc1cnc2ccccc2c1]

or a pharmaceutically acceptable salt thereof such that when the patient develops toxicity:
   a) the initial daily dose is withheld for at least 7 days and resumed at daily dose selected from 12 mg, 24 mg, 43.5 mg, 45 mg, 48 mg, 66 mg, 87 mg, 90 mg, 126 mg, 130.5 mg, 135 mg, 174 mg, 180 mg, 200 mg and 204 mg; or
   b) the initial daily dose is withheld for less than 7 days and if toxicity is resolved then resumed at daily dose selected from 12 mg, 24 mg, 43.5 mg, 45 mg, 48 mg, 66 mg, 87 mg, 90 mg, 126 mg, 130.5 mg, 135 mg, 174 mg, 180 mg, 200 mg and 204 mg; or
   c) an initial daily dose is withheld for at least 7 days and resumed at a reduced daily dose selected from 12 mg, 24 mg, 43.5 mg, 45 mg, 48 mg, 66 mg, 87 mg, 90 mg, 126 mg, 130.5 mg, 135 mg, 174 mg, and 180 mg on first instance and to 12 mg, 24 mg, 43.5 mg, 45 mg, 48 mg, 66 mg, 87 mg, 90 mg, 126 mg, 130.5 mg, and 135 mg on subsequent instances of recurrences of said toxicity.

In one particular embodiment, the present invention provides a method for the treatment of a patient with chronic myeloid leukemia (CML), wherein the method comprises administering to said patient an initial daily dose selected from 12 mg, 24 mg, 43.5 mg, 45 mg, 48 mg, 66 mg, 87 mg, 90 mg, 126 mg, 130.5 mg, 135 mg, 174 mg, 180 mg, 200 mg and 204 mg of a compound of Formula I:

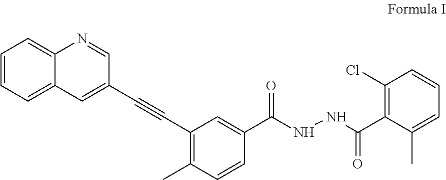 [SMILES: Cc1ccc(C(=O)NNC(=O)c2c(C)cccc2Cl)cc1C#Cc1cnc2ccccc2c1]

or a pharmaceutically acceptable salt thereof such that when the patient develops toxicity:
   a) the initial daily dose is withheld for at least 7 days and resumed at daily dose selected from 12 mg, 24 mg, 43.5 mg, 45 mg, 48 mg, 66 mg, 87 mg, 90 mg, 126 mg, 130.5 mg, 135 mg, 174 mg, 180 mg, 200 mg and 204 mg; or
   b) the initial daily dose is withheld for less than 7 days and if toxicity is resolved then resumed at daily dose selected from 12 mg, 24 mg, 43.5 mg, 45 mg, 48 mg, 66 mg, 87 mg, 90 mg, 126 mg, 130.5 mg, 135 mg, 174 mg, 180 mg, 200 mg and 204 mg; or
   c) an initial daily dose is withheld for at least 7 days and resumed at a reduced daily dose selected from 12 mg, 24 mg, 43.5 mg, 45 mg, 48 mg, 66 mg, 87 mg, 90 mg, 126 mg, 130.5 mg, 135 mg, 174 mg, and 180 mg on first instance and to 12 mg, 24 mg, 43.5 mg, 45 mg, 48 mg, 66 mg, 87 mg, 90 mg, 126 mg, 130.5 mg, and 135 mg on subsequent instances of recurrences of said toxicity;
and wherein, the reduced dose is optionally re-escalated to any one of the dose selected from 24 mg to 204 mg.

In one particular embodiment, the present invention provides a method for the treatment of a patient with chronic myeloid leukemia (CML), wherein the method comprises administering to said patient an initial daily dose selected from 12 mg, 24 mg, 43.5 mg, 45 mg, 48 mg, 66 mg, 87 mg, 90 mg, 126 mg, 130.5 mg, 135 mg, 174 mg, 180 mg, 200 mg and 204 mg of a compound of Formula I:

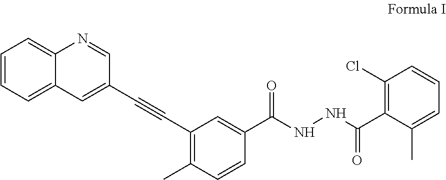 [SMILES: Cc1ccc(C(=O)NNC(=O)c2c(C)cccc2Cl)cc1C#Cc1cnc2ccccc2c1]

or a pharmaceutically acceptable salt thereof such that when the patient develops toxicity:
   a) the initial daily dose is withheld for at least 7 days and resumed at daily dose selected from 12 mg, 24 mg, 43.5 mg, 45 mg, 48 mg, 66 mg, 87 mg, 90 mg, 126 mg, 130.5 mg, 135 mg, 174 mg, 180 mg, 200 mg and 204 mg; or
   b) the initial daily dose is withheld for less than 7 days and if toxicity is resolved then resumed at daily dose selected from 12 mg, 24 mg, 43.5 mg, 45 mg, 48 mg, 66 mg, 87 mg, 90 mg, 126 mg, 130.5 mg, 135 mg, 174 mg, 180 mg, 200 mg and 204 mg; or
   c) an initial daily dose is withheld for at least 7 days and resumed at a reduced daily dose selected from 12 mg, 24 mg, 43.5 mg, 45 mg, 48 mg, 66 mg, 87 mg, 90 mg, 126 mg, 130.5 mg, 135 mg, 174 mg, and 180 mg on first instance and to 12 mg, 24 mg, 43.5 mg, 45 mg, 48 mg, 66 mg, 87 mg, 90 mg, 126 mg, 130.5 mg, and 135 mg on subsequent instances of recurrences of said toxicity;
and wherein, the reduced dose is optionally re-escalated to any one of the dose selected from 24 mg, 43.5 mg, 45 mg, 48 mg, 66 mg, 87 mg, 90 mg, 126 mg, 130.5 mg, 135 mg, 174 mg, 180 mg, 200 mg, and 204 mg.

In one particular embodiment, the present invention provides a method for the treatment of a patient with chronic myeloid leukemia (CML), wherein the method comprises administering to said patient an initial daily dose selected from 12 mg, 24 mg, 43.5 mg, 45 mg, 48 mg, 66 mg, 87 mg, 90 mg, 126 mg, 130.5 mg, 135 mg, 174 mg, 180 mg, 200 mg and 204 mg of a compound of Formula I:

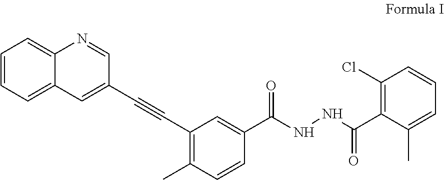 [SMILES: Cc1ccc(C(=O)NNC(=O)c2c(C)cccc2Cl)cc1C#Cc1cnc2ccccc2c1]

or a pharmaceutically acceptable salt thereof such that when the patient develops toxicity:
   a) the initial daily dose is withheld for at least 7 days and resumed at daily dose selected from 12 mg, 24 mg, 43.5 mg, 45 mg, 48 mg, 66 mg, 87 mg, 90 mg, 126 mg, 130.5 mg, 135 mg, 174 mg, 180 mg, 200 mg and 204 mg; or
   b) the initial daily dose is withheld for less than 7 days and if toxicity is resolved then resumed at daily dose selected from 12 mg, 24 mg, 43.5 mg, 45 mg, 48 mg, 66 mg, 87 mg, 90 mg, 126 mg, 130.5 mg, 135 mg, 174 mg, 180 mg, 200 mg and 204 mg; or
   c) an initial daily dose is withheld for at least 7 days and resumed at a reduced daily dose selected from 12 mg, 24 mg, 43.5 mg, 45 mg, 48 mg, 66 mg, 87 mg, 90 mg, 126 mg, 130.5 mg, 135 mg, 174 mg, and 180 mg on first instance and to 12 mg, 24 mg, 43.5 mg, 45 mg, 48 mg, 66 mg, 87 mg, 90 mg, 126 mg, 130.5 mg, and 135 mg on subsequent instances of recurrences of said toxicity;
and wherein, the reduced dose is optionally re-escalated to any one of the dose selected from 24 mg, 43.5 mg, 45 mg, 48 mg, 66 mg, 87 mg, 90 mg, 126 mg, 130.5 mg, 135 mg, 174 mg, 180 mg, 200 mg, and 204 mg to achieve or maintain at least one of (a) complete hematological response, (b) complete hematological response and partial cytogenetic response, (c) complete hematological response and complete cytogenetic response, or (d) complete hematological response, complete cytogenetic response, and major molecular response.

In another embodiment, the daily dose is selected from 12 mg, 24 mg, 43.5 mg, 45 mg, 48 mg, 66 mg, 87 mg, 90 mg, 126 mg, 130.5 mg, 135 mg, 174 mg, 180 mg, 200 mg and 204 mg.

In another particular embodiment, the present invention provides a method for the treatment of a patient with chronic myeloid leukemia (CML), wherein the method comprises orally administering to said patient a therapeutically effective amount of compound of Formula I:

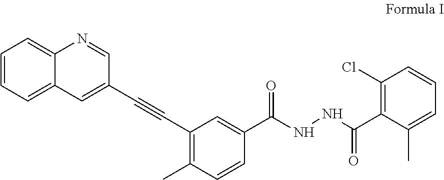 [SMILES: Cc1ccc(C(=O)NNC(=O)c2c(C)cccc2Cl)cc1C#Cc1cnc2ccccc2c1]

or a pharmaceutically acceptable salt thereof to achieve mean AUC₀₋₂₄ ranging from 6226±5827 ng*h/mL to 60373±55659 ng*h/mL.

In another particular embodiment, the present invention provides a method for the treatment of a patient with chronic myeloid leukemia (CML), wherein the method comprises orally administering to said patient a therapeutically effective amount of compound of Formula I:

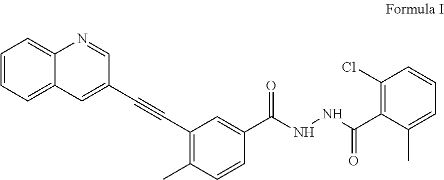 [SMILES: Cc1ccc(C(=O)NNC(=O)c2c(C)cccc2Cl)cc1C#Cc1cnc2ccccc2c1]

or a pharmaceutically acceptable salt thereof to achieve mean Cₘₐₓ ranging from 664±450 ng/ml to 5054±3051 ng/mL.

In another particular embodiment, the present invention provides a method for the treatment of a patient with chronic myeloid leukemia (CML), wherein the method comprises orally administering to said patient a therapeutically effective amount of compound of Formula I or a pharmaceutically acceptable salt thereof in daily dose selected from 12 mg, 24 mg, 43.5 mg, 45 mg, 48 mg, 66 mg, 87 mg, 90 mg, 126 mg, 130.5 mg, 135 mg, 174 mg, 180 mg, 200 mg and 204 mg:

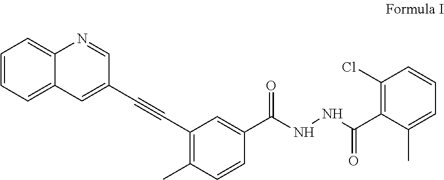 [SMILES: Cc1ccc(C(=O)NNC(=O)c2c(C)cccc2Cl)cc1C#Cc1cnc2ccccc2c1]

to achieve mean AUC₀₋₂₄ ranging from 6226±5827 ng*h/mL to 60373±55659 ng*h/mL.

In another particular embodiment, the present invention provides a method for the treatment of a patient with chronic myeloid leukemia (CML), wherein the method comprises orally administering to said patient a therapeutically effective amount of compound of Formula I or a pharmaceutically acceptable salt thereof in daily dose selected from 12 mg, 24 mg, 43.5 mg, 45 mg, 48 mg, 66 mg, 87 mg, 90 mg, 126 mg, 130.5 mg, 135 mg, 174 mg, 180 mg, 200 mg and 204 mg:

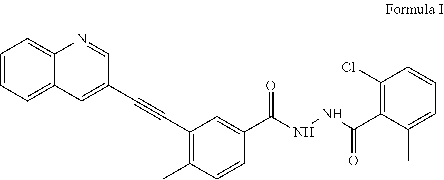 [SMILES: Cc1ccc(C(=O)NNC(=O)c2c(C)cccc2Cl)cc1C#Cc1cnc2ccccc2c1]

to achieve mean Cₘₐₓ ranging from 664±450 ng/ml to 5054±3051 ng/mL.

In another particular embodiment, the present invention provides a method for the treatment of a patient with chronic myeloid leukemia (CML), wherein the method comprises orally administering to said patient a therapeutically effective amount of compound of Formula I or a pharmaceutically acceptable salt thereof:

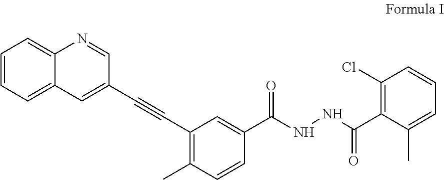 [SMILES: Cc1ccc(C(=O)NNC(=O)c2c(C)cccc2Cl)cc1C#Cc1cnc2ccccc2c1]

to achieve mean AUC₀₋₂₄ ranging from 6226±5827 ng*h/mL to 60373±55659 ng*h/mL and mean Cₘₐₓ ranging from 664±450 ng/ml to 5054±3051 ng/mL.

In another particular embodiment, the present invention provides a method for the treatment of a patient with chronic myeloid leukemia (CML), wherein the method comprises orally administering to said patient a therapeutically effective amount of compound of Formula I or a pharmaceutically acceptable salt thereof in daily dose selected from 12 mg, 24 mg, 43.5 mg, 45 mg, 48 mg, 66 mg, 87 mg, 90 mg, 126 mg, 130.5 mg, 135 mg, 174 mg, 180 mg, 200 mg and 204 mg:

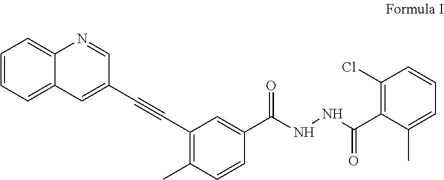 [SMILES: Cc1ccc(C(=O)NNC(=O)c2c(C)cccc2Cl)cc1C#Cc1cnc2ccccc2c1]

to achieve mean AUC₀₋₂₄ ranging from 6226±5827 ng*h/mL to 60373±55659 ng*h/mL and mean Cₘₐₓ ranging from 664±450 ng/ml to 5054±3051 ng/mL.

In another particular embodiment, the present invention provides a method for the treatment of a patient with chronic myeloid leukemia (CML), wherein the method comprises orally administering to said patient a therapeutically effective amount of compound of Formula I or a pharmaceutically acceptable salt thereof to achieve mean AUC₀₋₂₄ ranging from 6226±5827 ng*h/mL to 60373±55659 ng*h/mL and mean Cₘₐₓ ranging from 664±450 ng/ml to 5054±3051 ng/mL, at an initial daily dose selected from 12 mg, 24 mg, 43.5 mg, 45 mg, 48 mg, 66 mg, 87 mg, 90 mg, 126 mg, 130.5 mg, 135 mg, 174 mg, 180 mg, 200 mg and 204 mg:

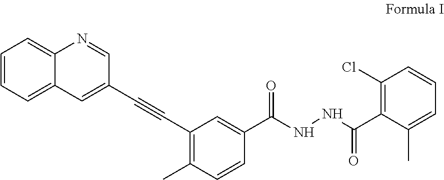 [SMILES: Cc1ccc(C(=O)NNC(=O)c2c(C)cccc2Cl)cc1C#Cc1cnc2ccccc2c1]

In another particular embodiment, the present invention provides a method for the treatment of a patient with chronic myeloid leukemia (CML), wherein the method comprises orally administering to said patient a therapeutically effective amount of compound of Formula I or a pharmaceutically acceptable salt thereof to achieve mean AUC₀₋₂₄ ranging from 6226±5827 ng*h/mL to 60373±55659 ng*h/mL and mean Cₘₐₓ ranging from 664±450 ng/ml to 5054±3051 ng/mL, at an initial daily dose selected from 12 mg, 24 mg, 43.5 mg, 45 mg, 48 mg, 66 mg, 87 mg, 90 mg, 126 mg, 130.5 mg, 135 mg, 174 mg, 180 mg, 200 mg and 204 mg:

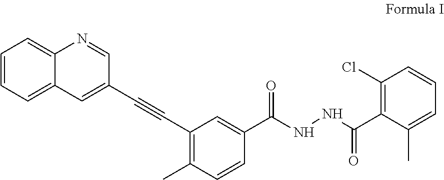 [SMILES: Cc1ccc(C(=O)NNC(=O)c2c(C)cccc2Cl)cc1C#Cc1cnc2ccccc2c1]

such that when the patient develops toxicity:
   a) the initial daily dose is withheld for at least 7 days and resumed at daily dose selected from 12 mg, 24 mg, 43.5 mg, 45 mg, 48 mg, 66 mg, 87 mg, 90 mg, 126 mg, 130.5 mg, 135 mg, 174 mg, 180 mg, 200 mg and 204 mg; or
   b) the initial daily dose is withheld for less than 7 days and if toxicity is resolved then resumed at daily dose selected from 12 mg, 24 mg, 43.5 mg, 45 mg, 48 mg, 66 mg, 87 mg, 90 mg, 126 mg, 130.5 mg, 135 mg, 174 mg, 180 mg, 200 mg and 204 mg; or
   c) an initial daily dose is withheld for at least 7 days and resumed at a reduced daily dose selected from 12 mg, 24 mg, 43.5 mg, 45 mg, 48 mg, 66 mg, 87 mg, 90 mg, 126 mg, 130.5 mg, 135 mg, 174 mg, and 180 mg on first instance and to 12 mg, 24 mg, 43.5 mg, 45 mg, 48 mg, 66 mg, 87 mg, 90 mg, 126 mg, 130.5 mg, and 135 mg on subsequent instances of recurrences of said toxicity;
and wherein, the reduced dose is optionally re-escalated to any one of the dose selected from 24 mg, 43.5 mg, 45 mg, 48 mg, 66 mg, 87 mg, 90 mg, 126 mg, 130.5 mg, 135 mg, 174 mg, 180 mg, 200 mg, and 204 mg.

In another particular embodiment, the present invention provides a method for the treatment of a patient with chronic myeloid leukemia (CML), wherein the method comprises orally administering to said patient a therapeutically effective amount of compound of Formula I or a pharmaceutically acceptable salt thereof to achieve mean AUC₀₋₂₄ ranging from 6226±5827 ng*h/mL to 60373±55659 ng*h/mL and mean Cₘₐₓ ranging from 664±450 ng/ml to 5054±3051 ng/mL, at an initial daily dose selected from 12 mg, 24 mg, 43.5 mg, 45 mg, 48 mg, 66 mg, 87 mg, 90 mg, 126 mg, 130.5 mg, 135 mg, 174 mg, 180 mg, 200 mg and 204 mg:

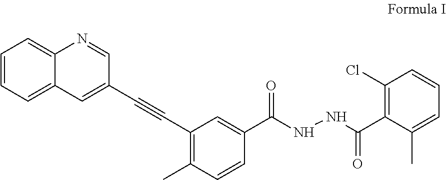 [SMILES: Cc1ccc(C(=O)NNC(=O)c2c(C)cccc2Cl)cc1C#Cc1cnc2ccccc2c1]

such that when the patient develops toxicity:
   a) the initial daily dose is withheld for at least 7 days and resumed at daily dose selected from 12 mg, 24 mg, 43.5 mg, 45 mg, 48 mg, 66 mg, 87 mg, 90 mg, 126 mg, 130.5 mg, 135 mg, 174 mg, 180 mg, 200 mg and 204 mg; or
   b) the initial daily dose is withheld for less than 7 days and if toxicity is resolved then resumed at daily dose selected from 12 mg, 24 mg, 43.5 mg, 45 mg, 48 mg, 66 mg, 87 mg, 90 mg, 126 mg, 130.5 mg, 135 mg, 174 mg, 180 mg, 200 mg and 204 mg; or
   c) an initial daily dose is withheld for at least 7 days and resumed at a reduced daily dose selected from 12 mg, 24 mg, 43.5 mg, 45 mg, 48 mg, 66 mg, 87 mg, 90 mg, 126 mg, 130.5 mg, 135 mg, 174 mg, and 180 mg on first instance and to 12 mg, 24 mg, 43.5 mg, 45 mg, 48 mg, 66 mg, 87 mg, 90 mg, 126 mg, 130.5 mg, and 135 mg on subsequent instances of recurrences of said toxicity;
and wherein, the reduced dose is optionally re-escalated to any one of the dose selected from 24 mg, 43.5 mg, 45 mg, 48 mg, 66 mg, 87 mg, 90 mg, 126 mg, 130.5 mg, 135 mg, 174 mg, 180 mg, 200 mg, and 204 mg to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction.

In one particular embodiment, the present invention provides a method for the treatment of a patient with refractory chronic myeloid leukemia (CML), comprising orally administering to said patient a therapeutically effective amount of compound of Formula I or a pharmaceutically acceptable salt thereof at an initial daily dose of 174 mg:

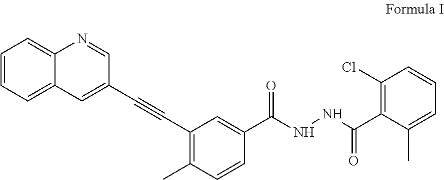 [SMILES: Cc1ccc(C(=O)NNC(=O)c2c(C)cccc2Cl)cc1C#Cc1cnc2ccccc2c1]

such that when the patient develops hematologic and/or non-hematologic toxicity:
   a) the initial daily dose is withheld for at least 7 days and resumed at 174 mg; or
   b) the initial daily dose is withheld for less than 7 days and if toxicity is resolved then resumed at 174 mg; or
   c) the initial daily dose is withheld for at least 7 days and resumed at a reduced daily dose selected from 43.5 mg, 87 mg, and 130.5 mg on subsequent instances of recurrences of said toxicity;
and wherein, the reduced dose is optionally re-escalated to any one of the dose selected from 87 mg, 130.5 mg, 174 mg, and 200 mg.

In another particular embodiment, the present invention provides a method for the treatment of a patient with refractory chronic myeloid leukemia (CML), comprising orally administering to said patient a therapeutically effective amount of compound of Formula I or a pharmaceutically acceptable salt thereof to achieve mean AUC₀₋₂₄ ranging from 28598 ng*h/mL to 42898±33827 ng*h/mL and mean Cₘₐₓ ranging from 3370 ng/ml to 5054±3051 ng/mL, at an initial daily dose of 174 mg or 180 mg:

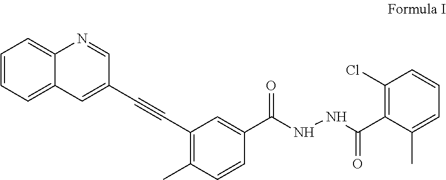 [SMILES: Cc1ccc(C(=O)NNC(=O)c2c(C)cccc2Cl)cc1C#Cc1cnc2ccccc2c1]

such that when the patient develops hematologic and/or non-hematologic toxicity:
   a) the initial daily dose is withheld for at least 7 days and resumed at 174 mg or 180 mg; or
   b) the initial daily dose is withheld for less than 7 days and if toxicity is resolved then resumed at 174 mg or 180 mg; or
   c) the initial daily dose is withheld for at least 7 days and resumed at a reduced daily dose selected from 43.5 mg, 45 mg, 48 mg, 66 mg, 87 mg, 90 mg, 126 mg, 130.5 mg, 135 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg.

According to any one of the embodiments described herein, when the patient develops toxicity the initial daily dose is withheld for at least 7 days to eight weeks and resumed at 174 mg.

According to any one of the embodiments described herein, when the patient develops toxicity the initial daily dose is withheld for at least 7 days to eight weeks and resumed at 180 mg.

According to any one of the embodiments described herein, when the patient develops toxicity the initial daily dose is withheld for at least 7 days and resumed at 174 mg or the initial daily dose is withheld for at least 7 days and resumed at a reduced daily dose selected from 43.5 mg, 87 mg and 130.5 mg.

According to any one of the embodiments described herein, when the patient develops toxicity the initial daily dose is withheld for at least 7 days and resumed at 180 mg or the initial daily dose is withheld for at least 7 days and resumed at a reduced daily dose selected from 45 mg, 90 mg and 135 mg.

According to any one of the embodiments described herein, when the patient develops toxicity the initial daily dose is withheld for at least 14 days and resumed at 174 mg or the initial daily dose is withheld for at least 14 days and resumed at a reduced daily dose selected from 43.5 mg, 87 mg and 130.5 mg.

According to any one of the embodiments described herein, when the patient develops toxicity the initial daily dose is withheld for at least 14 days and resumed at 180 mg or the initial daily dose is withheld for at least 14 days and resumed at a reduced daily dose selected from 45 mg, 90 mg and 135 mg.

According to any one of the embodiments described herein, when the patient develops toxicity the initial daily dose is withheld for at least four weeks and resumed at 174 mg or the initial daily dose is withheld for at least four weeks and resumed at a reduced daily dose selected from 43.5 mg, 87 mg and 130.5 mg.

According to any one of the embodiments described herein, when the patient develops toxicity the initial daily dose is withheld for at least four weeks and resumed at 180 mg or the initial daily dose is withheld for at least four weeks and resumed at a reduced daily selected from 45 mg, 90 mg and 135 mg.

According to any one of the embodiments described herein, when the patient develops toxicity the initial daily dose is withheld for at least eight weeks and resumed at 174 mg or the initial daily dose is withheld for at least eight weeks and resumed at a reduced daily dose selected from 43.5 mg, 87 mg and 130.5 mg.

According to any one of the embodiments described herein, when the patient develops toxicity the initial daily dose is withheld for at least eight weeks and resumed at 180 mg or the initial daily dose is withheld for at least eight weeks and resumed at a reduced daily dose selected from 45 mg, 90 mg and 135 mg.

According to any one of the embodiments described herein, the reduced daily dose is selected from 126 mg and 130.5 mg on first instance and to 90 mg, 66 mg, 48 mg and 43.5 mg on subsequent instances of recurrences of said toxicity.

According to any one of the embodiments described herein, the reduced daily dose is selected from 135 mg on the first instance and to 90 mg, and 45 mg on subsequent instances of recurrences of said toxicity.

According to any one of the embodiments described herein, the reduced dose is optionally re-escalated to any one of the dose selected from 66 mg, 87 mg, 90 mg, 126 mg, 130.5 mg, 135 mg, 174 mg, 180 mg, 200 mg, and 204 mg.

According to any one of the embodiments described herein, the reduced dose is optionally re-escalated to any one of the dose selected from 87 mg, 130.5 mg, 174 mg, and 200 mg.

According to any one of the embodiments described herein, the reduced dose is optionally re-escalated to any one of the dose selected from 48 mg, 66 mg, 90 mg, 126 mg and 174 mg.

According to any one of the embodiments described herein, the reduced dose is optionally re-escalated to any one of the dose selected from 66 mg, 90 mg, 126 mg and 174 mg.

According to any one of the embodiments described herein, the compound of Formula I or a pharmaceutically acceptable salt thereof is administered to achieve mean AUC₀₋₂₄ ranging from 28598 ng*h/mL to 42898±33827 ng*h/mL.

According to any one of the embodiments described herein, the compound of Formula I or a pharmaceutically acceptable salt thereof is administered to achieve mean Cₘₐₓ ranging from 3370 ng/mL to 5054±3051 ng/ml.

In one embodiment, the present invention provides, a method for the treatment of a patient with refractory chronic myeloid leukemia (CML), comprising orally administering to said patient a therapeutically effective amount of compound of Formula I or a pharmaceutically acceptable salt thereof to achieve mean AUC₀₋₂₄ ranging from 28598 ng*h/mL to 42898±33827 ng*h/mL and mean Cₘₐₓ ranging from 3370 ng/mL to 5054±3051 ng/ml, at an initial daily dose of 174 mg or 180 mg:

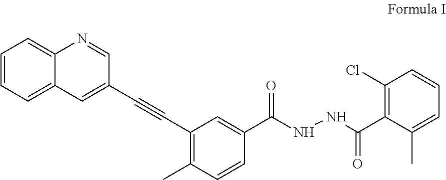 [SMILES: Cc1ccc(C(=O)NNC(=O)c2c(C)cccc2Cl)cc1C#Cc1cnc2ccccc2c1]

such that when the patient develops hematologic and/or non-hematologic toxicity:
   a) the initial daily dose is withheld for at least 7 days and resumed at 174 mg or 180 mg; or
   b) the initial daily dose is withheld for less than 7 days and if toxicity is resolved then resumed at 174 mg or 180 mg; or
   c) the initial daily dose is withheld for at least 7 days and resumed at a reduced daily dose selected from 43.5 mg, 45 mg, 48 mg, 66 mg, 87 mg, 90 mg, 126 mg, 130.5 mg, 135 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction and said daily dose;
   a) results in not less than 50 ng/ml of the compound of Formula I or a pharmaceutically acceptable salt thereof in the plasma; or
   b) achieves 15% higher AUC₀₋₂₄ when administered under fasted conditions as compared to when administered under fed conditions; or
   c) achieves 20% higher Cₘₐₓ when administered under fasted conditions as compared to when administered under fed conditions; or
   d) results in patient's QT interval of less than about 500 ms.

In one particular embodiment, the present invention provides a method for the treatment of a patient with refractory chronic myeloid leukemia (CML), comprising orally administering to said patient a therapeutically effective amount of compound of Formula I or a pharmaceutically acceptable salt thereof at an initial daily dose of 135 mg:

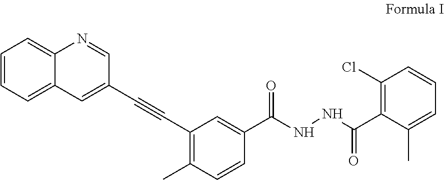 [SMILES: Cc1ccc(C(=O)NNC(=O)c2c(C)cccc2Cl)cc1C#Cc1cnc2ccccc2c1]

such that when the patient develops hematologic and/or non-hematologic toxicity:
   a) the initial daily dose is withheld for at least 7 days and resumed at 135 mg; or
   b) the initial daily dose is withheld for less than 7 days and if toxicity is resolved then resumed at 135 mg; or
   c) the initial daily dose is withheld for at least 7 days and resumed at a reduced daily dose selected from 90 mg and 45 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg.

In one embodiment, when the patient develops toxicity the initial daily dose is withheld for at least 7 days to eight weeks and resumed at 135 mg.

According to any one of the embodiments described herein, when the patient develops toxicity the initial daily dose is withheld for at least 7 days and resumed at 135 mg or the initial daily dose is withheld for at least 7 days and resumed at a reduced daily dose selected from 45 mg and 90 mg.

According to any one of the embodiments described herein, when the patient develops toxicity the initial daily dose is withheld for at least 14 days and resumed at 135 mg or the initial daily dose is withheld for at least 14 days and resumed at a reduced daily dose selected from 45 mg and 90 mg.

According to any one of the embodiments described herein, when the patient develops toxicity the initial daily dose is withheld for at least four weeks and resumed at 135 mg or the initial daily dose is withheld for at least four weeks and resumed at a reduced daily selected from 45 mg and 90 mg.

According to any one of the embodiments described herein, when the patient develops toxicity the initial daily dose is withheld for at least eight weeks and resumed at 135 mg or the initial daily dose is withheld for at least eight weeks and resumed at a reduced daily dose selected from 45 mg and 90 mg.

According to any one of the embodiments described herein, the reduced dose is optionally re-escalated to any one of the dose selected from 66 mg, 87 mg, 90 mg, 126 mg, 130.5 mg, 135 mg, 174 mg, 180 mg, 200 mg, and 204 mg.

According to any one of the embodiments described herein, the reduced dose is optionally re-escalated to any one of the dose selected from 90 mg to 180 mg.

According to any one of the embodiments described herein, the reduced dose is optionally re-escalated to 90 mg.

According to any one of the embodiments described herein, the reduced dose is optionally re-escalated to 135 mg.

According to any one of the embodiments described herein, the reduced dose is optionally re-escalated to 180 mg.

According to any one of the embodiments described herein, the reduced dose is optionally re-escalated to 200 mg.

In one particular embodiment, the present invention provides a method for the treatment of a patient with refractory chronic myeloid leukemia (CML), comprising orally administering to said patient a therapeutically effective amount of compound of Formula I or a pharmaceutically acceptable salt thereof at an initial daily dose of 135 mg:

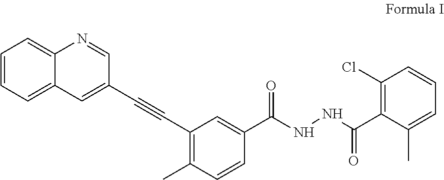 [SMILES: Cc1ccc(C(=O)NNC(=O)c2c(C)cccc2Cl)cc1C#Cc1cnc2ccccc2c1]

such that when the patient develops hematologic and/or non-hematologic toxicity:
   a) the initial daily dose is withheld for at least 7 days and resumed at 135 mg; or
   b) the initial daily dose is withheld for less than 7 days and if toxicity is resolved then resumed at 135 mg; or
   c) the initial daily dose is withheld for at least 7 days and resumed at a reduced daily dose selected from 90 mg and 45 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction and said daily dose;
   a) results in not less than 50 ng/mL of the compound of Formula I or a pharmaceutically acceptable salt thereof in the plasma; or
   b) achieves 15% higher AUC₀₋₂₄ when administered under fasted conditions as compared to when administered under fed conditions; or
   c) achieves 20% higher Cₘₐₓ when administered under fasted conditions as compared to when administered under fed conditions; or
   d) results in patient's QT interval of less than about 500 ms.

In one particular embodiment, the present invention provides, a method for the treatment of a patient with refractory chronic myeloid leukemia (CML), comprising orally administering to said patient a therapeutically effective amount of compound of Formula I or a pharmaceutically acceptable salt thereof at an initial daily dose of 130.5 mg:

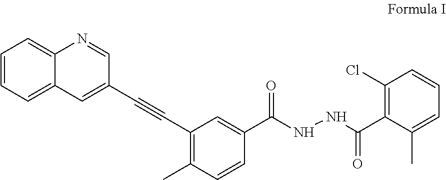 [SMILES: Cc1ccc(C(=O)NNC(=O)c2c(C)cccc2Cl)cc1C#Cc1cnc2ccccc2c1]

such that when the patient develops hematologic and/or non-hematologic toxicity:
   a) the initial daily dose is withheld for at least 7 days and resumed at 130.5 mg; or
   b) the initial daily dose is withheld for less than 7 days and if toxicity is resolved then resumed at 130.5 mg; or
   c) the initial daily dose is withheld for at least 7 days and resumed at a reduced daily dose selected from 43.5 mg and 87 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg.

In one embodiment, wherein when the patient develops toxicity the initial daily dose is withheld for at least 7 days to eight weeks and resumed at 130.5 mg.

According to any one of the embodiments described herein, when the patient develops toxicity the initial daily dose is withheld for at least 7 days and resumed at 130.5 mg or the initial daily dose is withheld for at least 7 days and resumed at a reduced daily dose selected from 43.5 mg and 87 mg.

According to any one of the embodiments described herein, when the patient develops toxicity the initial daily dose is withheld for at least 14 days and resumed at 130.5 mg or the initial daily dose is withheld for at least 14 days and resumed at a reduced daily dose selected from 43.5 mg and 87 mg.

According to any one of the embodiments described herein, when the patient develops toxicity the initial daily dose is withheld for at least four weeks and resumed at 130.5 mg or the initial daily dose is withheld for at least four weeks and resumed at a reduced daily dose selected from 43.5 mg and 87 mg.

According to any one of the embodiments described herein, when the patient develops toxicity the initial daily dose is withheld for at least eight weeks and resumed at 130.5 mg or the initial daily dose is withheld for at least eight weeks and resumed at a reduced daily dose selected from 43.5 mg and 87 mg.

According to any one of the embodiments described herein, the reduced daily dose is 43.5 mg.

According to any one of the embodiments described herein, the reduced daily dose is 87 mg.

According to any one of the embodiments described herein, the reduced dose is optionally re-escalated to any one of the dose selected from 66 mg, 87 mg, 90 mg, 126 mg, 130.5 mg, 135 mg, 174 mg, 180 mg, 200 mg, and 204 mg.

According to any one of the embodiments described herein, the reduced dose is optionally re-escalated to dose of 87 mg.

According to any one of the embodiments described herein, the reduced dose is optionally re-escalated to dose of 130.5 mg.

According to any one of the embodiments described herein, the reduced dose is optionally re-escalated to dose of 174 mg.

According to any one of the embodiments described herein, the reduced dose is optionally re-escalated to dose of 200 mg.

In one particular embodiment, the present invention provides, a method for the treatment of a patient with refractory chronic myeloid leukemia (CML), comprising orally administering to said patient a therapeutically effective amount of compound of Formula I or a pharmaceutically acceptable salt thereof at an initial daily dose of 130.5 mg:

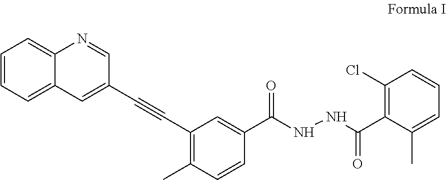 [SMILES: Cc1ccc(C(=O)NNC(=O)c2c(C)cccc2Cl)cc1C#Cc1cnc2ccccc2c1]

such that when the patient develops hematologic and/or non-hematologic toxicity:
   a) the initial daily dose is withheld for at least 7 days and resumed at 130.5 mg; or
   b) the initial daily dose is withheld for less than 7 days and if toxicity is resolved then resumed at 130.5 mg; or
   c) the initial daily dose is withheld for at least 7 days and resumed at a reduced daily dose selected from 43.5 mg and 87 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction and said daily dose;
   a) results in not less than 50 ng/ml of the compound of Formula I or a pharmaceutically acceptable salt thereof in the plasma; or
   b) achieves 15% higher AUC₀₋₂₄ when administered under fasted conditions as compared to when administered under fed conditions; or
   c) achieves 20% higher Cₘₐₓ when administered under fasted conditions as compared to when administered under fed conditions; or
d) results in patient's QT interval of less than about 500 ms.

In one particular embodiment, the present invention provides, a method for the treatment of a patient with refractory chronic myeloid leukemia (CML), comprising orally administering to said patient a therapeutically effective amount of compound of Formula I or a pharmaceutically acceptable salt thereof to achieve mean AUC₀₋₂₄ ranging from 40249 ng*h/mL to 60373±55659 ng*h/mL and mean Cₘₐₓ ranging from 3282 ng/ml to 4924±3289 ng/mL, at an initial daily dose of 126 mg:

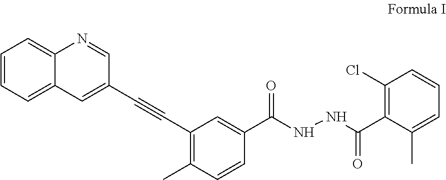 [SMILES: Cc1ccc(C(=O)NNC(=O)c2c(C)cccc2Cl)cc1C#Cc1cnc2ccccc2c1]

such that when the patient develops hematologic and/or non-hematologic toxicity:
   a) the initial daily dose is withheld for at least 7 days and resumed at 126 mg; or
   b) the initial daily dose is withheld for less than 7 days and if toxicity is resolved then resumed at 126 mg; or
   c) the initial daily dose is withheld for at least 7 days and resumed at a reduced daily dose selected from 48 mg, 66 mg and 90 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg.

According to any one of the embodiments described herein, when the patient develops toxicity the initial daily dose is withheld for at least 7 days to eight weeks and resumed at 126 mg.

According to any one of the embodiments described herein, when the patient develops toxicity the initial daily dose is withheld for at least 7 days and resumed at 126 mg or the initial daily dose is withheld for at least 7 days and resumed at a reduced daily dose selected from 48 mg, 66 mg and 90 mg.

According to any one of the embodiments described herein, when the patient develops toxicity the initial daily dose is withheld for at least 14 days and resumed at 126 mg or the initial daily dose is withheld for at least 14 days and resumed at a reduced daily dose selected from 48 mg, 66 mg and 90 mg.

According to any one of the embodiments described herein, when the patient develops toxicity the initial daily dose is withheld for at least four weeks and resumed at 126 mg or the initial daily dose is withheld for at least four weeks and resumed at a reduced daily dose selected from 48 mg, 66 mg and 90 mg.

According to any one of the embodiments described herein, when the patient develops toxicity the initial daily dose is withheld for at least eight weeks and resumed at 126 mg or the initial daily dose is withheld for at least eight weeks and resumed at a reduced daily dose selected from 48 mg, 66 mg and 90 mg.

According to any one of the embodiments described herein, the reduced daily dose is 48 mg.

According to any one of the embodiments described herein, the reduced daily dose is 66 mg.

According to any one of the embodiments described herein, the reduced daily dose is 90 mg.

According to any one of the embodiments described herein, the reduced dose is optionally re-escalated to any one of the dose selected from 66 mg, 87 mg, 90 mg, 126 mg, 130.5 mg, 135 mg, 174 mg, 180 mg, 200 mg, and 204 mg.

According to any one of the embodiments described herein, the reduced dose is optionally re-escalated to dose of 66 mg.

According to any one of the embodiments described herein, the reduced dose is optionally re-escalated to dose of 90 mg.

According to any one of the embodiments described herein, the reduced dose is optionally re-escalated to dose of 174 mg.

According to any one of the embodiments described herein, the reduced dose is optionally re-escalated to dose of 204 mg.

According to any one of the embodiments described herein, the compound of Formula I or a pharmaceutically acceptable salt thereof is administered to achieve mean AUC₀₋₂₄ ranging from 40249 to 60373±55659 ng*h/mL.

According to any one of the embodiments described herein, the compound of Formula I or a pharmaceutically acceptable salt thereof is administered to achieve mean Cₘₐₓ ranging from 3282 to 4924±3289 ng/mL.

In one particular embodiment, the present invention provides a method for the treatment of a patient with refractory chronic myeloid leukemia (CML), comprising orally administering to said patient a therapeutically effective amount of compound of Formula I or a pharmaceutically acceptable salt thereof to achieve mean AUC₀₋₂₄ ranging from 40249 ng*h/mL to 60373±55659 ng*h/mL and mean Cₘₐₓ ranging from 3282 ng/ml to 4924±3289 ng/mL, at an initial daily dose of 126 mg:

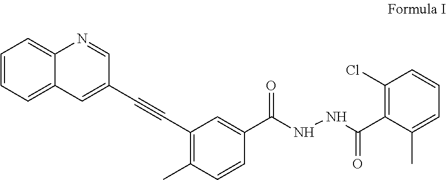 [SMILES: Cc1ccc(C(=O)NNC(=O)c2c(C)cccc2Cl)cc1C#Cc1cnc2ccccc2c1]

such that when the patient develops hematologic and/or non-hematologic toxicity:
   a) the initial daily dose is withheld for at least 7 days and resumed at 126 mg; or
   b) the initial daily dose is withheld for less than 7 days and if toxicity is resolved then resumed at 126 mg; or
   c) the initial daily dose is withheld for at least 7 days and resumed at a reduced daily dose selected from 48 mg, 66 mg and 90 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction and said daily dose;
   a) results in not less than 50 ng/ml of the compound of Formula I or a pharmaceutically acceptable salt thereof in the plasma; or
   b) achieves 15% higher AUC₀₋₂₄ when administered under fasted conditions as compared to when administered under fed conditions; or
   c) achieves 20% higher Cₘₐₓ when administered under fasted conditions as compared to when administered under fed conditions; or
   d) results in patient's QT interval of less than about 500 ms.

In one particular embodiment, the present invention provides a method for the treatment of a patient with refractory chronic myeloid leukemia (CML), comprising orally administering to said patient a therapeutically effective amount of compound of Formula I or a pharmaceutically acceptable salt thereof to achieve mean AUC₀₋₂₄ ranging from 18237 ng*h/mL to 27355±2782 ng*h/mL and mean Cₘₐₓ ranging from 2277 ng/mL to 3415±1566 ng/ml, at an initial daily dose of 90 mg:

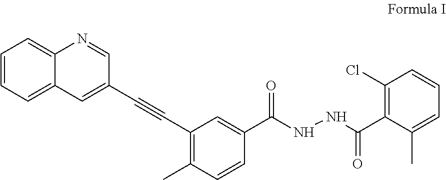 [SMILES: Cc1ccc(C(=O)NNC(=O)c2c(C)cccc2Cl)cc1C#Cc1cnc2ccccc2c1]

such that when the patient develops hematologic and/or non-hematologic toxicity:
   a) the initial daily dose is withheld for at least 7 days and resumed at 90 mg; or
   b) the initial daily dose is withheld for less than 7 days and if toxicity is resolved then resumed at 90 mg; or
   c) the initial daily dose is withheld for at least 7 days and resumed at a reduced daily dose selected from 45 mg, 48 mg and 66 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg.

According to any one of the embodiments described herein, when the patient develops toxicity the initial daily dose is withheld for at least 7 days to eight weeks and resumed at 90 mg.

According to any one of the embodiments described herein, when the patient develops toxicity the initial daily dose is withheld for at least 7 days and resumed at 90 mg or the initial daily dose is withheld for at least 7 days and resumed at a reduced daily dose selected from 45 mg, 48 mg and 66 mg.

According to any one of the embodiments described herein, when the patient develops toxicity the initial daily dose is withheld for at least 14 days and resumed at 90 mg or the initial daily dose is withheld for at least 14 days and resumed at a reduced daily dose selected from 45 mg, 48 mg and 66 mg.

According to any one of the embodiments described herein, when the patient develops toxicity the initial daily dose is withheld for at least four weeks and resumed at 90 mg or the initial daily dose is withheld for at least four weeks and resumed at a reduced daily dose selected from 45 mg, 48 mg and 66 mg.

According to any one of the embodiments described herein, when the patient develops toxicity the initial daily dose is withheld for at least eight weeks and resumed at 90 mg or the initial daily dose is withheld for at least eight weeks and resumed at a reduced daily dose selected from 45 mg, 48 mg and 66 mg.

According to any one of the embodiments described herein, when the patient develops toxicity the initial daily dose is withheld for at least 7 days and resumed at 90 mg or the initial daily dose is withheld for at least 7 days and resumed at a reduced daily dose selected from 45 mg, 48 mg and 66 mg.

According to any one of the embodiments described herein, when the patient develops toxicity the initial daily dose is withheld for at least 14 days and resumed at 90 mg or the initial daily dose is withheld for at least 14 days and resumed at a reduced daily dose selected from 45 mg, 48 mg and 66 mg.

According to any one of the embodiments described herein, when the patient develops toxicity the initial daily dose is withheld for at least four weeks and resumed at 90 mg or the initial daily dose is withheld for at least four weeks and resumed at a reduced daily dose selected from 45 mg, 48 mg and 66 mg.

According to any one of the embodiments described herein, when the patient develops toxicity the initial daily dose is withheld for at least eight weeks and resumed at 90 mg or the initial daily dose is withheld for at least eight weeks and resumed at a reduced daily dose selected from 45 mg, 48 mg and 66 mg. In another embodiment, the reduced daily dose is 45 mg.

According to any one of the embodiments described herein, the reduced daily dose is 48 mg.

According to any one of the embodiments described herein, the reduced daily dose is 66 mg.

According to any one of the embodiments described herein, the reduced dose is optionally re-escalated to any one of the dose selected from 66 mg, 87 mg, 90 mg, 126 mg, 130.5 mg, 135 mg, 174 mg, 180 mg, 200 mg, and 204 mg.

According to any one of the embodiments described herein, the reduced dose is optionally re-escalated to any one of the dose selected from 90 mg to 204 mg.

According to any one of the embodiments described herein, the reduced dose is optionally re-escalated to 66 mg.

According to any one of the embodiments described herein, the reduced dose is optionally re-escalated to 90 mg.

According to any one of the embodiments described herein, the reduced dose is optionally re-escalated to 126 mg.

According to any one of the embodiments described herein, the reduced dose is optionally re-escalated to 130.5 mg.

According to any one of the embodiments described herein, the reduced dose is optionally re-escalated to 174 mg.

According to any one of the embodiments described herein, the reduced dose is optionally re-escalated to 180 mg.

According to any one of the embodiments described herein, the reduced dose is optionally re-escalated to 204 mg.

According to any one of the embodiments described herein, the compound of Formula I or a pharmaceutically acceptable salt thereof is administered to achieve mean AUC₀₋₂₄ ranging from 18237 ng*h/mL to 27355±2782 ng*h/mL.

According to any one of the embodiments described herein, the compound of Formula I or a pharmaceutically acceptable salt thereof is administered to achieve mean Cₘₐₓ ranging from 2277 ng/ml to 3415±1566 ng/mL.

In one embodiment, the present invention provides a method for the treatment of a patient with refractory chronic myeloid leukemia (CML), comprising orally administering to said patient a therapeutically effective amount of compound of Formula I or a pharmaceutically acceptable salt thereof to achieve mean AUC₀₋₂₄ ranging from 18237 ng*h/mL to 27355±2782 ng*h/mL and mean Cₘₐₓ ranging from 2277 ng/mL to 3415±1566 ng/ml, at an initial daily dose of 90 mg:

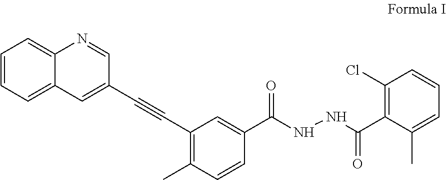 [SMILES: Cc1ccc(C(=O)NNC(=O)c2c(C)cccc2Cl)cc1C#Cc1cnc2ccccc2c1]

such that when the patient develops hematologic and/or non-hematologic toxicity:
   a) the initial daily dose is withheld for at least 7 days and resumed at 90 mg; or
   b) the initial daily dose is withheld for less than 7 days and if toxicity is resolved then resumed at 90 mg; or
   c) the initial daily dose is withheld for at least 7 days and resumed at a reduced daily dose selected from 45 mg, 48 mg and 66 mg on subsequent instances of recurrences of said toxicity;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction and said daily dose;
   a) results in not less than 50 ng/ml of the compound of Formula I or a pharmaceutically acceptable salt thereof in the plasma; or
   b) achieves 15% higher AUC₀₋₂₄ when administered under fasted conditions as compared to when administered under fed conditions; or
   c) achieves 20% higher Cₘₐₓ when administered under fasted conditions as compared to when administered under fed conditions; or
   d) results in patient's QT interval of less than about 500 ms.

In one particular embodiment, the present invention provides a method for the treatment of a patient with refractory chronic myeloid leukemia (CML), comprising orally administering to said patient a therapeutically effective amount of compound of Formula I or a pharmaceutically acceptable salt thereof at an initial daily dose of 87 mg:

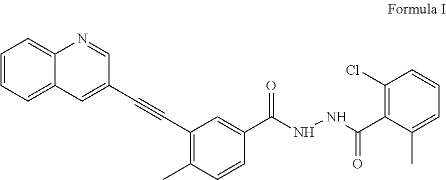 [SMILES: Cc1ccc(C(=O)NNC(=O)c2c(C)cccc2Cl)cc1C#Cc1cnc2ccccc2c1]

such that when the patient develops hematologic and/or non-hematologic toxicity:
   a) the initial daily dose is withheld for at least 7 days and resumed at 87 mg; or
   b) the initial daily dose is withheld for less than 7 days and if toxicity is resolved then resumed at 87 mg; or
   c) the initial daily dose is withheld for at least 7 days and resumed at a reduced daily dose of 43.5 mg;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg.

According to any one of the embodiments described herein, when the patient develops toxicity the initial daily dose is withheld for at least 7 days to eight weeks and resumed at 87 mg.

According to any one of the embodiments described herein, when the patient develops toxicity the initial daily dose is withheld for at least 7 days and resumed at 87 mg or the initial daily dose is withheld for at least 7 days and resumed at a reduced daily dose of 43.5 mg.

According to any one of the embodiments described herein, when the patient develops toxicity the initial daily dose is withheld for at least 14 days and resumed at 87 mg or the initial daily dose is withheld for at least 14 days and resumed at a reduced daily dose of 43.5 mg.

According to any one of the embodiments described herein, when the patient develops toxicity the initial daily dose is withheld for at least four weeks and resumed at 87 mg or the initial daily dose is withheld for at least four weeks and resumed at a reduced daily dose 43.5 mg.

According to any one of the embodiments described herein, when the patient develops toxicity the initial daily dose is withheld for at least eight weeks and resumed at 87 mg or the initial daily dose is withheld for at least eight weeks and resumed at a reduced daily dose of 43.5 mg.

According to any one of the embodiments described herein, the reduced dose is optionally re-escalated to any one of the dose selected from 66 mg, 87 mg, 90 mg, 126 mg, 130.5 mg, 135 mg, 174 mg, 180 mg, 200 mg, and 204 mg.

According to any one of the embodiments described herein, the reduced dose is optionally re-escalated to 87 mg.

According to any one of the embodiments described herein, the reduced dose is optionally re-escalated to 130.5 mg.

According to any one of the embodiments described herein, the reduced dose is optionally re-escalated to 174 mg.

According to any one of the embodiments described herein, the reduced dose is optionally re-escalated to 200 mg.

In one particular embodiment, the present invention provides a method for the treatment of a patient with refractory chronic myeloid leukemia (CML), comprising orally administering to said patient a therapeutically effective amount of compound of Formula I or a pharmaceutically acceptable salt thereof at an initial daily dose of 87 mg:

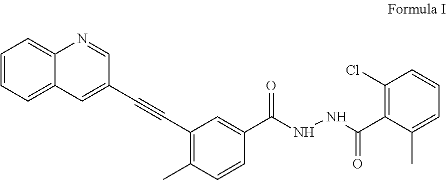 [SMILES: Cc1ccc(C(=O)NNC(=O)c2c(C)cccc2Cl)cc1C#Cc1cnc2ccccc2c1]

such that when the patient develops hematologic and/or non-hematologic toxicity:
   a) the initial daily dose is withheld for at least 7 days and resumed at 87 mg; or
   b) the initial daily dose is withheld for less than 7 days and if toxicity is resolved then resumed at 87 mg; or
   c) the initial daily dose is withheld for at least 7 days and resumed at a reduced daily dose of 43.5 mg;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction and said daily dose;
   a) results in not less than 50 ng/ml of the compound of Formula I or a pharmaceutically acceptable salt thereof in the plasma; or
   b) achieves 15% higher AUC₀₋₂₄ when administered under fasted conditions as compared to when administered under fed conditions; or
   c) achieves 20% higher Cₘₐₓ when administered under fasted conditions as compared to when administered under fed conditions; or
   d) results in patient's QT interval of less than about 500 ms.

In one particular embodiment, the present invention provides a method for the treatment of a patient with refractory chronic myeloid leukemia (CML), comprising orally administering to said patient a therapeutically effective amount of compound of Formula I or a pharmaceutically acceptable salt thereof to achieve mean AUC₀₋₂₄ ranging from 6226 ng*h/mL to 9338±5827 ng*h/mL and mean Cₘₐₓ ranging from 664 ng/ml to 996±450 ng/mL, at an initial daily dose of 66 mg:

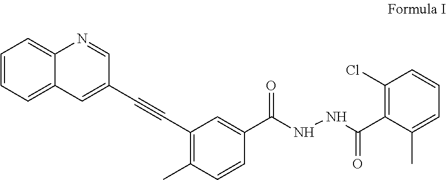 [SMILES: Cc1ccc(C(=O)NNC(=O)c2c(C)cccc2Cl)cc1C#Cc1cnc2ccccc2c1]

such that when the patient develops hematologic and/or non-hematologic toxicity:
   a) the initial daily dose is withheld for at least 7 days and resumed at 66 mg; or
   b) the initial daily dose is withheld for less than 7 days and if toxicity is resolved then resumed at 66 mg; or
   c) the initial daily dose is withheld for at least 7 days and resumed at a reduced daily dose of 48 mg;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg.

According to any one of the embodiments described herein, when the patient develops toxicity the initial daily dose is withheld for at least 7 days to eight weeks and resumed at 66 mg.

According to any one of the embodiments described herein, when the patient develops toxicity the initial daily dose is withheld for at least 7 days and resumed at 66 mg or the initial daily dose is withheld for at least 7 days and resumed at a reduced daily dose of 48 mg.

According to any one of the embodiments described herein, when the patient develops toxicity the initial daily dose is withheld for at least 14 days and resumed at 66 mg or the initial daily dose is withheld for at least 14 days and resumed at a reduced daily dose of 48 mg.

According to any one of the embodiments described herein, when the patient develops toxicity the initial daily dose is withheld for at least four weeks and resumed at 66 mg or the initial daily dose is withheld for at least four weeks and resumed at a reduced daily dose of 48 mg.

According to any one of the embodiments described herein, when the patient develops toxicity the initial daily dose is withheld for at least eight weeks and resumed at 66 mg or the initial daily dose is withheld for at least eight weeks and resumed at a reduced daily dose of 48 mg.

According to any one of the embodiments described herein, the reduced dose is optionally re-escalated to any one of the dose selected from 66 mg, 87 mg, 90 mg, 126 mg, 130.5 mg, 135 mg, 174 mg, 180 mg, 200 mg, and 204 mg.

According to any one of the embodiments described herein, the reduced dose is optionally re-escalated to 66 mg.

According to any one of the embodiments described herein, the reduced dose is optionally re-escalated to 90 mg.

According to any one of the embodiments described herein, the reduced dose is optionally re-escalated to 126 mg.

According to any one of the embodiments described herein, the reduced dose is optionally re-escalated to 174 mg.

According to any one of the embodiments described herein, the reduced dose is optionally re-escalated to 204 mg.

According to any one of the embodiments described herein, the compound of Formula I or a pharmaceutically acceptable salt thereof is administered to achieve mean AUC₀₋₂₄ ranging from 6226 ng*h/mL to 9338±5827 ng*h/mL.

According to any one of the embodiments described herein, the compound of Formula I or a pharmaceutically acceptable salt thereof is administered to achieve mean Cₘₐₓ ranging from 664 ng/ml to 996±450 ng/mL.

In one particular embodiment, the present invention provides a method for the treatment of a patient with refractory chronic myeloid leukemia (CML), comprising orally administering to said patient a therapeutically effective amount of compound of Formula I or a pharmaceutically acceptable salt thereof to achieve mean AUC₀₋₂₄ ranging from 6226 ng*h/mL to 9338±5827 ng*h/mL and mean Cₘₐₓ ranging from 664 ng/mL to 996±450 ng/mL, at an initial daily dose of 66 mg:

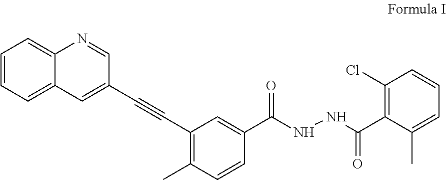 [SMILES: Cc1ccc(C(=O)NNC(=O)c2c(C)cccc2Cl)cc1C#Cc1cnc2ccccc2c1]

such that when the patient develops hematologic and/or non-hematologic toxicity:
   a) the initial daily dose is withheld for at least 7 days and resumed at 66 mg; or
   b) the initial daily dose is withheld for less than 7 days and if toxicity is resolved then resumed at 66 mg; or
   c) the initial daily dose is withheld for at least 7 days and resumed at a reduced daily dose of 48 mg;
and wherein, the reduced dose is optionally re-escalated to any one of the dose selected from 66 mg, 87 mg, 90 mg, 126 mg, 130.5 mg, 135 mg, 174 mg, 180 mg, 200 mg, and 204 mg, to achieve or maintain at least one of (a) complete hematological response, (b) complete hematological response and partial cytogenetic response, (c) complete hematological response and complete cytogenetic response, or (d) complete hematological response, complete cytogenetic response, and major molecular response, with no severe adverse reaction and said daily dose;
   a) results in not less than 50 ng/mL of the compound of Formula I or a pharmaceutically acceptable salt thereof in the plasma; or
   b) achieves 15% higher AUC₀₋₂₄ when administered under fasted conditions as compared to when administered under fed conditions; or
   c) achieves 20% higher Cₘₐₓ when administered under fasted conditions as compared to when administered under fed conditions; or
   d) results in patient's QT interval of less than about 500 ms.

According to any one of the embodiments described herein, chronic myeloid leukemia is newly diagnosed.

According to any one of the embodiments described herein, chronic myeloid leukemia is refractory.

According to any one of the embodiments described herein, chronic myeloid leukemia is in chronic phase.

According to any one of the embodiments described herein, the patient is in the accelerated phase of chronic myeloid leukemia (CML).

According to any one of the embodiments described herein, the patient is in blast phase of chronic myeloid leukemia (CML).

According to any one of the embodiments described herein, the patient is an adult patient.

According to any one of the embodiments described herein, the patient has an age of more than 18 years.

According to any one of the embodiments described herein, the chronic phase patient has at least one following characteristic before treatment:
a) 15% blasts in peripheral blood and bone marrow;
b) <30% blasts plus promyelocytes in peripheral blood and bone marrow;
c) <20% basophils in the peripheral blood;
d) ≥50×10⁹/L (≥50,000/mm³) platelets;
e) Transient prior therapy related thrombocytopenia (<50,000/mm³ for ≤30 days prior to screening); and
f) No evidence of extramedullary leukemic involvement, with the exception of hepatosplenomegaly.

According to any one of the embodiments described herein, the accelerated phase patient has at least one following characteristic before treatment:
a) ≥15 to <30% blasts in peripheral blood or bone marrow;
b) ≥20% basophils in peripheral blood or bone marrow;
c) ≥30% (blasts+promyelocytes) in peripheral blood or bone marrow (but <30% blasts);
d)≤100×109 platelets/L in peripheral blood unrelated to therapy; and
e) Additional clonal cytogenetic abnormalities in Ph+ cells.

According to any one of the embodiments described herein, the blast phase patient has at least one following characteristic before treatment:
a) ≥30% blasts in peripheral blood, bone marrow or both; and
b) extra-medullary disease.

According to any one of the embodiments described herein, the BCR-ABL1 ratio is ≥0.1% at the time of screening for patients.

According to any one of the embodiments described herein, the daily dose is withheld up to 14 days.

According to any one of the embodiments described herein, the daily dose is withheld up to 28 days.

According to any one of the embodiments described herein, the daily dose is withheld up to four weeks.

According to any one of the embodiments described herein, the daily dose is withheld up to 56 days.

According to any one of the embodiments described herein, the daily dose is withheld up to eight weeks.

According to any one of the embodiments described herein, the daily dose is withheld in case of grade 1 or grade 2 toxicities in patients.

According to any one of the embodiments described herein, the daily dose is withheld in case of grade 3 toxicities in patients.

According to any one of the embodiments described herein, the daily dose is withheld in case of grade 1 or grade 2 toxicities and in case of grade 3 toxicities in patients.

According to any one of the embodiments described herein, the reduced dose is optionally re-escalated to achieve or maintain at least one of (a) complete hematological response, (b) complete hematological response and partial cytogenetic response, (c) complete hematological response and complete cytogenetic response, or (d) complete hematological response, complete cytogenetic response, and major molecular response.

According to any one of the embodiments described herein, toxicity or severe adverse reaction include hematologic toxicities.

According to any one of the embodiments described herein, toxicity or severe adverse reaction include non-hematologic toxicities.

According to any one of the embodiments described herein, toxicity or severe adverse reaction include hematologic and non-hematologic toxicities.

According to any one of the embodiments described herein, hematologic toxicity is measured by any one or more of the following total and differential WBC count, absolute neutrophil count (ANC), absolute basophils count, absolute eosinophil count, absolute monocyte count, absolute lymphocyte count (ALC), platelet count, hemoglobin, hematocrit, and mean corpuscular volume (MCV).

According to any one of the embodiments described herein, non-hematologic toxicity includes any one or more of the following diarrhea, anemia, cough, nausea, constipation, fatigue, and headache.

According to any one of the embodiments described herein, non-hematologic toxicity includes cardiovascular toxicities.

According to any one of the embodiments described herein, the patient is said to have developed hematologic toxicity when the administration of the compound of Formula I or a pharmaceutically acceptable salt thereof results in patient's absolute neutrophil count of less than 500/mm³.

According to any one of the embodiments described herein, the patient is said to have developed hematologic toxicity when the administration of the compound of Formula I or a pharmaceutically acceptable salt thereof results in patient's absolute neutrophil count of less than 1000/mm³.

According to any one of the embodiments described herein, the patient is said to have developed hematologic toxicity when the administration of the compound of Formula I or a pharmaceutically acceptable salt thereof results in patient's absolute neutrophil count of less than 1500/mm³.

According to any one of the embodiments described herein, the patient is said to have developed hematologic toxicity when the administration of the compound of Formula I or a pharmaceutically acceptable salt thereof results in platelet count of less than 1,00,000/mm³.

According to any one of the embodiments described herein, the patient is said to have developed hematologic toxicity when the administration of the compound of Formula I or a pharmaceutically acceptable salt thereof results in platelet count of less than 50,000/mm³.

According to any one of the embodiments described herein, the patient is said to have developed hematologic toxicity when the administration of the compound of Formula I or a pharmaceutically acceptable salt thereof results in platelet count of less than 75,000/mm³.

According to any one of the embodiments described herein, the patient is said to have developed hematologic toxicity when the administration of the compound of Formula I or a pharmaceutically acceptable salt thereof results in platelet count of less than 25,000/mm³.

According to any one of the embodiments described herein, the patient is said to have developed hematologic toxicity when the administration of the compound of Formula I or a pharmaceutically acceptable salt thereof results in patient's absolute neutrophil count of less than 1500/mm³ and/or platelet count of less than 75,000/mm³ and wherein such toxicities are unrelated to the underlying chronic myeloid leukemia (CML) disease.

According to any one of the embodiments described herein, the patient is said to have developed hematologic toxicity when the administration of the compound of Formula I or a pharmaceutically acceptable salt thereof results in patient's absolute neutrophil count of less than 1000/mm³ and/or platelet count of less than 50,000/mm³ and wherein such toxicities are unrelated to the underlying chronic myeloid leukemia (CML) disease.

According to any one of the embodiments described herein, the patient is said to have developed hematologic toxicity when the administration of the compound of Formula I or a pharmaceutically acceptable salt thereof results in patient's absolute neutrophil count of less than 500/mm³ and/or platelet count of less than 25,000/mm³ and wherein such toxicities are unrelated to the underlying chronic myeloid leukemia (CML) disease.

According to any one of the embodiments described herein, the patient is said to have developed severe adverse reaction when the non-hematologic toxicity is a grade 3 or 4 toxicity while on therapy and/or with persistent grade 2 toxicity, unresponsive to optimal management including dose adjustments to the lowest doses.

According to any one of the embodiments described herein, the patient is said to have developed severe adverse reaction when the non-hematologic toxicity is a grade 3 or 4 toxicity and/or with persistent grade 2 toxicity, unresponsive to optimal management, wherein grade 3 and 4 toxicities are as listed in Common Terminology Criteria for Adverse Events (CTCAE).

According to any one of the embodiments described herein, the patient is said to have developed severe adverse reaction when the non-hematologic toxicity is a grade 3 toxicity and/or an intolerable grade 1 or grade 2 toxicity that is persistent for at least 7 days despite optimal management, wherein grade 1, 2 and 3 toxicities are as listed in Common Terminology Criteria for Adverse Events (CTCAE).

According to any one of the embodiments described herein, the non-hematologic toxicity is an intolerable grade 1 or grade 2 toxicity that is persistent for over 7 days despite optimal management and/or grade 3 toxicity, wherein grade 1, 2 and 3 toxicities as listed in Common Terminology Criteria for Adverse Events (CTCAE).

According to any one of the embodiments described herein, the hematologic toxicity is a grade 3 toxicity as listed in Common Terminology Criteria for Adverse Events (CTCAE).

According to any one of the embodiments described herein, the hematologic toxicity is a grade 3 or 4 toxicity as listed in Common Terminology Criteria for Adverse Events (CTCAE).

According to any one of the embodiments described herein, the daily dose of the compound of Formula I or a pharmaceutically acceptable salt thereof in-patient results in not less than 100 ng/ml of compound of Formula I in the plasma.

According to any one of the embodiments described herein, the daily dose of compound of Formula I or a pharmaceutically acceptable salt thereof in patient results in not less than 50 ng/ml of compound of Formula I in the plasma.

According to any one of the embodiments described herein, the plasma concentration of compound of Formula I or a pharmaceutically acceptable salt thereof is not less than 70 ng/mL.

According to any one of the embodiments described herein, the plasma concentration of compound of Formula I or a pharmaceutically acceptable salt thereof is not less than 90 ng/mL.

According to any one of the embodiments described herein, the plasma concentration of compound of Formula I or a pharmaceutically acceptable salt thereof is not less than 100 ng/mL.

According to any one of the embodiments described herein, the compound of Formula I or a pharmaceutically acceptable salt thereof is used after the patient received at least one tyrosine kinase inhibitor.

According to any one of the embodiments described herein, the compound of Formula I or a pharmaceutically acceptable salt thereof is used after the patient received two tyrosine kinase inhibitors.

According to any one of the embodiments described herein, the compound of Formula I or a pharmaceutically acceptable salt thereof is used after the patient received three tyrosine kinase inhibitors.

According to any one of the embodiments described herein, the refractory chronic myeloid leukemia (CML) patient is resistant or intolerant to, or has relapsed after the treatment using at least one tyrosine kinase inhibitor prior to administration of compound of Formula I or a pharmaceutically acceptable salt thereof.

According to any one of the embodiments described herein, the refractory chronic myeloid leukemia (CML) patient is resistant or intolerant to, or has relapsed after the treatment using two tyrosine kinase inhibitors prior to administration of compound of Formula I or a pharmaceutically acceptable salt thereof.

According to any one of the embodiments described herein, the refractory chronic myeloid leukemia (CML) patient is resistant or intolerant to, or has relapsed after the treatment using three tyrosine kinase inhibitors prior to administration of compound of Formula I or a pharmaceutically acceptable salt thereof.

According to any one of the embodiments described herein, the patient has undergone prior treatment using at least one tyrosine kinase inhibitor.

According to any one of the embodiments described herein, the patient has undergone prior treatment using two tyrosine kinase inhibitors.

According to any one of the embodiments described herein, the patient has undergone prior treatment using three tyrosine kinase inhibitors.

According to any one of the embodiments described herein, one of the tyrosine kinase inhibitor is ponatinib.

According to any one of the embodiments described herein, one of the tyrosine kinase inhibitor is asciminib.

According to any one of the embodiments described herein, one of the tyrosine kinase inhibitor is bosutinib.

According to any one of the embodiments described herein, one of the tyrosine kinase inhibitor is dasatinib.

According to any one of the embodiments described herein, one of the tyrosine kinase inhibitor is nilotinib.

According to any one of the embodiments described herein, one of the tyrosine kinase inhibitor is radotinib.

According to any one of the embodiments described herein, one of the tyrosine kinase inhibitor is imatinib.

According to any one of the embodiments described herein, the patient does not have T315I mutations. In another embodiment, the patient does not have T315I mutations (e.g., the patient does not have a T315I mutation and is resistant or intolerant to, or has relapsed after the treatment using three tyrosine kinase inhibitors prior to administration of the compound of Formula I or a pharmaceutically acceptable salt thereof).

According to any one of the embodiments described herein, the compound of Formula I or a pharmaceutically acceptable salt thereof is administered under fasting conditions.

According to any one of the embodiments described herein, the compound of Formula I or a pharmaceutically acceptable salt thereof is administered at least once daily.

According to any one of the embodiments described herein, the compound of Formula I or a pharmaceutically acceptable salt thereof is administered once daily.

According to any one of the embodiments described herein, the compound of Formula I or a pharmaceutically acceptable salt thereof is administered twice daily.

According to any one of the embodiments described herein, the AUC₀₋₂₄ is increased by at least 15% by administering the compound of Formula I or a pharmaceutically acceptable salt thereof to the patient under fasting conditions.

According to any one of the embodiments described herein, the AUC₀₋₂₄ is increased by at least 15% by administering the compound of Formula I or a pharmaceutically acceptable salt thereof to the patient under fasting conditions as compared to the AUC₀₋₂₄ obtained by administering the compound of Formula I or a pharmaceutically acceptable salt thereof under fed conditions.

According to any one of the embodiments described herein, the Cₘₐₓ is increased by at least 20% by administering the compound of Formula I or a pharmaceutically acceptable salt thereof to the patient under fasting conditions as compared to the Cₘₐₓ obtained by administering the compound of Formula I or a pharmaceutically acceptable salt thereof under fed conditions.

According to any one of the embodiments described herein, the fasting conditions include overnight fasting or at least about 2 hours of fasting before and after administration of the compound of Formula I or a pharmaceutically acceptable salt thereof.

According to any one of the embodiments described herein, chronic myeloid leukemia (CML) is a Philadelphia Chromosome Positive Chronic Myeloid Leukemia in chronic phase (Ph+ CML-CP) or Philadelphia Chromosome positive acute lymphoblastic leukemia (Ph+ ALL).

According to any one of the embodiments described herein, chronic myeloid leukemia (CML) is a Philadelphia Chromosome Positive Chronic Myeloid Leukemia in chronic phase (Ph+ CML-CP).

According to any one of the embodiments described herein, the Philadelphia Chromosome Positive Chronic Myeloid Leukemia patient (Ph+ CML) is a patient suffering from chronic phase chronic myeloid leukemia (CP-CML), accelerated phase chronic myeloid leukemia (AP-CML), blast phase chronic myeloid leukemia (BP-CML).

According to any one of the embodiments described herein, the patient with chronic myeloid leukemia (CML) is a newly diagnosed chronic myeloid leukemia (CML) patient or a refractory chronic myeloid leukemia (CML) patient who has undergone prior treatment using at least one tyrosine kinase inhibitor and is resistant or intolerant to, or has relapsed from said prior treatment.

According to any one of the embodiments described herein, the patient with chronic myeloid leukemia (CML) is a refractory chronic myeloid leukemia (CML) patient who has undergone prior treatment using at least one tyrosine kinase inhibitor and is resistant or intolerant to, or has relapsed from said prior treatment.

According to any one of the embodiments described herein, the patient with chronic myeloid leukemia (CML) is a refractory chronic myeloid leukemia (CML) patient with Philadelphia Chromosome Positive Chronic Myeloid Leukemia in Chronic Phase (Ph+ CML-CP), who has undergone prior treatment using more than one tyrosine kinase inhibitor and/or is resistant and/or intolerant to, and/or has relapsed from said prior treatment.

According to any one of the embodiments described herein, the patient who has undergone prior treatment using at least one tyrosine kinase inhibitor and is resistant or intolerant to, or has relapsed from said prior treatment, has at least one following characteristics:
a) Failure to achieve complete hematologic response (CHR) after 3 months of treatment initiation;
b) No cytogenetic response (>95% Ph+ cells) in three months after the initiation of treatment;
c) Less than a minor cytogenetic response (>65% Ph+) in six months after the initiation of treatment;
d) Less than a Partial Cytogenetic Response (>35% Ph+) in twelve months after the initiation of treatment;
e) Loss of cytogenetic response at any time after initiation of therapy;
f) BCR-ABL1 ratio >10% in three months after the initiation of therapy;
g) BCR-ABL1 ratio >1% in twelve months after the initiation of therapy;
h) Confirmed loss of Major Molecular Response (MMR) in 2 consecutive tests, at any time during therapy;
i) Loss of complete hematologic response (CHR), complete cytogenetic response (CCyR) or Partial Cytogenetic Response (PCyR) at any time after the initiation of therapy; and
j) Development of new clonal chromosome abnormalities in Ph+ cells or development of new BCR-ABL1 mutations with the potential to cause resistance to study treatment or with loss of previously obtained response on the TKI at any time after the initiation of therapy.

According to any one of the embodiments described herein, wherein daily dose does not result in patient's QT interval of more than about 500 ms.

According to any one of the embodiments described herein, daily dose does not result in patient's QT interval of more than about 450 ms.

According to any one of the embodiments described herein, the daily dose;
a) results in not less than 50 ng/mL of the compound of Formula I or a pharmaceutically acceptable salt thereof in the plasma;
b) results in mean AUC₀₋₂₄ ranging from 28598 ng*h/mL to 42898±33827 ng*h/mL;
c) achieves a mean Cₘₐₓ ranging from 3370 ng/mL to 5054±3051 ng/ml;
d) achieves 15% higher AUC₀₋₂₄ when administered under fasted conditions as compared to when administered under fed conditions;
e) achieves 20% higher Cₘₐₓ when administered under fasted conditions as compared to when administered under fed conditions; and/or
f) results in patient's QT interval of less than about 500 ms.

According to any one of the embodiments described herein, the daily dose is escalated to achieve or maintain complete hematologic response or the daily dose is reduced when the patient experiences a severe adverse reaction, and wherein the daily dose:
a) results in not less than 100 ng/ml of the compound of Formula I or a pharmaceutically acceptable salt thereof in the plasma;
b) results in mean AUC₀₋₂₄ ranging from 28598 ng*h/mL to 42898±33827 ng*h/mL;
c) achieves a mean Cₘₐₓ ranging from 3370 ng/ml to 5054±3051 ng/ml;
d) achieves 15% higher AUC₀₋₂₄ when administered under fasted conditions as compared to when administered under fed conditions;
e) achieves 20% higher Cₘₐₓ when administered under fasted conditions as compared to when administered under fed conditions; and/or
f) results in patient's QT interval to less than about 500 ms.

According to any one of the embodiments described herein, the initial dose is escalated to achieve or maintain complete hematologic response (CHR) with no severe adverse reaction.

According to any one of the embodiments described herein, the dose is escalated when the patient did not have grade 3 or higher toxicity.

According to any one of the embodiments described herein, the dose is escalated based on one or more following characteristics:
a) failure to achieve complete hematological response (CHR) by week 8;
b) failure to reach complete cytogenetic response (CCyR) by week 12; and
c) loss of a previously achieved hematologic or cytogenetic response.

According to any one of the embodiments described herein, the dose is re-escalated based on one or more following characteristics:
a) no grade 3 and/or grade 4 hematologic and non-hematological toxicities;
b) grade 2 toxicity that is persistent for days despite optimal management;
c) toxicity leading to dose reduction is not recurrent or persistent and tolerated by the patient with optimal management; and
d) loss of previously achieved response.

According to any one of the embodiments described herein, the present invention provides an oral pharmaceutical composition for the treatment of a patient with chronic myeloid leukemia (CML) comprising a therapeutic effective amount of compound of Formula I or a pharmaceutically acceptable salt thereof to achieve mean AUC₀₋₂₄ ranging from 6226±5827 ng*h/mL to 60373±55659 ng*h/mL and mean Cₘₐₓ ranging from 664±450 ng/ml to 5054±3051 ng/ml, at an initial daily dose selected from 66 mg, 87 mg, 90 mg, 126 mg, 130.5 mg, 135 mg, 174 mg, and 180 mg, and escalating or de-escalating the dose to achieve or maintain complete hematologic response, with no severe adverse reaction.

According to any one of the embodiments described herein, the pharmaceutical composition comprises the compound of Formula I or a pharmaceutically acceptable salt thereof in amorphous form.

According to any one of the embodiments described herein, the pharmaceutical composition further comprises one or more pharmaceutically acceptable excipients.

According to any one of the embodiments described herein, the pharmaceutical composition comprises 43.5 mg of compound of Formula I or a pharmaceutically acceptable salt thereof.

According to any one of the embodiments described herein, the pharmaceutical composition comprises 87 mg of compound of Formula I or a pharmaceutically acceptable salt thereof.

According to any one of the embodiments described herein, the pharmaceutical composition comprises 130.5 mg of compound of Formula I or a pharmaceutically acceptable salt thereof.

According to any one of the embodiments described herein, the pharmaceutical composition comprises 174 mg of compound of Formula I or a pharmaceutically acceptable salt thereof.

In one particular embodiment, the present invention provides a clinical study to establish the maximum tolerated dose (MTD) and proposed recommended dose (RP2D) for compound of Formula I or a pharmaceutically acceptable salt thereof.

In another particular embodiment, there is provided a phase 2 or phase 3, multicenter, open-label, randomized study of Vodobatinib versus Bosutinib in Patients with Philadelphia Chromosome Positive Chronic Myeloid Leukemia in chronic phase (Ph+ CML-CP) resistant or/and intolerant to ≥1 prior tyrosine kinase inhibitors.

According to any one of the embodiments described herein, prior tyrosine kinase inhibitors are first generation tyrosine kinase inhibitors.

According to any one of the embodiments described herein, prior tyrosine kinase inhibitors are second generation tyrosine kinase inhibitors.

According to any one of the embodiments described herein, the study includes patients or subjects participation for approximately 60 months.

According to any one of the embodiments described herein, selected patients or subjects remain on the assigned study treatment for approximately 60 months (i.e.: 5 years from first dosing), or until intolerance, patient withdrawal from the study, disease progression or death whichever comes first.

According to any one of the embodiments described herein, patients can return for a follow-up visit within 30±5 days after the last treatment administration.

According to any one of the embodiments described herein, patients who discontinue their study treatment at any time during the study can follow up for survival and for progression to AP/BC for up to 5 years from the date the last patient received the first study dose.

In one particular embodiment, the present invention provides a method of treating a treatment-resistant patient having chronic myeloid leukemia (CML) comprising a therapeutically effective amount of compound of Formula I or a pharmaceutically acceptable salt thereof to the patient, wherein:
i. the method includes orally administering 174 to 204 mg of compound of Formula I or a pharmaceutically acceptable salt thereof daily (e.g., once daily), and
ii. the patient is resistant or intolerant to at least one tyrosine kinase inhibitor prior to administration of compound of Formula I or a pharmaceutically acceptable salt thereof (e.g., the patient is resistant or intolerant to at least three tyrosine kinase inhibitors prior to administration of compound of Formula I or a pharmaceutically acceptable salt thereof).

In another particular embodiment, the present provides a method of treating a treatment-resistant patient having chronic myeloid leukemia (CML) comprising a therapeutically effective amount of compound of Formula I or a pharmaceutically acceptable salt thereof to the patient, wherein
i. the method includes:
   a) orally administering a therapeutic effective amount of compound of Formula I or a pharmaceutically acceptable salt thereof to provide a mean AUC₀₋₂₄ ranging from 6226±5827 ng*h/mL to 60373±55659 ng*h/mL, a mean Cₘₐₓ ranging from 664±450 ng/ml to 5054±3051 ng/mL, or a both, or
   b) orally administering 174 mg of compound of Formula I or a pharmaceutically acceptable salt thereof daily (e.g., once daily), and
ii. the patient (a) does not have a T315I mutation, and (b) is resistant or intolerant to, or has relapsed after the treatment using three tyrosine kinase inhibitors prior to administration of compound of Formula I or a pharmaceutically acceptable salt thereof.

According to any one of the embodiments described herein, the patient has Ph+ CML.

According to any one of the embodiments described herein, the patient is in the chronic phase of CML.

According to any one of the embodiments described herein, at least one tyrosine kinase inhibitor is a second-generation tyrosine kinase inhibitor.

According to any one of the embodiments described herein, the second-generation tyrosine kinase inhibitor is selected from dasatinib, nilotinib, radotinib and bosutinib.

According to any one of the embodiments described herein, at least one tyrosine kinase inhibitor is a third-generation tyrosine kinase inhibitor.

According to any one of the embodiments described herein, the third-generation tyrosine kinase inhibitor is selected from ponatinib and asciminib.

According to any one of the embodiments described herein, the patient has one or more of the following mutations in the BCR-ABL1 kinase domain: M244V, L248V, E499E, Q252H, P223S, A337T, F359C, E255V, Y253F, F317L, Y253H, V299L, E255V, F317L, E255V, F359V, G250E, E255K, F359V, M351T, G250E, H369R, H396R, and V359I.

According to any one of the embodiments described herein, the Bcr-Abl in the CML cells in the patient does not have a T315I mutation.

According to any one of the embodiments described herein, the compound of Formula I is orally administered in the form of capsules.

According to any one of the embodiments described herein, the compound of Formula I is in the form of oral capsules.

According to any one of the embodiments described herein, the capsules each contain 43.5 mg, 45 mg, 48 mg or 50 mg of compound of Formula I.

According to any one of the embodiments described herein, the compound of Formula I is administered each morning and optionally with two hours of fasting before and after administration of the compound of Formula I.

According to any one of the embodiments described herein, when upon the patient exhibits a grade 1 or grade 2 non-hematological adverse event which was intolerable due to clinical symptoms or interference with daily activities, the treatment is withheld for a period of time and then reinitiated.

According to any one of the embodiments described herein, the period of time during which the treatment is withheld is 7 days.

According to any one of the embodiments described herein, the period of time during which the treatment is withheld is 14 days.

According to any one of the embodiments described herein, when upon the patient exhibits a grade 3 hematological or non-hematological adverse event or grade 4 asymptomatic hematological adverse event, the treatment is withheld for a period of time and then reinitiated.

According to any one of the embodiments described herein, the treatment is withheld for up to 56 days for recovery to a grade 1 adverse event or less; and where the patient does not recover to a grade 1 adverse event or less, treatment with compound of Formula I is discontinued.

According to any one of the embodiments described herein, the method includes orally administering one or more oral capsules daily, where each capsule contains the same amount of compound of Formula I, and the amount is selected from 43.5 mg, 45 mg, 48 mg and 50 mg, and upon the occurrence of certain adverse events not requiring discontinuation or temporary withholding treatment, the daily dosage is reduced by administering a fewer number of the same oral capsules daily.

According to any one of the embodiments described herein, prior to a dose reduction 4 oral capsules are administered daily.

According to any one of the embodiments described herein, patients or subjects are selected or included in the study based on one or more following characteristics:
a) male or non-pregnant non-lactating females with a diagnosis of Ph+ CML-CP, aged ≥18 years with Eastern Cooperative Oncology Group performance status 0, or 1 who are resistant and/or intolerant to ≥1 prior 2^{nd} generation tyrosine kinase inhibitor (i.e. resistant to at least one of the following: dasatinib, nilotinib, radotinib, asciminib or ponatinib);
b) documented evidence of Ph+ CML-CP meeting criteria defined by one or more following characteristics:
   i. <15% blasts in peripheral blood and bone marrow;
   ii. <30% blasts plus promyelocytes in peripheral blood and bone marrow;
   iii. <20% basophils in the peripheral blood;
   iv. ≥50×10⁹/L (≥50,000/mm³) platelets;
   v. transient prior therapy-related thrombocytopenia (<50,000/mm³ for ≤30 days prior to screening); and
   vi. no evidence of extramedullary leukemic involvement with the exception of hepatosplenomegaly.

According to any one of the embodiments described herein, resistance or intolerance is defined wherein the patient needs to meet at least one following criteria for eligibility:
a) failure to achieve complete hematologic response (CHR) after 3 months of treatment initiation;
b) no cytogenetic response (>95% Ph+ cells) in three months after the initiation of therapy;
c) less than a minor cytogenetic response (>65% Ph+) in six months after the initiation of therapy;
d) less than a Partial Cytogenetic Response (PCyR) (>35% Ph+) in twelve months after the initiation of therapy;
e) loss of cytogenetic response at any time after initiation of therapy such that shift of the cytogenetic response to at least 1 grade worse from the patient's most recently performed bone marrow cytogenetics;
f) BCR-ABL1 ratio is >10% in three months after the initiation of therapy;
g) BCR-ABL1 ratio is >1% in twelve months after the initiation of therapy;
h) confirmed loss of Major Molecular Response (MMR) in 2 consecutive tests, at any time during therapy;
i) loss of complete hematologic response (CHR), complete cytogenetic response (CCyR) or Partial Cytogenetic Response (PCyR) at any time after the initiation of therapy;
j) development of new clonal chromosome abnormalities in Ph+ cells; and
k) development of new BCR-ABL1 mutations with potential to cause resistance to study treatment or with loss of previously obtained response on the TKI at any time after the initiation of therapy.

In one particular embodiment, non-hematological intolerance includes patients with grade 3 or 4 toxicity while on therapy and/or with persistent grade 2 toxicity, unresponsive to optimal management including dose adjustments to the lowest doses. In another particular embodiment, hematologic intolerance includes patients with grade 3 and/or 4 toxicity (absolute neutrophil count and/or platelets) while on therapy that is recurrent after dose reduction to the lowest doses.

In one embodiment, patients or subjects included in a clinical study have adequate organ and immune system functions as given below in table 06, which may be determined ≤2 weeks prior to administration of the compound of Formula I or a pharmaceutically acceptable salt thereof:

TABLE 06

| | |
|---|---|
| System | Laboratory value |

Hematology

| | |
|---|---|
| Absolute neutrophil count (ANC) | ≥1500/mm³ |

Renal

| | |
|---|---|
| Creatinine clearance (calculated | ≥50 mL/min |
| using the Cockcroft-Gault formula) | |

Hepatic

| | |
|---|---|
| Total bilirubin | ≤1.5 mg × ULN with exception of |
| | Gilbert's syndrome* |
| Aspartate aminotransferase (AST) | ≤3 × upper limit of normal (ULN). |
| Alanine aminotransferase (ALT) | ≤3 × ULN. |
| Alkaline phosphatase | ≤5.0 × ULN |

Pancreatic

| | |
|---|---|
| Serum lipase | ≤1.5 × ULN |
| Serum amylase | ≤1.5 × ULN |

*Total bilirubin ≤3 × upper limit of normal (ULN) or direct bilirubin ≤1.5 × upper limit of normal (ULN) in patients with Gilbert's syndrome.

In another embodiment, the present invention provides medically acceptable methods of birth control for patients or subjects included in study having childbearing potential to use any following of effective birth control methods:
a) combined (estrogen and progestogen-containing) hormonal contraception associated with inhibition of ovulation (oral, intravaginal, transdermal);
b) progestogen-only hormonal contraception associated with inhibition of ovulation;
c) placement of an IUD/intra-uterine hormone-releasing system; and
d) same-sex partner, a vasectomized partner/bilateral tubal occlusion.

In one particular embodiment, patients or subjects are excluded from the study based on one or more following characteristics:
a) presence of T315I mutation;
b) patients in Major Molecular Response (MMR) per central laboratory assessment at baseline;
c) history of accelerated or blast phase CML;
d) history of any relevant allergy/hypersensitivity (including known immediate or delayed hypersensitivity reaction or idiosyncrasy to drugs chemically related to compound of Formula I or a pharmaceutically acceptable salt thereof);
e) evidence of clinically significant organ dysfunction or any clinically relevant deviation from normal in physical examination, ECG findings, vital signs, or clinical laboratory test findings which in the opinion of the investigator may jeopardize the safety of the patient during the study or may interfere with the evaluation of the study drug;
f) any major surgery within 4 weeks of administration;
g) female participant with positive urine pregnancy tests;
h) known history of active hepatitis B or hepatitis C or cirrhosis and positive Human immunodeficiency virus (HIV) test;
i) use of concomitant medication that might reasonably influence the study results of the study prior to administration and at any time during the study;
j) radiotherapy or cytotoxic chemotherapy within 24 days (42 days for nitrosoureas or mitomycin C), interferon or cytarabine or immunotherapy within 14 days prior to the first administration visit; or a targeted therapy such tyrosine kinase inhibitors (TKIs) or investigational therapy not completing wash out of 5 half-lives prior to the first administration visit;
k) known or suspected history of significant drug abuse (amphetamines, barbiturates, cannabinoids, cocaine metabolites, and opiates); known or suspected history of alcohol abuse or excessive alcohol intake in the 12 months prior to study entry;
l) patients not recovered to Grade ≤1 toxicity;
m) malabsorption syndrome or other illnesses that could affect oral absorption;
n) clinically significant, uncontrolled, or active cardiovascular disease, specifically including one or more following, but not restricted to:
   i. recent history of myocardial infarction e.g. ≤6 months from screening, unstable angina, coronary heart disease, cerebrovascular accident, or transient ischemic attack;
   ii. congestive heart failure within 6 months from screening, or left ventricular ejection fraction less than 35% within 6 months prior to the screening visit;
   iii. history of clinically significant arrhythmia;
iv. prolonged rate-corrected QT Interval (QT_{c}F) interval on the screening ECG (>450 msec for males and >470 msec for females) or history of long QT syndrome/family history of sudden deaths or congenital long QT syndrome;
   v. risk factors for Torsades de Pointes (TdP) including uncorrected electrolyte derangements (i.e.: hypokalemia or hypomagnesemia, history of cardiac failure, or history of cardiac arrhythmias, concomitant medications associated with known risk of TdP which cannot be discontinued;
   vi. clinically significant symptomatic bradycardia;
   vii. inability to calculate QTcF interval;
   viii. hyper-eosinophilic syndrome with cardiac involvement;
o. uncontrolled intercurrent illness including, but not limited to, ongoing or active infection, uncontrolled hypertension, uncontrolled diabetes, uncontrolled seizure disorder, acute or chronic liver or pancreatic disease, severe renal disease, psychiatric or social circumstances that would limit compliance with study requirements as per the investigator's discretion;
p. active or symptomatic central nervous system (CNS) disease involvement due to CML;
q. history of significant bleeding disorder; and
r. autologous or allogenic stem cell transplant ≤3 months prior to screening; any evidence of ongoing graft versus host disease (GVHD) or GVHD requiring immunosuppressive therapy ≤28 days prior to the first administration visit or planning to undergo allogeneic stem cell transplant.

### Examples

### Statistical Analysis

All derivations, statistical analyses, summaries, and listings were generated using SAS® version 9.4 (SAS Institute, Inc., Cary, North Carolina). Non-compartmental PK parameter calculations were performed using Phoenix® WinNonlin® 6.4 (Certara, St. Louis, Missouri). Graphics were prepared using the same versions of SAS, or Phoenix WinNonlin, or with SigmaPlot 12.5 (Systat Software, Inc., San Jose, California).

### Statistical Methods

Efficacy Parameters: Parts B and C: Descriptive summaries were presented for the safety and efficacy variables collected. Continuous variables were summarized using mean, standard deviation, minimum, median, and maximum. Categorical variables were summarized using frequency counts and percentages. For time-to-event variables, Kaplan-Meier plots, median time-to-event, and other percentiles were presented.

Safety Parameters: Parts B and C: Descriptive summaries were provided for the safety variables collected. Continuous variables were summarized using mean, standard deviation, minimum, median, and maximum. Categorical variables were summarized using frequency counts and percentages. For time-to-event variables, other percentiles were presented. In addition, summaries of AEs by intensity, seriousness, frequency, and relationship to IMP are presented. Adverse events (AE) s were coded using the Medical Dictionary for Regulatory Activities (MedDRA) and summarized by System Organ Class (SOC) and Preferred Term (PT).

In-vitro studies revealed that the compound of Formula I selectively and potently inhibits ABL1 kinase and its mutant forms at nanomolar IC₅₀ values with growth inhibition of cell lines expressing Ph+ wild-type and mutant forms of BCR-ABL1. In vivo studies of the compound of Formula I including single- and repeat-dose studies in preclinical models (mice, rats, and dogs) using the clinically relevant oral route and daily scheduled dosing indicate systemic bioavailability and no specific adverse events at multiple efficacious doses. The starting human equivalent dose for this study was calculated after the application of the appropriate conversion factors and safety factors as recommended in the US Food and Drug Administration Guidance for Industry (2005). The risk-benefit of using the compound of Formula I in healthy volunteers was assessed after analysis of data from toxicology studies conducted in accordance with International Council for Harmonisation (ICH) guidelines.

An oral toxicity study of the compound of Formula I in Wistar Rats showed a NOAEL of 100 mg/kg/day (approximately 12 mg/kg/day). In a 14-day study conducted in Beagle dogs, no mortality or severe clinical signs were observed up to a dose of 625 mg/kg/day. The NOAEL was observed to be 83 mg/kg/day and the human equivalent dose was calculated from the NOAEL of 100 mg/kg/day.

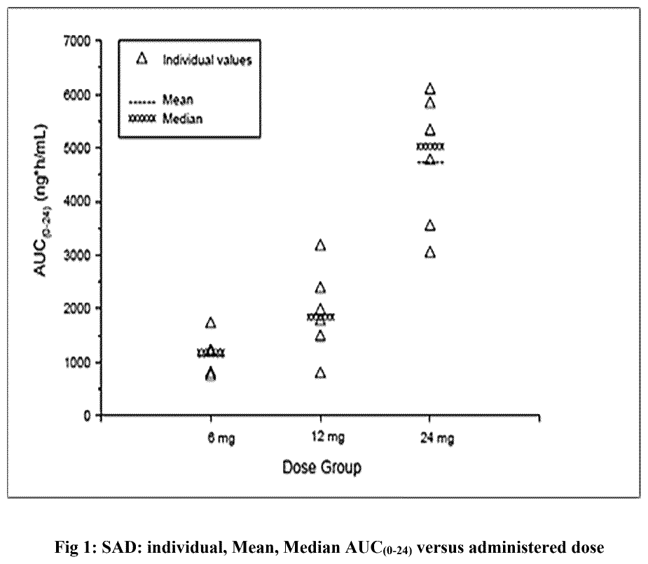

Initially in the single ascending dose (SAD) study the safety, tolerability, and pharmacokinetics of the compound of Formula I at 6 mg, 12 mg, and 24 mg doses in healthy human male volunteers were evaluated. In this study, the healthy male volunteers within the mean age group of 28.7-35 years were orally administered an investigational medicinal product (IMP) or a placebo after an overnight fast (of at least 8 hours). The IMP used in this study was a hard gelatin capsule comprising the compound of Formula I of varying sizes containing either 6 mg or 24 mg of the compound of Formula I and the excipients. For the 12 mg dose, two capsules of 6 mg were used. The placebo formulation containing the excipients matched the compound of Formula I capsules in shape, size, and color. Compound of Formula I was rapidly absorbed after oral administration of a single dose (6 mg, 12 mg, or 24 mg). The median time of maximum concentration (Tₘₐₓ: 2.5-3 hours, across dose range) was independent of the dose level. Dose-related increases in exposure (AUC and Cₘₐₓ) were observed. The mean AUC in plasma from zero (predose) until time 24 hours (AUC₍₀₋₂₄₎) at the highest dose level (24 mg) was 4.755 μg/mL. The maximum value of AUC₍₀₋₂₄₎ (6080 μg/mL) was less than 6.675 μg/mL (2-times lower than 13.35 μg/mL, the AUC_{(0 24)} at the no observed adverse-effect-level [NOAEL] toxicology dose), thus maintaining exposure within the established boundary for safety. Across dose range, apparent systemic clearance (CL/F: 3.708-4.956 L/hour), apparent volume of distribution (Vz/F: 69.76-91.58 L), and apparent terminal half-life (t1/2:12.69-17.12 hour) were independent of the dose (6 mg, 12 mg, and 24 mg). The inter-subject variability, for the PK parameters, was generally higher in the 12 mg dose group when compared with that in the 6 mg and 24 mg dose groups (Table 07, and FIG. 1)

TABLE 07
Summary of pharmacokinetic parameters of Compound of formula I

| | Compound of formula I Dose | | | | | |
|---|---|---|---|---|---|---|
| PK Parameter | 6 mg (N = 6) | | 12 mg (N = 6) | | 24 mg (N = 6) | |
| AUC₍₀₋₂₄₎ (ng/mL × hour) | 1135 | (31.1) | 1916 | (42.2) | 4755 | (26.1) |
| AUC_{(0-inf)} (ng/mL × hour) | 1735 | (43) | 2766 | (50.6) | 6585 | (30.5) |
| AUC_{(0-inf)}/D ([ng/mL × hour]/mg) | 289 | (43) | 230.3 | (50.6) | 274.3 | (30.5) |
| AUC₍₀₋ₗₐₛₜ₎ (ng/mL × hour) | 1567 | (41.1) | 2640 | (50.2) | 6235 | (28) |
| Cₘₐₓ (ng/mL) | 234.2 | (36.4) | 284.7 | (28.6) | 908.8 | (32.1) |
| Cₘₐₓ/D ([ng/mL × hour]/mg) | 39.02 | (36.4) | 23.73 | (28.5) | 37.88 | (32.1) |
| tₘₐₓ (hour) | 2.5 | (1.5-3) | 3 | (2-4) | 2.75 | (1.03-2.75) |
| V_{z}/F (L) | 91.58 | (46.6) | 90.84 | (36.3) | 69.76 | (54.1) |
| t_{1/2} (hour) | 17.12 | (48) | 12.69 | (33.1) | 12.73 | (57.4) |
| CL/F (L/hour) | 3.708 | (41.5) | 4.956 | (67.5) | 3.8 | (32.7) |

Pharmacokinetic parameters of AUC and Cₘₐₓ are expressed as mean (CV %), tₘₐₓ as median (range), and all others as geometric mean (GCV %). AUC, area under the concentration-time curve; AUC₍₀₋₂₄₎, AUC in plasma from zero (predose) until time 24 h; AUC_{(0-inf)}, AUC in plasma from zero (predose) extrapolated to infinite time; AUC₍₀₋ₗₐₛₜ₎, AUC in plasma from zero (predose) to time of last quantifiable concentration; CL/F, apparent systemic clearance after extravascular dosing; Cₘₐₓ, the maximum concentration in plasma; CV %, coefficient of variation; D, Dose; GCV % = geometric coefficient of variation; SD, Standard deviation; t_{1/2}, apparent terminal half-life; tₘₐₓ, time of maximum concentration; V_{z}/F, apparent volume of distribution following extravascular dosing.

Referring to FIG. 1, The dose proportionality assessment using ANCOVA was assessed in the PK analysis set. Following single-dose administration of escalating the compound of Formula I doses from 6 mg to 24 mg, the point estimates for the slopes obtained from the power model were close to 1 for Cₘₐₓ and total exposures [AUC_{(0-inf)} and AUC₍₀₋ₗₐₛₜ₎], suggesting a proportional increase in exposure with fold increase in dose. The estimates for the slope for all exposure parameters were between 0.9833 and 1.016. However, the slope estimates had a wide 90% Cis.

TABLE 08
Summary of dose proportionality of the compound of Formula I

| Parameter | | Slope | | | Intercept | | | Coefficient of |
|---|---|---|---|---|---|---|---|---|
| (unit) | n | Estimate | SE | 90% CI | Estimate | SE | 90% CI | determination |
| AUC_{(0-inf)} | 18 | 0.9833 | 0.1947 | 0.6433, | 5.534 | 0.4963 | 4.667, | 0.6145 |
| (ng*h/mL) | | | | 1.323 | | | 6.400 | |
| AUC₍₀₋ₗₐₛₜ₎ | 18 | 1.016 | 0.1895 | 0.6853, | 5.389 | 0.4829 | 4.546, | 0.6425 |
| (ng*h/mL) | | | | 1.347 | | | 6.232 | |
| Cₘₐₓ | 18 | 0.9884 | 0.1683 | 0.6946, | 3.470 | 0.4288 | 2.721, | 0.6832 |
| (ng/mL) | | | | 1.282 | | | 4.219 | |

AUC, area under the concentration-time curve; AUC_{(0-inf)}, AUC in plasma from zero (predose) extrapolated to infinite time; AUC₍₀₋ₗₐₛₜ₎, AUC in plasma from zero (predose) to time of last quantifiable concentration; CI, confidence interval; Cₘₐₓ, the maximum concentration in plasma; n, number of subjects; SE, standard error.

Further, single-dose administrations of the compound of Formula I at 6 mg, 12 mg and 24 mg were found to be safe and well tolerated in healthy subjects. There were no clinically significant changes in laboratory parameters, and no clinically significant changes in vital signs or electrocardiogram were recorded for any subject. No subject reached the subject withdrawal criteria based on QTcF prolongation or safety laboratory tests.

Based on the results from SAD study, a multiple ascending dose (MAD) was conducted on patients with Ph+ CML/ALL to determine maximum tolerated dose (MTD) and a recommended Phase 2 dose (RP2D).

Recruitment of patients with Ph+ CML/ALL in the Phase 1 study in this study was expected to be challenging due to the availability of other effective treatment options. Additionally, as there was no existing human PK profile available to suggest that planned doses of the compound of Formula I could be associated with efficacy, it would have led to administration of sub-therapeutic doses to the patients. Traditional anti-cancer agents such as chemotherapeutics have considerable safety risks, whereas molecularly targeted agents have adequate safety margins allowing for their evaluation in healthy volunteers. Although a single-dose study in healthy volunteers would not be predictive of toxicities in cancer patients receiving prolonged therapy, it would be indicative of any unexpected acute toxicity associated with a novel agent that may warrant discontinuation of therapy. The aim of this study was to use the safety and PK information from this single-dose study to design an appropriate dosing regimen and enable a more rapid advancement into the target patient population.

### Study Objective:

This was a Phase I/2, open-label, single-agent, multicenter, multi-dose, dose-escalation study in subjects with Philadelphia chromosome-positive Ph+ CML (CP CML, AP CML and BP CML) and Ph+ ALL. The primary objectives of this study were to determine the maximum tolerated dose (MTD; or recommended Phase 2 dose [RP2D]) of the compound of Formula I when administered orally and to evaluate its safety.

### Study Eligibility:

For patient selection, key inclusion criteria included men or women aged ≥18 years with Eastern Cooperative Oncology Group performance status 0, 1, or 2 with Ph+ CML (CP CML, AP CML, or BP CML) who are resistant and/or intolerant to ≥3 prior TKIs, having adequate organ and immune system function as indicated by Table 09, obtained ≤2 weeks prior to the administration of the compound of Formula I. Subjects with presence of T3151 mutations were not included in the study.

TABLE 09
organ and immune system function of subject ≤2 weeks
prior to administration of the compound of Formula
I for inclusion in the study:

| | |
|---|---|
| System | Laboratory value |

Hematology

| | |
|---|---|
| ANC | ≥1000/mm³ |
| Platelets | ≥50,000/mm³ |
| Prothrombin time and activated | ≤1.5 × ULN |
| partial thromboplastin time (aPTT) | |

Renal

| | |
|---|---|
| Creatinine clearance | Calculated creatinine |
| | clearance >60 mL/min (based |
| | on the Cockroft-Gault formula) |

Hepatic

| | |
|---|---|
| Total bilirubin | ≤1.5 × ULN with exception of |
| | Gilbert's syndrome* |
| Aspartate aminotransferase (AST) | ≤2.5 × ULN |
| Alanine aminotransferase (ALT) | ≤2.5 × ULN |
| Alkaline phosphatase | ≤5 × ULN |

Pancreatic

| | |
|---|---|
| Serum amylase and lipase | ≤1.5 × ULN |

*Total bilirubin: ≤2 × ULN in subjects with Gilbert's syndrome

The inclusion criteria further included: females must not be pregnant, or breast-feeding, and those of childbearing potential must use a medically accepted effective method of birth control with a negative pregnancy test. Males should not father a child and are advised to use an acceptable method to prevent the passage of semen to their sexual partner during intercourse from enrollment until 3 months after the last dose of the compound of Formula I.

Medically acceptable methods of birth control included the use of any of the effective birth control methods listed below:
a) Combined (estrogen and progestogen-containing) hormonal contraception associated with inhibition of ovulation (oral, intravaginal, transdermal);
b) Progestogen-only hormonal contraception associated with inhibition of ovulation;
c) Placement of an IUD/intra-uterine hormone-releasing system. Consideration should be given to the type of device or system being used, as there are higher failure rates quoted for certain types, e.g., steel or copper wire;
d) intrauterine hormone-releasing system; and
e) same-sex partner, a vasectomized partner/bilateral tubal occlusion.

Key exclusion criteria: the subjects were excluded from the study if there is presence of T315I mutation; any major surgery in the 4 weeks prior to first dose of compound of formula I; evidence of clinically significant organ dysfunction or any clinically relevant deviation from normal in physical examination, ECG findings, vital signs, or clinical laboratory test findings which may jeopardize the safety of the patient during the study or may interfere with the evaluation of the study drug; positive pregnancy or HIV test; history of any relevant allergy/hypersensitivity (including known immediate or delayed hypersensitivity reaction or idiosyncrasy to drugs chemically related to the compound of Formula I or its excipients); known history of active hepatitis B or C; receipt of any other investigational agent within 30 days or a washout of ≥5 half-lives, whichever is longer; active CNS disease; malabsorption syndrome or other illness that could affect the oral absorption of the compound of Formula I; history of acute pancreatitis within 1 year of study or history of chronic pancreatitis; clinically significant, uncontrolled, or active cardiovascular disease; uncontrolled intercurrent illness that might compromise patient safety or interfere with the evaluation of the safety of the compound of Formula I; patients eligible for potentially curative therapy (e.g. hematopoietic stem cell transplantation); autologous or allogeneic stem transplantation ≤3 months prior to screening; any evidence of ongoing graft versus host disease (GVHD) or GVHD requiring immunosuppressive therapy ≤28 days prior to the first compound of Formula I administration; another primary malignancy within the past 3 years or earlier apart from non-melanoma skin cancer or cervical carcinoma in situ. Further, the following were not permitted prior to the compound of Formula I administration: radiotherapy or cytotoxic chemotherapy within 24 days (42 days for nitrosoureas or mitomycin C); interferon, cytarabine, or immunotherapy within 14 days prior; or a targeted therapy such TKIs not completing washout of ≥5 half-lives. Patients who have not recovered to Grade ≤1 from AEs due to agents administered earlier with the exception of alopecia were not permitted to enroll. Patients with contraindications for repeated bone marrow sample collection and those who were exposed to the compound of Formula I in previous studies, patients having difficulty in swallowing oral medications and patients who were not able to undergo venipuncture and/or tolerate venous access were also excluded from the current study.

Total subjects enrolled for this study were 51 out of which 41 subjects were for dose escalation and 10 were for dose expansion study.

### Disease History and Prior CML Treatment

In the dose escalation study, majority of the subjects enrolled were CP CML (n=30, 73.2%) followed by AP CML (n=5, 12.2%), BP CML (n=4, 9.8%) and Ph+ ALL (n=2, 4.9%). The median time from initial diagnosis for the study population was 10.64 years (range, 0.68 to 22.85). Baseline mutation status was available for all the subjects at baseline, 31.7% of subjects had 1 mutation and 4.9% had ≥2 BCR-ABL mutations. Eight (20.5%) subjects entered the study in complete cytogenetic response (1 subject each at 24 and 240 mg; 2 subjects at 48, 174 and 204 mg). Two subjects were missing baseline cytogenetic assessments. For most of the subjects, the response to last TKI prior at screening was refractory CML (n=21, 51.2%), followed by intolerant CML (n=15, 36.6%), and refractory and intolerant CML (n=5, 12.2%).

The enrolled subjects were heavily pre-treated with TKI and conventional therapies for CML. Of the 41 subjects enrolled in dose-escalation, 18 (43.9%) subjects received ≥4 prior BCR-ABL1 TKI therapies, 33 (80.5%) subjects received ≥3 prior BCR-ABL1 TKIs. Five (12.2%) had received one prior TKI therapy. Majority of the subjects had either failed or become intolerant to imatinib (85.4%), dasatinib (78.0%), ponatinib (53.7%), nilotinib (51.2%), bosutinib (41.5%), radotinib (22.0%), and asciminib (7.3%). Sixteen subjects (39%) across the dose-escalation cohorts received hydroxycarbamide for controlling their white blood cell counts and 4 subjects had bone marrow transplants. Additionally, subjects had received alternative treatments such as chemotherapy, investigational agents, and biologics for their CML.

In the dose expansion study, only CP CML subjects were enrolled. The median time from the time of initial diagnosis for the study population was 11.66 years (range, 1.43 to 23.00). Baseline mutation status was available for 8 of the 10 enrolled subjects at baseline. Two (20%) subjects had 1 mutation and 1 subject (10%) had ≥2 BCR-ABL mutations. Baseline cytogenetic assessments were available for all 10 enrolled subjects and none of the subjects entered the study in complete cytogenetic response. For most of the subjects, the response to last TKI prior at screening was refractory CML (n=6, 60%), followed by intolerant CML, and refractory and intolerant CML (n=2, 20.0%).

Of the 10 subjects enrolled in the study 5 (50%) had received one, 4 (40%) subjects had received 2 prior TKI therapy while 1 (10%) received 3 prior TKI. Prior TKI therapies in the study population include imatinib (100%), nilotinib (50%), and dasatinib (10%). Four (40%) subjects received hydroxycarbamide for controlling their white blood cell counts. The enrolled subjects were less heavily pre-treated in comparison to the dose-escalation cohort reflecting population from geographies wherein access to second-generation [2G] BCR-ABL1 TKIs is limited.

### Study Design:

In this study, the dose-escalation cohort started with an accelerated titration study design (ATD) and shifted to a 3±3 design when subjects in the preceding ATD cohort experienced either grade 2 or higher adverse events (AEs) or dose-limiting toxicity (DLT) during Cycle 1 that was not clearly and incontrovertibly related to the underlying disease or concomitant medications. The DLT period consisted of 28 days (Cycle 1). Subjects experiencing DLTs were managed with dose delays and/or discontinuation and continued in the study until disease progression. Decisions to advance to a succeeding dose level occurred after the evaluation of safety and tolerability data for a given dose level.

Accordingly, the eligible subjects were treated in 4-week cycles of once daily self-administration of the compound of Formula I. The administration was after an overnight fast, or after maintaining approximately 2 hours of fasting before and after the dosing in all cycles.

For the cohorts, where the 3±3 design applies, 3 subjects were enrolled initially. If 2 of these subjects experienced a Grade 2 or more toxicity (that was not clearly and incontrovertibly related to the underlying disease or concomitant medications), subsequent cohorts were escalated according to smaller increments. Further, if there were no DLTs in the first 3 subjects, dose escalation to the next higher dose level was allowed to proceed. If 1 of the 3 subjects experienced a DLT, that dose level was expanded to 6 subjects (3 additional subjects were enrolled). If 2 subjects at a given dose level experienced a DLT, the MTD was considered to have been exceeded. The next lower dose level was expanded to 6 subjects, or if 6 subjects had already been enrolled and no more than 1 of the 6 subjects experienced a DLT, then this lower dose level was considered the MTD. Additional subjects were enrolled in dose escalation cohorts for further assessment of safety and efficacy of the compound of Formula 1 at doses that were tolerated (i.e., doses that have <2 DLTs in 6 enrolled subjects or <1 DLT in 3 enrolled subjects). Thus 204 mg was arrived at as MTD. A subject experiencing a DLT was allowed to continue therapy with a reduced dose. Subjects who experienced a DLT and recovered were treated at the next lower dose level until disease progression or other events requiring further dose reduction existed. For cohorts at Dose Level 2 or higher, a further dose reduction (i.e., up to 2 levels lower than their originally assigned dose) was implemented in the case of a further DLT or other non-DLT events requiring a dose reduction, if and when the event leading to dose reduction returned to Grade 1 or lower in severity. The reduced dose was re-escalated to the original or higher dose (i.e., the dose for which safety had been established in the dose escalation cohort) considering subject safety and response to the compound of Formula I. The subject was discontinued from treatment using the compound of Formula I, if dose reduction below 48 mg was required.

Of all the doses evaluated (from 12 mg to 240 mg) in the multicenter, multi-dose, dose-escalation study, the 174 mg dose was found to be well-tolerated and associated with improvement in cytogenetic and molecular responses hence it was selected as a clinical dose. 19 subjects enrolled at 174 mg dose in Phase 1 study, out of which all subjects had a major hematological response (MaHR) as their best response, 10 subjects had a major cytogenetic response (MCyR) as their best response 9 subjects achieved complete cytogenetic response (CCyR) and 1 achieved partial cytogenetic response (PCyR). Apart from this, 4 subjects achieved major molecular response as their best response. In addition to these 19 subjects, four subjects from lower doses were dose escalated to 174 mg to achieve a deepening of response.

Further, the dose expansion study was carried out by adding up to 12 additional subjects in each of the subgroups as expansion cohorts starting at the MTD or RP2D for each subset of subjects. Three subjects were enrolled into each of the subgroup expansion cohorts and monitored for DLTs. The second set of up to 9 subjects was enrolled if the dose escalation criteria of DLTs in less than or equal to 1 out of 3 subjects was met.

In this study, the subjects were admitted based on prior screening of subject's disease diagnosis and status. During screening the bone marrow aspiration, BCR-ABL mutational analysis, and BCR-ABL transcript analysis were performed to re-confirm the disease status of the subject if not done within the previous 3 months. The screening was performed within 21 days prior to the first dose of the study drug.

The study was conducted over a period of 60 months (5 years) which was divided in 28-days cycle. In the first two cycles, the dose administration was started on day 1 and the subject's assessments were performed on day 1, day 2, day 3, day 8, day 15 and day 22. From Cycle 3 onwards, the assessments were performed every 3 months (i.e., on day 28 of Cycle 3, followed by day 28 of Cycle 6 which was further followed by day 28 of Cycle 9). The assessment of the subject included a physical examination to check subject's performance status according to Eastern Cooperative Oncology Group (ECOG), checking of vital signs such as temperature, heart rate, respiratory rate, and diastolic and systolic blood pressure, performing blood and urine analysis, confirming adverse events (AEs) due to the compound of Formula I. Apart from such analysis, the disease response to the compound of Formula I was assessed wherein a Hematological response (HR) assessment was performed on 1^{st} day of every cycle in during cycle 1 and 2 and on 28^{th} day from 3^{rd} cycle and every 3^{rd} month thereafter. Similarly, cytogenetic response was done by bone marrow aspirates and molecular response was assessed on 28^{th} day of 3^{rd} cycle and every 3^{rd} month thereafter.

The PK sampling time points included 24-hour serial sampling on Day 1 of Cycle 1 and Cycle 2 to characterize single and multiple-dose PK. Specifically, the PK sampling in Cycle 1 and/or Cycle 2 and subsequent cycles were included: Day 1 of Cycle 1: Predose, 0.5, 1, 2, 2.5, 4, 8, 24 hours (i.e., Day 2) postdose. A±5 minutes window permitted till 4 hours PK sample collection. A±10 minutes window permitted for subsequent PK sample collection. Days 3, 8, 15, and 22 of Cycle 1:30 (±10) minutes Predose only. Day 1 of Cycle 2 (Day 29 from start of treatment): Predose, 0.5, 1, 2, 2.5, 4, 8, 24 hours (i.e., Day 30) postdose. A #5 minutes window permitted till 4 hours PK sample collection. A #10 minutes window permitted for subsequent PK sample collection. Days 8, 15, and 22 of Cycle 2:30 (±10) minutes Predose only. Cycle 3 onwards: Predose samples on Day 1.

Pharmacokinetic analysis was performed using non-compartmental analysis. The actual elapsed time from dose was used in the final PK parameter calculations. Plasma concentrations and PK parameters for the compound of Formula I were listed and summarized by dose cohort using descriptive statistics (n, mean, standard deviation, minimum, median, and maximum). Geometric mean and geometric coefficient of variation were calculated for PK parameters. Additionally, mean concentration profiles by dose cohort and cycle were illustrated. Dose proportionality of Cₘₐₓ and AUC₍₀₋₂₄₎ was assessed graphically for Cycle 1 and Cycle 2. Dose proportionality was statistically evaluated using the power model approach, whenever the number of patients was at least 3 for a given cohort. Time to steady-state (C_{trough}) was evaluated graphically. Differences among dose levels in CL/F and V/F, and dose-normalized Cₘₐₓ and AUC₍₀₋₂₄₎ were explored using an analysis of variance.

Concentrations of the compound of Formula I in plasma were measured using a validated LC-MS/MS method with a lower limit of quantitation of 2.5 ng/ml. The PK parameters for the study were summarized by treatment using descriptive (N, n, mean, standard deviation [SD], coefficient of variation [CV] % minimum, median, maximum, geometric mean and CV %) statistics, except for Tₘₐₓ. For Tₘₐₓ, the number of subjects, minimum, median, and maximum are reported.

Whenever the subject experienced DLTs or AEs due to the compound of Formula I, their treatment was continued with a reduced dose. In certain cases, the treatment was discontinued and resumed at a lower dose after the subject recovered from the DLT or AEs. The reduced dose was re-escalated to the original or higher dose. Whenever subjects experienced grade 3 or grade 4 toxicities attributable to the compound of Formula I, the treatment was continued at a reduced dose. In some cases, such as grade 3 and 4 hematologic toxicities, the treatment was discontinued for 2 weeks for the subject to recover and then resumed at a lower dose. The subject was said to be recovered when the toxicity levels were reduced to grade 1 or were completely resolved. The treatment was discontinued permanently if a dose reduction below 48 mg was required.

### Results:

52 subjects were enrolled in dose escalation and dose expansion cohorts. Forty-one of these subjects that were enrolled for dose escalation studies received doses from 12 mg to 240 mg. Among these subjects, 32 were in chronic phase CML (CP CML), 3 in accelerated phase CML (AP CML), 4 in blast phase CML (BP CML), and 2 were suffering from Ph+ ALL. Also, 11 CP CML patients were enrolled in the dose expansion cohort at 174 mg dose. The baseline demographics and disease history are set forth in Table 10:

TABLE 10
Baseline Demographics and Disease History

| | Dose | | Dose | |
|---|---|---|---|---|
| | Escalation N | | Expansion N | |
| Parameter | (%) | | (%) | |
| Number of Patients | 41 | | 10 | |
| Median age in years (range) | 61 | (23-85) | 47 | (35-60) |

Response to prior therapy

| | | | | |
|---|---|---|---|---|
| Refractory to prior TKI | 24 | (58%) | 7 | (64%) |
| Intolerant to prior TKI | 17 | (42%) | 4 | (36%) |

Previous TKI therapy

| | | | | |
|---|---|---|---|---|
| Median number of TKIs received (range) | 3 | (1-6) | 2 | (1-3) |

| | | | |
|---|---|---|---|
| Patients receiving ≥4 TKIs | 19 | (46%) | — |
| Patients receiving prior Ponatinib | 22 | (54%) | — |

Other previous therapies

| | | | |
|---|---|---|---|
| Omacetaxine | 5 | (12%) | NA |
| Chemotherapy (excluding hydroxyurea) | 9 | (22%) | NA |

Of the 41 subjects enrolled across the dose escalation cohorts, 11 subjects were discontinued from the study: 4 subjects due to AEs (1 subject each at 48, 126, 174 and 204 mg), 4 subjects due to disease progression (1 subject each at 48 mg and 240 mg, 2 subjects at 66 mg), and 1 subject each due to loss to follow up (at 66 mg), death (at 126 mg) and other reason (at 66 mg).

Of the 10 subjects enrolled for dose expansion at 174 mg in dose expansion cohort, only 1 subject was discontinued from the study due to AE.

TABLE 11
Efficacy Results in CP CML Subjects

| | 12 mg | 24 mg | 48 mg | 66 mg | 90 mg | 126 mg | 174 mg | 204 mg | 240 mg | Total | 174 mg |
|---|---|---|---|---|---|---|---|---|---|---|---|
| | (N = 1) | (N = 0) | (N = 3) | (N = 4) | (N = 2) | (N = 4) | (N = 7) | (N = 6) | (N = 3) | (N = 30) | (N = 10) |
| | ESC | | | | | | | | | | EXP |
| Best Hematologic Response | | | | | | | | | | | |
| Major Hematologic | 1(100) | 0 | 2(66.7) | 3(75.0) | 2(100) | 4(100) | 4(57.1) | 6(100) | 2(66.7) | 24(80.0) | 10(100) |
| Response | | | | | | | | | | | |
| Complete Response | 1(100) | 0 | 2(66.7) | 3(75.0) | 2(100) | 4(100) | 4(57.1) | 6(100) | 2(66.7) | 24(80.0) | 10(100) |
| No Evidence of Leukemia | 0 | 0 | 0 | 0 | 0 | 0 | 0 | 0 | 0 | 0 | 0 |
| No Response | 0 | 0 | 1(33.3) | 1(25.0) | 0 | 0 | 2(28.6) | 0 | 0 | 4(13.3) | 0 |
| Missing | 0 | 0 | 0 | 0 | 0 | 0 | 1(14.3) | 0 | 1(33.3) | 2(6.7) | 0 |
| Best Cytogenetic Response | | | | | | | | | | | |
| Major Cytogenetic | 1(100) | 0 | 2(66.7) | 2(50.0) | 1(50.0) | 2(50.0) | 3(42.9) | 6(100) | 3(100) | 20(66.7) | 7(70.0) |
| Response | | | | | | | | | | | |
| Complete Response | 1(100) | 0 | 2(66.7) | 2(50.0) | 0 | 2(50.0) | 3(42.9) | 4(66.7) | 3(100) | 17(56.7) | 6(60.0) |
| Partial Response | 0 | 0 | 0 | 0 | 1(50.0) | 0 | 0 | 2(33.3) | 0 | 3(10.0) | 1 (10.0) |
| Minor Response | 0 | 0 | 0 | 0 | 0 | 0 | 1(14.3) | 0 | 0 | 1(3.3) | 0 |
| Minimal Response | 0 | 0 | 0 | 1(25.0) | 0 | 1(25.0) | 2(28.6) | 0 | 0 | 4(13.3) | 3(30.0) |
| No Response | 0 | 0 | 0 | 1(25.0) | 1(50.0) | 1(25.0) | 0 | 0 | 0 | 3(10.0) | 0 |
| Missing | 0 | 0 | 1(33.3) | 0 | 0 | 0 | 1(14.3) | 0 | 0 | 2(6.7) | 0 |
| Best Molecular Response | | | | | | | | | | | |
| Major Response | 0 | — | 2(66.7) | 1(25.0) | 1(50.0) | 1(25.0) | 3(42.9) | 3(50.0) | 3(100) | 14(46.7) | 1(10.0) |
| Complete Response | 0 | 0 | 2(66.7) | 1(25.0) | 0 | 1(25.0) | 1(14.3) | 1(16.7) | 1(33.3) | 7(23.3) | 1(10.0) |
| No Response | (100) | 0 | 0 | 3(75.0) | 1(50.0) | 3(75.0) | 3(42.9) | 3(50.0) | 0 | 14(46.7) | 9(90.0) |
| Missing | 0 | 0 | 1(33.3) | 0 | 0 | 0 | 1(14.3) | 0 | 0 | 2(6.7) | 0 |

ESC = Part B Dose Escalation Cohort;
EXP = Part B Dose Expansion Cohort

Two of the 3, AP CML patients had baseline hematological response (CHR) with absence of cytogenetic and molecular response. The 3 patients further deepened their responses with 1 patient achieving CCyR with MMR and 2 patients in PCyR. Of the 4 BP CML patients, 2 achieved complete hematological response (CHR) and 2 patients had disease progression as their best response; Of the 2 Ph+ ALL pts, 1 pt maintained CCyR and MMR while the other reported disease progression as the best response (See Table 12).

TABLE 12
Efficacy Results in AP CML, BP CML and Ph + ALL Subjects

| Part B (Escalation and expansion cohort) | | | | | | | | | | |
|---|---|---|---|---|---|---|---|---|---|---|
| 12 mg | 24 mg | 48 mg | 66 ang | 90 mg | 126 mg | 174 mg | 204 mg | 240 mg | | 174 mg |
| (N = 0) | (N = 1) | (N = 3) | (N = 3) | (N = 1) | (N = 3) | (N = 0) | (N = 0) | (N = 0) | Total | (N = 0) |

| | |
|---|---|
| ESC | EXP |

Best Hematologic Response

| | | | | | | | | | | | |
|---|---|---|---|---|---|---|---|---|---|---|---|
| Major Hematologic | 0 | 1(100) | 3(100) | 2(66.7) | 1(100) | 0 | 0 | 0 | 0 | 7(63.6) | 0 |
| Response | | | | | | | | | | | |
| Complete Response | 0 | 1(100) | 3(100) | 0 | 1(100) | 0 | 0 | 0 | 0 | 5(45.5) | 0 |
| No Evidence of Leukemia | 0 | 0 | 0 | 2(66.7) | 0 | 0 | 0 | 0 | 0 | 2(18.2) | 0 |
| No Response | 0 | 0 | 0 | 1(33.3) | 0 | 1(33.3) | 0 | 0 | 0 | 2(18.2) | 0 |
| Missing | 0 | 0 | 0 | 0 | 0 | 2(66.7) | 0 | 0 | 0 | 2(18.2) | 0 |

Best Cytogenetic Response

| | | | | | | | | | | | |
|---|---|---|---|---|---|---|---|---|---|---|---|
| Major Cytogenetic Response | 0 | 1(100) | 2(66.7) | 3(100) | 1(100) | 0 | 0 | 0 | 0 | 7(63.6) | 0 |
| Complete Response | 0 | 1(100) | 2(66.7) | 2(66.7) | 1(100) | 0 | 0 | 0 | 0 | 6(54.5) | 0 |
| Partial Response | 0 | 0 | 0 | 1(33.3) | 0 | 0 | 0 | 0 | 0 | 1(9.1) | 0 |
| Minor Response | 0 | 0 | 0 | 0 | 0 | 1(33.3) | 0 | 0 | 0 | 1(9.1) | 0 |
| Minimal Response | 0 | 0 | 0 | 0 | 0 | 0 | 0 | 0 | 0 | 0 | 0 |
| No Response | 0 | 0 | 1(33.3) | 0 | 0 | 1(33.3) | 0 | 0 | 0 | 2(18.2) | 0 |
| Missing | 0 | 0 | 0 | 0 | 0 | 1(33.3) | 0 | 0 | 0 | 1(9.1) | 0 |

Best Molecular Response

| | | | | | | | | | | | |
|---|---|---|---|---|---|---|---|---|---|---|---|
| Major Response | 0 | 1(100) | 1(33.3) | 1(33.3) | 1(100) | 0 | 0 | 0 | 0 | 4(36.4) | 0 |
| Complete Response | 0 | 0 | 1(33.3) | 1(33.3) | 1(100) | 0 | 0 | 0 | 0 | 3(27.3) | 0 |
| No Response | 0 | 0 | 2(66.7) | 2(66.7) | 0 | 2(66.7) | 0 | 0 | 0 | 6(54.5) | 0 |
| Missing | 0 | 0 | 0 | 0 | 0 | 1(33.3) | 0 | 0 | 0 | 1(9.1) | 0 |

ESC = Part B Dose Escalation Cohort;
EXP = Part B Dose Expansion Cohort

Efficacy Responses in CP CML with prior ponatinib treatment are tabulated in Table 13. 16 CP CML patients had prior treatment involving ponatinib. Out of 16, 11 (68.8%) patients had Major Cytogenetic Response (MCyR, both PCyR+ CCyR) in which 6 patients achieved MCyR, 5 maintained MCyR. MMR was achieved by 8 (47%) patients out of 17 (See table 13).

TABLE 13
Efficacy Responses in CP CML with prior Ponatinib treatment: Best
Cytogenetic and Molecular Response (Achieved or Maintained)

| | Part B (Escalation and expansion cohort) | | | | | | | | | | |
|---|---|---|---|---|---|---|---|---|---|---|---|
| CML-CP subjects | 12 mg | 24 mg | 48 mg | 66 mg | 90 mg | 126 mg | 174 mg | 204 mg | 240 mg | | 174 mg |
| with ponatinib | (N = 0) | (N = 0) | (N = 1) | (N = 2) | (N = 2) | (N = 1) | (N = 5) | (N = 3) | (N = 2) | Total | (N = 10) |
| treatment | ESC | | | | | | | | | | EXP |
| MCyR | NA | NA | 1(100) | 1(50.0) | 1(50.0) | 0 | 3(60.0) | 3(100) | 2(100) | 11(68.8) | NA |
| Achieved MCyR | | | 0 | 1(50.0) | 1(50.0) | 0 | 1(20.0) | 2(66.7) | 1(50.0) | 6(37.5) | |
| Maintained MCyR | | | 1(100) | 0 | 0 | 0 | 2(40.0) | 1(33.3) | 1(50.0) | 5(31.3) | |
| Achieved MMR | | | 1(100) | 0 | 1(50.0) | 0 | 3(60.0) | 1(33.3) | 2(100) | 8(50.0) | |
| Maintained MMR | | | 0 | 0 | 0 | 0 | 0 | 0 | 0 | 0 | |

Median duration of treatment was 23 (0.5-51) months [CP CML 23 (0.5-51); AP CML 36 (9-40); BP CML 3 (0.5-18) and Ph+ ALL 4 (0.7-7.3) months].

Efficacy response in CP CML patients who were previously treated using more than 3 TKIs including ponatinib and in patients previously treated using more than 3 TKIs including ponatinib and asciminib, showed CCyR as best response in 46.7% and 33.3%, respectively. Major cytogenetic response as best response was reported in 66.7% and 33.3% of patients in ponatinib and ponatinib plus asciminib group (see Table 14).

TABLE 14
Efficacy outcome in patients that were previously
treated using ponatinib and ponatinib and asciminib

| | PON | | | | ASC | | |
|---|---|---|---|---|---|---|---|
| | (N = 15) | | | | (N = 3) | | |
| | Baseline | | Best Response | | Baseline | Best Response | |
| Hematological response | | | | | | | |
| CHR | 9 | (60.0) | 12 | (80.0) | 2 (66.7) | 3 | (100.0) |
| No response | 5 | (33.3) | 3 | (20.0) | 0 | 0 | |
| Missing | 1 | (6.7) | 0 | | 1 (33.3) | 0 | |
| Cytogenetic response | | | | | | | |
| Major cytogenetic response | 5 | (33.3) | 10 | (66.7) | 0 | 1 | (33.3) |
| Complete cytogenetic response | 4 | (26.7) | 7 | (46.7) | 0 | 1 | (33.3) |
| Partial cytogenetic response | 1 | (6.7) | 3 | (20.0) | 0 | 0 | |
| Minor response | 3 | (20.0) | 1 | (6.7) | 2 (66.7) | 1 | (33.3) |
| Minimal response | 1 | (6.7) | 1 | (6.7) | 0 | 0 | |
| No response | 4 | (26.7) | 2 | (13.3) | 0 | 1 | (33.3) |
| Missing | 2 | (13.3) | 1 | (6.7) | 1 (33.3) | 0 | |
| Molecular response | | | | | | | |
| Major molecular response | 0 | | 8 | (53.3) | 0 | 0 | |
| Complete Molecular Response | 0 | | 4 | (26.7) | 0 | 0 | |
| No response | 13 | (86.7) | 7 | (46.7) | 1 (33.3) | 3 | (100.0) |

| | | | | | |
|---|---|---|---|---|---|
| Missing | 2 | (13.3) | 0 | 2 (66.7) | 0 |

| | | |
|---|---|---|
| Average dose received per day | 123.7 | 123.7 |
| across all cycles (median, range) | (66-0-215.1) | (66.0-167.7) |

A multicenter open-label study in subjects with CML (CP CML, AP CML, and BP CML) who documented resistance or intolerance to more than or equal to 3 prior TKIs one of which included ponatinib was performed. The subjects for this were grouped as follows:
a) Cohort A—Ph+ CML: Chronic phase (CP CML) cohort
b) Cohort B—Ph+ CML: Accelerated phase (AP) cohort
c) Cohort C—Ph+ CML: Blast phase (BP) cohort.

The study was conducted according to globally accepted standards of Good Clinical Practice as defined in the International Council for Harmonisation E6 Guideline in agreement with the latest revision of the Declaration of Helsinki and in keeping with local regulations. This study and its relevant supplementary information were implemented after approval by the institutional review board at each participating site.

### Objective of Study:

The primary objective of this study was to evaluate the efficacy of the compound of Formula I in CP CML patients by cytogenetic outcomes and in AP CML and BP CML patients by hematologic outcomes. Secondary objectives included evaluation of anti-leukemic efficacy of compound of Formula I by molecular outcomes, hematological outcomes in CP CML patients, and by cytogenetic outcomes in AP CML and BP CML; time to first hematologic response; duration of response; progression-free survival (PFS) and overall survival (OS), population PK of the compound of Formula I and safety.

In this study, the patient population of each cohort was evaluated separately for efficacy and safety. The subjects were treated in 4-week cycles using once-daily, oral administration of the compound of Formula I and evaluated for anti-CML responses (hematological, cytogenetic and molecular response). The Adverse events (AEs) were assessed throughout the study until 30 days after discontinuation of administration of the compound of Formula I and were graded as per National Cancer Institute, Common Terminology Criteria for Adverse Events (NCI-CTCAE) Version 5.0.

### Selection Criteria:

The selection criteria for subjects (Both inclusion and exclusion) were the same as those set forth above study, except for the following: Subjects had to be previously treated with ≥3 prior TKIs including the 3^{rd} generation TKI, ponatinib; subjects with Ph+ ALL were not included in this study; subjects with presence of T3151 mutations were excluded in the study. Limited to no response on a TKI therapy is reported in CML patients post-ponatinib treatment (Leuk Lymphoma 2018 June; 59 (6): 1312-1322).

### Disease History and Prior CML Treatment

For subjects enrolled at 174 mg of the compound of Formula I, the majority were CP CML (n=5, 71.4%) followed by AP CML (n=2, 28.6%). The median time from the time of initial diagnosis for the study population was 19.69 years (range, 2.91 to 29.33). One subject in CP CML was enrolled at 90 mg of the compound of Formula I. Baseline mutation status was available for all the subjects at baseline, 3 (42.9%) of subjects had 1 mutation and 1 subject (14.3%) had ≥2 BCR-ABL mutations. No subjects entered the study in complete cytogenetic response. None of the subjects were missing baseline cytogenetic assessments. For most of the subjects, the response to last TKI prior at the screening was refractory CML (n=5, 71.4%) and intolerant CML (n=2, 28.6%).

All 7 subjects dosed at 174 mg received ≥3 prior TKI which included imatinib, dasatanib and ponatinib in all 7 subjects. Other common TKIs in these subjects included bosutinib (71.4%), nilotinib (57.1%), and asciminib (42.9%). Three (42.9%) subjects received hydroxycarbamide for controlling their white blood cell counts.

An additional enrollment of 12 subjects was completed in this study (CP CML=10; AP CML=2). All 12 subjects were dosed at 174 mg and received ≥3 prior TKI one of which was ponatinib. The combined information for all 19 subjects (12+7 subjects) represents CML population resistant or intolerant to at least 3 prior TKIs, one of which included ponatinib. One subject was enrolled at a dose of 90 mg.

Overall, of the subjects enrolled at 174 mg of the compound of Formula I, the majority were CP CML (n=15, 78.9%) followed by AP CML (n=4, 21.1%). The median time from the time of initial diagnosis for the study population was 6.3 years (range, 2.1 to 29.3). Baseline mutation status was available for all the subjects at baseline, 5 (26.3%) of subjects had 1 mutation and 1 subject (5.2%) had >2 BCR-ABL mutations. No subjects entered the study in complete cytogenetic or molecular response. None of the subjects were missing baseline cytogenetic assessments. The details are summarized in Table 15. Overall the study population was more heavily pre-treated with limited to no treatment options.

TABLE 15
Demographics and baseline characteristics of CML Subjects

| | | | CML-AP and |
|---|---|---|---|
| | All Subjects | CML-CP | CML-BP |
| | (N = 19) | (N = 15) | (N = 4), |
| | n (%) | n (%) | n (%) |
| Median age in years (range) | 61(35, 77) | 61(35, 77) | 55.5(49, 73) |
| Male, n (%) | 9(47.4) | 6(40) | 3(75) |
| Median time since diagnosis in | 6.3(2.1, 29.3) | 4.4(2.1, 29.3) | 14.1(6.3, 19.8) |
| years (Range) | | | |
| Baseline Mutations, n (%)¹ | | | |
| 1 Mutation | 5(26.3) | 5(33.3) | 0 |
| Number of Prior TKIs Received, n (%) | | | |
| ≥3 | 19(100) | 15(100) | 4(100) |
| Prior Ponatinib | 18(94.7) | 14(93.3) | 4(100) |
| Prior Asciminib | 3(15.8) | 3(20) | 0 |
| Other therapies Received, n (%) | | | |
| Interferon | 5(26.3) | 3(20) | 2(50) |
| Omacetaxine | 1(5.3) | 1(6.7) | 0 |

¹Data for 1 subject with 2 mutations at baseline not entered in the database.

### Study Procedures:

All patients underwent baseline assessments within 24 days after signing the informed consent form. A unique number was assigned to each patient immediately after informed consent was obtained and served as the patient's identifier.

Data protection and privacy regulations were followed in capturing, forwarding, processing, and storing patient data.

Study assessments monitored the efficacy and safety of the compound of Formula I. The efficacy assessments at baseline included cytogenetic, molecular, and hematology evaluations and sequencing for identification of the BCR-ABL kinase domain mutation, if any. Cytogenetic and molecular assessments were carried out by central labs. Hematologic assessments were completed within the −7 to −1 days of the patient was dosed by the site's local laboratory. Safety assessments at baseline included clinical evaluations, electrocardiograms, complete blood counts, and serum chemistry tests.

The following mutations were seen in subjects across the study: 1 M244V, 1 L248V, 1 E499E, 2 Q252H, 1 (P223S, A337T), 1 F359C, 1 E255V, 2 T315I, 1 Y253F, 3 F317L, 1 Y253H, 2 V299L, 1 (E255V, F317L), 1 (E255V, F359V), 2 G250E, 1 E255K, 1 F359V, 1 M351T, 1 (G250E, H369R), 1 H396R, and 1 V359I.

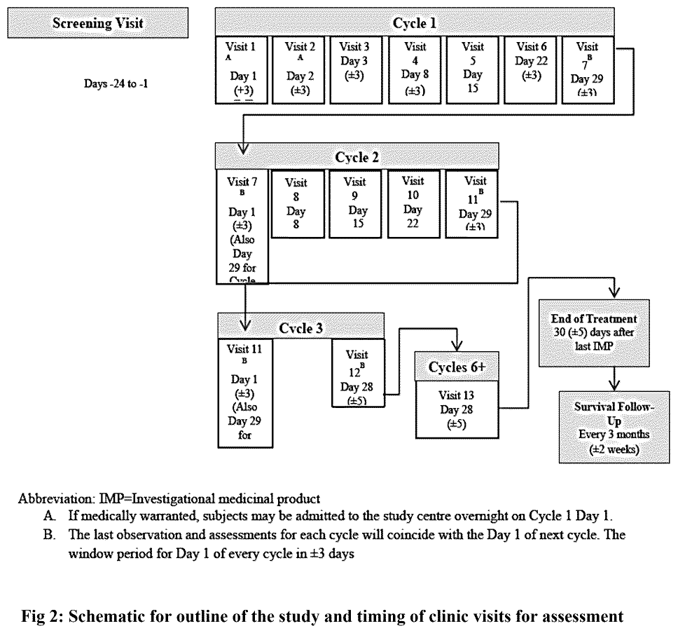

A schematic for the outline of the study and the timing of clinic visits for assessment is given in FIG. 2.

Patients were evaluated every 7 days in 4-week cycles up until Cycle 3 day 1, after which they were required to visit the site on Cycle 3 day 28; and once every 3 cycles thereafter for complete blood counts, chemistry analyses, and efficacy assessments. Each cycle visit included clinical evaluations, electrocardiograms, complete blood counts, and serum chemistry tests. Efficacy assessments consisting of bone marrow aspiration for cytogenetic analysis with molecular testing to measure the BCR/ABL1 transcript levels were carried out every 3 months. On discontinuation, patients were required to visit the site for an End of Treatment (EoT) visit which happened 30 days after the date of discontinuation of therapy. Patients were followed for survival by telephone survey.

### Study Treatment:

The Compound of Formula I with 174 mg as capsules was administered orally once daily continuously in 4-week cycles. Patients self-administered the compound of Formula I on an outpatient basis at approximately the same time each morning after overnight fasting or fasting ˜2 hours before and after dosing.

### Dosage and Administration:

The compound of Formula I was orally administered as a hard gelatin capsule, wherein the capsule may further comprise pharmaceutically acceptable excipients. The compound of Formula I was self-administered at the same time each morning preferably after overnight fasting or after maintaining approximately two hours of fasting before and after administration. Water was permitted ad-libitum until 1 hour before administration of the compound of Formula I and again from 1 hour after administration. Whenever the patient forgot to take his/her daily dose, he/she was allowed to take it within 6 hours after the missed dosing time. In cases where 6 hours had passed, the dose was omitted and the patient continued the treatment with the next scheduled dose. Similarly, whenever the patient vomited during the course of treatment, there was no re-dosing allowed, instead, the patient continued the treatment with next scheduled dose.

Usually, the patient started the treatment with an initial daily dose of 174 mg. However, depending on the patient's disease history, prior treatments and the history of adverse events towards prior treatments the initial dose was varied between 12 mg to 204 mg. In cases of newly diagnosed patients, the initial daily dose is 174 mg, which is then withheld, reduced or escalated based on the disease response and adverse events. For refractory patients, the initial dose varied between 48 mg to 174 mg. More particularly for refractory patients, the initial daily dose was selected from 48 mg, 66 mg, 90 mg, 126 mg and 174 mg.

Dose delays or reductions or both were implemented for patients who experienced adverse drug reactions due to the compound of Formula I:
a) For the patients who experienced grade 1 or 2 hematological or non-hematological toxicity due to the compound of Formula I the treatment was continued without any modifications if these toxicities were managed with supportive care or were non-interfering with normal daily activities of the patient. In such cases, the dose was not withheld or reduced;
b) In case the patient experienced Grade 1 or 2 non-hematological toxicities that were intolerable either due to clinical symptoms or due to interference with daily activities and if such toxicities were not controlled by optimal supportive care or optimal management, the treatment was withheld for up to 14 days (dose delay) to allow for amelioration or resolution of the event to Grade 1 or baseline. The patient resumed the treatment at the same dose or lower dose depending on the toxicities, time of recovery from the toxicities and the response towards the dose. When the toxicities in any patient were not controlled within 14 days despite of optimal care and management, the treatment was discontinued. However, for patients that showed evidence of disease response or a developing disease response, the treatment was withheld for up to 28 days. In such cases, the dose was delayed or withheld for more than 14 days, and the patient resumed the treatment at a lower dose after amelioration or resolution of the event to Grade 1 or baseline. Here, the patient was said to be showing evidence of disease response when there was a hematologic response or partial or complete cytogenetic response or Major molecular response. In cases when the patient had a history or response to the previous treatments, the disease response using the compound of Formula I was compared with the patient's previous responses. The toxicity events were said to be resolved to baseline when the patient resumed the same conditions (both hematologic and non-hematologic) as those before the treatment with the compound of Formula I was initiated;
c) In the event of recurrences of the grade 1 or 2 non-hematological or hematologic toxicities, b) was repeated. In case of every incidence of recurrences of toxicities, the treatment was resumed with a lower dose than the previous one. The refractory patients having grade 1 or 2 toxicities due to 48 mg dose and the newly diagnosed patients having such toxicities due to 12 mg dose of the compound of Formula I, were discontinued from the treatment, if the patient did not recover from such toxicities with supportive care and if the toxicities interfered with the daily activities;
d) For patients with chronic phase (CP) CML, in the event of grade 3 hematological or non-hematological toxicity or grade 4 asymptomatic hematological toxicities and for patients with acute phase (AP) CML and blast phase (BP) CML in the event of grade 3 non-hematological toxicity, the treatment was withheld for up to 28 days to allow for recovery of patient to baseline or at least to grade 1 toxicity. The treatment was resumed at the same dose after the patient recovered from said toxicities. The treatment was discontinued in subjects whose toxicities were not resolved to grade 1 or baseline by 28 days. However, in cases of patients showing evidence of disease response or a developing disease response the time for recovery or resolution of toxicities to grade 1 or baseline was extended until 56 days. Patients recovering within 56 days continued with the treatment and those who did not recover within 56 days were discontinued. Here, the patient was said to be showing evidence of disease response when there was complete hematologic response (CHR) or partial or complete cytogenetic response;
e) In the event of grade 3 hematological or grade 4 asymptomatic hematological toxicities in patients with acute phase (AP) CML and blast phase (BP) CML, tests were conducted to confirm that the toxicities were attributable to the compound of Formula I and not to the underlying disease. The treatment was discontinued in subjects whose toxicities were not resolved to grade 1 or baseline by four weeks. However, in cases of patients showing evidence of disease response or a developing disease response the time for recovery or resolution of toxicities to grade 1 or baseline was extended until 56 days. Patients recovering within 56 days continued with the treatment and those who did not recover within 56 days were discontinued. In the case of AP CML and BP CML patients, the patient was said to be showing evidence of disease response when there was CHR;
f) For every event of recurrences of toxicities in CP CML, AP CML and BP CML patients the protocol given in d) and e) was repeated as per the conditions of the patient and the treatment was resumed with the next lower dose. The refractory patient having toxicities due to the 48 mg dose and the newly diagnosed patient having toxicities due to the 12 mg dose of the compound of Formula I, was discontinued from the treatment.

### Assessment of Cytogenetic, Major Molecular, and Hematological Responses.

Bone marrow aspirate samples were sent for review of cytogenetics by conventional Giemsa staining method (karyotyping). Fluorescence in situ hybridization (FISH) was performed on bone marrow aspirates only in cases of subjects who had achieved complete cytogenetic response (confirmed by karyotyping) with the studied drug and who had minimum residual disease. FISH was also used if a sufficient sample was not available for conducting the Giemsa staining method (karyotyping).

For subjects who demonstrated complete cytogenetic response on two repeated assessments, bone marrow aspirate samples could be collected at 6 monthly intervals thereafter.

Standard cytogenetic methods were used to determine the presence of the Ph+ and its presence (percentage) in the bone marrow. At least 20 cells in metaphase were evaluated for karyotyping. Bone marrow cytogenetics was scheduled to be performed within 7 days of the scheduled date irrespective of missed doses or disease or dose-associated AEs.

Peripheral blood cells were not used for conventional cytogenetic response monitoring.

Molecular response monitoring (MMR) using PCR for BCR-ABL copy number was performed on the collected blood samples and was assessed using the international scale (IS).

The hematologic response was evaluated by peripheral blood utilizing local laboratory results.

The hematologic response was usually anticipated within the first 3 months of treatment, however, some subjects took longer to achieve this level. PK parameters were studied similarly to the earlier mentioned study.

### Results:

Major hematological response (MaHR) was reported as the best response in 6 (85.7%) of the 7 enrolled subjects in the study. Of these 6, 1 subject came into the study in MaHR and maintained it, while 5 achieved MaHR in the study. All 6 subjects with MaHR, had a complete hematological response.

Major cytogenetic response (MCyR) was reported as the best response in 2 (28.6%) of the 7 enrolled subjects. Of these 2, 1 subject came into the study in PCyR and deepened response to achieve CCyR; while the other developed PCyR in the study. So, the best response in the 2 subjects, was CCyR and PcyR respectively. Both the subjects demonstrating MCyR belonged to the CP CML cohort.

Major molecular response (MMR) was reported as the best response in 1 (14.3%) of the 7 enrolled subjects.

TABLE 16
Efficacy Results in all patients registered:

| | |
|---|---|
| Disease Response | 174 mg (N = 7) |

Best Hematologic Response

| | |
|---|---|
| Major Hematologic Response | 6 (85.7%) |
| Complete Hematologic Response | 6 (85.7%) |
| No evidence of Leukemia | 0 |
| No response | 1 (14.3%) |
| Missing Response | 0 |

Best Cytogenetic Response

| | |
|---|---|
| Major Cytogenetic Response | 2 (28.6%) |
| Complete Cytogenetic Response | 1 (14.3%) |
| Partial Cytogenetic Response | 1 (14.3%) |
| Minor Cytogenetic Response | 0 |
| Minimal Cytogenetic Response | 0 |
| No Response | 2 (28.6%) |
| Missing | 3 (42.9%) |

Best Molecular Response

| | |
|---|---|
| Major Response | 1 (14.3%) |
| Complete Response | 0 |
| No Response | 4 (57.1%) |
| Missing | 2 (28.6%) |

An additional enrollment of 12 subjects was completed in this study (CP CML=10; AP CML=2). The responses obtained by the overall 19 subjects (12+7) are summarized in Tables 12 and 13. Briefly, the median range of follow-up was 13.9 months (range 3.6-26.9 months). At the time of analysis, 9 (60%) of subjects were receiving treatment.

TABLE 17
Efficacy Responses in CP CML Subjects:

| | CP CML (N = 15) | | |
|---|---|---|---|
| | Overall | | |
| | Response | Achieved | Maintained |
| Median Follow-up duration | 13.9 | | |
| in Months (Range) | (3.6, 26.9) | | |
| Receiving treatment at time | 9(60) | | |
| of analysis, n (%) | | | |
| Hematological Response | | | |
| Complete Hematological | 13/15(86.7) | 4/4(100) | 9/11(81.8) |
| Response, n/m (%) | | | |
| Cytogenetic Response | | | |
| Major Cytogenetic | 11/13(84.6) | 4/6(66.7) | 7/7(100) |
| Response*, n/m (%) | | | |
| Complete Cytogenetic | 8/13(61.5) | 8/13(61.5) | |
| Response*, n/m (%) | | | |
| Molecular Response | | | |
| Major Molecular | 6/13(46.2) | 6/13(46.2) | |
| Response, n/m (%) | | | |

TABLE 18
Efficacy Responses in AP CML Subjects

| | AP CML (N = 4) | | |
|---|---|---|---|
| | Overall | | |
| | Response | Achieved | Maintained |
| Median Follow-up duration in | 16.6(1.4, | | |
| Months (Range) | 25.8) | | |
| Receiving treatment at time of | 4(100) | | |
| analysis, n (%) | | | |
| Hematological Response | | | |
| Complete Hematological | 2/2(100) | 2/2(100) | |
| Response, n/m (%) | | | |

Complete hematological response (CHR) was reported as the best response in 13 (86.7%) of the 15 CP-CML enrolled subjects in the study. Of these 13, 4 subjects came into the study in CHR and maintained it, while 9 achieved CHR in study. All 4 subjects with MaHR, had complete hematological response. Complete hematological response (CHR) was reported as the best response in 2 (100%) of the 2 AP-CML enrolled subjects in the study. Two subjects had not yet completed the required assessment period at this data cut-off.

Major cytogenetic response (MCyR) was reported as the best response in 11 (84.6%) of the CP-CML enrolled subjects. Of the 13 subjects, 11 (61.5%) developed CCyR.

Major molecular response (MMR) was reported as the best response in 6 (46.2%) of the 13 enrolled CP-CML subjects. Major cytogenetic and molecular responses were observed in heavily pre-treated CML subjects resistant or intolerant to at least 3 prior TKIs including ponatinib.

### Safety Evaluation Results Studies:

As can be seen in Table 19 in the overall 53 patients, any grade TEAEs were reported by 96.2% of patients and grade 3 or grade 4 TEAEs were reported in 64.1% of patients. Out of these 52, grade 3 grade 4 AEs were seen in 6 (33.4%) in patients that had undergone prior treatments with two TKIs, 28 (80%) in patients that had undergone prior treatments with three TKIs, 19 (86.3%) in patients that had undergone prior treatments with ponatinib as one of the TKI. Most frequently reported TEAEs included thrombocytopenia, which as observed in 41.5% out of which 18.8% were of grade 3/4, diarrhea was observed in 22% of patients with only 2% having grade 3 or grade 4 diarrhea, anemia was observed in 20.8% with 13% grade 3 or grade 4 and 20.8% suffered from cough. Hematological AEs of thrombocytopenia and neutropenia were similarly reported across the sub-groups.

TABLE 19
Summary of all TEAEs by preferred terms (>10%)

| | ≤2 prior TKIs | | ≥3 prior TKIs | | Prior Ponatinib | | Overall | |
|---|---|---|---|---|---|---|---|---|
| | (N = 18) | | (N = 35) | | (N = 22) | | (N = 53) | |
| | Any | Grade | Any | Grade | Any | Grade | Any | Grade |
| | grade | 3/4 | grade | 3/4 | grade | 3/4 | grade | 3/4 |
| All TEAEs | 16 | 6 | 35 | 28 | 22 | 19 | 51 | 34 |
| No of pts (N %) | (88.9%) | (33.4%) | (100%) | (80%) | (100%) | (86.3%) | (96.2%) | (64.1%) |
| Related to comp of | 12 | 3 | 23 | 11 | 15 | 7 | 35 | 14 |
| Formula I | (66.7%) | (16.7%) | (65.7%) | (31.4%) | (68.1%) | (31.8%) | (66.0%) | (26.4%) |
| Thrombocytopenia | 7 | 3 | 17 | 7 | 7 | 3 | 22 | 10 |
| | (38.8%) | (16.7%) | (48.5%) | (20%) | (31.8%) | (13.6%) | (41.5%) | (18.8%) |
| Diarrhoea | 1 | 0 | 11 | 1 | 7 | 1 | 12 | 1 |
| | (5.5%) | | (31.4%) | (2.8%) | (31.8%) | (4.5%) | (22.6%) | (1.8%) |
| Anemia | 2 | 2 | 9 | 5 | 6 | 4 | 11 | 7 |
| | (11.2%) | (11.2%) | (25.7%) | (14.2%) | (27.2%) | (18.1%) | (20.8%) | (13.2%) |
| Cough | 3 | 0 | 8 | 0 | 5 | 0 | 11 | 0 |
| | (16.7%) | | (22.8%) | | (22.7%) | | (20.8%) | |
| Constipation | 2 | 0 | 6 | 0 | 3 | 0 | 8 | 0 |
| | (11.2%) | | (17.1%) | | (13.6%) | | (15.1%) | |
| Headache | 1 | 0 | 7 | 0 | 4 | 0 | 8 | 0 |
| | (5.5%) | | (20%) | | (18.1%) | | (15.1%) | |
| Neutropenia | 2 | 2 | 6 | 5 | 3 | 2 | 8 | 7 |
| | (11.2%) | (11.2%) | (17.1%) | (14.2%) | (13.6%) | (9.1%) | (15.1%) | (13.2%) |
| Nausea | 0 | 0 | 8 | 0 | 7 | 0 | 8 | 0 |
| | | | (22.8%) | | (31.8%) | | (15.1%) | |
| Pyrexia | 2 | 0 | 6 | 1 | 4 | 1 | 8 | 1 |
| | (11.2%) | | (17.1%) | (2.8%) | (18.1%) | (4.5%) | (15.1%) | (1.8%) |
| Back pain | 1 | 0 | 6 | 0 | 3 | 0 | 7 | 0 |
| | (5.5%) | | (17.1%) | | (13.6%) | | (13.2%) | |
| Fatigue | 2 | 0 | 5 | 0 | 4 | 0 | 7 | 0 |
| | (11.2%) | | (14.2%) | | (18.1%) | | (13.2%) | |
| Myalgia | 1 | 0 | 6 | 0 | 2 | 0 | 7 | 0 |
| | (5.5%) | | (17.1%) | | (9.1%) | | (13.2%) | |
| Vomiting | 0 | 0 | 7 | 0 | 4 | 0 | 7 | 0 |
| | | | (20%) | | (18.1%) | | (13.2%) | |
| Decreased appetite | 1 | 0 | 5 | 0 | 3 | 0 | 6 | 0 |
| | (5.5%) | | (14.2%) | | (13.6%) | | (11.3%) | |
| Nasopharyngitis | 3 | 0 | 3 | 0 | 2 | 0 | 6 | 0 |
| | (16.7%) | | (8.5%) | | (9.1%) | | (11.3%) | |
| Pruritis | 2 | 0 | 4 | 0 | 4 | 0 | 6 | 0 |
| | (11.2%) | | (11.4%) | | (18.1%) | | (11.3%) | |
| Rhinitis | 2 | 0 | 4 | 0 | 4 | 0 | 6 | 0 |
| | (11.2%) | | (11.4%) | | (18.1%) | | (11.3%) | |
| Amylase increased | 0 | 0 | 5 | 4 | 3 | 2 | 5 | 4 |
| | | | (14.2%) | (11.4%) | (13.6%) | (9.1%) | (9.4%) | (7.5%) |
| Lipase increased | 0 | 0 | 5 | 4 | 2 | 2 | 5 | 4 |
| | | | (14.2%) | (11.4%) | (9.1%) | (9.1%) | (9.4%) | (7.5%) |
| SAEs | 6 | 2 | 14 | 10 | 10 | 7 | 20 | 12 |
| | (33.4%) | (11.2%) | (40%) | (28.5%) | (45.4%) | (31.8%) | (37.7%) | (22.6%) |
| Dose modifications | 1 | 1 | 10 | 5 | 8 | 4 | 11 | 6 |
| | (5.5%) | (5.5%) | (28.5%) | (14.2%) | (36.3%) | (18.1%) | (20.7%) | (11.3%) |
| Dose discontinued | 5 | 2 | 15 | 10 | 12 | 7 | 20 | 12 |
| and restarted | (27.2%) | (11.2%) | (42.8%) | (28.5%) | (54.5%) | (31.8%) | (37.7%) | (22.6%) |
| Leading to drug | 3 | 0 | 3 | 2 | 2 | 2 | 6 | 2 |
| withdrawal | (16.7%) | | (8.5%) | (5.7%) | (9.1%) | (9.1%) | (11.3%) | (3.7%) |
| Death | 3 | 0 | 2 | 0 | 2 | 0 | ^{#}5 | 0 |
| | (16.7%) | | (5.7%) | | (9.1%) | | (9.4%) | |

^{#}One death out of the 5 deaths recorded was a Grade 5 adverse event

An additional enrollment of 12 subjects was completed in the study (CP CML=10; AP CML=2). The safety data for the overall 19 subjects (12+7) who have failed ≥3 prior TKIs including ponatinib are summarized in Table 20. The most commonly observed TEAEs seen in subjects were thrombocytopenia (31.6%), nausea and rash (26.3% each), cough, dyspnea, fatigue and neutropenia (21.1% each). The most commonly observed Grade 3 or 4 TEAEs were thrombocytopenia (44.4%) and neutropenia (33.3%); all other Grade 3 or 4 events occurring in a single subject. The compound of Formula I was well tolerated in this heavily pre-treated CML population. There were no unusual or unanticipated safety signals observed.

TABLE 20
Most Frequent Adverse Events
(Incidence ≥10%) in CML Subjects

| | All Grades | Grade 3 or 4 | |
|---|---|---|---|
| | (N = 19) | (N = 9) | |
| | n (%) | n (%) | |
| Thrombocytopenia | 6 (31.6) | 4 | (44.4) |
| Nausea | 5 (26.3) | 1 | (11.1) |
| Rash | 5 (26.3) | 0 | (0) |
| Cough | 4 (21.1) | 0 | (0) |
| Dyspnoea | 4 (21.1) | 1 | (11.1) |
| Fatigue | 4 (21.1) | 0 | (0) |
| Neutropenia | 4 (21.1) | 3 | (33.3) |
| Anaemia | 3 (15.8) | 1 | (11.1) |
| Constipation | 3 (15.8) | 0 | (0) |
| Diarrhoea | 3 (15.8) | 1 | (11.1) |
| Dry skin | 3 (15.8) | 0 | (0) |
| Headache | 3 (15.8) | 0 | (0) |
| Hypertension | 3 (15.8) | 0 | (0) |
| Abdominal pain | 2 (10.5) | 0 | (0) |
| Arthralgia | 2 (10.5) | 0 | (0) |
| Back pain | 2 (10.5) | 0 | (0) |
| COVID-19 | 2 (10.5) | 1 | (11.1) |
| Chest pain | 2 (10.5) | 0 | (0) |
| Contusion | 2 (10.5) | 0 | (0) |
| Dizziness | 2 (10.5) | 0 | (0) |
| Flank pain | 2 (10.5) | 0 | (0) |
| Herpes zoster | 2 (10.5) | 0 | (0) |
| Hyperuricaemia | 2 (10.5) | 0 | (0) |
| Hypocalcaemia | 2 (10.5) | 1 | (11.1) |
| Hypokalaemia | 2 (10.5) | 1 | (11.1) |
| Hypophosphataemia | 2 (10.5) | 0 | (0) |
| Muscle spasms | 2 (10.5) | 0 | (0) |
| Nasopharyngitis | 2 (10.5) | 0 | (0) |
| Oropharyngeal pain | 2 (10.5) | 0 | (0) |
| Pancytopenia | 2 (10.5) | 1 | (11.1) |

The treatment using the compound of Formula I was found to be highly safe with respect to Cardiotoxicity. Table 21 indicates that overall, 14 (26.4%) patients out of 53 had any grade of cardiac and vascular adverse events (CAVEs) out of which only 3 (5.6%) were of grade 3 or 4. Two CVAEs considered related to the compound of Formula I: one Grade 2 hypertension and one fatal intracranial hemorrhage (ICH) with concomitant disease progression to BP CML. No dose modifications and 1 permanent discontinuation were reported due to CVAEs.

TABLE 21
Summary of all treatment-emergent cardiovascular AEs

| | ≤2 prior TKIs | | ≥3 prior TKIs | | Prior Ponatinib | | Overall | |
|---|---|---|---|---|---|---|---|---|
| | (N = 18) | | (N = 35) | | (N = 22) | | (N = 53) | |
| | Any | Grade | Any | Grade | Any | Grade | Any | Grade |
| | grade | 3/4 | grade | 3/4 | grade | 3/4 | grade | 3/4 |
| Adverse Events | 1 | 1 | 13 | 2 | 10 | 3 | 14 | 3 |
| Cluster - Cardiac | (5.5%) | (5.5%) | (37.1%) | (5.7%) | (45.4%) | (13.6%) | (26.4%) | (5.6%) |
| and vascular | | | | | | | | |
| events* (N %) | | | | | | | | |
| Hypertension^{a} | 0 | 0 | 4 | 1 | 2 | 1 | 4 | 1 |
| | | | (11.4%) | (2.8%) | (9.1%) | (4.5%) | (7.5%) | (1.8%) |
| Angina Pectoris^{b, c} | 0 | 0 | 2 | 0 | 1 | 0 | 2 | 0 |
| | | | (5.7%) | | (4.5%) | | (3.7%) | |
| Arteriosclerosis^{b} | 0 | 0 | 1 | 0 | 0 | 0 | 1 | 0 |
| | | | (2.8%) | | | | (1.8%) | |
| Cerebral | 0 | 0 | 1 | 0 | 1 | 0 | 1 | 0 |
| haemorrhage | | | (2.8%) | | (4.5%) | | (1.8%) | |
| Congestive heart | 1 | 1 | 0 | 0 | 1 | 1 | 1 | 1 |
| failure | (5.5%) | (5.5%) | | | (4.5%) | (4.5%) | (1.8%) | (1.8%) |
| Hot flush | 0 | 0 | 1 | 0 | 1 | 0 | 1 | 0 |
| | | | (2.8%) | | (4.5%) | | (1.8%) | |
| Hypotension^{a} | 0 | 0 | 1 | 0 | 1 | 0 | 1 | 0 |
| | | | (2.8%) | | (4.5%) | | (1.8%) | |
| Intermittent | 0 | 0 | 1 | 0 | 1 | 0 | 1 | 0 |
| claudication | | | (2.8%) | | (4.5%) | | (1.8%) | |
| Intracranial | 0 | 0 | 1 | 0 | 0 | 0 | 1 | 0 |
| haemorrhage^{d} | | | (2.8%) | | | | (1.8%) | |
| Mouth haemorrhage^{d} | 0 | 0 | 1 | 0 | 0 | 0 | 1 | 0 |
| | | | (2.8%) | | | | (1.8%) | |
| Palpitations^{b} | 0 | 0 | 1 | 0 | 0 | 0 | 1 | 0 |
| | | | (2.8%) | | | | (1.8%) | |
| Transient ischaemic | 0 | 0 | 1 | 0 | 1 | 0 | 1 | 0 |
| attack | | | (2.8%) | | (4.5%) | | (1.8%) | |
| Troponin I increased | 0 | 0 | 1 | 0 | 1 | 0 | 1 | 0 |
| | | | (2.8%) | | (4.5%) | | (1.8%) | |
| Syncope^{c} | 0 | 0 | 1 | 1 | 1 | 1 | 1 | 1 |
| | | | (2.8%) | (2.8%) | (4.5%) | (4.5%) | (1.8%) | (1.8%) |
| Ventricular | 0 | 0 | 1 | 0 | 0 | 0 | 0 | 0 |
| Extrasystoles | | | (2.8%) | | | | | |
| Serious Adverse | 0 | 0 | 4 | 1 | 3 | 1 | 4 | 1 |
| Events | | | (11.4%) | (2.8%) | (13.6%) | (4.5%) | (7.5%) | (1.8%) |
| Dose modifications | 0 | 0 | 0 | 0 | 0 | 0 | 0 | 0 |
| Dose interruption | 0 | 0 | 2 | 1 | 0 | 0 | 2 | 1 |
| and restart | | | (5.7%) | (2.8%) | | | (3.7%) | (1.8%) |
| Leading to drug | 0 | 0 | 1 | 0 | 0 | 0 | 1 | 0 |
| withdrawal | | | (2.8%) | | | | (1.8%) | |
| Deaths^{e} | 0 | 0 | 1 | 0 | 0 | 0 | 1 | 0 |
| | | | (2.8%) | | | | (1.8%) | |
| Related to study | 0 | 0 | 2 | 0 | 0 | 0 | 2 | 0 |
| drug | | | (5.7%) | | | | (3.7%) | |

*AE listing were gathered from study EDC with programmatic MedDRA coding and were run through following SMQs (Standard MedDRA Query)- 1) Cardiac failure(SMQ), 2) Central nervous system vascular disorders (SMQ), 3) Embolic and thrombotic events (SMQ), 4) Hypertension (SMQ), 5) Ischaemic heart disease (SMQ), 6) Cardiac arrhythmias (SMQ) to generate the above list of cardiac and vascular events . . . ;
^{a, b, c, d}these events took place in one pt each respectively;
^{e}One death on study was a Grade V adverse event

Table 22 below lists the medical history of patients and CVAEs that were reported in the patient during treatment. Thirty patients had a medical history of CVAEs of which 12 pts reported CVAEs in the study (10 CVAEs unrelated to the compound of Formula I and 2 related to the compound of Formula I).

TABLE 22
Subjects Cardiac and vascular medical history versus
Treatment-Emergent Adverse Events in Phase 1 study

| Patient | History of Cardiac and | Prior TKI | Ponatinib | CVAEs during |
|---|---|---|---|---|
| number | vascular disorders^{a} | therapy | received | treatment |
| 05600101 | Arteriosclerosis, Hypertension | 5 | Yes | Intermittent |
| | | | | claudication |
| 25000101 | Hypertension, Peripheral arterial | 4 | No | — |
| | occlusive disease | | | |
| 25000102 | Deep vein thrombosis | 5 | Yes | Troponin I |
| | | | | increased |
| 25000601 | Myocardial infarction | 3 | Yes | — |
| 25000602 | Mitral valve incompetence | 2 | Yes | — |
| 25000603 | Hypertension, Myocardial | 3 | No | Hypertension |
| | ischaemia | | | |
| 25000606 | Peripheral artery stenosis, | 3 | No | — |
| | Peripheral artery thrombosis, | | | |
| | Transient ischemic attack | | | |
| 35600105 | Hypertension | 2 | No | — |
| 35600503 | Intracranial hemorrhage | 3 | No | Intracranial |
| | | | | hemorrhage, |
| | | | | Oral hemorrhage |
| 35600507 | Hypertension | 2 | No | — |
| 35600705 | Ventricular extrasystoles | 3 | No | — |
| 35600706 | Acute myocardial infarction, | 3 | No | Hypertension |
| | myocardial ischemia | | | |
| 38000202 | Hypertension, Bicuspid aortic | 3 | Yes | — |
| | valve | | | |
| 38000401 | Hypertension, Myocardial | 5 | Yes | — |
| | infarction | | | |
| 41000101 | Hypertension | 3 | No | Angina pectoris, |
| | | | | Palpitations, |
| | | | | Arteriosclerosis |
| 41000104 | Sinus tachycardia | 3 | No | — |
| 41000106 | Bundle branch block left, Sinus | 4 | No | — |
| | bradycardia, Sinus tachycardia | | | |
| 41000108 | Hypertension | 3 | Yes | — |
| 41000109 | Hypertension | 6 | Yes | — |
| 82600202 | Hypertension, Myocardial | 5 | Yes | — |
| | ischemia | | | |
| 82600303 | Hypertension | 5 | Yes | Cerebral |
| | | | | haemorrhage |
| 84000201 | Aortic thrombosis, Hypertension, | 5 | Yes | — |
| | Hypotension, Palpitations, | | | |
| | Peripheral ischemia, | | | |
| | Tachycardia, Cardiac murmur, | | | |
| | Chronic mesenteric ischemia, | | | |
| | Renal infarction, Splenic | | | |
| | infarction | | | |
| 84000202 | Hypertension | 4 | Yes | Hypertension, |
| | | | | Hypotension |
| 84000203 | Hypertension | 6 | Yes | Hot flush |
| 84000204 | Arteriosclerosis coronary artery, | 4 | yes | — |
| | Hypertension, Pericarditis | | | |
| 84000205 | Hypertension | 3 | no | — |
| 84000206 | Bradycardia, Hypertension | 5 | Yes | Angina pectoris |
| 84000302 | Atrial fibrillation, Cardiac failure | 2 | Yes | Cardiac failure |
| | congestive, Hypertension, | | | congestive |
| | Myocardial infarction, | | | |
| | Cerebrovascular accident, | | | |
| | Transient ischemic attack | | | |
| 84000902 | Hypertension | 4 | No | |
| 84001001 | Hot flush, Pericarditis | 5 | yes | Transient |
| | | | | ischaemic attack |

^{a, b}All MH and AE listing were gathered from the study EDC with programmatic MedDRA coding and were run through following SMQs (Standard MedDRA Query)- 1) Cardiac failure (SMQ), 2) Central nervous system vascular disorders (SMQ), 3) Embolic and thrombotic events (SMQ), 4) Hypertension (SMQ), 5) Ischaemic heart disease (SMQ), 6) Cardiac arrhythmias (SMQ) to generate the above list of cardiac and vascular events.

Based on the clinical data, a dose range of 48 mg to 204 mg was found to provide a therapeutically efficacious and well-tolerated dose. This wide dose range was found to be necessary in view of the immense diversity observed in the patient population being studied including the elderly, heavily pre-treated (80%>3 prior TKI) and baseline mutation-positive patients. Higher drug doses were required to achieve disease response in patients that were heavily pre-treated and had difficult-to-treat mutations and additional chromosome anomalies (ACA). Simultaneously, intolerance was commonly observed in this patient population. The most common intolerance reported was hematological toxicity, which is a class effect of TKIs, and is almost invariably reported at higher doses and during the early phase (initial 3-4 months) of TKI therapy. Hematological intolerance usually resolves with drug hold or dose reductions. Thus, a lower dose that is well tolerated and therapeutically efficacious is required in such a patient population. Clinical data that supports the use of the compound of Formula I in a dose range from 48 mg to 204 mg is summarized below:

One AP CML human subject with a baseline mutation of Y253F was enrolled in the study at 48 mg. To achieve disease response after being on the therapy with the compound of Formula I at 48 mg for 12 cycles, the subject's dose was escalated in a sequential manner from 48 mg to 66 mg, 90 mg, 126 mg, and 174 mg prior to escalating to a 204 mg dose. With the initial lower dose escalation, there was an improvement in cytogenetic and molecular disease response, which improved with the further increase in dose to deepen the response. The subject's best response was demonstrated at 204 mg (Cytogenetic: 39 cycles Ph+10%, PCyR, and, molecular: 36 cycles BCR-ABL 0.931% IS). This subject was withdrawn from the study due to the progression of the disease. Another CP CML subject with a baseline mutation of M351T was enrolled in the study at 174 mg. The subject was required to be dose escalated to 204 mg at the start of cycle 7 to achieve adequate disease response. The subject completed cycle 22 and tolerated the higher dose well, maintaining a complete hematological response and stable disease.

One subject entered the study at a dose of 66 mg with a baseline mutation of F317L along with additional chromosomal anomalies (ACA). The subject responded at 66 mg with CCyR in cycle 7 and MMR in cycle 4 respectively. This subject developed SAE of back pain in C6D15 and was dose reduced to 48 mg. This subject maintained a disease response at the lower dose for about 2.5 years prior to discontinuation of the study drug due to disease progression.

### Dose-Escalation Up to 204 mg:

A 42-year-old, Asian male with AP CML entered the study at a 48 mg dose of the compound of Formula I. At study entry, the subject was refractory to Imatinib and had entered the study in minimal cytogenetic response and no molecular response. The subjects underwent dose-escalation sequentially from 66 mg to 204 mg for improving the responses. At ˜24 months, the subject attained MCyR at a daily dose of 174 mg with an improvement of molecular response. At ˜27 months daily dose of the compound of Formula I was further increased to 204 mg to optimize the cytogenetic and molecular response. Increase in the dose to 204 mg, resulted in decrease in BCR-ABL1, improving molecular response. Dose-escalations were tolerated well across all the doses. No serious adverse events were reported.

A Caucasian male, with CP CML entered the study at a 48 mg daily dose of compound of Formula I. At study entry, the subject was intolerant/refractory to 3 prior BCR-ABL1 TKIs including Ponatinib and entered the study with no cytogenetic response. At 48 mg, the subject developed a major cytogenetic response and achieved a major molecular response. This response was lost after 18 months of treatment, at which point daily dose of compound of Formula I was escalated to 174 mg. At dose-escalation to 174 mg, the subject re-attained major molecular and cytogenetic responses and maintained the deepened responses.

A 67-year-old, Asian female with CP CML entered the study at a daily dose of 174 mg of the compound of Formula I. At study entry, the subject was intolerant/refractory to 6 prior BCR-ABL1 TKIs and entered the study with minimal cytogenetic response. The daily dose escalated to 204 mg. At 204 mg, the subject demonstrated improvement in cytogenetic response to minor cytogenetic response, in a setting wherein no cytogenetic responses were reported on any of the 6 prior TKIs subject had received. Subject tolerated the dose-escalation well.

A 37-year-old Asian female with CP CML entered the study at a daily dose of 174 mg of the compound of Formula I. At study entry, the subject was intolerant/refractory to 2 prior BCR-ABL1 TKIs and entered the study with minimal cytogenetic response and no molecular response. The subject was dose-escalated to 204 mg. At 204 mg, the subject demonstrated improvement in cytogenetic response to partial cytogenetic response and improved molecular response. Subject tolerated the dose-escalation well.

A 48-year-old Asian female with CP CML entered the study at a 174 mg dose of the compound of Formula I. At study entry, the subject was resistant/intolerant to 2 prior BCR-ABL1 TKIs and entered the study with no cytogenetic and molecular response. The daily dose in the subject was escalated from 174 mg to 204 mg to achieve disease response. The subject continues to be on treatment with the compound of Formula I maintaining a hematological response and the compound of Formula I is well tolerated.

A 60-year-old Asian male with CP CML entered the study at a 12 mg daily dose of the compound of Formula I. At study entry, the subject was resistant to one prior BCR-ABL1 TKIs and no cytogenetic and molecular response. The subject underwent dose escalation from 12 mg to 174 mg to develop and further deepen the cytogenetic and molecular responses. The subject achieved and maintained CCyR at doses ≥48 mg. Further deepening of molecular response was demonstrated at 174 mg. The subject tolerated the higher doses and dose escalations well and continues to be on treatment with the compound of Formula I at 174 mg while maintaining CCyR.

A 38-year-old Asian male in CP CML entered the study at a 24 mg dose of the compound of Formula I. At study entry, the subject was resistant to one prior BCR-ABL1 TKIs and had achieved complete cytogenetic response (CCyR) but no molecular response in previous treatment. The subject underwent daily dose escalation to 126 mg and 174 mg to achieve and maintain molecular responses. The subject tolerated a higher dose of 174 mg of the compound of Formula I well and continues to maintain CCyR and molecular response between MMR and M2.

A 49-year-old male with CP CML entered the study at a 48 mg dose of the compound of Formula I. At study entry, the subject was resistant and/or intolerant to 4 prior TKIs and had achieved partial cytogenetic response (PCyR) but no molecular response. Until approximately 18 months, subject achieved and maintained CCyR and MMR with the 48 mg daily dose of the compound of Formula I, thereafter due to an increase in BCR-ABL ABL percentage the daily dose was increased to 90 mg. After an initial decline in BCR-ABL ABL percentage noted at 90 mg, due to loss of CCyR & MMR the dose was further increased to 174 mg. The subjects continue to tolerate the higher dose of 174 mg of the compound of Formula I well and maintain complete cytogenetic and major molecular responses.

A 56-year-old black or African-American male with CP CML entered the study at a 66 mg daily dose of the compound of Formula I. At study entry, the subject was resistant and/or intolerant to 4 prior TKIs and had no cytogenetic and molecular responses. The subject underwent dose escalation to 90 mg during which Ph+ cells decreased to minimal cytogenetic response. Thereafter again due to rising Ph+ cells, the dose was further increased to 126 mg and 174 mg. The subject tolerated the higher doses well and at ˜17 months the subject discontinued from treatment with the compound of Formula I due to disease progression.

### Dose De-Escalation:

A 76-year-old female with CP CML with F317L mutation entered the study at a 66 mg daily dose of the compound of Formula I. At study entry, the subject was intolerant/refractory to 4 prior BCR-ABL1 TKIs and entered the study with minimal cytogenetic response and no molecular response. At the 66 mg dose, the subject achieved CCyR and MMR. Later during treatment, the daily dose was reduced to 48 mg as the subject developed back pain of grade 2. The subject tolerated the reduced dose well and maintained CCyR and MMR at 48 mg dose for ˜2.5 years.

### Dose Reduction and Re-Escalation:

A 68-year-old, white female with CP CML entered the study at a 126 mg daily dose of the compound of Formula I. At study entry, the subject was intolerant/refractory to 4 prior BCR-ABL1 TKIs and entered the study with no cytogenetic and molecular response. The subject developed thrombocytopenia during cycle 2 of treatment due to which the treatment was withheld for ˜9 weeks. Upon restart of treatment using the compound of Formula I at a lower dose of 90 mg, there was a recurrence of thrombocytopenia and the treatment was put on hold again for 5 weeks, and restarted at a lower dose of 66 mg. The subject tolerated the lower dose of 66 mg and upon AE stabilization the drug was re-escalated to 126 mg and 174 mg in a stepwise manner to achieve cytogenetic and molecular responses. The subject tolerated the re-escalated higher dose well. The subject was discontinued at 174 mg at cycle due to disease progression.

### Dose Reduction:

A 60-year-old, white male with CP CML entered the study at a daily dose of 174 mg of the compound of Formula I. At study entry, the subject was intolerant/refractory to 3 prior BCR-ABL1 TKIs and entered the study with major cytogenetic response and no molecular response. Due to the development of grade 3/4 deranged pancreatic enzymes in cycle 2 the daily dose was reduced to 126 mg. The subject tolerated the lower dose of 126 mg well and is in complete cytogenetic response and major molecular response.

A 74-year-old male with CP CML entered the study at a 204 mg dose of the compound of Formula I. At study entry, the subject was intolerant/refractory to 4 prior BCR-ABL1 TKIs and entered the study with no cytogenetic and molecular response. Due to severe thrombocytopenia observed in cycle 2, the treatment was put on hold for one week and restarted at a lower dose of 174 mg. The subject continues to tolerate the compound of Formula I well at the lower dose of 174 mg and is shown partial cytogenetic response and improved molecular response.

An 82 years old, white female with CP CML entered the study at 204 mg dose of the compound of Formula I. At study entry, the subject was intolerant/refractory to 3 prior BCR-ABL1 TKIs and entered the study with major cytogenetic response and no molecular response. Due to the worsening of memory loss in cycle 13, the subject drug dose was reduced to 174 mg. The subject continues to tolerate the lower dose of 174 mg well and maintains a complete cytogenetic response and major molecular response.

The patient population studied in the Phase 1 clinical trial for the compound of Formula 1 is reflective of heavily pre-treated CML with 100% refractory/intolerant to at least 1 TKI, 81% patients receiving at least 2 TKIs, 64% receiving at least 3 TKIs and 43% patients receiving chemotherapy for their disease.

The patient population studied in clinical trial for the compound of Formula I is reflective of patient population with exhausted existing therapeutic options, with 36% of patients who had received at least three tyrosine kinase inhibitors prior one of which includes ponatinib. In the Phase 1 clinical study with the compound of Formula I, out of 53 enrolled subjects, 22 subjects had intolerance or resistance to ponatinib; whereas in Phase 2 clinical study with Formula I, out of 17 enrolled subjects, 16 subjects had failed ponatinib.

Based upon the above examples, it was found that although a higher dose of 204 mg is therapeutically efficacious in patients with prior TKI exposure, mutations and ACAs, there are lower dose levels that are therapeutically efficacious and well tolerated and which may be required in cases of poor drug tolerability. In the case of the compound of Formula I, the highest efficacious dose was observed to be 204 mg, and the lowest dose that was observed to be therapeutically efficacious was 48 mg. Doses below 48 mg were associated with limited therapeutic efficacy.

Overall results based on the Above 2 studies:

### Pharmacokinetics:

Upon oral administration to CML patients, the compound of Formula I was quickly absorbed with a median Tₘₐₓ of 1.5 to 2.5 hours in the majority of subjects across doses. Over all the inter-subject variability was higher. Based on trough concentrations, the steady state appeared to have reached by day 3 in majority of the subjects. With once-daily dosing, there was no low or minimal accumulation of the compound of Formula I at a steady state. In general, mean exposures (AUC) have increased with dose with sub-proportional increase or plateauing of exposures at doses above 174 mg. The PK parameters by dose cohort for cycle 2 are provided in Table 23 below:

TABLE 23
PK parameters by dose cohort for cycle 2

| Cohort | Cmin | | Cmax | | AUC (0-24) | |
|---|---|---|---|---|---|---|
| 12 | 61.8 | SD | 685 | SD | 3332 | SD |
| 24 | 114 | — | 589 | — | 5066 | — |
| 48 | 261 | 441 | 1087 | 545 | 13709 | 15217 |
| 66 | 102.7 | 180.7 | 830 | 450 | 7782 | 5827 |
| 90 | 363 | 434 | 2856 | 1566 | 22796 | 2782 |
| 126 | 831 | 1276 | 4103 | 3289 | 50311 | 55659 |
| 174 | 254 | 240 | 4212 | 3051 | 35748 | 33827 |
| 204 | 427 | 646 | 3311 | 1875 | 27532 | 14826 |

### Efficacy:

The MTD of the compound of Formula I was 204 mg while RP2D was 174 mg QD. Anti-leukemic activity in the form of hematological, cytogenetic and molecular responses was reported across Parts B and C (n=59). Major cytogenetic responses were reported in 61% of subjects while major molecular responses were reported in 33.9% of subjects across doses. Of note, responses including major cytogenetic (n=15 of 29, 51.7%) and molecular (n=11 of 29, 37.93%) responses were observed in subjects who failed/were intolerant to all available TKI therapies including ponatinib. At the RP2D of 174 mg, major cytogenetic response was observed in 50% of subjects independent of prior ponatinib exposure. The responses were noted in all phases of CML (i.e.: CP CML, AP CML, and BP CML). There is limited evidence of activity in the CML literature in such a heavily pre-treated population.

### Safety:

The compound of Formula I was well tolerated across doses. Fifty-five of the 59 subjects (93.2%) experienced an AE during the study, of which 62.7% of AEs (n=37) were suspected to be related to the compound of Formula I treatment. Most frequently reported AEs related to the compound of Formula I included thrombocytopenia, neutropenia, nausea, diarrhea, pruritus, fatigue and myalgia. The compound of Formula I-related AEs was generally low grade and resolved with drug hold or dose reduction. All cardiac AEs reported were considered unrelated to the compound of Formula I treatment. The AEs were distributed across all the dosing cohorts without any safety trends or association with dose(s). The DLTs (except for 1 event of fatal intracranial hemorrhage) were non-serious AEs and managed well by dose delays and/or reductions. Approximately 64.4% (n=38) of subjects experienced grade 3 or higher-grade AEs and 34% of subjects experienced an SAE (n=20). A total of 5 subjects died during the study or within 30 days after the last dose of the study drug, which is not unexpected given that all patients enrolled in the study had advanced and heavily pretreated CML. The AEs related to the compound of Formula I treatment were consistent with the advanced CML population with multiple comorbidities and concomitant medications during the study.

The compound of Formula I demonstrated clinically important anti-leukemic activity in heavily pretreated CML and was well tolerated up to MTD of 204 mg in this advanced disease setting.

1. A method for the treatment of a human patient with chronic myeloid leukemia (CML) comprising administering to the patient a therapeutically effective amount of compound of Formula I: 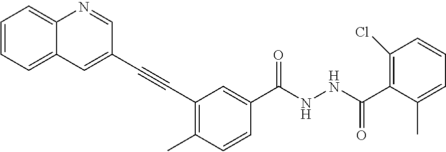
or a pharmaceutically acceptable salt thereof,
wherein the chronic myeloid leukemia (CML) is chronic, accelerated, or blast phase Philadelphia Chromosome Positive Chronic Myeloid Leukemia (Ph+ CML).

2. The method as claimed in claim 1, wherein the therapeutically effective amount of the compound of Formula I or its pharmaceutically salt is sufficient to achieve a mean AUC₀₋₂₄ ranging from 6226±5827 ng*h/mL to 60373±55659 ng*h/mL and/or a mean Cₘₐₓ ranging from 664±450 ng/ml to 5054±3051 ng/mL.

3. The method as claimed in claim 1, wherein the compound of Formula I or its pharmaceutically salt is administered at an initial daily dose of 10 mg to 204 mg.

4. The method as claimed in claim 1, wherein the compound of Formula I or its pharmaceutically salt is administered at an initial daily dose selected from 66 mg, 87 mg, 90 mg, 126 mg, 130.5 mg, 135 mg, 174 mg and 180 mg.

5. The method as claimed in claim 1, wherein the initial daily dose is escalated or de-escalated to achieve or maintain at least one of (a) complete hematological response, (b) complete hematological response and partial cytogenetic response, (c) complete hematological response and complete cytogenetic response, or (d) complete hematological response, complete cytogenetic response, and major molecular response.

6. The method as claimed in claim 1, wherein the initial daily dose is escalated or de-escalated with no severe adverse reaction.

7. The method as claimed in claim 1, wherein the patient is resistant or intolerant to at least one tyrosine kinase inhibitor.

8. (canceled)

9. (canceled)

10. (canceled)

11. (canceled)

12. The method as claimed in claim 1, wherein the patient has one or more of the following mutations in the BCR-ABL1 kinase domain: M244V, L248V, E499E, Q252H, P223S, A337T, F359C, E255V, Y253F, F317L, Y253H, V299L, E255V, F317L, E255V, F359V, G250E, E255K, F359V, M351T, G250E, H369R, H396R, and V359I.

13. The method as claimed in claim 1, wherein the patient has one or more of the following characteristics:
a) 15% blasts in peripheral blood and bone marrow;
b) <30% blasts plus promyelocytes in peripheral blood and bone marrow;
c) <20% basophils in the peripheral blood;
d) ≥50×10⁹/L (≥50,000/mm³) platelets;
e) Transient prior therapy related thrombocytopenia (<50,000/mm³ for ≤30 days prior to screening);
f) No evidence of extramedullary leukemic involvement, with the exception of hepatosplenomegaly;
g) ≥15 to <30% blasts in peripheral blood or bone marrow;
h) ≥20% basophils in peripheral blood or bone marrow;
i) ≥30% (blasts+promyelocytes) in peripheral blood or bone marrow (but <30% blasts);
j) ≤100×109 platelets/L in peripheral blood unrelated to therapy;
k) Additional clonal cytogenetic abnormalities in Ph+ cells;
l) ≥30% blasts in peripheral blood, bone marrow or both; and
m) extra-medullary disease.

14. The method as claimed in claim 1, wherein the patient does not have T315I-positive CML.

15. The method as claimed in claim 1, wherein
(i) the treatment is withheld for a period of at least 7 days, when the patient exhibits a grade 1 or grade 2 non-hematological adverse event, and then reinitiated;
(ii) the treatment is withheld for a period of up to 56 days, when the patient exhibits a grade 3 hematological or non-hematological adverse event or grade 4 asymptomatic hematological adverse event, and then reinitiated; or
(iii) the treatment is discontinued when the patient does not recover to a grade 1 adverse event or less after a withholding period of 56 days.

16. (canceled)

17. (canceled)

18. A method of treating a treatment-resistant human patient having chronic myeloid leukemia (CML) comprising administering a therapeutically effective amount of a compound of Formula I: 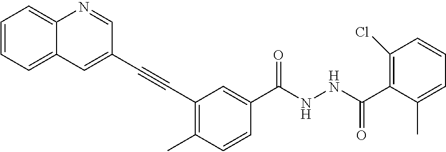
or a pharmaceutically acceptable salt thereof to the patient, wherein
i. the method comprises orally administering 174 to 200 mg of the compound of Formula I daily, and
ii. the patient is resistant or intolerant to at least one tyrosine kinase inhibitor prior to the administration of the compound of Formula I.

19. The method as claimed in claim 18, wherein the patient has Ph+ CML.

20. The method as claimed in claim 18, wherein the patient is in the chronic phase of CML.

21. (canceled)

22. (canceled)

23. (canceled)

24. (canceled)

25. The method as claimed in claim 18, wherein the patient has one or more of the following mutations in the BCR-ABL1 kinase domain: M244V, L248V, E499E, Q252H, P223S, A337T, F359C, E255V, Y253F, F317L, Y253H, V299L, E255V, F317L, E255V, F359V, G250E, E255K, F359V, M351T, G250E, H369R, H396R, and V359I.

26. (canceled)

27. (canceled)

28. (canceled)

29. (canceled)

30. The method as claimed in claim 18, wherein
(i) upon the patient exhibiting a grade 1 or grade 2 non-hematological adverse event which was intolerable due to clinical symptoms or interference with daily activities, the treatment is withheld for a period of time and then reinitiated; or
(ii) upon the patient exhibiting a grade 3 hematological or non-hematological adverse event or grade 4 asymptomatic hematological adverse event, the treatment is withheld for a period of time and then reinitiated.

31. (canceled)

32. (canceled)

33. (canceled)

34. (canceled)

35. (canceled)

36. (canceled)

37. A method for the treatment of a human patient with chronic myeloid leukemia (CML) comprising administering to the patient a therapeutically effective amount of compound of Formula I: 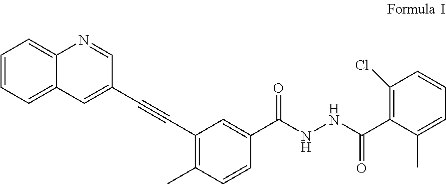
or a pharmaceutically acceptable salt thereof at an initial daily dose of 50 mg to 200 mg, wherein when the patient develops hematologic and/or non-hematologic toxicity:
a) the initial daily dose is withheld for at least 7 days and resumed at the initial daily dose of 50 mg to 200 mg;
b) the initial daily dose is withheld for less than 7 days and if the toxicity is resolved, then the treatment is resumed at the initial daily dose of 50 mg to 200 mg;
c) the initial daily dose is withheld for at least 7 days and resumed at a reduced daily dose of 43.5 mg to 192 mg and the reduced daily dose is optionally re-escalated to a re-escalated daily dose of 50 mg to 200 mg; or
d) the initial daily dose is withheld for at least 7 days and resumed at a reduced daily dose of 43.5 mg to 192 mg, the reduced daily dose is further reduced to a subsequent reduced daily dose of 43.5 mg to 180 mg on subsequent instances of recurrences of the toxicity, and the reduced daily dose or the subsequent reduced daily dose is optionally re-escalated to a re-escalated daily dose of 50 mg to 200 mg.

38. A method for the treatment of a human patient with chronic myeloid leukemia (CML) comprising administering to the patient a therapeutically effective amount of compound of Formula I: 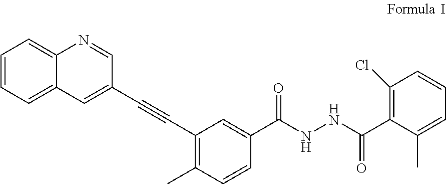
or a pharmaceutically acceptable salt thereof:
(I) at an initial daily dose of 174 mg wherein when the patient develops hematologic and/or non-hematologic toxicity:
a) the initial daily dose is withheld for at least 7 days and resumed at the initial daily dose of 174 mg;
b) the initial daily dose is withheld for less than 7 days and if the toxicity is resolved, then the treatment is resumed at the initial daily dose of 174 mg;
c) the initial daily dose is withheld for at least 7 days and resumed at a reduced daily dose of 43.5 mg to 135 mg and the reduced daily dose is optionally re-escalated to a re-escalated daily dose up to 200 mg; or
d) the initial daily dose is withheld for at least 7 days and resumed at a reduced daily dose of 43.5 mg to 135 mg, the reduced daily dose is optionally further reduced to a subsequent reduced daily dose of 43.5 mg to 130.5 mg on subsequent instances of recurrences of the toxicity, and the reduced daily dose or the subsequent reduced daily dose is optionally re-escalated to a re-escalated daily dose up to 200 mg; or
(II) at an initial daily dose of 87 mg such that when the patient develops hematologic and/or non-hematologic toxicity:
a) the initial daily dose is withheld for at least 7 days and resumed at the initial daily dose of 87 mg; or
b) the initial daily dose is withheld for less than 7 days and if the toxicity is resolved, then the treatment is resumed at the initial daily dose of 87 mg; or
c) the initial daily dose is withheld for at least 7 days and resumed at a reduced daily dose of 43.5 mg or 50 mg, and the reduced daily dose is optionally re-escalated to a re-escalated daily dose up to 200 mg.

39. (canceled)

40. (canceled)

41. The method as claimed in claim 37, wherein the patient is resistant or intolerant to at least one tyrosine kinase inhibitor.

42. (canceled)

43. (canceled)

44. (canceled)

45. (canceled)

46. The method as claimed in claim 37, wherein the patient has one or more of the following mutations in the BCR-ABL1 kinase domain: M244V, L248V, E499E, Q252H, P223S, A337T, F359C, E255V, Y253F, F317L, Y253H, V299L, E255V, F317L, E255V, F359V, G250E, E255K, F359V, M351T, G250E, H369R, H396R, and V359I.

47. The method as claimed in claim 37, wherein the chronic myeloid leukemia (CML) is chronic, accelerated, or blast phase Philadelphia Chromosome Positive Chronic Myeloid Leukemia (Ph+ CML).
